(19) United States
(12) Patent Application Publication
(10) Pub. No.: US 2006/0199204 A1
(43) Pub. Date: Sep. 7, 2006

(54) Genetic testing for male factor infertility

(75) Inventors: David Jacob Dix, Raleigh, NC (US); Stephen A. Krawetz, Detroit, MI (US); David Miller, Belmont Grove (GB)

Correspondence Address: BROWDY AND NEIMARK, P.L.L.C.;624 NINTH STREET, NW, SUITE 300, WASHINGTON, DC, 20001-5303, US

(73) Assignees: U.S. EPA, Washington, DC (US); Wayne State University, Detroit, MI (US); University of Leeds, Belmont Grove (GB)

(21) Appl. No.: 11/357,423

(22) Filed: Feb. 21, 2006

Related U.S. Application Data
(63) Continuation-in-part of application No. 10/264,321, filed on Oct. 4, 2002, now abandoned.
(60) Provisional application No. 60/327,525, filed on Oct. 5, 2001.

(51) Int. Cl. C12Q 1/68

(52) U.S. Cl. 435/6

(57) ABSTRACT

RNA in sperm can be used as a diagnostic to distinguish between normal and affected individuals. A list of specific diagnostic transcripts is provided which is compared with transcripts obtained from the sperm of a subject. Correlation between the two transcripts is used to identify normal sperm or affected sperm. Addition, genetic testing for male infertility or damage to spermatozoa is accomplished by providing a microarray of DNA probes with a sample of spermatozoa to determine the mRNA fingerprints of the sample, and comparing the mRNA fingerprints of the sample with the mRNA fingerprints of normal fertile male spermatozoa.

### CROSS-REFERENCE TO RELATED APPLICATIONS

The present invention is a continuation-in-part of application Ser. No. 10/264,321, filed Oct. 5, 2002, which application claims priority from provisional application Ser. No. 60/327,525, filed Oct. 5, 2001, the entire contents of both of which are hereby incorporated by reference.

### FIELD OF THE INVENTION

The present invention relates to methods, kits, and tools for distinguishing between sperm of normal and affected individuals.

### BACKGROUND OF THE INVENTION

Predicting the fertility of a male is very useful in a variety of contexts. For example, the artificial insemination industry is interested in knowing the likelihood that fertilization will occur if a female is artificially inseminated with a particular male's semen. Alternatively, human fertility clinics are concerned with achieving impregnation, and evaluating the sperm count of a male is one step in this procedure. Thus, whether in the context of animal breeding, the artificial insemination industry, or human fertility clinics, determination of the fertility of the male is very important.

Ten percent of the male population have abnormally low sperm counts, and approximately one in six couples experiences difficulty in conceiving a child. Male factor infertility accounts for 40-50% of the cases in which assisted reproductive techniques are recommended. The great majority (>98%) of infertile men actually produce sperm, but, for some reason, those sperm are often unable to fertilize an egg. Chromosomal anomalies are associated with approximately one third of non-obstructive male factor infertility affecting some 2% of the infertile male population. Half of these men (15%) present with abnormal karyotypes, which the other half (12-15%) present with microdeletions in the Azoospermic Factor (AZF) region of Yq (i.e., DAZY/RBM).

Couples having difficulty starting a family must undergo an extensive battery of tests, including a testicular biopsy. However, it has not yet been possible to identify which couples will never conceive, so that these couples can forgo the lengthy, expensive, and ultimately futile infertility therapy and begin considering other options, such as sperm donors.

Testes-specific defects have only been demonstrated in men with sub-microscopic microdeletions of the Y chromosome encompassing one or more genes. It is reasonable to expect that as new testes-specific genes are discovered, more testes-restricted abnormalities will be revealed. Lesions affecting the X and Y chromosome, as well as autosomal recessive and imprinted genes, have been associated with oligozoospermia. These types of abnormalities, however, are rarely observed in clinics. The underlying causes of infertility in the remaining 98% of men with non-obstructive defects in spermatogenesis remain unknown. Accordingly, the majority of male factor infertilities are classified as idiopathic, indicating that other genetic factors should be considered.

With the exception of obvious defects such as azoospermia, globozoospermia, and immotile ciliary syndrome, the extreme heterogeneity of normal fertile human semen suggests that most idiopathic male factor infertility is not a result of monogenic disorders. Moreover, all known monogenic disorders that affect the testes affect other tissues to an equal or greater extent. Accordingly, it is reasonable to assume that the majority of idiopathic male factor infertility that has testes-restricted phenotypes is not monogenic, but oligo- or poly-genic in origin.

Two recent developments offer considerable promise towards identifying oligo- and/or poly-genic factors that influence male fertility. First, the discovery of mRNAs in ejaculate spermatozoa makes it possible to obtain transcriptional information from male germ cells using non-invasive procedures. It is expected that these mRNAs provide a window to past events of spermatogenesis, echoing tests for gene expression. Interestingly, data mining and functional assessment suggests that in addition to delivering the haploid male genome, spermatozoa also deliver a critical complement of mRNAs to the oocyte. Secondly, microassays make it possible to construct detailed gene expression profiles.

Most laboratory investigations of semen quality are relatively poor indicators of fertility because they are subjective and predominantly rely on physiological and morphological criteria. This is exemplified in the recent study that compared the predictive results from over 250 independent studies (Gynecol Obstet Invest. 2005:59(2) 86-91). Only 4 could be could be directly compared as the variation among the others was too great. The consequences of using immature spermatids or other compromised germ cells in intra-cytoplasmic spermatozoa induction (CISI) procedures need careful reappraisal, considering that spermatozoal mRNA is required for the production of normal offspring.

Thus, there is a need for an effective, efficient and accurate method and/or device for determining male fertility. More specifically, there is a need for a method for determining if a male is fertile using microarrays in analyzing mRNAs of spermatozoa.

### SUMMARY OF THE INVENTION

It is an object of the present invention to overcome the aforementioned difficulties of the prior art.

It is another object of the present invention to provide a method for detecting if a male is a normal fertile male using microarrays in analyzing the mRNA of spermatozoa.

It is a further object of the present invention to provide a kit for detecting normal fertile males using microarrays in analyzing the mRNA of spermatozoa.

It is yet another object of the present invention to provide markers for performing tests for detecting normal fertile males using microarrays in analyzing the mRNA of spermatozoa.

It is another object of the present invention to provide a test for determining if exposure to a toxin has adversely affected sperm.

It is a further object of the present invention to distinguish between sperm of normal and affected individuals.

It is another object of the present invention to provide diagnostic transcripts for identifying and classifying sperm of normal and affected individuals.

Microarray based platforms are used to show clear differences between the populations of RNA transcripts present in the sperm of normal fertile individuals and affected individuals. The present invention includes a list of specific transcripts of diagnostic value. A differential heat map is used to identify differences between the transcripts present in normal and affected individuals. This provides a present/absent measure that can be used as a unique classifier.

A core set of transcripts that are consistently observed as present in normal fertile sperm has been validated across three microarray platforms. The relative levels of this stable core set of transcripts can distinguish normal fertile sperm from affected sperm. The subset of specific and robust transcripts provided herein will yield a differential heat map emphasizing the differences between normal and affected individuals.

The present invention also provides a microarray-based diagnostic to detect normalcy of human spermatozoa. This method provides an easy to interpret result of clinically diagnostic value from a diagnostic test that can be assayed in a routine clinical laboratory.

The present invention provides a test, kit, and method for determining male fertility. This invention provides a genome-wide analysis to define the spermatozoal RNA fingerprint of a normal fertile male. The sperm-microarray methods outlined herein provide mechanisms for identifying fertile and infertile males.

The present invention uses microarray technology to monitor, in a sample of spermatozoa, the presence of transcripts (messenger RNAs) from over 2700 genes that the inventors have determined to be critical to normal fertility. A microarray chip is created by depositing onto slides microscopic quantities of the genetic material from these genes, and then overlaying onto the slides the genetic material extracted from a sample ejaculate. If complementary genetic material is present in the sample, it will bind to the genetic sites on the chip and be detected through laser excitation of bound fluorescence probes.

In a similar manner, the invention can be used as a toxicological/epidemiological screen to determine the presence of permanent or temporary damage to the spermatozoa of males exposed to environmental toxicants, as well as the identity of paternally derived messenger RNAs that are critical to early human development.

A suite of microarrays containing 27,016 expressed sequence tags (ESTs) was interrogated using cDNAs from a pool of nineteen testes; cDNAs from a pool of nine individual ejaculate spermatozoal mRNAs and cDNAs constructed-from a single ejaculate's spermatozoal mRNAs.

The testes, pooled and single ejaculate DNAs hybridized to 7157, 3281, and 2784 ESTs, respectively. The testes population contained all of the ESTs identified by the cDNAs from the pooled and individual-ejaculate. The pooled ejaculate population contained all but 4 ESTs identified from the individual ejaculate.

Accordingly, profiling can be used to monitor past events, such as gene expression of spermatogenesis. Moreover, the data suggest that, in addition to delivering-the paternal genome, spermatozoa provide the zygote with a unique suite of paternal mRNAs. Ejaculate spermatozoa can now be used as a non-invasive proxy for testes infertility investigations.

Current research supports the diagnosis of idiopathic infertility via spermatozoal mRNA fingerprints, and suggests that spermatozoal transcripts complementing those of oocytes are important for embryo development. Male gametes deliver more to the oocyte than the haploid male genome, and possess a greater role in orchestrating normal embryo development than has heretofore been recognized.

Microarrays were developed containing tiny sites that trap specific mRNA. When sperm is added, color changes at each trap site indicate whether the sperm includes each bit of mRNA. Almost immediately, one can scan the sperm to tell which mRNA, and which associated genes, are present.

The present invention can be used in various settings, including, but not limited to, hospitals, fertility clinics, artificial insemination and animal breeding facilities, and any other similar settings that can use a test for determining fertility of a male. Although the present invention is illustrated in a human model, one skilled in the art can appreciate that the invention is also applicable to and useful for animals other than humans.

In one application of the present invention, fertile spermatozoa are determined by determining the presence of mRNA. Thus, if mRNA is not present, then the spermatozoa are deemed to be non-functional. Additionally, the assay of the present invention is useful in toxicological screening and risk assessment to determine if a male species has suffered permanent or temporary damage to spermatozoa populations.

Additionally, the assay of the present invention can be used to identify specific mRNAs that are paternally derived and are critical to early human development. These parentally derived mRNAs include, but are not limited to, the following human uni-Gene: Hs.27695; Hs.19500; Hs.8867; Hs.46925; Hs.2714; Hs.152213; Hs.18195; Hs.274402; Hs.250899; Hs.2128; Hs.75106; Hs.86368; Hs.97633; and any other similar mRNA sequences known to those of skill in the art. Since these paternally derived mRNAs are essential to development, they serve as excellent markers.

### BRIEF DESCRIPTION OF THE DRAWINGS

FIG. 1 is a fingerprint of human testes and sperm RNAs.

FIG. 2 shows a distribution of testes and spermatozoal RNAs.

FIG. 3 illustrates spermatozoal RNA ontogeny.

FIG. 4A-E show isolation of spermatozoal RNA.

FIG. 4F illustrates fidelity of spermatozoal RNA preparations.

FIG. 5 shows genetic profiling of ejaculate spermatozoa.

FIG. 6 is an example of chip-on-chip fold change between normal individuals

FIG. 7 is an example of chip-on-chip change between affected individuals.

FIG. 8 shows the partitioning of transcripts present in the normal individuals data set (left), both datasets (middle) and affected-individuals' data set (right) above-the threshold value.

FIG. 9 shows analysis of RNA transcripts present in sperm from thirteen normal and eight affected samples. The heat map shows that the levels of transcripts present in normal and affected individuals that can be distinguished. Six strikingly different regions from the analyses are shown that can be used to classify and diagnose individuals.

FIG. 10 shows clustering of sperm RNA samples based on Illumina Bead Chip array transcript data. Sperm transcripts from normal individuals are clearly distinguished from affected individuals.

FIG. 11 shows clustering of the 661 transcripts consistently detected across all three array platforms, using data from Affymetrix arrays. FIG. 11A is an example of cluster 3.0 ad TreeView consistently clustering normal individuals separate from affected individuals. FIG. 11B shows a segment of the corresponding 661 transcript heat map generated visually, demonstrating the diagnostic capacity of the arrays.

### DETAILED DESCRIPTION OF THE INVENTION

The present invention provides a window into the male reproductive system so that it is possible to monitor overall reproductive health with precision. The assays of the present invention can be used not only for predicting whether an individual is fertile or not, but also to obtain a detailed description of the gene-environment interaction for that individual. In the latter case, microarrays target genetic differences between the normal male model and men who have been exposed to suspected toxins. The microarray test provides a quick determination of whether a man's sperm had been adversely affected by a toxin. This approach takes into account not only how most people respond and the exposure limits that have been set on how most people seem to respond, but also how each individual responds. This knowledge is particularly beneficial to men who are at high risk of environmental toxin exposure through the workplace. In one example, a company could use the microarray test to monitor its male employees for overall exposure-induced changes in fertility. In another instance, an employee who was trying to start a family might request a test to ensure he was fertile. In the latter case, if a problem did arise, the employee could curtail his exposure and simply wait the average 60-90 days for his body to replace the old sperm with new, unexposed sperm.

In animal husbandry, particularly with beef production, cattle have a long gestation time. If one could ensure that every pregnancy outcome would be successful, beef production-could be increased, thus increasing profit margins.

The use of microarray technology allows for the study of complex interplay of genes and other genetic material simultaneously. As is known, the-pattern of genes expressed in a cell is characteristic of its state. Additionally, virtually all differences in cell states correlate with changes in mRNA levels of genes. Generally, microarray technology involves obtaining complementary genetic material to genetic material of interest and laying out the complementary genetic material in microscopic quantities on solid surfaces at defined positions. Genetic material from samples is then eluted over the surface, and complementary genetic material binds thereto. The presence of bound genetic material is detected by fluorescence following laser excitation. However, other detection means can also be used.

As known to those skilled in the art, spermatogenesis is a multifaceted developmental program beginning with mitotic divisions of diploid spermatogonia. These divisions give rise to spermatocytes, which undergo meiosis to produce haploid round spermatids. The final stage of spermatogenesis, termed spermiogenesis, is highlighted by the differentiation of round spermatides into spermatozoa. Once spermatogenesis is complete, spermatozoa are released from their chaperones, the Sertli cells, through a process known as spermiation. Throughout the elaborate process of spermatogenesis, many testes-specific mRNAs are synthesized and placed under stringent translational control to ensure appropriate temporal and spatial expression. The mRNAs observed in mature spermatozoa are remnants of untranslated stores that provide a historic record or fingerprint of spermatogenesis. Hence, the spermatazoal mRNA fingerprint representing the normal fertile male serves as a standard for identifying the causes of idiopathic infertility. Despite the obvious wealth of information contained within these repositories, the complexity and function of spermatozoal mRNAs have not been characterized. Once defined, however, this fingerprint provides information on the underlying causes of male factor infertility and the reasons mRNAs remain in mature spermatozoa, while rRNAs are most likely lost.

Several studies support the conclusion that spermatozoa contain a complex repertoire of mRNAs. Even though these mRNAs are thought to provide a window to past events of spermatogenesis, their complexity and function have yet to be established.

RNA in sperm can be used to clearly distinguish between normal and affected individuals. A database was constructed of sperm characteristics from semen analysis and donor lifestyle variables. Two sets of test samples characteristic of normal and affected individuals were selected and compared using the Affymetrix Human U133 plus 2 and Illumina Centrix Human Expression BeadChip platforms. The results obtained for both platforms for the normal fertile individuals were then compared to the results obtained in Ser. No. 10.264,231 based upon the Research Genetics spotted cDNA microarray platform, published in Ostermeier et al., Spermatozoal Lancet 360(8335): 772-7, 2002. Sperm from reproductively normal individuals were analyzed on 16 Affymetrix Human U133 plus 2 chips and two Illumina Centrix Human-6 Expression BeadChip arrays. A total of 16 normal samples were analyzed using the Affymetrix platform, of which 13 were of sufficient quality to permit analysis. An additional two normal samples were then analyzed using the Illumina Centrix Human-6 Expression BeadChip platform. Both were of sufficient quality to permit analysis.

Sperm samples from nine tetatozoospermia affected individuals were analyzed using both the Affymetrix Human U133 plus 2 chips and the Illumina Centrix Human-6 Expression BeadChips. Of the nine samples, eight that were analyzed using the Affymetrix Human U133 plus 2 platform were of sufficient quality to permit analysis. All nine analyzed using the Illumina Centrix Human-6 expression BeadChip platform were of sufficient quality to permit analysis.

Data Obtained are Shown in Table 1

TABLE 1
Gene Title and Probe Set ID

| | Gene | | |
|---|---|---|---|
| Gene Title | Symbol | UniGene ID | Probe Set ID |
| 1-acylglycerol-3-phosphate O-acyltransferase 5 | AGPAT5 | Hs.490899 | 218096_at |
| (lysophosphatidic acid acyltransferase, epsilon) | | | |
| 24-dehydrocholesterol reductase | DHCR24 | Hs.498727 | 200862_at |
| 2-aminoadipic 6-semialdehyde dehydrogenase | AASDH | Hs.104347 | 235435_at |
| 3-hydroxyisobutyryl-Coenzyme A hydrolase | HIBCH | Hs.236642 | 213374_x_at |
| 3-oxoacid CoA transferase 2 | OXCT2 | Hs.472491 | 233301_at |
| 3-phosphoinositide dependent protein kinase-1 | PDPK1 | Hs.459691 | 32029_at |
| 4-aminobutyrate aminotransferase | ABAT | Hs.336768 | 209459_s_at |
| 5,10-methenyltetrahydrofolate synthetase (5- | MTHFS | Hs.459049 | 210242_x_at |
| formyltetrahydrofolate cyclo-ligase) | | | |
| 5,10-methylenetetrahydrofolate reductase | MTHFR | Hs.214142 | 239035_at |
| (NADPH) | | | |
| 5-azacytidine induced 2 | AZI2 | Hs.444724 | 218043_s_at |
| 5-azacytidine induced 2 | AZI2 | Hs.444724 | 223846_at |
| 5-hydroxytryptamine (serotonin) receptor 2A | HTR2A | Hs.424980 | 207135_at |
| 5′-nucleotidase, cytosolic IB | NT5C1B | Hs.120319 | 1554368_at |
| 5′-nucleotidase, cytosolic IB | NT5C1B | Hs.120319 | 243100_at |
| 5′-nucleotidase, cytosolic II | NT5C2 | Hs.158209 | 243158_at |
| 5′-nucleotidase, cytosolic III | NT5C3 | Hs.487933 | 223298_s_at |
| 5′-nucleotidase, cytosolic II-like 1 | NT5C2L1 | Hs.520341 | 223177_at |
| 5′-nucleotidase, cytosolic II-like 1 | NT5C2L1 | Hs.520341 | 223178_s_at |
| 6-pyruvoyl-tetrahydropterin synthase/dimerization | DCOHM | Hs.527213 | 224372_at |
| cofactor of hepatocyte nuclear factor 1 alpha | | | |
| (TCF1) 2 /// 6-pyruvoyl-tetrahydropterin | | | |
| synthase/dimerization cofactor of hepatocyte | | | |
| nuclear factor 1 alpha (TCF1) 2 | | | |
| 6-pyruvoyl-tetrahydropterin synthase/dimerization | DCOHM | Hs.527213 | 224373_s_at |
| cofactor of hepatocyte nuclear factor 1 alpha | | | |
| (TCF1) 2 /// 6-pyruvoyl-tetrahydropterin | | | |
| synthase/dimerization cofactor of hepatocyte | | | |
| nuclear factor 1 alpha (TCF1) 2 | | | |
| 82-kD FMRP Interacting Protein | 182-FIP | Hs.462598 | 224938_at |
| 82-kD FMRP Interacting Protein | 182-FIP | Hs.462598 | 224939_at |
| a disintegrin and metalloproteinase domain 17 | ADAM17 | Hs.404914 | 205745_x_at |
| (tumor necrosis factor, alpha, converting enzyme) | | | |
| a disintegrin and metalloproteinase domain 18 | ADAM18 | Hs.127930 | 207597_at |
| a disintegrin and metalloproteinase domain 20 | ADAM20 | Hs.177984 | 207423_s_at |
| a disintegrin and metalloproteinase domain 21 | ADAM21 | Hs.178748 | 1552579_a_at |
| a disintegrin and metalloproteinase domain 21 | ADAM21 | Hs.178748 | 207665_at |
| a disintegrin and metalloproteinase domain 28 | ADAM28 | Hs.528304 | 208268_at |
| a disintegrin and metalloproteinase domain 29 | ADAM29 | Hs.126838 | 221337_s_at |
| a disintegrin and metalloproteinase domain 3a | ADAM3A | Hs.98848 | 216276_s_at |
| (cyritestin 1) | | | |
| a disintegrin and metalloproteinase domain 6 | ADAM6 | Hs.97508 | 237909_at |
| A disintegrin-like and metalloprotease (reprolysin | ADAMTS5 | Hs.58324 | 235368_at |
| type) with thrombospondin type 1 motif, 5 | | | |
| (aggrecanase-2) | | | |
| a disintegrin-like and metalloprotease (reprolysin | ADAMTS9 | Hs.549184 | 220287_at |
| type) with thrombospondin type 1 motif, 9 | | | |
| A kinase (PRKA) anchor protein 1 | AKAP1 | Hs.463506 | 201674_s_at |
| A kinase (PRKA) anchor protein 1 | AKAP1 | Hs.463506 | 201675_at |
| A kinase (PRKA) anchor protein 1 | AKAP1 | Hs.463506 | 240495_at |
| A kinase (PRKA) anchor protein 10 | AKAP10 | Hs.462457 | 236007_at |
| A kinase (PRKA) anchor protein 11 | AKAP11 | Hs.105105 | 203156_at |
| A kinase (PRKA) anchor protein 11 | AKAP11 | Hs.105105 | 215336_at |
| A kinase (PRKA) anchor protein 13 | AKAP13 | Hs.459211 | 209534_x_at |
| A kinase (PRKA) anchor protein 13 | AKAP13 | Hs.459211 | 222024_s_at |
| A kinase (PRKA) anchor protein 13 | AKAP13 | Hs.459211 | 243450_at |
| A kinase (PRKA) anchor protein 14 | AKAP14 | Hs.184993 | 237281_at |
| A kinase (PRKA) anchor protein 2 /// PALM2- | AKAP2 | Hs.259461 | 202760_s_at |
| AKAP2 protein | /// | | |
| | PALM2- | | |
| | AKAP2 | | |
| A kinase (PRKA) anchor protein 4 | AKAP4 | Hs.97633 | 207019_s_at |
| A kinase (PRKA) anchor protein 6 | AKAP6 | Hs.509083 | 205359_at |
| Abelson helper integration site | AHI1 | Hs.386684 | 221569_at |
| abhydrolase domain containing 1 | ABHD1 | Hs.375791 | 236461_at |
| abhydrolase domain containing 2 | ABHD2 | Hs.122337 | 205566_at |
| abl-interactor 1 | ABI1 | Hs.508148 | 209027_s_at |
| Abl-interactor 1 | ABI1 | Hs.508148 | 238563_at |
| absent in melanoma 1 | AIM1 | Hs.486074 | 212543_at |
| absent in melanoma 2 | AIM2 | Hs.281898 | 206513_at |
| acetyl-Coenzyme A acetyltransferase 1 | ACAT1 | Hs.232375 | 205412_at |
| (acetoacetyl Coenzyme A thiolase) | | | |
| acetyl-Coenzyme A acetyltransferase 2 | ACAT2 | Hs.363137 | 209608_s_at |
| (acetoacetyl Coenzyme A thiolase) | | | |
| acheron | FLJ11196 | Hs.416755 | 236565_s_at |
| acid phosphatase 1, soluble | ACP1 | Hs.130873 | 215227_x_at |
| Acidic (leucine-rich) nuclear phosphoprotein 32 | ANP32A | Hs.458747 | 201051_at |
| family, member A | | | |
| acidic (leucine-rich) nuclear phosphoprotein 32 | ANP32B | Hs.494604 | 201305_x_at |
| family, member B | | | |
| Acidic (leucine-rich) nuclear phosphoprotein 32 | ANP32E | Hs.385913 | 229128_s_at |
| family, member E | | | |
| acrosin binding protein | ACRBP | Hs.123239 | 223717_s_at |
| acrosomal vesicle protein 1 | ACRV1 | Hs.169222 | 207969_x_at |
| acrosomal vesicle protein 1 | ACRV1 | Hs.169222 | 207973_x_at |
| acrosomal vesicle protein 1 | ACRV1 | Hs.169222 | 208013_s_at |
| actin related protein 2/3 complex, subunit 2, 34 kDa | ARPC2 | Hs.529303 | 207988_s_at |
| actin related protein 2/3 complex, subunit 2, 34 kDa | ARPC2 | Hs.529303 | 208679_s_at |
| actin related protein 2/3 complex, subunit 2, 34 kDa | ARPC2 | Hs.529303 | 213513_x_at |
| actin related protein 2/3 complex, subunit 5-like | ARPC5L | Hs.132499 | 223101_s_at |
| Actin related protein 2/3 complex, subunit 5-like | ARPC5L | Hs.132499 | 236780_at |
| actin, beta | ACTB | Hs.520640 | 200801_x_at |
| actin, beta | ACTB | Hs.520640 | 213867_x_at |
| actin, beta | ACTB | Hs.520640 | 224594_x_at |
| actin, gamma 1 | ACTG1 | Hs.514581 | 201550_x_at |
| actin, gamma 1 | ACTG1 | Hs.514581 | 211970_x_at |
| actin, gamma 1 | ACTG1 | Hs.514581 | 211983_x_at |
| actin, gamma 1 | ACTG1 | Hs.514581 | 211995_x_at |
| actin, gamma 1 | ACTG1 | Hs.514581 | 212363_x_at |
| actin, gamma 1 | ACTG1 | Hs.514581 | 212988_x_at |
| actin, gamma 1 | ACTG1 | Hs.514581 | 213214_x_at |
| actin, gamma 1 | ACTG1 | Hs.514581 | 221607_x_at |
| actin, gamma 1 | ACTG1 | Hs.514581 | 224585_x_at |
| Activated leukocyte cell adhesion molecule | ALCAM | Hs.150693 | 240655_at |
| activated RNA polymerase II transcription cofactor 4 | PC4 | Hs.229641 | 212857_x_at |
| activating signal cointegrator 1 complex subunit 1 | ASCC1 | Hs.500007 | 1554627_a_at |
| Activating signal cointegrator 1 complex subunit 3- | ASCC3L1 | Hs.246112 | 232931_at |
| like 1 | | | |
| activating signal cointegrator 1 complex subunit 3- | ASCC3L1 | Hs.246112 | 200058_s_at |
| like 1 /// activating signal cointegrator 1 complex | | | |
| subunit 3-like 1 | | | |
| activating transcription factor 2 | ATF2 | Hs.425104 | 205446_s_at |
| activating transcription factor 3 | ATF3 | Hs.460 | 202672_s_at |
| activating transcription factor 4 (tax-responsive | ATF4 | Hs.496487 | 200779_at |
| enhancer element B67) | | | |
| activating transcription factor 5 | ATF5 | Hs.9754 | 204998_s_at |
| activating transcription factor 7 interacting protein | ATF7IP | Hs.546406 | 218987_at |
| activating transcription factor 7 interacting protein | ATF7IP | Hs.546406 | 231825_x_at |
| Activating transcription factor 7 interacting protein 2 | ATF7IP2 | Hs.513343 | 228381_at |
| activin A receptor, type IB | ACVR1B | Hs.438918 | 213198_at |
| activity-dependent neuroprotector | ADNP | Hs.293736 | 201773_at |
| Activity-dependent neuroprotector | ADNP | Hs.293736 | 222366_at |
| Activity-dependent neuroprotector | ADNP | Hs.293736 | 226426_at |
| acyl-CoA synthetase long-chain family member 3 | ACSL3 | Hs.471461 | 201661_s_at |
| acyl-CoA synthetase long-chain family member 6 | ACSL6 | Hs.14945 | 211207_s_at |
| Acyl-CoA synthetase long-chain family member 6 | ACSL6 | Hs.14945 | 216409_at |
| acyl-Coenzyme A dehydrogenase family, member 9 | ACAD9 | Hs.541841 | 224160_s_at |
| acyl-malonyl condensing enzyme 1 | AMAC1 | Hs.514814 | 1553471_at |
| acyl-malonyl condensing enzyme 1-like 2 /// acyl- | AMAC1L2 | Hs.514814 | 1553340_s_at |
| malonyl condensing enzyme 1 | /// | | |
| | AMAC1 | | |
| acylphosphatase 2, muscle type | ACYP2 | Hs.516173 | 206833_s_at |
| ADAM-like, decysin 1 | ADAMDEC1 | Hs.521459 | 206134_at |
| ADAMTS-like 3 | ADAMTSL3 | Hs.459162 | 213974_at |
| Adaptin-ear-binding coat-associated protein 1 | NECAP1 | Hs.527839 | 1559495_at |
| adaptin-ear-binding coat-associated protein 2 | NECAP2 | Hs.437385 | 230123_at |
| Adaptor-related protein complex 1, sigma 2 subunit | AP1S2 | Hs.121592 | 228415_at |
| adaptor-related protein complex 3, sigma 1 subunit | AP3S1 | Hs.406191 | 202442_at |
| adaptor-related protein complex 3, sigma 2 subunit | AP3S2 | Hs.546334 | 202399_s_at |
| adaptor-related protein complex 4, beta 1 subunit | AP4B1 | Hs.515048 | 231714_s_at |
| additional sex combs like 1 (Drosophila) | ASXL1 | Hs.374043 | 212237_at |
| additional sex combs like 1 (Drosophila) | ASXL1 | Hs.374043 | 212238_at |
| additional sex combs like 2 (Drosophila) | ASXL2 | Hs.119815 | 218659_at |
| adducin 1 (alpha) /// adducin 1 (alpha) | ADD1 | Hs.183706 | 208030_s_at |
| adducin 2 (beta) | ADD2 | Hs.188528 | 206807_s_at |
| adducin 3 (gamma) | ADD3 | Hs.501012 | 201752_s_at |
| adducin 3 (gamma) | ADD3 | Hs.501012 | 201753_s_at |
| adenosylmethionine decarboxylase 1 | AMD1 | Hs.159118 | 201196_s_at |
| adenosylmethionine decarboxylase 1 | AMD1 | Hs.159118 | 201197_at |
| adenylate cyclase 2 (brain) | ADCY2 | Hs.481545 | 217687_at |
| adenylate cyclase 3 | ADCY3 | Hs.467898 | 209320_at |
| Adenylate kinase 3 | AK3 | Hs.10862 | 225342_at |
| adenylosuccinate synthase | ADSS | Hs.498313 | 221761_at |
| ADP-ribosylation factor 1 | ARF1 | Hs.286221 | 208750_s_at |
| ADP-ribosylation factor 4 | ARF4 | Hs.148330 | 201097_s_at |
| ADP-ribosylation factor 4-like | ARF4L | Hs.183153 | 203586_s_at |
| ADP-ribosylation factor 6 | ARF6 | Hs.525330 | 203312_x_at |
| ADP-ribosylation factor 6 | ARF6 | Hs.525330 | 214182_at |
| ADP-ribosylation factor 6 | ARF6 | Hs.525330 | 224788_at |
| ADP-ribosylation factor GTPase activating protein 1 | ARFGAP1 | Hs.25584 | 217888_s_at |
| ADP-ribosylation factor guanine nucleotide- | ARFGEF1 | Hs.411848 | 216266_s_at |
| exchange factor 1(brefeldin A-inhibited) | | | |
| ADP-ribosylation factor guanine nucleotide- | ARFGEF1 | Hs.411848 | 236742_at |
| exchange factor 1(brefeldin A-inhibited) | | | |
| ADP-ribosylation factor guanine nucleotide- | ARFGEF2 | Hs.62578 | 222518_at |
| exchange factor 2 (brefeldin A-inhibited) | | | |
| ADP-ribosylation factor-like | LOC51326 | Hs.549159 | 210718_s_at |
| ADP-ribosylation factor-like 10C | ARL10C | Hs.250009 | 217852_s_at |
| ADP-ribosylation factor-like 2 binding protein | ARL2BP | Hs.513631 | 202092_s_at |
| ADP-ribosylation factor-like 3 | ARL3 | Hs.182215 | 202641_at |
| ADP-ribosylation factor-like 4A | ARL4A | Hs.245540 | 205020_s_at |
| ADP-ribosylation factor-like 5 | ARL5 | Hs.470233 | 218150_at |
| ADP-ribosylation factor-like 6 interacting protein | ARL6IP | Hs.75249 | 211935_at |
| ADP-ribosylation factor-like 8 | ARL8 | Hs.25362 | 226345_at |
| ADP-ribosylation-like factor 6 interacting protein 5 | ARL6IP5 | Hs.518060 | 200761_s_at |
| ADP-ribosylhydrolase like 2 | ADPRHL2 | Hs.18021 | 223097_at |
| adrenergic, beta, receptor kinase 2 | ADRBK2 | Hs.517493 | 204183_s_at |
| adrenomedullin | ADM | Hs.441047 | 202912_at |
| adrenomedullin receptor | ADMR | Hs.483909 | 1552440_at |
| adrenomedullin receptor | ADMR | Hs.483909 | 214506_at |
| adult retina protein | LOC153222 | Hs.484195 | 1554229_at |
| adult retina protein | LOC153222 | Hs.484195 | 225956_at |
| aftiphilin protein | AFTIPHILIN | Hs.468760 | 217939_s_at |
| aftiphilin protein | AFTIPHILIN | Hs.468760 | 222472_at |
| AG1 protein | AE01 | Hs.445080 | 237917_at |
| alanyl-tRNA synthetase | AARS | Hs.315137 | 201000_at |
| alcohol dehydrogenase 4 (class II), pi polypeptide | ADH4 | Hs.1219 | 223781_x_at |
| alcohol dehydrogenase 5 (class III), chi | ADH5 | Hs.78989 | 208847_s_at |
| polypeptide | | | |
| Aldehyde dehydrogenase 6 family, member A1 | ALDH6A1 | Hs.293970 | 221589_s_at |
| aldehyde dehydrogenase 7 family, member A1 | ALDH7A1 | Hs.483239 | 208950_s_at |
| Aldehyde dehydrogenase 7 family, member A1 | ALDH7A1 | Hs.483239 | 208951_at |
| aldo-keto reductase family 1, member C1 | AKR1C1 | Hs.460260 | 204151_x_at |
| (dihydrodiol dehydrogenase 1; 20-alpha (3-alpha)- | | | |
| hydroxysteroid dehydrogenase) | | | |
| aldo-keto reductase family 7, member A2 (aflatoxin | AKR7A2 | Hs.512807 | 214259_s_at |
| aldehyde reductase) | | | |
| Aldolase A, fructose-bisphosphate pseudogene 2 | ALDOAP2 | Hs.148259 | 1556133_s_at |
| ALL1 fused gene from 5q31 | AF5Q31 | Hs.519313 | 219199_at |
| ALL1 fused gene from 5q31 | AF5Q31 | Hs.519313 | 232865_at |
| ALL1-fused gene from chromosome 1q /// ALL1- | AF1Q | Hs.75823 | 211071_s_at |
| fused gene from chromosome 1q | | | |
| all-trans-13,14-dihydroretinol saturase | RetSat | Hs.440401 | 218124_at |
| alpha thalassemia/mental retardation syndrome X- | ATRX | Hs.533526 | 208861_s_at |
| linked (RAD54 homolog, S. cerevisiae) | | | |
| alpha-1,3(6)-mannosylglycoprotein beta-1,6-N- | LOC90693 | Hs.232296 | 228061_at |
| acetyl-glucosaminyltransferase-like | | | |
| alpha-2-macroglobulin | A2M | Hs.212838 | 1558450_at |
| alpha-kinase 2 | ALPK2 | Hs.388674 | 228367_at |
| alpha-methylacyl-CoA racemase | AMACR | Hs.508343 | 209426_s_at |
| Alport syndrome, mental retardation, midface | AMMECR1 | Hs.211021 | 204976_s_at |
| hypoplasia and elliptocytosis chromosomal region, | | | |
| gene 1 | | | |
| Alstrom syndrome 1 | ALMS1 | Hs.184720 | 214220_s_at |
| Alstrom syndrome 1 | ALMS1 | Hs.184720 | 214707_x_at |
| Alstrom syndrome 1 | ALMS1 | Hs.184720 | 233788_at |
| amelogenin (amelogenesis imperfecta 1, X-linked) | AMELX | Hs.46329 | 208410_x_at |
| amiloride binding protein 1 (amine oxidase | ABP1 | Hs.521296 | 203559_s_at |
| (copper-containing)) | | | |
| amine oxidase, copper containing 2 (retina- | AOC2 | Hs.143102 | 207064_s_at |
| specific) | | | |
| amino acid transporter | FLJ10815 | Hs.10499 | 218727_at |
| Amino acid transporter | FLJ10815 | Hs.10499 | 228951_at |
| amino acid transporter | FLJ10815 | Hs.10499 | 56821_at |
| aminoacylase 1-like 2 | ACY1L2 | Hs.356247 | 225431_x_at |
| aminoadipate-semialdehyde dehydrogenase- | AASDHPPT | Hs.524009 | 202169_s_at |
| phosphopantetheinyl transferase | | | |
| aminoadipate-semialdehyde dehydrogenase- | AASDHPPT | Hs.524009 | 202170_s_at |
| phosphopantetheinyl transferase | | | |
| aminomethyltransferase (glycine cleavage system | AMT | Hs.102 | 204294_at |
| protein T) | | | |
| amplified in osteosarcoma | OS-9 | Hs.527861 | 200714_x_at |
| amylase, alpha 1A; salivary /// amylase, alpha 1B; | AMY1A | Hs.514463 | 208498_s_at |
| salivary /// amylase, alpha 1C; salivary /// amylase, | /// | | |
| alpha 2A; pancreatic /// amylase, alpha 2B; | AMY1B | | |
| pancreatic | /// | | |
| | AMY1C | | |
| | /// | | |
| | AMY2A | | |
| | /// | | |
| | AMY2B | | |
| amylo-1,6-glucosidase, 4-alpha- | AGL | Hs.904 | 203566_s_at |
| glucanotransferase (glycogen debranching | | | |
| enzyme, glycogen storage disease type III) | | | |
| amyloid beta (A4) precursor protein-binding, family | APBB2 | Hs.479602 | 40148_at |
| B, member 2 (Fe65-like) | | | |
| amyloid beta (A4) precursor protein-binding, family | APBB3 | Hs.529449 | 204650_s_at |
| B, member 3 | | | |
| amyloid beta (A4) precursor-like protein 2 | APLP2 | Hs.370247 | 208248_x_at |
| amyloid beta precursor protein (cytoplasmic tail) | APPBP2 | Hs.84084 | 202631_s_at |
| binding protein 2 | | | |
| amyotrophic lateral sclerosis 2 (juvenile) | ALS2CR11 | Hs.335788 | 1553260_s_at |
| chromosome region, candidate 11 | | | |
| amyotrophic lateral sclerosis 2 (juvenile) | ALS2CR11 | Hs.335788 | 1554831_x_at |
| chromosome region, candidate 11 | | | |
| amyotrophic lateral sclerosis 2 (juvenile) | ALS2CR13 | Hs.471130 | 1553220_at |
| chromosome region, candidate 13 | | | |
| amyotrophic lateral sclerosis 2 (juvenile) | ALS2CR16 | Hs.546480 | 1554106_at |
| chromosome region, candidate 16 | | | |
| Amyotrophic lateral sclerosis 2 (juvenile) | ALS2CR3 | Hs.152774 | 235983_at |
| chromosome region, candidate 3 | | | |
| amyotrophic lateral sclerosis 2 (juvenile) | ALS2CR4 | Hs.12319 | 1553956_at |
| chromosome region, candidate 4 | | | |
| AN1, ubiquitin-like, homolog (Xenopus laevis) | ANUBL1 | Hs.89029 | 242650_at |
| AN1, ubiquitin-like, homolog (Xenopus laevis) | ANUBL1 | Hs.89029 | 244206_at |
| Anaphase promoting complex subunit 1 | ANAPC1 | Hs.436527 | 229267_at |
| anaphase promoting complex subunit 1 | ANAPC1 | Hs.436527 | 231973_s_at |
| anaphase promoting complex subunit 13 | ANAPC13 | Hs.106909 | 209001_s_at |
| anaphase promoting complex subunit 5 | ANAPC5 | Hs.7101 | 208721_s_at |
| anaplastic lymphoma kinase (Ki-1) | ALK | Hs.196534 | 208212_s_at |
| Angiomotin like 1 | AMOTL1 | Hs.503594 | 240877_x_at |
| angiotensin II receptor, type 1 | AGTR1 | Hs.477887 | 205357_s_at |
| anillin, actin binding protein (scraps homolog, | ANLN | Hs.62180 | 1552619_a_at |
| Drosophila) | | | |
| Ankyrin 1, erythrocytic | ANK1 | Hs.491558 | 240363_at |
| ankyrin repeat and KH domain containing 1 | ANKHD1 | Hs.434219 | 219081_at |
| ankyrin repeat and KH domain containing 1 /// | ANKHD1 | Hs.434219 | 208773_s_at |
| MASK-4E-BP3 alternate reading frame gene | /// | | |
| | MASK- | | |
| | BP3 | | |
| ankyrin repeat and MYND domain containing 2 | ANKMY2 | Hs.157378 | 212798_s_at |
| ankyrin repeat and SOCS box-containing 12 | ASB12 | Hs.56281 | 236033_at |
| ankyrin repeat and SOCS box-containing 17 | ASB17 | Hs.125423 | 231622_at |
| Ankyrin repeat domain 12 | ANKRD12 | Hs.464585 | 239450_at |
| ankyrin repeat domain 15 | ANKRD15 | Hs.493272 | 213005_s_at |
| ankyrin repeat domain 15 | ANKRD15 | Hs.493272 | 238789_at |
| ankyrin repeat domain 20A /// chromosome 21 | ANKRD20A | Hs.522178 | 1569607_s_at |
| open reading frame 81 /// similar to ankyrin repeat | /// | | |
| domain 20A /// OTTHUMP00000015360 /// | C21orf81 | | |
| OTTHUMP00000064580 | /// | | |
| | LOC440841 | | |
| | /// | | |
| | LOC441425 | | |
| | /// | | |
| | LOC441430 | | |
| ankyrin repeat domain 28 | ANKRD28 | Hs.335239 | 229307_at |
| ankyrin repeat domain 29 | ANKRD29 | Hs.374774 | 238332_at |
| ankyrin repeat domain 5 | ANKRD5 | Hs.70903 | 222951_s_at |
| ankyrin repeat domain 7 | ANKRD7 | Hs.371820 | 206112_at |
| ankyrin repeat domain 9 | ANKRD9 | Hs.432945 | 230972_at |
| annexin A1 | ANXA1 | Hs.494173 | 201012_at |
| annexin A2 | ANXA2 | Hs.511605 | 1568126_at |
| annexin A2 | ANXA2 | Hs.511605 | 201590_x_at |
| annexin A2 | ANXA2 | Hs.511605 | 210427_x_at |
| annexin A5 | ANXA5 | Hs.480653 | 200782_at |
| anterior pharynx defective 1 homolog A (C. elegans) | APH1A | Hs.108408 | 218389_s_at |
| anterior pharynx defective 1 homolog B (C. elegans) | APH1B | Hs.511703 | 221036_s_at |
| /// anterior pharynx defective 1 homolog B | | | |
| (C. elegans) | | | |
| Anthrax toxin receptor 1 | ANTXR1 | Hs.165859 | 232808_at |
| antizyme inhibitor 1 | AZIN1 | Hs.459106 | 201772_at |
| AP1 gamma subunit binding protein 1 | AP1GBP1 | Hs.101480 | 222715_s_at |
| AP1 gamma subunit binding protein 1 | AP1GBP1 | Hs.101480 | 239678_at |
| AP2 associated kinase 1 | AAK1 | Hs.552584 | 205434_s_at |
| APG12 autophagy 12-like (S. cerevisiae) | APG12L | Hs.264482 | 213026_at |
| APG3 autophagy 3-like (S. cerevisiae) | APG3L | Hs.477126 | 221492_s_at |
| APG4 autophagy 4 homolog A (S. cerevisiae) | APG4A | Hs.8763 | 213115_at |
| APG5 autophagy 5-like (S. cerevisiae) | APG5L | Hs.486063 | 210639_s_at |
| APG7 autophagy 7-like (S. cerevisiae) | APG7L | Hs.38032 | 1569827_at |
| apobec-1 complementation factor | ACF | Hs.499643 | 220951_s_at |
| apolipoprotein B mRNA editing enzyme, catalytic | APOBE | Hs.441124 | 209584_x_at |
| polypeptide-like 3C | C3C | | |
| Apolipoprotein E | APOE | Hs.515465 | 212884_x_at |
| apolipoprotein M | APOM | Hs.534468 | 205682_x_at |
| apoptosis inhibitor 5 | API5 | Hs.435771 | 201686_x_at |
| apoptosis-related protein PNAS-1 | FLJ39616 | Hs.333120 | 1553987_at |
| aprataxin | APTX | Hs.20158 | 218527_at |
| aquaporin 3 | AQP3 | Hs.234642 | 39248_at |
| aquarius homolog (mouse) | AQR | Hs.510958 | 212584_at |
| Arachidonate 12-lipoxygenase | ALOX12 | Hs.422967 | 229833_at |
| arginine vasopressin-induced 1 | AVPI1 | Hs.23918 | 218631_at |
| Arginine-glutamic acid dipeptide (RE) repeats | RERE | Hs.463041 | 232877_at |
| Arginine-glutamic acid dipeptide (RE) repeats | RERE | Hs.463041 | 244625_at |
| arginyl-tRNA synthetase-like | RARSL | Hs.485910 | 225264_at |
| ariadne homolog, ubiquitin-conjugating enzyme E2 | ARIH1 | Hs.268787 | 201881_s_at |
| binding protein, 1 (Drosophila) | | | |
| Ariadne homolog, ubiquitin-conjugating enzyme E2 | ARIH1 | Hs.268787 | 242431_at |
| binding protein, 1 (Drosophila) | | | |
| armadillo repeat containing 1 | ARMC1 | Hs.269542 | 222550_at |
| armadillo repeat containing 2 | ARMC2 | Hs.282925 | 223866_at |
| armadillo repeat containing 8 | ARMC8 | Hs.266826 | 203486_s_at |
| armadillo repeat containing, X-linked 1 | ARMCX1 | Hs.9728 | 218694_at |
| armadillo repeat containing, X-linked 6 | ARMCX6 | Hs.83530 | 214749_s_at |
| ARP2 actin-related protein 2 homolog (yeast) | ACTR2 | Hs.393201 | 1554390_s_at |
| ARP2 actin-related protein 2 homolog (yeast) | ACTR2 | Hs.393201 | 1558015_s_at |
| ARP2 actin-related protein 2 homolog (yeast) | ACTR2 | Hs.393201 | 200727_s_at |
| ARP2 actin-related protein 2 homolog (yeast) | ACTR2 | Hs.393201 | 200729_s_at |
| ARP3 actin-related protein 3 homolog (yeast) | ACTR3 | Hs.433512 | 200996_at |
| ARP3 actin-related protein 3 homolog (yeast) | ACTR3 | Hs.433512 | 213101_s_at |
| ARP3 actin-related protein 3 homolog (yeast) | ACTR3 | Hs.433512 | 213102_at |
| ARP3 actin-related protein 3 homolog (yeast) | ACTR3 | Hs.433512 | 228603_at |
| ARP6 actin-related protein 6 homolog (yeast) | ACTR6 | Hs.115088 | 239679_at |
| ARP8 actin-related protein 8 homolog (yeast) | ACTR8 | Hs.412186 | 222707_s_at |
| arrestin domain containing 4 | ARRDC4 | Hs.6093 | 225283_at |
| Arrestin, beta 2 | ARRB2 | Hs.435811 | 1559436_x_at |
| arsenate resistance protein ARS2 | ARS2 | Hs.111801 | 222047_s_at |
| Aryl hydrocarbon receptor nuclear translocator | ARNT | Hs.131494 | 231016_s_at |
| ASF1 anti-silencing function 1 homolog A (S. cerevisiae) | ASF1A | Hs.292316 | 203428_s_at |
| Ash1 (absent, small, or homeotic)-like (Drosophila) | ASH1L | Hs.491060 | 1554057_at |
| ash1 (absent, small, or homeotic)-like (Drosophila) | ASH1L | Hs.491060 | 222667_s_at |
| asparaginase like 1 | ASRGL1 | Hs.535326 | 218857_s_at |
| asparagine-linked glycosylation 5 homolog (yeast, | ALG5 | Hs.507769 | 218203_at |
| dolichyl-phosphate beta-glucosyltransferase) | | | |
| asparagine-linked glycosylation 8 homolog (yeast, | ALG8 | Hs.503368 | 203545_at |
| alpha-1,3-glucosyltransferase) | | | |
| aspartyl aminopeptidase | DNPEP | Hs.258551 | 215135_at |
| aspartyl aminopeptidase | DNPEP | Hs.258551 | 38703_at |
| aspartylglucosaminidase | AGA | Hs.207776 | 204332_s_at |
| astrotactin 2 | ASTN2 | Hs.209217 | 215407_s_at |
| AT rich interactive domain 1B (SWI1-like) | ARID1B | Hs.291587 | 238043_at |
| AT rich interactive domain 4B (RBP1-like) /// AT | ARID4B | Hs.533633 | 221230_s_at |
| rich interactive domain 4B (RBP1-like) | | | |
| AT rich interactive domain 5B (MRF1-like) | ARID5B | Hs.546755 | 1563469_at |
| AT rich interactive domain 5B (MAF1-like) | ARID5B | Hs.546755 | 241837_at |
| Ataxin 1 | ATXN1 | Hs.434961 | 1559249_at |
| ataxin 10 | ATXN10 | Hs.475125 | 208832_at |
| ataxin 10 | ATXN10 | Hs.475125 | 208833_s_at |
| Ataxin 2 | ATXN2 | Hs.76253 | 237184_at |
| Ataxin 2 | ATXN2 | Hs.76253 | 238589_s_at |
| ataxin 7 | ATXN7 | Hs.476595 | 204516_at |
| Atonal homolog 8 (Drosophila) | ATOH8 | Hs.135569 | 1558706_a_at |
| ATP citrate lyase | ACLY | Hs.387567 | 201127_s_at |
| ATP synthase, H+ transporting, mitochondrial F0 | ATP5F1 | Hs.514870 | 211755_s_at |
| complex, subunit b, isoform 1 /// ATP synthase, H+ | | | |
| transporting, mitochondrial F0 complex, subunit b, | | | |
| isoform 1 | | | |
| ATP synthase, H+ transporting, mitochondrial F0 | ATP5I | Hs.85539 | 207335_x_at |
| complex, subunit e | | | |
| ATP synthase, H+ transporting, mitochondrial F0 | ATP5I | Hs.85539 | 209492_x_at |
| complex, subunit e | | | |
| ATP synthase, H+ transporting, mitochondrial F0 | ATP5J | Hs.246310 | 202325_s_at |
| complex, subunit F6 | | | |
| ATP synthase, H+ transporting, mitochondrial F0 | ATP5S | Hs.438489 | 206993_at |
| complex, subunit s (factor B) | | | |
| ATP synthase, H+ transporting, mitochondrial F0 | ATP5S | Hs.438489 | 213995_at |
| complex, subunit s (factor B) | | | |
| ATP synthase, H+ transporting, mitochondrial F1 | ATP5A1 | Hs.298280 | 213738_s_at |
| complex, alpha subunit, isoform 1, cardiac muscle | | | |
| ATP synthase, H+ transporting, mitochondrial F1 | ATP5D | Hs.418668 | 203926_x_at |
| complex, delta subunit | | | |
| ATP synthase, H+ transporting, mitochondrial F1 | ATP5E | Hs.546238 | 217801_at |
| complex, epsilon subunit | | | |
| ATP synthase, H+ transporting, mitochondrial F1 | ATP5C1 | Hs.271135 | 208870_x_at |
| complex, gamma polypeptide 1 | | | |
| ATP/GTP binding protein 1 | AGTPBP1 | Hs.494321 | 204500_s_at |
| ATP/GTP binding protein 1 | AGTPBP1 | Hs.494321 | 242008_at |
| ATP/GTP-binding protein | HEAB | Hs.523687 | 204370_at |
| ATPase family, AAA domain containing 2 | ATAD2 | Hs.370834 | 218782_s_at |
| ATPase inhibitory factor 1 | ATPIF1 | Hs.241336 | 218671_s_at |
| ATPase inhibitory factor 1 | ATPIF1 | Hs.241336 | 223338_s_at |
| ATPase type 13A5 | ATP13A5 | — | 1553567_s_at |
| ATPase, Ca++ transporting, cardiac muscle, slow | ATP2A2 | Hs.506759 | 209186_at |
| twitch 2 | | | |
| ATPase, Ca++ transporting, type 2C, member 1 | ATP2C1 | Hs.546361 | 212255_s_at |
| ATPase, Ca++ transporting, type 2C, member 1 | ATP2C1 | Hs.546361 | 230387_at |
| ATPase, Class I, type 8B, member 1 | ATP8B1 | Hs.216623 | 214594_x_at |
| ATPase, Class II, type 9B | ATP9B | Hs.465475 | 243615_at |
| ATPase, Class VI, type 11B | ATP11B | Hs.478429 | 1564064_a_at |
| ATPase, Class VI, type 11B | ATP11B | Hs.478429 | 212536_at |
| ATPase, Class VI, type 11B | ATP11B | Hs.478429 | 238811_at |
| ATPase, H+ transporting, lysosomal 13 kDa, V1 | ATP6V1G1 | Hs.388654 | 208737_at |
| subunit G isoform 1 | | | |
| ATPase, H+ transporting, lysosomal 13 kDa, V1 | ATP6V1G1 | Hs.388654 | 238765_at |
| subunit G isoform 1 | | | |
| ATPase, H+ transporting, lysosomal 31 kDa, V1 | ATP6V1E2 | Hs.534515 | 1552286_at |
| subunit E isoform 2 | | | |
| ATPase, H+ transporting, lysosomal 34 kDa, V1 | ATP6V1D | Hs.272630 | 208899_x_at |
| subunit D | | | |
| ATPase, H+ transporting, lysosomal 34 kDa, V1 | ATP6V1D | Hs.272630 | 214923_at |
| subunit D | | | |
| ATPase, H+ transporting, lysosomal 38 kDa, V0 | ATP6V0D1 | Hs.106876 | 239633_at |
| subunit d isoform 1 | | | |
| ATPase, H+ transporting, lysosomal 42 kDa, V1 | ATP6V1C1 | Hs.86905 | 202874_s_at |
| subunit C, isoform, 1 | | | |
| ATPase, H+ transporting, lysosomal 50/57 kDa, V1 | ATP6V1H | Hs.491737 | 1557586_s_at |
| subunit H | | | |
| ATPase, H+ transporting, lysosomal 70 kDa, V1 | ATP6V1A | Hs.477155 | 201971_s_at |
| subunit A | | | |
| ATPase, H+ transporting, lysosomal 70 kDa, V1 | ATP6V1A | Hs.477155 | 201972_at |
| subunit A | | | |
| ATPase, H+ transporting, lysosomal 9 kDa, V0 | ATP6V0E | Hs.484188 | 201172_x_at |
| subunit e | | | |
| ATPase, H+ transporting, lysosomal 9 kDa, V0 | ATP6V0E | Hs.484188 | 200096_s_at |
| subunit e /// ATPase, H+ transporting, lysosomal | | | |
| 9 kDa, V0 subunit e | | | |
| ATPase, H+ transporting, lysosomal V0 subunit a | ATP6V0A1 | Hs.463074 | 212383_at |
| isoform 1 | | | |
| ATPase, Na+/K+ transporting, beta 1 polypeptide | ATP1B1 | Hs.291196 | 201242_s_at |
| ATPase, Na+/K+ transporting, beta 3 polypeptide | ATP1B3 | Hs.477789 | 208836_at |
| ATPase, Na+/K+ transporting, beta 3 polypeptide | ATP1B3 | Hs.477789 | 229709_at |
| ATP-binding cassette, sub-family A (ABC1), | ABCA1 | Hs.429294 | 203504_s_at |
| member 1 | | | |
| ATP-binding cassette, sub-family A (ABC1), | ABCA1 | Hs.429294 | 203505_at |
| member 1 | | | |
| ATP-binding cassette, sub-family C (CFTR/MRP), | ABCC1 | Hs.391464 | 202804_at |
| member 1 | | | |
| ATP-binding cassette, sub-family C (CFTR/MRP), | ABCC5 | Hs.368563 | 209380_s_at |
| member 5 | | | |
| ATP-binding cassette, sub-family C (CFTR/MRP), | ABCC9 | Hs.446050 | 1557374_at |
| member 9 | | | |
| ATP-binding cassette, sub-family D (ALD), | ABCD3 | Hs.76781 | 202850_at |
| member 3 | | | |
| ATP-binding cassette, sub-family E (OABP), | ABCE1 | Hs.12013 | 201872_s_at |
| member 1 | | | |
| ATP-binding cassette, sub-family G (WHITE), | ABCG1 | Hs.124649 | 204567_s_at |
| member 1 | | | |
| ATP-binding cassette, sub-family G (WHITE), | ABCG2 | Hs.480218 | 209735_at |
| member 2 | | | |
| ATP-binding cassette, sub-family G (WHITE), | ABCG4 | Hs.126378 | 207593_at |
| member 4 | | | |
| Atrophin 1 | DRPLA | Hs.143766 | 236961_at |
| AU RNA binding protein/enoyl-Coenzyme A | AUH | Hs.175905 | 205052_at |
| hydratase | | | |
| aurora-A kinase interacting protein | AKIP | Hs.472535 | 225552_x_at |
| autism susceptibility candidate 2 | AUTS2 | Hs.21631 | 212599_at |
| AXIN1 up-regulated 1 | AXUD1 | Hs.370950 | 225557_at |
| AYP1 protein | AYP1 | Hs.397010 | 226453_at |
| bA255A11.10 (putative novel T cell receptor beta | bA255A11.1 | Hs.521441 | 234686_at |
| chain V region protein) | | | |
| baculoviral IAP repeat-containing 6 (apollon) | BIRC6 | Hs.150107 | 224635_s_at |
| baculoviral IAP repeat-containing 6 (apollon) | BIRC6 | Hs.150107 | 233093_s_at |
| Baculoviral IAP repeat-containing 6 (apollon) | BIRC6 | Hs.150107 | 242068_at |
| BAI1-associated protein 1 | BAIAP1 | Hs.169441 | 1559256_at |
| BAI1-associated protein 1 | BAIAP1 | Hs.169441 | 1559257_a_at |
| BAI1-associated protein 1 | BAIAP1 | Hs.169441 | 206144_at |
| BAI1-associated protein 2-like 1 | BAIAP2L1 | Hs.489237 | 227372_s_at |
| Bardet-Biedl syndrome 2 | BBS2 | Hs.333738 | 223227_at |
| Bardet-Biedl syndrome 4 | BBS4 | Hs.208681 | 212744_at |
| Bardet-Biedl syndrome 7 | BBS7 | Hs.58974 | 235007_at |
| basic leucine zipper and W2 domains 1 | BZW1 | Hs.355983 | 200777_s_at |
| basic leucine zipper and W2 domains 2 | BZW2 | Hs.487635 | 217809_at |
| basic transcription factor 3 | BTF3 | Hs.529798 | 211939_x_at |
| basic transcription factor 3 /// similar to | BTF3 /// | Hs.529798 | 214800_x_at |
| Transcription factor BTF3 (RNA polymerase B | LOC345829 | | |
| transcription factor 3) | | | |
| BAT2 domain containing 1 | BAT2D1 | Hs.494614 | 211946_s_at |
| BAT2 domain containing 1 | BAT2D1 | Hs.494614 | 211948_x_at |
| BAT2 domain containing 1 | BAT2D1 | Hs.494614 | 214055_x_at |
| B-box and SPRY domain containing | BSPRY | Hs.494870 | 222746_s_at |
| BBP-like protein 2 /// BBP-like protein 2 | BLP2 | Hs.288912 | 221702_s_at |
| B-cell CLL/lymphoma 10 | BCL10 | Hs.549082 | 1557257_at |
| B-cell CLL/lymphoma 10 | BCL10 | Hs.549082 | 1557258_a_at |
| B-cell CLL/lymphoma 9 | BCL9 | Hs.415209 | 222350_at |
| B-cell receptor-associated protein 29 | BCAP29 | Hs.303787 | 225674_at |
| B-cell receptor-associated protein 29 | BCAP29 | Hs.303787 | 241646_s_at |
| B-cell translocation gene 1, anti-proliferative | BTG1 | Hs.255935 | 200920_s_at |
| B-cell translocation gene 1, anti-proliferative | BTG1 | Hs.255935 | 200921_s_at |
| BCL2/adenovirus E1B 19 kDa interacting protein 3 | BNIP3 | Hs.144873 | 201849_at |
| BCL2/adenovirus E1B 19 kDa interacting protein 3- | BNIP3L | Hs.131226 | 221479_s_at |
| like /// BCL2/adenovirus E1B 19 kDa interacting | | | |
| protein 3-like | | | |
| BCL2-associated athanogene | BAG1 | Hs.377484 | 211475_s_at |
| BCL2-associated athanogene /// BCL2-associated | BAG1 | Hs.377484 | 202387_at |
| athanogene | | | |
| BCL2-associated athanogene 3 | BAG3 | Hs.523309 | 217911_s_at |
| BCL2-associated athanogene 5 | BAG5 | Hs.5443 | 202984_s_at |
| BCL2-associated athanogene 5 | BAG5 | Hs.5443 | 202985_s_at |
| BCL2-associated transcription factor 1 | BCLAF1 | Hs.486542 | 201083_s_at |
| BCL2-associated transcription factor 1 | BCLAF1 | Hs.486542 | 201084_s_at |
| BCL2-associated transcription factor 1 | BCLAF1 | Hs.486542 | 214499_s_at |
| BCL2-like 11 (apoptosis facilitator) | BCL2L11 | Hs.469658 | 1553088_a_at |
| BCL2-like 11 (apoptosis facilitator) | BCL2L11 | Hs.469658 | 1553096_s_at |
| BCL2-like 11 (apoptosis facilitator) | BCL2L11 | Hs.469658 | 1555372_at |
| BCL2-like 11 (apoptosis facilitator) | BCL2L11 | Hs.469658 | 222343_at |
| BCL2-like 14 (apoptosis facilitator) /// BCL2-like 14 | BCL2L14 | Hs.549194 | 221241_s_at |
| (apoptosis facilitator) | | | |
| beclin 1 (coiled-coil, myosin-like BCL2 interacting | BECN1 | Hs.12272 | 208945_s_at |
| protein) | | | |
| beta-2-microglobulin | B2M | Hs.534255 | 201891_s_at |
| beta-amyloid binding protein precursor | BBP | Hs.276876 | 213883_s_at |
| beta-site APP-cleaving enzyme 2 | BACE2 | Hs.529408 | 217867_x_at |
| beta-transducin repeat containing | BTRC | Hs.500812 | 216091_s_at |
| bicaudal C homolog 1 (Drosophila) | BICC1 | Hs.158745 | 220580_at |
| biglycan | BGN | Hs.821 | 201261_x_at |
| Bladder cancer associated protein | BLCAP | Hs.472651 | 1557521_a_at |
| bladder cancer associated protein | BLCAP | Hs.472651 | 201032_at |
| blood vessel epicardial substance | BVES | Hs.221660 | 223853_at |
| BMS1-like, ribosome assembly protein (yeast) | BMS1L | Hs.10848 | 203082_at |
| Bobby sox homolog (Drosophila) | BBX | Hs.124366 | 1557240_a_at |
| bobby sox homolog (Drosophila) | BBX | Hs.124366 | 223135_s_at |
| bobby sox homolog (Drosophila) | BBX | Hs.124366 | 232008_s_at |
| bol, boule-like (Drosophila) | BOLL | Hs.169797 | 236954_at |
| bone morphogenetic protein receptor, type IA | BMPR1A | Hs.524477 | 213578_at |
| brain abundant, membrane attached signal protein 1 | BASP1 | Hs.201641 | 202391_at |
| brain acyl-CoA hydrolase | BACH | Hs.126137 | 208002_s_at |
| brain and reproductive organ-expressed | BRE | Hs.258314 | 205550_s_at |
| (TNFRSF1A modulator) | | | |
| brain protein 16 | LOC51236 | Hs.300224 | 219071_x_at |
| brain protein 44-like | BRP44L | Hs.172755 | 218024_at |
| brain protein I3 | BRI3 | Hs.410497 | 223376_s_at |
| branched chain aminotransferase 1, cytosolic | BCAT1 | Hs.438993 | 226517_at |
| breakpoint cluster region /// similar to breakpoint | BCR /// | Hs.531306 | 226602_s_at |
| cluster region isoform 1 | LOC440820 | | |
| breast carcinoma amplified sequence 1 | BCAS1 | Hs.400556 | 204378_at |
| breast carcinoma amplified sequence 2 | BCAS2 | Hs.22960 | 203053_at |
| BRF2, subunit of RNA polymerase III transcription | BRF2 | Hs.274136 | 218955_at |
| initiation factor, BRF1-like | | | |
| bromodomain adjacent to zinc finger domain, 1A | BAZ1A | Hs.509140 | 217985_s_at |
| Bromodomain adjacent to zinc finger domain, 1B | BAZ1B | Hs.488671 | 1569444_at |
| bromodomain containing 2 | BRD2 | Hs.75243 | 208685_x_at |
| bromodomain containing 2 | BRD2 | Hs.75243 | 214911_s_at |
| bromodomain containing 3 | BRD3 | Hs.522472 | 203825_at |
| Bromodomain containing 3 | BRD3 | Hs.522472 | 212547_at |
| bromodomain containing 4 | BRD4 | Hs.187763 | 202102_s_at |
| Bromodomain containing 4 | BRD4 | Hs.187763 | 232551_at |
| bromodomain containing 8 | BRD8 | Hs.519337 | 202227_s_at |
| bruno-like 5, RNA binding protein (Drosophila) | BRUNOL5 | Hs.501423 | 230497_at |
| bruno-like 6, RNA binding protein (Drosophila) | BRUNOL6 | Hs.348342 | 227775_at |
| BS 3076 | — | Hs.170421 | 1555823_at |
| BS 3076 | — | Hs.170421 | 1555824_a_at |
| BTAF1 RNA polymerase II, B-TFIID transcription | BTAF1 | Hs.500526 | 209430_at |
| factor-associated, 170 kDa (Mot1 homolog, S. cerevisiae) | | | |
| BTB (POZ) domain containing 14B | BTBD14B | Hs.531614 | 215587_x_at |
| BTB (POZ) domain containing 15 | BTBD15 | Hs.178499 | 1554469_at |
| BTB (POZ) domain containing 15 | BTBD15 | Hs.178499 | 226148_at |
| BTB (POZ) domain containing 5 | BTBD5 | Hs.174682 | 235727_at |
| BTB (POZ) domain containing 6 | BTBD6 | Hs.7367 | 225389_at |
| BTB (POZ) domain containing 9 | BTBD9 | Hs.116233 | 233041_x_at |
| BTB and CNC homology 1, basic leucine zipper | BACH1 | Hs.154276 | 210818_s_at |
| transcription factor 1 | | | |
| BTB and CNC homology 1, basic leucine zipper | BACH1 | Hs.154276 | 234315_at |
| transcription factor 1 | | | |
| BTB and CNC homology 1, basic leucine zipper | BACH2 | Hs.269764 | 236796_at |
| transcription factor 2 | | | |
| BUB3 budding uninhibited by benzimidazoles 3 | BUB3 | Hs.418533 | 201456_s_at |
| homolog (yeast) | | | |
| BUB3 budding uninhibited by benzimidazoles 3 | BUB3 | Hs.418533 | 209974_s_at |
| homolog (yeast) | | | |
| bubblegum related protein | BGR | Hs.465720 | 221716_s_at |
| Butyryl Coenzyme A synthetase 1 | BUCS1 | Hs.306812 | 234643_x_at |
| C114 SLIT-like testicular protein | LOC474170 | Hs.463264 | 1569180_at |
| C114 SLIT-like testicular protein | LOC474170 | Hs.463264 | 1569181_x_at |
| C114 SLIT-like testicular protein | LOC474170 | Hs.463264 | 233519_at |
| C1q and tumor necrosis factor related protein 3 /// | C1QTNF3 | Hs.171929 | 220988_s_at |
| C1q and tumor necrosis factor related protein 3 | | | |
| Cadherin 11, type 2, OB-cadherin (osteoblast) | CDH11 | Hs.116471 | 239286_at |
| cadherin, EGF LAG seven-pass G-type receptor 2 | CELSR2 | Hs.57652 | 204029_at |
| (flamingo homolog, Drosophila) | | | |
| cadherin-like 26 | CDH26 | Hs.54973 | 233391_at |
| calcitonin gene-related peptide-receptor | RCP9 | Hs.300684 | 203898_at |
| component protein | | | |
| calcium and integrin binding family member 2 | CIB2 | Hs.129867 | 205007_s_at |
| calcium binding protein 39-like | CAB39L | Hs.87159 | 225914_s_at |
| calcium channel, voltage-dependent, P/Q type, | CACNA1A | Hs.408449 | 214933_at |
| alpha 1A subunit | | | |
| calcium homeostasis endoplasmic reticulum | CHERP | Hs.386465 | 1568704_a_at |
| protein | | | |
| calcium modulating ligand | CAMLG | Hs.529846 | 203538_at |
| calcium/calmodulin-dependent protein kinase II | CaMKIIN | Hs.197922 | 218309_at |
| | alpha | | |
| calcium/calmodulin-dependent protein kinase IV | CAMK4 | Hs.220629 | 241871_at |
| calcium/calmodulin-dependent serine protein | CASK | Hs.495984 | 211208_s_at |
| kinase (MAGUK family) | | | |
| calcium-binding tyrosine-(Y)-phosphorylation | CABYR | Hs.511983 | 219928_s_at |
| regulated (fibrousheathin 2) | | | |
| calcium-binding tyrosine-(Y)-phosphorylation | CABYR | Hs.511983 | 224279_s_at |
| regulated (fibrousheathin 2) | | | |
| caldesmon 1 | CALD1 | Hs.490203 | 205525_at |
| caldesmon 1 | CALD1 | Hs.490203 | 212077_at |
| calmin (calponin-like, transmembrane) | CLMN | Hs.301478 | 221042_s_at |
| calmodulin 1 (phosphorylase kinase, delta) | CALM1 | Hs.282410 | 209563_x_at |
| calmodulin 2 (phosphorylase kinase, delta) | CALM2 | Hs.468442 | 207243_s_at |
| Calmodulin 2 (phosphorylase kinase, delta) | CALM2 | Hs.468442 | 229571_at |
| Calmodulin 2 (phosphorylase kinase, delta) | CALM2 | Hs.468442 | 235190_at |
| calmodulin binding transcription activator 1 | CAMTA1 | Hs.397705 | 1555370_a_at |
| calmodulin binding transcription activator 1 | CAMTA1 | Hs.397705 | 225692_at |
| calmodulin binding transcription activator 2 | CAMTA2 | Hs.373952 | 212948_at |
| calmodulin regulated spectrin-associated protein 1 | CAMSAP1 | Hs.522493 | 212710_at |
| calnexin | CANX | Hs.529890 | 208852_s_at |
| calnexin | CANX | Hs.529890 | 208853_s_at |
| calnexin /// calnexin | CANX | Hs.529890 | 200068_s_at |
| calpain 2, (m/II) large subunit | CAPN2 | Hs.350899 | 214888_at |
| calpain 3, (p94) | CAPN3 | Hs.143261 | 210944_s_at |
| Calpain 7 | CAPN7 | Hs.7145 | 239778_x_at |
| calpastatin | CAST | Hs.440961 | 207467_x_at |
| calsequestrin 1 (fast-twitch, skeletal muscle) | CASQ1 | Hs.552568 | 219645_at |
| cAMP responsive element binding protein 1 | CREB1 | Hs.516646 | 204313_s_at |
| cAMP responsive element binding protein 1 | CREB1 | Hs.516646 | 204314_s_at |
| cAMP responsive element binding protein 3-like 2 | CREB3L2 | Hs.490273 | 212345_s_at |
| cAMP responsive element modulator | CREM | Hs.200250 | 228092_at |
| cancer susceptibility candidate 1 | CASC1 | Hs.407771 | 220168_at |
| cancer susceptibility candidate 2 | CASC2 | Hs.89387 | 1564372_s_at |
| cancer susceptibility candidate 5 | CASC5 | Hs.181855 | 228323_at |
| CAP, adenylate cyclase-associated protein 1 | CAP1 | Hs.370581 | 213798_s_at |
| (yeast) | | | |
| CAP-binding protein complex interacting protein 1 | FLJ23588 | Hs.368507 | 1562815_at |
| capping protein (actin filament) muscle Z-line, | CAPZA1 | Hs.514934 | 208374_s_at |
| alpha 1 | | | |
| capping protein (actin filament) muscle Z-line, | CAPZA2 | Hs.446123 | 201238_s_at |
| alpha 2 | | | |
| capping protein (actin filament) muscle Z-line, | CAPZA3 | Hs.131288 | 236888_at |
| alpha 3 | | | |
| capping protein (actin filament) muscle Z-line, beta | CAPZB | Hs.432760 | 37012_at |
| carbamoyl-phosphate synthetase 1, mitochondrial | CPS1 | Hs.149252 | 204920_at |
| carbohydrate (chondroitin) synthase 1 | CHSY1 | Hs.110488 | 203044_at |
| carbohydrate (N-acetylglucosamine 6-O) | CHST5 | Hs.420796 | 64900_at |
| sulfotransferase 5 /// hypothetical protein | /// | | |
| MGC15429 | MGC15429 | | |
| carbonic anhydrase II | CA2 | Hs.155097 | 209301_at |
| carbonic anhydrase XII | CA12 | Hs.210995 | 203963_at |
| carbonic reductase 4 | CBR4 | Hs.481166 | 1553693_s_at |
| Carbonic reductase 4 | CBR4 | Hs.481166 | 232347_x_at |
| carboxylesterase 2 (intestine, liver) | CES2 | Hs.282975 | 209668_x_at |
| carcinoembryonic antigen-related cell adhesion | CEACAM5 | Hs.220529 | 201884_at |
| molecule 5 | | | |
| carcinoembryonic antigen-related cell adhesion | CEACAM6 | Hs.466814 | 211657_at |
| molecule 6 (non-specific cross reacting antigen) /// | | | |
| carcinoembryonic antigen-related cell adhesion | | | |
| molecule 6 (non-specific cross reacting antigen) | | | |
| carcinoembryonic antigen-related cell adhesion | CEACAM7 | Hs.74466 | 206199_at |
| molecule 7 | | | |
| Cardiotrophin-like cytokine factor 1 | CLC | Hs.502977 | 1556385_at |
| carnitine acetyltransferase | CRAT | Hs.12068 | 209522_s_at |
| carnitine deficiency-associated, expressed in | CDV1 | Hs.528382 | 219372_at |
| ventricle 1 | | | |
| carnitine O-octanoyltransferase | CROT | Hs.125039 | 204573_at |
| carnitine palmitoyltransferase 1A (liver) | CPT1A | Hs.503043 | 203634_s_at |
| Cas-Br-M (murine) ecotropic retroviral transforming | CBLB | Hs.430589 | 209682_at |
| sequence b | | | |
| casein kinase 1, alpha 1 | CSNK1A1 | Hs.442592 | 208865_at |
| casein kinase 1, alpha 1 | CSNK1A1 | Hs.442592 | 208866_at |
| casein kinase 1, alpha 1 | CSNK1A1 | Hs.442592 | 213086_s_at |
| casein kinase 1, alpha 1 | CSNK1A1 | Hs.442592 | 213860_x_at |
| casein kinase 1, gamma 1 | CSNK1G1 | Hs.254335 | 221673_s_at |
| casein kinase 1, gamma 2 | CSNK1G2 | Hs.181390 | 202573_at |
| casein kinase 1, gamma 3 | CSNK1G3 | Hs.129206 | 220768_s_at |
| Casein kinase 1, gamma 3 | CSNK1G3 | Hs.129206 | 227767_at |
| casein kinase 2, alpha prime polypeptide | CSNK2A2 | Hs.82201 | 203575_at |
| Casein kinase 2, alpha prime polypeptide | CSNK2A2 | Hs.82201 | 224922_at |
| CASP2 and RIPK1 domain containing adaptor with | CRADD | Hs.38533 | 1563706_at |
| death domain | | | |
| CASP2 and RIPK1 domain containing adaptor with | CRADD | Hs.38533 | 209833_at |
| death domain | | | |
| CASP8 and FADD-like apoptosis regulator | CFLAR | Hs.390736 | 211316_x_at |
| CASP8 and FADD-like apoptosis regulator | CFLAR | Hs.390736 | 239629_at |
| CASP8 and FADD-like apoptosis regulator /// | CFLAR | Hs.390736 | 209508_x_at |
| CASP8 and FADD-like apoptosis regulator | | | |
| caspase 2, apoptosis-related cysteine protease | CASP2 | Hs.368982 | 208050_s_at |
| (neural precursor cell expressed, developmentally | | | |
| down-regulated 2) | | | |
| caspase 2, apoptosis-related cysteine protease | CASP2 | Hs.368982 | 226032_at |
| (neural precursor cell expressed, developmentally | | | |
| down-regulated 2) | | | |
| caspase 6, apoptosis-related cysteine protease | CASP6 | Hs.3280 | 211464_x_at |
| caspase 9, apoptosis-related cysteine protease | CASP9 | Hs.329502 | 203984_s_at |
| caspase 9, apoptosis-related cysteine protease | CASP9 | Hs.329502 | 210775_x_at |
| caspase recruitment domain family, member 10 | CARD10 | Hs.57973 | 210026_s_at |
| cat eye syndrome chromosome region, candidate 5 | CECR5 | Hs.26890 | 218592_s_at |
| catalase | CAT | Hs.502302 | 201432_at |
| Catenin (cadherin-associated protein), alpha 1, | CTNNA1 | Hs.445981 | 240262_at |
| 102 kDa | | | |
| catenin (cadherin-associated protein), alpha 2 | CTNNA2 | Hs.167368 | 205373_at |
| catenin (cadherin-associated protein), alpha-like 1 | CTNNAL1 | Hs.58488 | 202468_s_at |
| Catenin (cadherin-associated protein), beta 1, | CTNNB1 | Hs.476018 | 223679_at |
| 88 kDa | | | |
| catenin (cadherin-associated protein), delta 2 | CTNND2 | Hs.314543 | 209617_s_at |
| (neural plakophilin-related arm-repeat protein) | | | |
| cathepsin B | CTSB | Hs.520898 | 213274_s_at |
| cathepsin K (pycnodysostosis) | CTSK | Hs.523594 | 202450_s_at |
| cathepsin O | CTSO | Hs.75262 | 203758_at |
| cation channel, sperm associated 2 | CATSPER2 | Hs.389181 | 217588_at |
| CCAAT/enhancer binding protein(C/EBP), beta | CEBPB | Hs.517106 | 212501_at |
| CCAAT/enhancer binding protein zeta | CEBPZ | Hs.135406 | 229746_x_at |
| CCR4-NOT transcription complex, subunit 1 | CNOT1 | Hs.460923 | 200860_s_at |
| CCR4-NOT transcription complex, subunit 2 | CNOT2 | Hs.133350 | 222182_s_at |
| CCR4-NOT transcription complex, subunit 2 | CNOT2 | Hs.133350 | 244197_x_at |
| CCR4-NOT transcription complex, subunit 3 | CNOT3 | Hs.343571 | 229143_at |
| CCR4-NOT transcription complex, subunit 6-like | CNOT6L | Hs.183085 | 226153_s_at |
| CCR4-NOT transcription complex, subunit 7 | CNOT7 | Hs.170553 | 218250_s_at |
| CCR4-NOT transcription complex, subunit 8 | CNOT8 | Hs.26703 | 202162_s_at |
| CD109 antigen (Gov platelet alloantigens) | CD109 | Hs.399891 | 229900_at |
| CD164 antigen, sialomucin | CD164 | Hs.520313 | 208405_s_at |
| CD164 antigen, sialomucin | CD164 | Hs.520313 | 208653_s_at |
| CD164 antigen, sialomucin | CD164 | Hs.520313 | 208654_s_at |
| CD2 antigen (p50), sheep red blood cell receptor | CD2 | Hs.523500 | 205831_at |
| /// CD2 antigen (p50), sheep red blood cell | | | |
| receptor | | | |
| CD24 antigen (small cell lung carcinoma cluster 4 | CD24 | Hs.375108 | 208650_s_at |
| antigen) | | | |
| CD24 antigen (small cell lung carcinoma cluster 4 | CD24 | Hs.375108 | 209771_x_at |
| antigen) | | | |
| CD24 antigen (small cell lung carcinoma cluster 4 | CD24 | Hs.375108 | 216379_x_at |
| antigen) | | | |
| CD24 antigen (small cell lung carcinoma cluster 4 | CD24 | Hs.375108 | 266_s_at |
| antigen) | | | |
| CD274 antigen | CD274 | Hs.521989 | 223834_at |
| CD27-binding (Siva) protein | SIVA | Hs.112058 | 222030_at |
| CD36 antigen (collagen type I receptor, | CD36 | Hs.120949 | 236923_x_at |
| thrombospondin receptor) | | | |
| CD40 antigen (TNF receptor superfamily member | CD40 | Hs.472860 | 35150_at |
| 5) | | | |
| CD47 antigen (Rh-related antigen, integrin- | CD47 | Hs.446414 | 213857_s_at |
| associated signal transducer) | | | |
| CD47 antigen (Rh-related antigen, integrin- | CD47 | Hs.446414 | 211075_s_at |
| associated signal transducer) /// CD47 antigen | | | |
| (Rh-related antigen, integrin-associated signal | | | |
| transducer) | | | |
| CD53 antigen | CD53 | Hs.443057 | 203416_at |
| CD58 antigen, (lymphocyte function-associated | CD58 | Hs.34341 | 243931_at |
| antigen 3) | | | |
| CD59 antigen p18-20 (antigen identified by | CD59 | Hs.278573 | 200983_x_at |
| monoclonal antibodies 16.3A5, EJ16, EJ30, EL32 | | | |
| and G344) | | | |
| CD59 antigen p18-20 (antigen identified by | CD59 | Hs.278573 | 200984_s_at |
| monoclonal antibodies 16.3A5, EJ16, EJ30, EL32 | | | |
| and G344) | | | |
| CD80 antigen (CD28 antigen ligand 1, B7-1 | CD80 | Hs.838 | 207176_s_at |
| antigen) | | | |
| CD9 antigen (p24) | CD9 | Hs.114286 | 201005_at |
| CD99 antigen | CD99 | Hs.495605 | 201029_s_at |
| CDC14 cell division cycle 14 homolog A (S. cerevisiae) | CDC14A | Hs.533582 | 243640_x_at |
| CDC14 cell division cycle 14 homolog B (S. cerevisiae) | CDC14B | Hs.40582 | 221555_x_at |
| CDC14 cell division cycle 14 homolog B (S. cerevisiae) | CDC14B | Hs.40582 | 221556_at |
| CDC14 cell division cycle 14 homolog B (S. cerevisiae) | CDC14B | Hs.40582 | 234605_at |
| CDC14 cell division cycle 14 homolog B (S. cerevisiae) | CDC14B | Hs.40582 | 208022_s_at |
| /// CDC14 cell division cycle 14 | | | |
| homolog B (S. cerevisiae) | | | |
| CDC16 cell division cycle 16 homolog (S. cerevisiae) | CDC16 | Hs.374127 | 202717_s_at |
| CDC23 (cell division cycle 23, yeast, homolog) | CDC23 | Hs.153546 | 202892_at |
| CDC28 protein kinase regulatory subunit 1B | CKS1B | Hs.374378 | 201897_s_at |
| CDC28 protein kinase regulatory subunit 2 | CKS2 | Hs.83758 | 204170_s_at |
| CDC2-related protein kinase 7 | CRK7 | Hs.416108 | 213557_at |
| CDC2-related protein kinase 7 | CRK7 | Hs.416108 | 219226_at |
| CDC42 binding protein kinase alpha (DMPK-like) | CDC42BPA | Hs.35433 | 214464_at |
| CDC42 effector protein (Rho GTPase binding) 3 | CDC42EP3 | Hs.369574 | 225685_at |
| CDC42 small effector 1 | CDC42SE1 | Hs.22065 | 222537_s_at |
| CDC42 small effector 1 | CDC42SE1 | Hs.22065 | 229120_s_at |
| CDC5 cell division cycle 5-like (S. pombe) | CDC5L | Hs.485471 | 209056_s_at |
| CDC5 cell division cycle 5-like (S. pombe) | CDC5L | Hs.485471 | 209057_x_at |
| CDC-like kinase 1 /// CDC-like kinase 4 | CLK1 /// | Hs.433732 | 210346_s_at |
| | CLK4 | | |
| CDC-like kinase 4 | CLK4 | Hs.406557 | 241403_at |
| CDK2-associated protein 1 | CDK2AP1 | Hs.433201 | 201938_at |
| CDNA clone IMAGE: 2988896 | — | Hs.158545 | 1566228_at |
| CDNA clone IMAGE: 3346533, containing frame- | — | Hs.478854 | 243293_at |
| shift errors | | | |
| CDNA clone IMAGE: 3542720 | — | Hs.547037 | 226025_at |
| CDNA clone IMAGE: 3895112, containing frame- | — | Hs.552683 | 1570165_at |
| shift errors | | | |
| CDNA clone IMAGE: 3944752, partial cds | — | Hs.437283 | 1555206_at |
| CDNA clone IMAGE: 4151535, partial cds | — | Hs.535917 | 1560826_at |
| CDNA clone IMAGE: 4288461, partial cds | — | Hs.270098 | 237581_at |
| CDNA clone IMAGE: 4333081, partial cds | — | Hs.336989 | 239376_at |
| CDNA clone IMAGE: 4557810, partial cds | — | Hs.98587 | 236098_at |
| CDNA clone IMAGE: 4620359, partial cds | — | Hs.418285 | 1568813_at |
| CDNA clone IMAGE: 4698949, partial cds | — | Hs.551727 | 234020_x_at |
| CDNA clone IMAGE: 4753178, containing frame- | — | Hs.552097 | 1570567_at |
| shift errors | | | |
| CDNA clone IMAGE: 4791593, partial cds | — | Hs.147878 | 1559102_at |
| CDNA clone IMAGE: 4792502, partial cds | — | Hs.283396 | 241487_at |
| CDNA clone IMAGE: 4795866, partial cds | — | Hs.513661 | 233017_x_at |
| CDNA clone IMAGE: 4796102, partial cds | — | Hs.539926 | 1561604_at |
| CDNA clone IMAGE: 4797120, partial cds | — | Hs.537004 | 226671_at |
| CDNA clone IMAGE: 4815736, partial cds | — | Hs.21816 | 228494_at |
| CDNA clone IMAGE: 4829245, partial cds | — | Hs.130189 | 231485_at |
| CDNA clone IMAGE: 4841343, partial cds | — | Hs.86508 | 230672_at |
| CDNA clone IMAGE: 5169389, with apparent | — | Hs.380689 | 237794_at |
| retained intron | | | |
| CDNA clone IMAGE: 5206119, partial cds | — | Hs.318639 | 238953_at |
| CDNA clone IMAGE: 5217021, with apparent | — | Hs.550698 | 221973_at |
| retained intron | | | |
| CDNA clone IMAGE: 5243069, partial cds | — | Hs.535500 | 212049_at |
| CDNA clone IMAGE: 5259778, partial cds | — | Hs.97129 | 236349_at |
| CDNA clone IMAGE: 5263531, partial cds | — | Hs.399763 | 224778_s_at |
| CDNA clone IMAGE: 5265199, partial cds | — | Hs.538599 | 1566504_at |
| CDNA clone IMAGE: 5265356, partial cds | — | Hs.180330 | 1556976_s_at |
| CDNA clone IMAGE: 5265747, partial cds | — | Hs.126405 | 1555995_a_at |
| CDNA clone IMAGE: 5266242, partial cds | — | Hs.187018 | 238559_at |
| CDNA clone IMAGE: 5266307, partial cds | — | Hs.520453 | 1557891_s_at |
| CDNA clone IMAGE: 5266689, partial cds | — | Hs.140444 | 241144_at |
| CDNA clone IMAGE: 5268125, partial cds | — | Hs.461412 | 1560957_at |
| CDNA clone IMAGE: 5272727, partial cds | — | Hs.379472 | 1566809_a_at |
| CDNA clone IMAGE: 5277089, partial cds | — | Hs.547150 | 238244_at |
| CDNA clone IMAGE: 5277680, partial cds | — | Hs.381993 | 1566763_at |
| CDNA clone IMAGE: 5286005, partial cds | — | Hs.549531 | 242364_x_at |
| CDNA clone IMAGE: 5286843, partial cds | — | Hs.302631 | 235174_s_at |
| CDNA clone IMAGE: 5287121, partial cds | — | Hs.375744 | 243904_at |
| CDNA clone IMAGE: 5295357, partial cds | — | Hs.382012 | 1560954_at |
| CDNA clone IMAGE: 5296106, partial cds | — | Hs.548301 | 1556834_at |
| CDNA clone IMAGE: 5296106, partial cds | — | Hs.548301 | 1556835_s_at |
| CDNA clone IMAGE: 5296164, partial cds | — | Hs.369473 | 237272_at |
| CDNA clone IMAGE: 5296164, partial cds | — | Hs.369473 | 237734_s_at |
| CDNA clone IMAGE: 5296164, partial cds | — | Hs.369473 | 237735_at |
| CDNA clone IMAGE: 5297843, partial cds | — | Hs.398070 | 1563241_at |
| CDNA clone IMAGE: 5742524, partial cds | — | Hs.147170 | 237738_at |
| CDNA clone IMAGE: 5744875, partial cds | — | Hs.333059 | 1560638_a_at |
| CDNA clone IMAGE: 5756190, partial cds | — | Hs.446324 | 229720_at |
| CDNA FLJ11397 fis, clone HEMBA1000622 | — | Hs.470871 | 236251_at |
| CDNA FLJ11489 fis, clone HEMBA1001915 | — | Hs.526415 | 228661_s_at |
| CDNA FLJ11723 fis, clone HEMBA1005314 | — | Hs.196008 | 227497_at |
| CDNA FLJ11900 fis, clone HEMBA1007341 | — | Hs.550165 | 233214_at |
| CDNA FLJ11994 fis, clone HEMBB1001436 | — | Hs.156518 | 232834_at |
| CDNA FLJ12030 fis, clone HEMBB1001868 | — | Hs.160572 | 232439_at |
| CDNA FLJ12302 fis, clone MAMMA1001864 | — | Hs.287512 | 233027_at |
| CDNA FLJ13267 fis, clone OVARC1000964 /// | — | Hs.57079 | 212829_at |
| CDNA FLJ13267 fis, clone OVARC1000964 | | | |
| CDNA FLJ13313 fis, clone OVARC1001489 | — | Hs.287562 | 233249_at |
| CDNA FLJ13876 fis, clone THYRO1001401 | — | Hs.471778 | 239379_at |
| CDNA FLJ20009 fis, clone ADKA03183 | — | Hs.544189 | 1566938_at |
| CDNA FLJ23675 fis, clone HEP08462 | — | Hs.352657 | 1563884_at |
| CDNA FLJ25684 fis, clone TST04185 | — | Hs.210013 | 242824_at |
| CDNA FLJ25790 fis, clone TST06909 | — | Hs.149769 | 240938_at |
| CDNA FLJ25794 fis, clone TST07014 | — | Hs.122417 | 240216_at |
| CDNA FLJ25810 fis, clone TST07303 | — | Hs.535454 | 231627_at |
| CDNA FLJ25849 fis, clone TST08968 | — | Hs.535610 | 1556671_s_at |
| CDNA FLJ26073 fis, clone RCT01314 | — | Hs.521235 | 222090_at |
| CDNA FLJ26120 fis, clone SYN00419 | — | Hs.433995 | 225239_at |
| CDNA FLJ26461 fis, clone KDN03867 | — | Hs.191850 | 239600_at |
| CDNA FLJ30340 fis, clone BRACE2007411 | — | Hs.7572 | 226457_at |
| CDNA FLJ30541 fis, clone BRAWH2001355 | — | Hs.533401 | 239333_x_at |
| CDNA FLJ30779 fis, clone FEBRA2000815 | — | Hs.4204 | 228329_at |
| CDNA FLJ30913 fis, clone FEBRA2006354 | — | Hs.344170 | 1561369_at |
| CDNA FLJ31511 fis, clone NT2RI1000035 | — | Hs.193191 | 242966_x_at |
| CDNA FLJ31513 fis, clone NT2RI1000127 | — | Hs.30977 | 228088_at |
| CDNA FLJ31626 fis, clone NT2RI2003317 | — | Hs.520251 | 217833_at |
| CDNA FLJ31750 fis, clone NT2RI2007406 | — | Hs.27048 | 226049_at |
| CDNA FLJ32412 fis, clone SKMUS2000690 | — | Hs.348514 | 225492_at |
| CDNA FLJ32420 fis, clone SKMUS2000898 | — | Hs.439563 | 1559867_at |
| CDNA FLJ32429 fis, clone SKMUS2001014 | — | Hs.508823 | 240344_x_at |
| CDNA FLJ32438 fis, clone SKMUS2001402 | — | Hs.552759 | 228275_at |
| CDNA FLJ32438 fis, clone SKMUS2001402 | — | Hs.552759 | 236798_at |
| CDNA FLJ32664 fis, clone TESTI1000088 | — | Hs.123393 | 1566823_a_at |
| CDNA FLJ32691 fis, clone TESTI2000221 | — | Hs.319071 | 1561346_at |
| CDNA FLJ32774 fis, clone TESTI2002002 | — | Hs.350592 | 237344_at |
| CDNA FLJ32878 fis, clone TESTI2004111 | — | Hs.99510 | 238188_at |
| CDNA FLJ32967 fis, clone TESTI2008715 | — | Hs.498479 | 1552727_s_at |
| CDNA FLJ33255 fis, clone ASTRO2005553 | — | Hs.289958 | 1557302_at |
| CDNA FLJ33351 fis, clone BRACE2005063 | — | Hs.27096 | 1560924_at |
| CDNA FLJ33443 fis, clone BRALZ1000103 | — | Hs.531632 | 244259_s_at |
| CDNA FLJ34029 fis, clone FCBBF2003636 | — | Hs.372457 | 235800_at |
| CDNA FLJ34374 fis, clone FEBRA2017502 | — | Hs.432315 | 236856_x_at |
| CDNA FLJ34585 fis, clone KIDNE2008758 | — | Hs.202577 | 226250_at |
| CDNA FLJ34585 fis, clone KIDNE2008758 | — | Hs.202577 | 226252_at |
| CDNA FLJ34896 fis, clone NT2NE2018180 | — | Hs.507978 | 225356_at |
| CDNA FLJ35001 fis, clone OCBBF2011887 | — | Hs.107418 | 227579_at |
| CDNA FLJ35886 fis, clone TESTI2009091 | — | Hs.253209 | 1561415_at |
| CDNA FLJ35886 fis, clone TESTI2009091 | — | Hs.253209 | 1561417_x_at |
| CDNA FLJ35982 fis, clone TESTI2013604 | — | Hs.129543 | 1562671_s_at |
| CDNA FLJ36008 fis, clone TESTI2015647 | — | Hs.544798 | 1557222_at |
| CDNA FLJ36043 fis, clone TESTI2017582 | — | Hs.269382 | 1559316_at |
| CDNA FLJ36183 fis, clone TESTI2026854 | — | Hs.86636 | 243783_at |
| CDNA FLJ36375 fis, clone THYMU2008226 | — | Hs.396544 | 1557270_at |
| CDNA FLJ36734 fis, clone UTERU2012890 | — | Hs.549896 | 1559467_at |
| CDNA FLJ37010 fis, clone BRACE2009732 | — | Hs.547908 | 1562598_at |
| CDNA FLJ37115 fis, clone BRACE2022158 | — | Hs.364299 | 1562849_at |
| CDNA FLJ37485 fis, clone BRAWH2014379 | — | Hs.24181 | 228314_at |
| CDNA FLJ38107 fis, clone D3OST2001669 | — | Hs.145411 | 240435_at |
| CDNA FLJ39459 fis, clone PROST2011439 | — | Hs.491069 | 222368_at |
| CDNA FLJ39947 fis, clone SPLEN2024232 | — | Hs.550177 | 1557116_at |
| CDNA FLJ40450 fis, clone TESTI2040944 | — | Hs.550440 | 1564338_at |
| CDNA FLJ40764 fis, clone TRACH2002954 | — | Hs.482589 | 1562009_a_at |
| CDNA FLJ40901 fis, clone UTERU2003704 | — | Hs.44898 | 227663_at |
| CDNA FLJ41270 fis, clone BRAMY2036387 | — | Hs.445414 | 228528_at |
| CDNA FLJ41452 fis, clone BRSTN2010363 | — | Hs.530543 | 227922_x_at |
| CDNA FLJ42077 fis, clone SYNOV2019280 | — | Hs.444521 | 236776_at |
| CDNA FLJ42310 fis, clone TRACH2007733 | — | Hs.444400 | 239718_at |
| CDNA FLJ42313 fis, clone TRACH2019425 | — | Hs.386042 | 235028_at |
| CDNA FLJ42357 fis, clone UTERU2022773 | — | Hs.547723 | 216527_at |
| CDNA FLJ43181 fis, clone FCBBF3016134 | — | Hs.98553 | 243007_at |
| CDNA FLJ45384 fis, clone BRHIP3021987 | — | Hs.61558 | 229699_at |
| CDNA FLJ46224 fis, clone TESTI4014262 | — | Hs.542318 | 240507_at |
| CDNA FLJ90571 fis, clone OVARC1001725, highly | — | Hs.23754 | 227745_at |
| similar to Homo sapiens patched related protein | | | |
| TRC8 (TRC8) gene. | | | |
| cDNA sequence BC022357 | BC022357 | — | 224957_at |
| CDNA: FLJ20874 fis, clone ADKA02818 | — | Hs.539236 | 216739_at |
| CDNA: FLJ20891 fis, clone ADKA03345 | — | Hs.537488 | 1566954_at |
| CDNA: FLJ20892 fis, clone ADKA03430 | — | Hs.386421 | 230683_at |
| CDNA: FLJ20908 fis, clone ADSE00417 | — | Hs.545857 | 216730_at |
| CDNA: FLJ20946 fis, clone ADSE01819 | — | Hs.306696 | 233702_x_at |
| CDNA: FLJ20949 fis, clone ADSE01902 | — | Hs.380443 | 222207_x_at |
| CDNA: FLJ21226 fis, clone COL00721 | — | Hs.550156 | 216751_at |
| CDNA: FLJ21228 fis, clone COL00739 | — | Hs.306716 | 234723_x_at |
| CDNA: FLJ21452 fis, clone COL04505 | — | Hs.502812 | 232476_at |
| CDNA: FLJ21881 fis, clone HEP02746 | — | Hs.550197 | 232876_at |
| CDNA: FLJ22144 fis, clone HEP21732 | — | Hs.413944 | 233428_at |
| CDNA: FLJ22256 fis, clone HRC02860 | — | Hs.288741 | 214036_at |
| CDNA: FLJ22539 fis, clone HRC13227 | — | Hs.17519 | 227491_at |
| CDNA: FLJ22769 fis, clone KAIA1316 | — | Hs.120127 | 232781_at |
| CDNA: FLJ22994 fis, clone KAT11918 | — | Hs.98073 | 227792_at |
| CDNA: FLJ23566 fis, clone LNG10880 | — | Hs.532596 | 234675_x_at |
| CDP-diacylglycerol synthase (phosphatidate | CDS1 | Hs.444924 | 226185_at |
| cytidylyltransferase) 1 | | | |
| CDP-diacylglycerol-inositol 3- | CDIPT | Hs.121549 | 201253_s_at |
| phosphatidyltransferase (phosphatidylinositol | | | |
| synthase) | | | |
| cell death-regulatory protein GRIM19 | GRIM19 | Hs.534453 | 220864_s_at |
| cell division cycle 25A | CDC25A | Hs.1634 | 1555772_a_at |
| cell division cycle 26 | CDC26 | Hs.530284 | 225422_at |
| cell division cycle 27 | CDC27 | Hs.463295 | 217879_at |
| cell division cycle 2-like 1 (PITSLRE proteins) /// | CDC2L1 | Hs.192316 | 212401_s_at |
| cell division cycle 2-like 2 (PITSLRE proteins) | /// | | |
| | CDC2L2 | | |
| Cell division cycle 2-like 5 (cholinesterase-related | CDC2L5 | Hs.233552 | 232266_x_at |
| cell division controller) | | | |
| cell division cycle 2-like 6 (CDK8-like) | CDC2L6 | Hs.193251 | 212899_at |
| cell division cycle 2-like 6 (CDK8-like) | CDC2L6 | Hs.193251 | 235226_at |
| cell division cycle 37 homolog (S. cerevisiae)-like 1 | CDC37L1 | Hs.493361 | 219343_at |
| cell division cycle 40 homolog (yeast) | CDC40 | Hs.428147 | 203377_s_at |
| cell division cycle and apoptosis regulator 1 | CCAR1 | Hs.49853 | 224736_at |
| cell division cycle associated 3 | CDCA3 | Hs.524216 | 223307_at |
| cell growth regulator with ring finger domain 1 | CGRRF1 | Hs.59106 | 204605_at |
| Centaurin, beta 1 | CENTB1 | Hs.337242 | 242081_at |
| Centaurin, beta 2 | CENTB2 | Hs.478746 | 240759_at |
| centaurin, gamma 2 | CENTG2 | Hs.435039 | 204066_s_at |
| Centaurin, gamma-like family, member 1 | CTGLF1 | Hs.499496 | 1565620_at |
| centaurin, gamma-like family, member 1 | CTGLF1 | Hs.499496 | 221850_x_at |
| centromere protein J | CENPJ | Hs.533828 | 223513_at |
| centrosome protein cep290 | Cep290 | Hs.150444 | 205250_s_at |
| centrosome protein Cep63 | Cep63 | Hs.443301 | 222151_s_at |
| centrosome spindle pole associated protein | CSPP | Hs.370147 | 220072_at |
| centrosome spindle pole associated protein | CSPP | Hs.370147 | 242041_at |
| centrosome-associated protein 350 | CAP350 | Hs.413045 | 204373_s_at |
| centrosome-associated protein 350 | CAP350 | Hs.413045 | 213956_at |
| cerebellin 1 precursor | CBLN1 | Hs.458423 | 205747_at |
| cereblon | CRBN | Hs.18925 | 222533_at |
| ceroid-lipofuscinosis, neuronal 5 | CLN5 | Hs.30213 | 204084_s_at |
| CG016 | LOC88523 | — | 214748_at |
| CG10806-like | LOC150159 | Hs.445192 | 1553633_s_at |
| CG10806-like | LOC150159 | Hs.445192 | 1555141_a_at |
| CGG triplet repeat binding protein 1 | CGGBP1 | Hs.444818 | 224600_at |
| CGI-119 protein | CGI-119 | Hs.505934 | 219206_x_at |
| CGI-119 protein | CGI-119 | Hs.505934 | 222845_x_at |
| CGI-119 protein | CGI-119 | Hs.505934 | 223892_s_at |
| CGI-146 protein | PNAS-4 | Hs.498317 | 1558914_at |
| CGI-146 protein | PNAS-4 | Hs.498317 | 212371_at |
| CGI-146 protein | PNAS-4 | Hs.498317 | 221648_s_at |
| CGI-146 protein | PNAS-4 | Hs.498317 | 222158_s_at |
| CGI-96 protein /// similar to CGI-96 | CGI-96 | Hs.550515 | 202938_x_at |
| | /// | | |
| | dJ222E13.2 | | |
| CH5400 | — | Hs.325100 | 237475_x_at |
| chaperonin containing TCP1, subunit 2 (beta) | CCT2 | Hs.189772 | 201947_s_at |
| chaperonin containing TCP1, subunit 6B (zeta 2) | CCT6B | Hs.73072 | 206587_at |
| chaperonin containing TCP1, subunit 8 (theta) | CCT8 | Hs.125113 | 200873_s_at |
| Chaperonin containing TCP1, subunit 8 (theta) | CCT8 | Hs.125113 | 242380_at |
| Chemokine (C—C motif) ligand 27 | CCL27 | Hs.459590 | 230327_at |
| chemokine (C—X—C motif) ligand 1 (melanoma | CXCL1 | Hs.789 | 204470_at |
| growth stimulating activity, alpha) | | | |
| chemokine (C—X—C motif) ligand 10 | CXCL10 | Hs.413924 | 204533_at |
| chemokine (C—X—C motif) ligand 12 (stromal cell- | CXCL12 | Hs.522891 | 203666_at |
| derived factor 1) | | | |
| chemokine orphan receptor 1 | CMKOR1 | Hs.471751 | 212977_at |
| chemokine-like factor super family 1 | CKLFSF1 | Hs.549232 | 231219_at |
| chemokine-like factor super family 2 | CKLFSF2 | Hs.195685 | 229967_at |
| chemokine-like factor super family 6 | CKLFSF6 | Hs.380627 | 217947_at |
| chemokine-like factor super family 6 | CKLFSF6 | Hs.380627 | 223047_at |
| CHK1 checkpoint homolog (S. pombe) | CHEK1 | Hs.24529 | 205394_at |
| Chloride channel 3 | CLCN3 | Hs.481186 | 201734_at |
| chloride channel, nucleotide-sensitive, 1A /// | CLNS1A | Hs.107393 | 209143_s_at |
| chromosome 3 open reading frame 4 | /// C3orf4 | | |
| choline dehydrogenase | CHDH | Hs.126688 | 1559591_s_at |
| choline kinase beta /// carnitine | CHKB /// | Hs.439777 | 210069_at |
| palmitoyltransferase 1B (muscle) | CPT1B | | |
| choline kinase beta /// carnitine | CHKB /// | Hs.439777 | 210070_s_at |
| palmitoyltransferase 1B (muscle) | CPT1B | | |
| choline phosphotransferase 1 | CHPT1 | Hs.293077 | 221675_s_at |
| chondroitin sulfate GalNAcT-2 | GALNACT-2 | Hs.180758 | 218871_x_at |
| chondroitin sulfate proteoglycan 2 (versican) | CSPG2 | Hs.443681 | 204620_s_at |
| chondroitin sulfate proteoglycan 2 (versican) | CSPG2 | Hs.443681 | 221731_x_at |
| chondroitin sulfate proteoglycan 6 | CSPG6 | Hs.24485 | 209259_s_at |
| chromobox homolog 1 (HP1 beta homolog | CBX1 | Hs.77254 | 201518_at |
| Drosophila) | | | |
| chromobox homolog 3 (HP1 gamma homolog, | CBX3 | Hs.381189 | 201091_s_at |
| Drosophila) | | | |
| chromobox homolog 3 (HP1 gamma homolog, | CBX3 | Hs.381189 | 200037_s_at |
| Drosophila) /// chromobox homolog 3 (HP1 gamma | | | |
| homolog, Drosophila) | | | |
| chromobox homolog 4 (Pc class homolog, | CBX4 | Hs.405046 | 227558_at |
| Drosophila) | | | |
| chromobox homolog 5 (HP1 alpha homolog, | CBX5 | Hs.349283 | 209715_at |
| Drosophila) | | | |
| chromobox homolog 6 | CBX6 | Hs.511952 | 202047_s_at |
| chromodomain helicase DNA binding protein 1 | CHD1 | Hs.519474 | 204258_at |
| Chromodomain helicase DNA binding protein 1 | CHD1 | Hs.519474 | 235791_x_at |
| chromodomain helicase DNA binding protein 1-like | CHD1L | Hs.191164 | 212539_at |
| chromodomain helicase DNA binding protein 2 | CHD2 | Hs.546244 | 1554014_at |
| chromodomain helicase DNA binding protein 5 | CHD5 | Hs.522898 | 217250_s_at |
| chromodomain helicase DNA binding protein 9 | CHD9 | Hs.530936 | 212616_at |
| chromodomain helicase DNA binding protein 9 | CHD9 | Hs.530936 | 229586_at |
| Chromodomain protein, Y-like | CDYL | Hs.269092 | 240594_at |
| chromodomain protein, Y-like 2 | CDYL2 | Hs.373908 | 1553647_at |
| chromosome 1 open reading frame 16 | C1orf16 | Hs.270775 | 201794_s_at |
| chromosome 1 open reading frame 16 | C1orf16 | Hs.270775 | 217189_s_at |
| chromosome 1 open reading frame 22 | C1orf22 | Hs.523811 | 223243_s_at |
| chromosome 1 open reading frame 24 | C1orf24 | Hs.518662 | 217967_s_at |
| chromosome 1 open reading frame 25 | C1orf25 | Hs.107149 | 223404_s_at |
| chromosome 1 open reading frame 34 | C1orf34 | Hs.112949 | 210652_s_at |
| chromosome 1 open reading frame 37 | C1orf37 | Hs.497443 | 212165_at |
| chromosome 1 open reading frame 43 | C1orf43 | Hs.287471 | 223034_s_at |
| Chromosome 1 open reading frame 43 | C1orf43 | Hs.287471 | 233248_at |
| chromosome 1 open reading frame 48 | C1orf48 | Hs.497692 | 209484_s_at |
| Chromosome 1 open reading frame 48 | C1orf48 | Hs.497692 | 235799_at |
| chromosome 1 open reading frame 8 | C1orf8 | Hs.523262 | 200620_at |
| chromosome 1 open reading frame 9 | C1orf9 | Hs.204559 | 203429_s_at |
| chromosome 10 open reading frame 103 | C10orf103 | — | 1557827_at |
| chromosome 10 open reading frame 104 | C10orf104 | Hs.426296 | 224664_at |
| chromosome 10 open reading frame 104 | C10orf104 | Hs.426296 | 224667_x_at |
| chromosome 10 open reading frame 104 | C10orf104 | Hs.426296 | 229145_at |
| Chromosome 10 open reading frame 116 | C10orf116 | Hs.74120 | 232300_at |
| chromosome 10 open reading frame 118 | C10orf118 | Hs.159066 | 219844_at |
| chromosome 10 open reading frame 12 | C10orf12 | Hs.14555 | 226892_at |
| chromosome 10 open reading frame 128 | C10orf128 | Hs.385493 | 1561890_at |
| chromosome 10 open reading frame 137 | C10orf137 | Hs.468688 | 213410_at |
| chromosome 10 open reading frame 22 | C10orf22 | Hs.99821 | 212500_at |
| chromosome 10 open reading frame 22 | C10orf22 | Hs.99821 | 212502_at |
| chromosome 10 open reading frame 35 | C10orf35 | Hs.522992 | 226313_at |
| chromosome 10 open reading frame 45 | C10orf45 | Hs.446315 | 223058_at |
| chromosome 10 open reading frame 45 | C10orf45 | Hs.446315 | 223059_s_at |
| chromosome 10 open reading frame 51 | C10orf51 | Hs.529965 | 231196_x_at |
| chromosome 10 open reading frame 53 | C10orf53 | Hs.131287 | 239998_at |
| chromosome 10 open reading frame 58 | C10orf58 | Hs.500333 | 228155_at |
| chromosome 10 open reading frame 59 | C10orf59 | Hs.149849 | 223824_at |
| Chromosome 10 open reading frame 61 | C10orf61 | Hs.438991 | 244347_at |
| chromosome 10 open reading frame 65 | C10orf65 | Hs.180346 | 239093_at |
| chromosome 10 open reading frame 68 | C10orf68 | — | 220749_at |
| chromosome 10 open reading frame 70 | C10orf70 | Hs.370102 | 218597_s_at |
| chromosome 10 open reading frame 78 | C10orf78 | Hs.93667 | 236027_at |
| chromosome 10 open reading frame 82 | C10orf82 | Hs.121347 | 231609_at |
| chromosome 10 open reading frame 84 | C10orf84 | Hs.372309 | 222617_s_at |
| chromosome 10 open reading frame 9 | C10orf9 | Hs.14745 | 224651_at |
| chromosome 10 open reading frame 9 | C10orf9 | Hs.14745 | 224652_at |
| chromosome 11 open reading frame 17 /// | C11orf17 | Hs.131180 | 220987_s_at |
| chromosome 11 open reading frame 17 /// likely | /// | | |
| ortholog of rat SNF1/AMP-activated protein kinase | SNARK | | |
| /// likely ortholog of rat SNF1/AMP-activated | | | |
| protein kinase | | | |
| chromosome 12 open reading frame 11 | C12orf11 | Hs.505077 | 221652_s_at |
| chromosome 12 open reading frame 2 | C12orf2 | Hs.269941 | 235996_at |
| chromosome 12 open reading frame 4 | C12orf4 | Hs.302977 | 218374_s_at |
| chromosome 12 open reading frame 4 | C12orf4 | Hs.302977 | 222613_at |
| chromosome 13 open reading frame 10 | C13orf10 | Hs.131715 | 218422_s_at |
| chromosome 13 open reading frame 10 | C13orf10 | Hs.131715 | 222626_at |
| Chromosome 13 open reading frame 10 | C13sorf10 | Hs.131715 | 229433_at |
| chromosome 13 open reading frame 11 | C13orf11 | Hs.317593 | 220241_at |
| chromosome 13 open reading frame 12 | C13orf12 | Hs.268742 | 217769_s_at |
| chromosome 13 open reading frame 22 | C13orf22 | Hs.533831 | 204190_at |
| chromosome 13 open reading frame 3 | C13orf3 | Hs.88523 | 227165_at |
| chromosome 14 open reading frame 101 | C14orf101 | Hs.549170 | 219757_s_at |
| chromosome 14 open reading frame 101 | C14orf101 | Hs.549170 | 225675_at |
| Chromosome 14 open reading frame 103 | C14orf103 | Hs.168241 | 226684_at |
| chromosome 14 open reading frame 108 | C14orf108 | Hs.528653 | 218139_s_at |
| chromosome 14 open reading frame 111 | C14orf111 | Hs.343173 | 215567_at |
| Chromosome 14 open reading frame 112 | C14orf112 | Hs.137108 | 217645_at |
| chromosome 14 open reading frame 118 | C14orf118 | Hs.410231 | 229520_s_at |
| Chromosome 14 open reading frame 119 | C14orf119 | Hs.525238 | 243249_at |
| chromosome 14 open reading frame 131 | C14orf131 | Hs.106005 | 219086_at |
| chromosome 14 open reading frame 143 | C14orf143 | Hs.123232 | 214264_s_at |
| chromosome 14 open reading frame 148 | C14orf148 | Hs.97556 | 231566_at |
| chromosome 14 open reading frame 150 | C14orf150 | Hs.497301 | 238650_x_at |
| Chromosome 14 open reading frame 153 | C14orf153 | Hs.116029 | 232814_x_at |
| chromosome 14 open reading frame 154 | C14orf154 | Hs.510407 | 229940_at |
| chromosome 14 open reading frame 156 /// | C14orf156 | Hs.324521 | 221434_s_at |
| chromosome 14 open reading frame 156 | | | |
| chromosome 14 open reading frame 161 | C14orf161 | Hs.131755 | 220293_at |
| chromosome 14 open reading frame 166 | C14orf166 | Hs.534457 | 217768_at |
| chromosome 14 open reading frame 169 | C14orf169 | Hs.509916 | 219526_at |
| chromosome 14 open reading frame 174 | C14orf174 | Hs.298227 | 232316_at |
| chromosome 14 open reading frame 32 | C14orf32 | Hs.437831 | 212643_at |
| chromosome 14 open reading frame 35 | C14orf35 | Hs.165465 | 225679_at |
| chromosome 14 open reading frame 4 | C14orf4 | Hs.179260 | 223474_at |
| chromosome 14 open reading frame 43 | C14orf43 | Hs.509923 | 225980_at |
| chromosome 14 open reading frame 45 | C14orf45 | Hs.260555 | 220173_at |
| Chromosome 14 open reading frame 46 | C14orf46 | Hs.513392 | 242409_at |
| chromosome 14 open reading frame 48 | C14orf48 | Hs.143845 | 1570111_at |
| chromosome 14 open reading frame 49 | C14orf49 | Hs.354645 | 1553644_at |
| chromosome 14 open reading frame 50 | C14orf50 | Hs.144696 | 237654_at |
| chromosome 14 open reading frame 58 | C14orf58 | Hs.509966 | 222866_s_at |
| chromosome 14 open reading frame 8 | C14orf8 | Hs.406966 | 231140_at |
| chromosome 14 open reading frame 92 | C14orf92 | Hs.194035 | 201684_s_at |
| chromosome 15 open reading frame 23 | C15orf23 | Hs.525796 | 225300_at |
| chromosome 15 open reading frame 25 | C15orf25 | Hs.14347 | 220071_x_at |
| chromosome 15 open reading frame 27 | C15orf27 | Hs.458965 | 1552400_a_at |
| chromosome 15 open reading frame 29 | C15orf29 | Hs.510938 | 1559658_at |
| chromosome 16 open reading frame 34 | C16orf34 | Hs.513261 | 212109_at |
| chromosome 16 open reading frame 34 | C16orf34 | Hs.513261 | 212115_at |
| chromosome 16 open reading frame 45 | C16orf45 | Hs.460095 | 212736_at |
| chromosome 16 open reading frame 46 | C16orf46 | Hs.203594 | 230281_at |
| Chromosome 16 open reading frame 46 | FLJ32702 | Hs.203594 | 232696_at |
| chromosome 16 open reading frame 5 | C16orf5 | Hs.7765 | 223960_s_at |
| chromosome 16 open reading frame 52 | C16orf52 | Hs.549248 | 230721_at |
| chromosome 16 open reading frame 55 | C16orf55 | Hs.435398 | 230811_at |
| chromosome 17 open reading frame 39 | C17orf39 | Hs.187422 | 220058_at |
| chromosome 17 open reading frame 39 | C17orf39 | Hs.187422 | 228452_at |
| chromosome 18 open reading frame 10 | C18orf10 | Hs.22981 | 212055_at |
| Chromosome 18 open reading frame 11 | C18orf11 | Hs.444314 | 237262_at |
| chromosome 18 open reading frame 2 | C18orf2 | Hs.464442 | 223977_s_at |
| chromosome 18 open reading frame 22 | C18orf22 | Hs.532987 | 243001_at |
| chromosome 18 open reading frame 23 | C18orf23 | Hs.501114 | 1557545_s_at |
| chromosome 18 open reading frame 23 | C18orf23 | Hs.501114 | 230143_at |
| chromosome 18 open reading frame 37 | C18orf37 | Hs.464903 | 1559716_at |
| chromosome 18 open reading frame 45 | C18orf45 | Hs.137562 | 244495_x_at |
| chromosome 18 open reading frame 45 /// | C18orf45 | Hs.137562 | 224493_x_at |
| chromosome 18 open reading frame 45 | | | |
| chromosome 18 open reading frame 54 | C18orf54 | Hs.208701 | 229442_at |
| chromosome 18 open reading frame 8 | C18orf8 | Hs.529006 | 221190_s_at |
| chromosome 18 open reading frame 9 | C18orf9 | Hs.236940 | 52285_f_at |
| Chromosome 19 open reading frame 2 | C19orf2 | Hs.466391 | 222266_at |
| chromosome 19 open reading frame 22 | C19orf22 | Hs.388956 | 55705_at |
| chromosome 19 open reading frame 6 | C19orf6 | Hs.515003 | 213986_s_at |
| chromosome 19 open reading frame 7 | C19orf7 | Hs.104661 | 213390_at |
| chromosome 2 open reading frame 11 | C2orf11 | Hs.350388 | 238955_at |
| chromosome 2 open reading frame 16 | C2orf16 | Hs.131021 | 224000_at |
| chromosome 2 open reading frame 17 | C2orf17 | Hs.516707 | 221983_at |
| chromosome 2 open reading frame 17 | C2orf17 | Hs.516707 | 221984_s_at |
| chromosome 2 open reading frame 29 | C2orf29 | Hs.30819 | 224695_at |
| chromosome 2 open reading frame 3 | C2orf3 | Hs.303808 | 216305_s_at |
| chromosome 2 open reading frame 33 | C2orf33 | Hs.471528 | 219137_s_at |
| chromosome 2 open reading frame 33 | C2orf33 | Hs.471528 | 222832_s_at |
| chromosome 2 open reading frame 33 | C2orf33 | Hs.471528 | 223091_x_at |
| chromosome 2 open reading frame 33 | C2orf33 | Hs.471528 | 223354_x_at |
| chromosome 2 open reading frame 4 | C2orf4 | Hs.444969 | 219065_s_at |
| Chromosome 2 open reading frame 4 | C2orf4 | Hs.444969 | 243088_at |
| chromosome 20 open reading frame 10 | C20orf10 | Hs.401982 | 207482_at |
| chromosome 20 open reading frame 106 | C20orf106 | Hs.504907 | 233086_at |
| chromosome 20 open reading frame 108 | C20orf108 | Hs.143736 | 224690_at |
| chromosome 20 open reading frame 11 | C20orf11 | Hs.353013 | 218448_at |
| chromosome 20 open reading frame 111 | C20orf111 | Hs.75798 | 209020_at |
| Chromosome 20 open reading frame 111 | C20orf111 | Hs.75798 | 221954_at |
| chromosome 20 open reading frame 112 | C20orf112 | Hs.516977 | 225224_at |
| chromosome 20 open reading frame 128 | C20orf128 | Hs.552605 | 1556793_a_at |
| chromosome 20 open reading frame 129 | C20orf129 | Hs.472716 | 225687_at |
| chromosome 20 open reading frame 141 | C20orf141 | Hs.352187 | 1554042_s_at |
| chromosome 20 open reading frame 155 | C20orf155 | Hs.224764 | 223978_s_at |
| chromosome 20 open reading frame 160 | C20orf160 | Hs.382151 | 1570070_at |
| Chromosome 20 open reading frame 17 | C20orf17 | Hs.473117 | 235815_at |
| chromosome 20 open reading frame 173 | C20orf173 | Hs.158680 | 233143_at |
| chromosome 20 open reading frame 18 | C20orf18 | Hs.247280 | 221827_at |
| chromosome 20 open reading frame 19 | C20orf19 | Hs.187635 | 228291_s_at |
| chromosome 20 open reading frame 194 | C20orf194 | Hs.516853 | 225825_at |
| chromosome 20 open reading frame 22 | C20orf22 | Hs.441550 | 228123_s_at |
| chromosome 20 open reading frame 24 | C20orf24 | Hs.184062 | 217835_x_at |
| chromosome 20 open reading frame 24 | C20orf24 | Hs.184062 | 223880_x_at |
| Chromosome 20 open reading frame 26 | C20orf26 | Hs.176013 | 1564758_at |
| chromosome 20 open reading frame 3 | C20orf3 | Hs.472330 | 206656_s_at |
| chromosome 20 open reading frame 30 | C20orf30 | Hs.472024 | 224584_at |
| chromosome 20 open reading frame 43 | C20orf43 | Hs.517134 | 217737_x_at |
| chromosome 20 open reading frame 43 | C20orf43 | Hs.517134 | 233842_x_at |
| chromosome 20 open reading frame 59 | C20orf59 | Hs.512686 | 232922_s_at |
| chromosome 20 open reading frame 6 | C20orf6 | Hs.369284 | 218859_s_at |
| chromosome 20 open reading frame 65 | C20orf65 | Hs.447626 | 232820_s_at |
| chromosome 20 open reading frame 65 | C20orf65 | Hs.447626 | 232821_at |
| chromosome 20 open reading frame 7 | C20orf7 | Hs.472165 | 222894_x_at |
| chromosome 20 open reading frame 7 | C20orf7 | Hs.472165 | 227160_s_at |
| chromosome 20 open reading frame 71 | C20orf71 | Hs.360989 | 240359_at |
| chromosome 20 open reading frame 77 | C20orf77 | Hs.278839 | 225024_at |
| chromosome 20 open reading frame 79 | C20orf79 | Hs.112794 | 231134_at |
| chromosome 21 open reading frame 106 | C21orf106 | Hs.189585 | 1555301_a_at |
| chromosome 21 open reading frame 106 | C21orf106 | Hs.189585 | 1561286_a_at |
| chromosome 21 open reading frame 106 | C21orf106 | Hs.189585 | 215529_x_at |
| chromosome 21 open reading frame 114 | C21orf114 | — | 1555373_at |
| chromosome 21 open reading frame 121 | C21orf121 | Hs.143833 | 240809_at |
| chromosome 21 open reading frame 13 | C21orf13 | Hs.517284 | 232196_at |
| Chromosome 21 open reading frame 34 | C21orf34 | Hs.473394 | 239999_at |
| chromosome 21 open reading frame 4 | C21orf4 | Hs.433668 | 222907_x_at |
| chromosome 21 open reading frame 42 | C21orf42 | Hs.234016 | 231303_at |
| chromosome 21 open reading frame 45 | C21orf45 | Hs.190518 | 219004_s_at |
| chromosome 21 open reading frame 51 | C21orf51 | Hs.303798 | 228239_at |
| chromosome 21 open reading frame 57 | C21orf57 | Hs.58668 | 227421_at |
| chromosome 21 open reading frame 59 | C21orf59 | Hs.5811 | 218123_at |
| Chromosome 21 open reading frame 66 | C21orf66 | Hs.473635 | 1556789_a_at |
| chromosome 21 open reading frame 66 | C21orf66 | Hs.473635 | 221158_at |
| Chromosome 21 open reading frame 66 | C21orf66 | Hs.473635 | 239407_at |
| chromosome 21 open reading frame 77 | C21orf77 | Hs.272236 | 220826_at |
| Chromosome 21 open reading frame 87 | C21orf87 | Hs.190548 | 239602_at |
| chromosome 21 open reading frame 91 | C21orf91 | Hs.293811 | 220941_s_at |
| chromosome 22 open reading frame 13 | C22orf13 | Hs.9850 | 223039_at |
| chromosome 22 open reading frame 23 | C22orf23 | Hs.517612 | 223706_at |
| Chromosome 22 open reading frame 23 | C22orf23 | Hs.517612 | 243515_at |
| chromosome 22 open reading frame 9 | C22orf9 | Hs.369682 | 217118_s_at |
| chromosome 3 open reading frame 10 | C3orf10 | Hs.517792 | 224575_at |
| chromosome 3 open reading frame 14 | C3orf14 | Hs.47166 | 219288_at |
| chromosome 3 open reading frame 15 | C3orf15 | Hs.341906 | 1561430_s_at |
| chromosome 3 open reading frame 18 | C3orf18 | Hs.517860 | 219114_at |
| chromosome 3 open reading frame 22 | C3orf22 | Hs.178210 | 237894_at |
| chromosome 3 open reading frame 4 | C3orf4 | Hs.107393 | 1554149_at |
| chromosome 3 open reading frame 6 | C3orf6 | Hs.478682 | 225331_at |
| chromosome 4 open reading frame 8 | C4orf8 | Hs.125348 | 203600_s_at |
| chromosome 5 open reading frame 13 | C5orf13 | Hs.483067 | 201310_s_at |
| Chromosome 5 open reading frame 15 | C5orf15 | Hs.355177 | 243350_at |
| chromosome 5 open reading frame 3 | C5orf3 | Hs.166551 | 218588_s_at |
| chromosome 5 open reading frame 3 | C5orf3 | Hs.166551 | 222682_s_at |
| chromosome 5 open reading frame 5 | C5orf5 | Hs.483473 | 218518_at |
| chromosome 6 open reading frame 105 | C6orf105 | Hs.126409 | 215100_at |
| chromosome 6 open reading frame 105 | C6orf105 | Hs.126409 | 229070_at |
| chromosome 6 open reading frame 111 | C6orf111 | Hs.520287 | 212179_at |
| chromosome 6 open reading frame 12 | C6orf12 | Hs.302037 | 234445_at |
| chromosome 6 open reading frame 130 | C6orf130 | Hs.549281 | 213322_at |
| chromosome 6 open reading frame 130 | C6orf130 | Hs.549281 | 238860_at |
| chromosome 6 open reading frame 142 | C6orf142 | Hs.485588 | 235377_at |
| chromosome 6 open reading frame 146 | C6orf146 | Hs.546521 | 237619_at |
| Chromosome 6 open reading frame 153 | C6orf153 | Hs.309231 | 1569516_at |
| chromosome 6 open reading frame 160 | C6orf160 | — | 225155_at |
| chromosome 6 open reading frame 163 | C6orf163 | Hs.485902 | 233787_at |
| chromosome 6 open reading frame 192 | C6orf192 | Hs.347144 | 226301_at |
| chromosome 6 open reading frame 201 | C6orf201 | Hs.519855 | 231642_at |
| chromosome 6 open reading frame 209 | C6orf209 | Hs.271643 | 218191_s_at |
| chromosome 6 open reading frame 33 | C6orf33 | Hs.239388 | 227626_at |
| Chromosome 6 open reading frame 49 | C6orf49 | Hs.525899 | 229351_at |
| chromosome 6 open reading frame 55 /// | C6orf55 | Hs.431367 | 224437_s_at |
| chromosome 6 open reading frame 55 | | | |
| chromosome 6 open reading frame 62 | C6orf62 | Hs.519930 | 208809_s_at |
| Chromosome 6 open reading frame 62 | C6orf62 | Hs.519930 | 213872_at |
| chromosome 6 open reading frame 64 | C6orf64 | Hs.58382 | 222741_s_at |
| chromosome 6 open reading frame 72 | C6orf72 | Hs.438872 | 225576_at |
| chromosome 6 open reading frame 74 | C6orf74 | Hs.32826 | 203259_s_at |
| chromosome 6 open reading frame 82 | C6orf82 | Hs.520070 | 221488_s_at |
| chromosome 6 open reading frame 83 | C6orf83 | Hs.487143 | 225849_s_at |
| chromosome 6 open reading frame 85 | C6orf85 | Hs.132340 | 223194_s_at |
| chromosome 6 open reading frame 85 | C6orf85 | Hs.132340 | 233206_at |
| chromosome 7 open reading frame 11 | C7orf11 | Hs.129159 | 225419_at |
| chromosome 7 open reading frame 19 | C7orf19 | Hs.363308 | 1558426_x_at |
| chromosome 7 open reading frame 24 | C7orf24 | Hs.530024 | 215380_s_at |
| Chromosome 7 open reading frame 24 | C7orf24 | Hs.530024 | 239841_at |
| chromosome 7 open reading frame 30 | C7orf30 | Hs.87385 | 226385_s_at |
| chromosome 7 open reading frame 30 | C7orf30 | Hs.87385 | 226386_at |
| chromosome 7 open reading frame 31 | C7orf31 | Hs.122055 | 229146_at |
| chromosome 7 open reading frame 34 | C7orf34 | Hs.239676 | 231435_at |
| chromosome 7 open reading frame 35 | C7orf35 | Hs.19025 | 223396_at |
| chromosome 7 open reading frame 6 | C7orf6 | Hs.489118 | 226603_at |
| Chromosome 7 unknown mRNA | — | Hs.489556 | 1556306_at |
| chromosome 8 open reading frame 1 | C8orf1 | Hs.436445 | 1554414_a_at |
| chromosome 9 open reading frame 10 | C9orf10 | Hs.372003 | 200774_at |
| chromosome 9 open reading frame 11 | C9orf11 | Hs.163070 | 1554981_at |
| chromosome 9 open reading frame 11 | C9orf11 | Hs.163070 | 1554982_a_at |
| chromosome 9 open reading frame 11 | C9orf11 | Hs.163070 | 232868_at |
| chromosome 9 open reading frame 116 | C9orf116 | Hs.414028 | 59437_at |
| chromosome 9 open reading frame 117 | C9orf117 | Hs.362949 | 231172_at |
| Chromosome 9 open reading frame 126 | C9orf126 | Hs.59504 | 233246_at |
| chromosome 9 open reading frame 156 | C9orf156 | Hs.9196 | 47530_at |
| Chromosome 9 open reading frame 3 | C9orf3 | Hs.434253 | 237716_at |
| chromosome 9 open reading frame 36 | C9orf36 | Hs.123004 | 215935_at |
| chromosome 9 open reading frame 36 /// similar to | C9orf36 | Hs.123004 | 215320_at |
| hypothetical protein /// hypothetical LOC441452 | /// | | |
| | LOC441448 | | |
| | /// | | |
| | DKFZp4 | | |
| | 34M131 | | |
| chromosome 9 open reading frame 41 | C9orf41 | Hs.257556 | 241781_at |
| chromosome 9 open reading frame 45 | C9orf45 | Hs.21379 | 223522_at |
| chromosome 9 open reading frame 5 | C9orf5 | Hs.308074 | 223005_s_at |
| chromosome 9 open reading frame 5 | C9orf5 | Hs.308074 | 223006_s_at |
| chromosome 9 open reading frame 58 | C9orf58 | Hs.4944 | 223075_s_at |
| chromosome 9 open reading frame 64 | C9orf64 | Hs.208914 | 223697_x_at |
| chromosome 9 open reading frame 64 | C9orf64 | Hs.208914 | 235940_at |
| chromosome 9 open reading frame 68 | C9orf68 | Hs.179615 | 1554707_at |
| chromosome 9 open reading frame 7 | C9orf7 | Hs.62003 | 61874_at |
| chromosome 9 open reading frame 80 | C9orf80 | Hs.494854 | 223559_s_at |
| chromosome 9 open reading frame 9 | C9orf9 | Hs.62595 | 220050_at |
| chromosome 9 open reading frame 95 | C9orf95 | Hs.494186 | 219147_s_at |
| chromosome 9 open reading frame 97 /// | C9orf97 | Hs.494595 | 213161_at |
| chromosome 9 open reading frame 97 | | | |
| Chromosome 9 open reading frame 98 | C9orf98 | Hs.421340 | 1557666_s_at |
| chromosome condensation 1 | CHC1 | Hs.469723 | 215747_s_at |
| chromosome condensation-related SMC- | CNAP1 | Hs.5719 | 201774_s_at |
| associated protein 1 | | | |
| Chromosome X open reading frame 15 | CXorf15 | Hs.534464 | 1557954_at |
| chromosome X open reading frame 33 | CXorf33 | Hs.512181 | 235433_at |
| Chromosome X open reading frame 40 | CXorf40 | Hs.425383 | 228809_at |
| chromosome X open reading frame 40 /// | CXorf40 | Hs.549718 | 214112_s_at |
| hypothetical LOC541578 protein | /// | | |
| | LOC541578 | | |
| chromosome X open reading frame 44 /// similar to | CXorf44 | Hs.415414 | 227869_at |
| LOC90736 protein | /// | | |
| | LOC440474 | | |
| cisplatin resistance-associated overexpressed | CROP | Hs.130293 | 208835_s_at |
| protein | | | |
| clathrin, heavy polypeptide (Hc) | CLTC | Hs.491351 | 200614_at |
| Clathrin, heavy polypeptide (Hc) | CLTC | Hs.491351 | 238646_at |
| clathrin, light polypeptide (Lca) | CLTA | Hs.522114 | 200960_x_at |
| clathrin, light polypeptide (Lca) | CLTA | Hs.522114 | 216295_s_at |
| claudin 1 | CLDN1 | Hs.439060 | 222549_at |
| Claudin 1 | CLDN1 | Hs.439060 | 238967_at |
| claudin 10 | CLDN10 | Hs.534377 | 205328_at |
| cleavage and polyadenylation specific factor 2, | CPSF2 | Hs.435675 | 225986_x_at |
| 100 kDa | | | |
| Cleavage and polyadenylation specific factor 6, | CPSF6 | Hs.369606 | 238913_at |
| 68 kDa | | | |
| cleavage stimulation factor, 3′ pre-RNA, subunit 3, | CSTF3 | Hs.44402 | 203947_at |
| 77 kDa | | | |
| cleavage stimulation factor, 3′ pre-RNA, subunit 3, | CSTF3 | Hs.44402 | 229666_s_at |
| 77 kDa | | | |
| clock homolog (mouse) | CLOCK | Hs.436975 | 204980_at |
| Clone 114 tumor rejection antigen mRNA, | — | Hs.547549 | 226886_at |
| complete cds | | | |
| Clone 23641 mRNA sequence | — | Hs.535382 | 214148_at |
| Clone 23728 mRNA sequence | — | Hs.467866 | 213567_at |
| Clone 23948 mRNA sequence | — | Hs.159264 | 215304_at |
| Clone 24487 mRNA sequence | — | Hs.283819 | 215825_at |
| Clone A9A2BRB5 (CAC)n/(GTG)n repeat- | — | Hs.524171 | 211994_at |
| containing mRNA. /// Clone A9A2BRB5 | | | |
| (CAC)n/(GTG)n repeat-containing mRNA. | | | |
| Clone DT1P1A11 mRNA, CAG repeat region | — | Hs.98834 | 222072_at |
| Clone FLB2932 mRNA sequence | — | Hs.274405 | 233037_at |
| Clone IMAGE: 111714 mRNA sequence | — | Hs.15671 | 228466_at |
| ClpX caseinolytic protease X homolog (E. coli) | CLPX | Hs.113823 | 204809_at |
| clusterin (complement lysis inhibitor, SP-40, 40, | CLU | Hs.436657 | 208792_s_at |
| sulfated glycoprotein 2, testosterone-repressed | | | |
| prostate message 2, apolipoprotein J) | | | |
| Clusterin (complement lysis inhibitor, SP-40, 40, | CLU | Hs.436657 | 231499_s_at |
| sulfated glycoprotein 2, testosterone-repressed | | | |
| prostate message 2, apolipoprotein J) | | | |
| clusterin associated protein 1 | CLUAP1 | Hs.155995 | 204576_s_at |
| c-Maf-inducing protein | CMIP | Hs.461510 | 224992_s_at |
| c-Mpl binding protein | LOC113251 | Hs.26613 | 212714_at |
| c-myc binding protein | MYCBP | Hs.370040 | 203360_s_at |
| coactivator-associated arginine methyltransferase1 | CARM1 | Hs.371416 | 212512_s_at |
| coagulation factor VIII, procoagulant component | F8 | Hs.413083 | 205756_s_at |
| (hemophilia A) | | | |
| coagulation factor XIII, A1 polypeptide | F13A1 | Hs.335513 | 203305_at |
| coatomer protein complex, subunit beta | COPB | Hs.339278 | 201358_s_at |
| Coatomer protein complex, subunit beta | COPB | Hs.339278 | 233308_at |
| COBW domain containing 1 | CBWD1 | Hs.531330 | 226193_x_at |
| cofactor required for Sp1 transcriptional activation, | CRSP7 | Hs.466117 | 227538_at |
| subunit 7, 70 kDa | | | |
| cofilin 1 (non-muscle) /// cofilin 1 (non-muscle) | CFL1 | Hs.170622 | 200021_at |
| coiled-coil domain containing 13 | CCDC13 | Hs.191036 | 1553397_at |
| coiled-coil domain containing 15 | CCDC15 | Hs.512748 | 220466_at |
| coiled-coil domain containing 16 | CCDC16 | Hs.413678 | 226315_at |
| coiled-coil domain containing 7 | CCDC7 | Hs.436242 | 1553214_a_at |
| coiled-coil domain containing 7 | CCDC7 | Hs.436242 | 1553215_s_at |
| coilin | COIL | Hs.532795 | 203654_s_at |
| cold shock domain protein A | CSDA | Hs.221889 | 201160_s_at |
| collaborates/cooperates with ARF (alternate | CARF | Hs.32922 | 218929_at |
| reading frame) protein | | | |
| collagen, type I, alpha 2 | COL1A2 | Hs.489142 | 202403_s_at |
| collagen, type II, alpha 1 (primary osteoarthritis, | COL2A1 | Hs.408182 | 213492_at |
| spondyloepiphyseal dysplasia, congenital) | | | |
| collagen, type III, alpha 1 (Ehlers-Danlos syndrome | COL3A1 | Hs.443625 | 201852_x_at |
| type IV, autosomal dominant) | | | |
| collagen, type III, alpha 1 (Ehlers-Danlos syndrome | COL3A1 | Hs.443625 | 215076_s_at |
| type IV, autosomal dominant) | | | |
| collagen, type IV, alpha 3 (Goodpasture antigen) | COL4A3BP | Hs.270437 | 223466_x_at |
| binding protein | | | |
| Collagen, type IV, alpha 3 (Goodpasture antigen) | COL4A3BP | Hs.270437 | 238279_x_at |
| binding protein | | | |
| collagen, type IX, alpha 1 | COL9A1 | Hs.149809 | 1555527_at |
| collagen, type XI, alpha 2 | COL11A2 | Hs.390171 | 216993_s_at |
| collagen, type XXV, alpha 1 /// collagen, type XXV, | COL25A1 | Hs.112925 | 224388_s_at |
| alpha 1 | | | |
| collagen, type XXV, alpha 1 /// collagen, type XXV, | COL25A1 | Hs.112925 | 224389_s_at |
| alpha 1 | | | |
| collagen, type XXVII, alpha 1 | COL27A1 | Hs.494892 | 225293_at |
| colony stimulating factor 2 receptor, beta, low- | CSF2RB | Hs.285401 | 205159_at |
| affinity (granulocyte-macrophage) /// colony | | | |
| stimulating factor 2 receptor, beta, low-affinity | | | |
| (granulocyte-macrophage) | | | |
| COMM domain containing 3 | COMMD3 | Hs.534398 | 218048_at |
| complement component 1, q subcomponent | C1QBP | Hs.462086 | 214214_s_at |
| binding protein | | | |
| complement component 4A /// complement | C4A /// | Hs.534847 | 214428_x_at |
| component 4B /// complement component 4B, | C4B | | |
| telomeric | | | |
| component of oligomeric golgi complex 2 | COG2 | Hs.211800 | 203073_at |
| component of oligomeric golgi complex 5 | COG5 | Hs.239631 | 203630_s_at |
| congenital dyserythropoietic anemia, type I | CDAN1 | Hs.437819 | 228516_at |
| conserved helix-loop-helix ubiquitous kinase | CHUK | Hs.198998 | 209666_s_at |
| COP9 constitutive photomorphogenic homolog | COPS5 | Hs.491912 | 201652_at |
| subunit 5 (Arabidopsis) | | | |
| COP9 constitutive photomorphogenic homolog | COPS6 | Hs.15591 | 201405_s_at |
| subunit 6 (Arabidopsis) | | | |
| copine III | CPNE3 | Hs.191219 | 202118_s_at |
| copine VIII | CPNE8 | Hs.40910 | 243727_at |
| core 1 synthase, glycoprotein-N- | C1GALT1 | Hs.239666 | 219439_at |
| acetylgalactosamine 3-beta-galactosyltransferase, 1 | | | |
| core-binding factor, beta subunit | CBFB | Hs.460988 | 202370_s_at |
| core-binding factor, runt domain, alpha subunit 2; | CBFA2T2 | Hs.153934 | 238549_at |
| translocated to, 2 | | | |
| cornichon homolog (Drosophila) | CNIH | Hs.294603 | 201653_at |
| coronin 6 | CORO6 | Hs.143046 | 1552301_a_at |
| coronin, actin binding protein, 1C | CORO1C | Hs.330384 | 221676_s_at |
| COX11 homolog, cytochrome c oxidase assembly | COX11 | Hs.96530 | 203551_s_at |
| protein (yeast) | | | |
| COX11 homolog, cytochrome c oxidase assembly | COX11 | Hs.96530 | 211727_s_at |
| protein (yeast) /// COX11 homolog, cytochrome c | | | |
| oxidase assembly protein (yeast) | | | |
| COX15 homolog, cytochrome c oxidase assembly | COX15 | Hs.28326 | 219547_at |
| protein (yeast) | | | |
| COX15 homolog, cytochrome c oxidase assembly | COX15 | Hs.28326 | 235204_at |
| protein (yeast) | | | |
| coxsackie virus and adenovirus receptor | CXADR | Hs.473417 | 203917_at |
| CPX chromosome region, candidate 1 | CPXCR1 | Hs.458292 | 1560493_a_at |
| creatine kinase, brain | CKB | Hs.173724 | 200884_at |
| Crm, cramped-like (Drosophila) | CRAMP1L | — | 225172_at |
| crystallin, mu | CRYM | Hs.924 | 205489_at |
| crystallin, zeta (quinone reductase)-like 1 | CRYZL1 | Hs.352671 | 1552347_at |
| CSE1 chromosome segregation 1-like (yeast) | CSE1L | Hs.90073 | 201111_at |
| CSE1 chromosome segregation 1-like (yeast) | CSE1L | Hs.90073 | 201112_s_at |
| CSE1 chromosome segregation 1-like (yeast) | CSE1L | Hs.90073 | 210766_s_at |
| CTAGE family, member 5 | CTAGE5 | Hs.509200 | 204055_s_at |
| CTD (carboxy-terminal domain, RNA polymerase | CTDSP2 | Hs.524530 | 203445_s_at |
| II, polypeptide A) small phosphatase 2 | | | |
| CTD (carboxy-terminal domain, RNA polymerase | CTDSP2 | Hs.524530 | 208735_s_at |
| II, polypeptide A) small phosphatase 2 | | | |
| C-terminal binding protein 1 | CTBP1 | Hs.208597 | 203392_s_at |
| C-terminal binding protein 1 | CTBP1 | Hs.208597 | 212863_x_at |
| C-terminal binding protein 2 | CTBP2 | Hs.501345 | 201220_x_at |
| C-terminal binding protein 2 | CTBP2 | Hs.501345 | 210835_s_at |
| C-terminal binding protein 2 /// L0C440008 | CTBP2 | Hs.501345 | 201219_at |
| | /// | | |
| | LOC440008 | | |
| C-terminal modulator protein | CTMP | Hs.164070 | 229253_at |
| CTL2 protein | CTL2 | Hs.515134 | 224609_at |
| CTP synthase | CTPS | Hs.473087 | 202613_at |
| C-type lectin domain family 4, member A | CLEC4A | Hs.504657 | 219947_at |
| C-type lectin domain family 7, member A /// C-type | CLEC7A | Hs.143929 | 221698_s_at |
| lectin domain family 7, member A | | | |
| CUB and Sushi multiple domains 2 | CSMD2 | Hs.127736 | 242933_at |
| CUG triplet repeat, RNA binding protein 1 | CUGBP1 | Hs.530727 | 209489_at |
| CUG triplet repeat, RNA binding protein 2 | CUGBP2 | Hs.309288 | 1560049_at |
| CUG triplet repeat, RNA binding protein 2 | CUGBP2 | Hs.309288 | 202157_s_at |
| CUG triplet repeat, RNA binding protein 2 | CUGBP2 | Hs.309288 | 227178_at |
| cullin 1 | CUL1 | Hs.146806 | 207614_s_at |
| cullin 2 | CUL2 | Hs.82919 | 203079_s_at |
| cullin 3 | CUL3 | Hs.372286 | 201370_s_at |
| cullin 3 | CUL3 | Hs.372286 | 201371_s_at |
| Cullin 3 | CUL3 | Hs.372286 | 242362_at |
| cullin 4B | CUL4B | Hs.102914 | 210257_x_at |
| Cullin 5 | CUL5 | Hs.440320 | 203531_at |
| cutaneous T-cell lymphoma-associated antigen 1 | CTAGE1 | Hs.406709 | 220957_at |
| cut-like 1, CCAAT displacement protein | CUTL1 | Hs.438974 | 214743_at |
| (Drosophila) | | | |
| CXXC finger 1 (PHD domain) | CXXC1 | Hs.180933 | 48580_at |
| CXXC finger 5 | CXXC5 | Hs.189119 | 222996_s_at |
| CXXC finger 5 | CXXC5 | Hs.189119 | 233955_x_at |
| CXXC finger 5 /// CXXC finger 5 | CXXC5 | Hs.189119 | 224516_s_at |
| CXYorf1-related protein | FLJ25222 | Hs.459573 | 236841_at |
| cyclic AMP phosphoprotein, 19 kD | ARPP- | Hs.512908 | 214553_s_at |
| | 19 | | |
| cyclic AMP phosphoprotein, 19 kD | ARPP- | Hs.512908 | 221482_s_at |
| | 19 | | |
| cyclic AMP phosphoprotein, 19 kD | ARPP- | Hs.512908 | 221483_s_at |
| | 19 | | |
| cyclic AMP-regulated phosphoprotein, 21 kD | ARPP- | Hs.475902 | 231935_at |
| | 21 | | |
| cyclin A1 | CCNA1 | Hs.417050 | 205899_at |
| Cyclin A2 | CCNA2 | Hs.85137 | 213226_at |
| cyclin B1 interacting protein 1 | CCNB1\|P1 | Hs.107003 | 217988_at |
| cyclin B2 | CCNB2 | Hs.194698 | 202705_at |
| cyclin E2 | CCNE2 | Hs.408658 | 205034_at |
| cyclin E2 | CCNE2 | Hs.408658 | 211814_s_at |
| cyclin G associated kinase | GAK | Hs.369607 | 202281_at |
| cyclin G associated kinase | GAK | Hs.369607 | 40225_at |
| cyclin H | CCNH | Hs.146607 | 204093_at |
| Cyclin H | CCNH | Hs.146607 | 238243_at |
| Cyclin J | CCNJ | Hs.281348 | 1557830_at |
| cyclin M1 | CNNM1 | Hs.274579 | 220166_at |
| cyclin M3 | CNNM3 | Hs.150895 | 220739_s_at |
| Cyclin M3 | CNNM3 | Hs.150895 | 229031_at |
| Cyclin-dependent kinase 8 | CDK8 | Hs.382306 | 204831_at |
| Cyclin-dependent kinase inhibitor 1C (p57, Kip2) | CDKN1C | Hs.106070 | 213348_at |
| cyclin-dependent kinase inhibitor 3 (CDK2- | CDKN3 | Hs.84113 | 209714_s_at |
| associated dual specificity phosphatase) | | | |
| Cyclin-dependent kinase-like 1 (CDC2-related | CDKL1 | Hs.280881 | 241928_at |
| kinase) | | | |
| cylicin, basic protein of sperm head cytoskeleton 1 | CYLC1 | Hs.444230 | 216778_s_at |
| cylicin, basic protein of sperm head cytoskeleton 1 | CYLC1 | Hs.444230 | 216809_at |
| cylicin, basic protein of sperm head cytoskeleton 2 | CYLC2 | Hs.3232 | 207780_at |
| cystathionase (cystathionine gamma-lyase) | CTH | Hs.19904 | 217127_at |
| cystathionine-beta-synthase | CBS | Hs.533013 | 1553972_a_at |
| cystatin pseudogene | LOC164380 | Hs.400007 | 1558493_at |
| cysteine and glycine-rich protein 2 | CSRP2 | Hs.530904 | 207030_s_at |
| cysteine and histidine-rich domain (CHORD)- | CHORDC1 | Hs.22857 | 218566_s_at |
| containing, zinc binding protein 1 | | | |
| cysteine-rich hydrophobic domain 2 | CHIC2 | Hs.335393 | 219492_at |
| cysteine-rich secretory protein 1 | CRISP1 | Hs.109620 | 207032_s_at |
| cysteine-rich secretory protein 1 | CRISP1 | Hs.109620 | 216314_at |
| cysteine-rich secretory protein 2 | CRISP2 | Hs.2042 | 210262_at |
| cysteine-rich, angiogenic inducer, 61 | CYR61 | Hs.8867 | 201289_at |
| cysteinyl-tRNA synthetase | CARS | Hs.274873 | 212971_at |
| cystinosis, nephropathic | CTNS | Hs.187667 | 36566_at |
| cytidine and dCMP deaminase domain containing 1 | CDADC1 | Hs.388220 | 223527_s_at |
| cytidine and dCMP deaminase domain containing 1 | CDADC1 | Hs.388220 | 233647_s_at |
| cytochrome b, ascorbate dependent 3 | CYBASC3 | Hs.22546 | 224735_at |
| cytochrome b-5 | CYB5 | Hs.465413 | 207843_x_at |
| cytochrome b-5 | CYB5 | Hs.465413 | 209366_x_at |
| cytochrome b5 reductase b5R.2 | CYB5R2 | Hs.414362 | 220230_s_at |
| cytochrome c oxidase subunit 8C | COX8C | Hs.128935 | 231487_at |
| cytochrome c oxidase subunit IV isoform 1 | COX4I1 | Hs.433419 | 202698_x_at |
| cytochrome c oxidase subunit IV isoform 1 | COX4I1 | Hs.433419 | 213758_at |
| cytochrome c oxidase subunit IV isoform 1 /// | COX4I1 | Hs.433419 | 200086_s_at |
| cytochrome c oxidase subunit IV isoform 1 | | | |
| cytochrome c oxidase subunit Vb | COX5B | Hs.1342 | 202343_x_at |
| cytochrome c oxidase subunit Vb | COX5B | Hs.1342 | 213735_s_at |
| cytochrome c oxidase subunit VIc | COX6C | Hs.351875 | 201754_at |
| cytochrome c oxidase subunit VIIa polypeptide 2 | COX7A2 | Hs.70312 | 201597_at |
| (liver) | | | |
| cytochrome c oxidase subunit VIIc | COX7C | Hs.430075 | 217491_x_at |
| cytochrome c, somatic | CYCS | Hs.437060 | 208905_at |
| cytochrome P450, family 1, subfamily A, | CYP1A2 | Hs.1361 | 207608_x_at |
| polypeptide 2 | | | |
| cytochrome P450, family 1, subfamily B, | CYP1B1 | Hs.154654 | 202435_s_at |
| polypeptide 1 | | | |
| cytochrome P450, family 1, subfamily B, | CYP1B1 | Hs.154654 | 202436_s_at |
| polypeptide 1 | | | |
| cytochrome P450, family 2, subfamily A, | CYP2A6 | Hs.439056 | 207718_x_at |
| polypeptide 6 /// cytochrome P450, family 2, | /// | | |
| subfamily A, polypeptide 7 /// cytochrome P450, | CYP2A7 | | |
| family 2, subfamily A, polypeptide 7 pseudogene 1 | /// | | |
| /// cytochrome P450, family 2, subfamily A, | CYP2A7P1 | | |
| polypeptide 13 | /// | | |
| | CYP2A13 | | |
| cytochrome P450, family 2, subfamily C, | CYP2C8 | Hs.282871 | 208147_s_at |
| polypeptide 8 /// cytochrome P450, family 2, | | | |
| subfamily C, polypeptide 8 | | | |
| cytochrome P450, family 2, subfamily C, | CYP2C9 | Hs.282624 | 214421_x_at |
| polypeptide 9 | | | |
| cytochrome P450, family 2, subfamily R, | CYP2R1 | Hs.371427 | 227109_at |
| polypeptide 1 | | | |
| cytochrome P450, family 2, subfamily U, | CYP2U1 | Hs.109087 | 226393_at |
| polypeptide 1 | | | |
| cytochrome P450, family 4, subfamily V, | CYP4V2 | Hs.237642 | 226745_at |
| polypeptide 2 | | | |
| cytochrome P450, family 51, subfamily A, | CYP51A1 | Hs.417077 | 216607_s_at |
| polypeptide 1 | | | |
| cytokine induced apoptosis inhibitor 1 | CIAPIN1 | Hs.4900 | 208968_s_at |
| cytokine receptor-like factor 3 | CRLF3 | Hs.370168 | 205474_at |
| cytokine-like nuclear factor n-pac | N-PAC | Hs.387255 | 218506_x_at |
| cytoplasmic FMR1 interacting protein 1 | CYFIP1 | Hs.26704 | 208923_at |
| cytoplasmic linker associated protein 1 | CLASP1 | Hs.469840 | 212752_at |
| cytoplasmic polyadenylation element binding | CPEB1 | Hs.546423 | 222903_s_at |
| protein 1 | | | |
| cytoplasmic polyadenylation element binding | CPEB2 | Hs.374216 | 226939_at |
| protein 2 | | | |
| cytoplasmic polyadenylation element binding | CPEB2 | Hs.374216 | 235479_at |
| protein 2 | | | |
| cytoplasmic polyadenylation element binding | CPEB3 | Hs.131683 | 1555250_a_at |
| protein 3 | | | |
| cytoplasmic polyadenylation element binding | CPEB4 | Hs.127126 | 224829_at |
| protein 4 | | | |
| cytoskeleton-associated protein 4 | CKAP4 | Hs.74368 | 200999_s_at |
| DALR anticodon binding domain containing 3 | DALRD3 | Hs.280978 | 218808_at |
| DAZ associated protein 2 /// similar to DAZ- | DAZAP2 | Hs.369761 | 214334_x_at |
| associated protein 2 (Deleted in azoospermia- | /// | | |
| associated protein 2) | LOC401029 | | |
| DAZ interacting protein 1 | DZIP1 | Hs.157002 | 204556_s_at |
| DC13 protein | DC13 | Hs.388255 | 218447_at |
| DC2 protein | DC2 | Hs.445803 | 223001_at |
| DCP1 decapping enzyme homolog B (S. cerevisiae) | DCP1B | Hs.130934 | 226093_at |
| DCP2 decapping enzyme homolog (S. cerevisiae) | DCP2 | Hs.443875 | 244777_at |
| DDHD domain containing 1 | DDHD1 | Hs.513260 | 225965_at |
| DDHD domain containing 1 | DDHD1 | Hs.513260 | 231165_at |
| DDHD domain containing 1 | DDHD1 | Hs.513260 | 244154_at |
| DDHD domain containing 2 | DDHD2 | Hs.434966 | 212690_at |
| DEAD (Asp-Glu-Ala-Asp) box polypeptide 10 | DDX10 | Hs.525115 | 204977_at |
| DEAD (Asp-Glu-Ala-Asp) box polypeptide 17 | DDX17 | Hs.528305 | 208718_at |
| DEAD (Asp-Glu-Ala-Asp) box polypeptide 18 | DDX18 | Hs.363492 | 208895_s_at |
| DEAD (Asp-Glu-Ala-Asp) box polypeptide 20 | DDX20 | Hs.485810 | 223331_s_at |
| DEAD (Asp-Glu-Ala-Asp) box polypeptide 21 /// | DDX21 | Hs.223141 | 208152_s_at |
| DEAD (Asp-Glu-Ala-Asp) box polypeptide 21 | | | |
| DEAD (Asp-Glu-Ala-Asp) box polypeptide 21 /// | DDX21 | Hs.223141 | 224654_at |
| zinc finger protein 596 | /// | | |
| | ZNF596 | | |
| DEAD (Asp-Glu-Ala-Asp) box polypeptide 24 | DDX24 | Hs.510328 | 200694_s_at |
| DEAD (Asp-Glu-Ala-Asp) box polypeptide 25 | DDX25 | Hs.420263 | 219945_at |
| DEAD (Asp-Glu-Ala-Asp) box polypeptide 27 | DDX27 | Hs.65234 | 215693_x_at |
| DEAD (Asp-Glu-Ala-Asp) box polypeptide 3, X- | DDX3X | Hs.380774 | 201210_at |
| linked | | | |
| DEAD (Asp-Glu-Ala-Asp) box polypeptide 3, X- | DDX3X | Hs.380774 | 207617_at |
| linked | | | |
| DEAD (Asp-Glu-Ala-Asp) box polypeptide 3, X- | DDX3X | Hs.380774 | 212514_x_at |
| linked | | | |
| DEAD (Asp-Glu-Ala-Asp) box polypeptide 3, Y- | DDX3Y | Hs.99120 | 205001_s_at |
| linked | | | |
| DEAD (Asp-Glu-Ala-Asp) box polypeptide 31 | DDX31 | Hs.495410 | 1553181_at |
| DEAD (Asp-Glu-Ala-Asp) box polypeptide 4 | DDX4 | Hs.223581 | 221630_s_at |
| DEAD (Asp-Glu-Ala-Asp) box polypeptide 42 | DDX42 | Hs.8765 | 201788_at |
| DEAD (Asp-Glu-Ala-Asp) box polypeptide 48 | DDX48 | Hs.389649 | 201303_at |
| DEAD (Asp-Glu-Ala-Asp) box polypeptide 5 | DDX5 | Hs.279806 | 243206_at |
| DEAD (Asp-Glu-Ala-Asp) box polypeptide 5 /// | DDX5 | Hs.279806 | 200033_at |
| DEAD (Asp-Glu-Ala-Asp) box polypeptide 5 | | | |
| DEAD (Asp-Glu-Ala-Asp) box polypeptide 50 | DDX50 | Hs.522984 | 1568815_a_at |
| DEAD (Asp-Glu-Ala-Asp) box polypeptide 52 | DDX52 | Hs.408452 | 210320_s_at |
| DEAD (Asp-Glu-Ala-Asp) box polypeptide 52 | DDX52 | Hs.408452 | 212834_at |
| DEAD (Asp-Glu-Ala-Asp) box polypeptide 58 | DDX58 | Hs.190622 | 242961_x_at |
| DEAD (Asp-Glu-Ala-Asp) box polypeptide 6 | DDX6 | Hs.408461 | 225549_at |
| dead end homolog 1 (zebrafish) | DND1 | Hs.552623 | 222070_at |
| dead end homolog 1 (zebrafish) | DND1 | Hs.552623 | 57739_at |
| DEAD/H (Asp-Glu-Ala-Asp/His) box polypeptide 26 | DDX26 | Hs.439440 | 218819_at |
| DEAD/H (Asp-Glu-Ala-Asp/His) box polypeptide 26 | DDX26 | Hs.439440 | 222239_s_at |
| DEAD/H (Asp-Glu-Ala-Asp/His) box polypeptide 26 | DDX26 | Hs.439440 | 235283_at |
| DEAH (Asp-Glu-Ala-Asp/His) box polypeptide 57 | DHX57 | Hs.468226 | 239311_at |
| DEAH (Asp-Glu-Ala-His) box polypeptide 16 | DHX16 | Hs.485060 | 203694_s_at |
| DEAH (Asp-Glu-Ala-His) box polypeptide 29 | DHX29 | Hs.444208 | 212648_at |
| DEAH (Asp-Glu-Ala-His) box polypeptide 30 | DHX30 | Hs.517948 | 1556144_at |
| DEAH (Asp-Glu-Ala-His) box polypeptide 30 | DHX30 | Hs.517948 | 230871_at |
| DEAH (Asp-Glu-Ala-His) box polypeptide 32 | DHX32 | Hs.501379 | 218198_at |
| DEAH (Asp-Glu-Ala-His) box polypeptide 33 | DHX33 | Hs.250456 | 222875_at |
| DEAH (Asp-Glu-Ala-His) box polypeptide 36 | DHX36 | Hs.446270 | 223138_s_at |
| DEAH (Asp-Glu-Ala-His) box polypeptide 36 | DHX36 | Hs.446270 | 223139_s_at |
| DEAH (Asp-Glu-Ala-His) box polypeptide 40 | DHX40 | Hs.29403 | 222574_s_at |
| DEAH (Asp-Glu-Ala-His) box polypeptide 8 | DHX8 | Hs.463105 | 227079_at |
| DEAH (Asp-Glu-Ala-His) box polypeptide 9 | DHX9 | Hs.191518 | 202420_s_at |
| DEAH (Asp-Glu-Ala-His) box polypeptide 9 | DHX9 | Hs.191518 | 212107_s_at |
| DEAH (Asp-Glu-Ala-His) box polypeptide 9 | DHX9 | Hs.191518 | 215467_x_at |
| death associated transcription factor 1 | DATF1 | Hs.551527 | 213213_at |
| death-associated protein kinase 1 | DAPK1 | Hs.380277 | 203139_at |
| decay accelerating factor for complement (CD55, | DAF | Hs.527653 | 201926_s_at |
| Cromer blood group system) | | | |
| dedicator of cytokinesis 5 | DOCK5 | Hs.195403 | 1569590_at |
| defender against cell death 1 /// defender against | DAD1 | Hs.82890 | 200046_at |
| cell death 1 | | | |
| defensin, beta 106A | DEFB106A | Hs.511958 | 1552411_at |
| dehydrodolichyl diphosphate synthase | DHDDS | Hs.369385 | 218547_at |
| dehydrogenase/reductase (SDR family) member 2 | DHRS2 | Hs.272499 | 214079_at |
| dehydrogenase/reductase (SDR family) member 9 | DHRS9 | Hs.179608 | 219799_s_at |
| Deleted in lymphocytic leukemia, 2 | DLEU2 | Hs.508041 | 1556820_a_at |
| deleted in lymphocytic leukemia, 2 /// BCMS | DLEU2 | Hs.508041 | 215629_s_at |
| upstream neighbor-like | /// | | |
| | BCMSUNL | | |
| deleted in lymphocytic leukemia, 2 /// BCMS | DLEU2 | Hs.508041 | 216870_x_at |
| upstream neighbor-like | /// | | |
| | BCMSUNL | | |
| deleted in malignant brain tumors 1 | DMBT1 | Hs.279611 | 208250_s_at |
| Dendritic cell protein | GA17 | Hs.502244 | 215190_at |
| Dendritic cell-derived ubiquitin-like protein | DC-UbP | Hs.179852 | 224827_at |
| DEP domain containing 1 | DEPDC1 | Hs.445098 | 220295_x_at |
| DEP domain containing 6 | DEPDC6 | Hs.303788 | 218858_at |
| Der1-like domain family, member 3 | DERL3 | Hs.159971 | 228897_at |
| desmuslin | DMN | Hs.207106 | 212730_at |
| destrin-2 pseudogene | LOC171220 | — | 211325_x_at |
| development and differentiation enhancing factor 1 | DDEF1 | Hs.106015 | 224790_at |
| development and differentiation enhancing factor 1 | DDEF1 | Hs.106015 | 224791_at |
| development and differentiation enhancing factor 1 | DDEF1 | Hs.106015 | 224796_at |
| development and differentiation enhancing factor 1 | DDEF1 | Hs.106015 | 236533_at |
| development and differentiation enhancing factor 2 | DDEF2 | Hs.467662 | 206414_s_at |
| diablo homolog (Drosophila) | DIABLO | Hs.169611 | 219350_s_at |
| diacylglycerol O-acyltransferase homolog 2 | DGAT2 | Hs.334305 | 226064_s_at |
| (mouse) | | | |
| Diaphanous homolog 1 (Drosophila) | DIAPH1 | Hs.529451 | 1560080_at |
| diaphanous homolog 2 (Drosophila) | DIAPH2 | Hs.226483 | 205603_s_at |
| Dicer1, Dcr-1 homolog (Drosophila) | DICER1 | Hs.87889 | 213229_at |
| dickkopf homolog 1 (Xenopus laevis) | DKK1 | Hs.40499 | 204602_at |
| dickkopf homolog 3 (Xenopus laevis) | DKK3 | Hs.292156 | 214247_s_at |
| DiGeorge syndrome critical region gene 5 (non- | DGCR5 | Hs.551533 | 215244_at |
| coding) | | | |
| DiGeorge syndrome critical region gene 6 /// | DGCR6 | Hs.410965 | 208024_s_at |
| DiGeorge syndrome critical region gene 6-like | /// | | |
| | DGCR6L | | |
| dihydrofolate reductase | DHFR | Hs.83765 | 202534_x_at |
| dihydrolipoamide branched chain transacylase E2 | DBT | Hs.270570 | 205370_x_at |
| dihydropyrimidinase-like 5 | DPYSL5 | Hs.299315 | 224100_s_at |
| dipeptidylpeptidase 8 | DPP8 | Hs.458609 | 220939_s_at |
| disabled homolog 2, mitogen-responsive | DAB2 | Hs.481980 | 201280_s_at |
| phosphoprotein (Drosophila) | | | |
| discoidin domain receptor family, member 1 | DDR1 | Hs.520004 | 210749_x_at |
| Discoidin domain receptor family, member 2 | DDR2 | Hs.275757 | 227561_at |
| discs, large (Drosophila) homolog-associated | DLGAP4 | Hs.249600 | 233056_x_at |
| protein 4 | | | |
| discs, large homolog 1 (Drosophila) | DLG1 | Hs.292549 | 202515_at |
| discs, large homolog 5 (Drosophila) | DLG5 | Hs.500245 | 201681_s_at |
| Dishevelled associated activator of morphogenesis 1 | DAAM1 | Hs.19156 | 1555989_at |
| Dishevelled associated activator of morphogenesis 1 | DAAM1 | Hs.19156 | 226666_at |
| Dishevelled associated activator of morphogenesis 1 | DAAM1 | Hs.19156 | 244062_at |
| dJ39G22.2 (novel protein) | dJ39G22.2 | Hs.406265 | 231264_at |
| DKFZp434A0131 protein | DKFZP434A0131 | Hs.535037 | 221191_at |
| DKFZp434A0131 protein | DKFZP434A0131 | Hs.550749 | 229949_at |
| DKFZP434B0335 protein | DKFZP434B0335 | Hs.511979 | 227580_s_at |
| DKFZP434B0335 protein | DKFZP434B0335 | Hs.511979 | 227581_at |
| DKFZP434B061 protein | DKFZP434B061 | Hs.448583 | 233092_s_at |
| DKFZP434H132 protein | DKFZP434H132 | Hs.17936 | 215087_at |
| DKFZP564B167 protein | DKFZP564B167 | Hs.517768 | 202427_s_at |
| DKFZP564C152 protein | DKFZP564C152 | Hs.184216 | 216028_at |
| DKFZP564C196 protein | DKFZP564C196 | Hs.127384 | 222384_at |
| DKFZP564G2022 protein | DKFZP564G2022 | Hs.511138 | 212202_s_at |
| DKFZP586A0522 protein | DKFZP586A0522 | Hs.288771 | 209703_x_at |
| Dmx-like 1 | DMXL1 | Hs.181042 | 241472_at |
| DNA (cytosine-5-)-methyltransferase 3 beta | DNMT3B | Hs.251673 | 220668_s_at |
| DNA cross-link repair 1C (PSO2 homolog, S. cerevisiae) | DCLRE1C | Hs.524156 | 219678_x_at |
| DNA damage repair and recombination protein | — | Hs.179697 | 215312_at |
| RAD52 pseudogene | | | |
| DNA polymerase-transactivated protein 6 | DNAPTP6 | Hs.120323 | 241812_at |
| DNA polymerase-transactivated protein 6 | DNAPTP6 | Hs.120323 | 244597_at |
| DNA segment on chromosome 4 (unique) 234 | D4S234E | Hs.518595 | 209569_x_at |
| expressed sequence | | | |
| DNA-damage inducible protein 1 | DDI1 | — | 239299_at |
| DnaJ (Hsp40) homolog, subfamily A, member 1 | DNAJA1 | Hs.445203 | 200880_at |
| DnaJ (Hsp40) homolog, subfamily A, member 1 | DNAJA1 | Hs.445203 | 200881_s_at |
| DnaJ (Hsp40) homolog, subfamily A, member 2 | DNAJA2 | Hs.368078 | 226994_at |
| DnaJ (Hsp40) homolog, subfamily A, member 3 | DNAJA3 | Hs.459779 | 1554078_s_at |
| DnaJ (Hsp40) homolog, subfamily A, member 3 | DNAJA3 | Hs.459779 | 205963_s_at |
| DnaJ (Hsp40) homolog, subfamily A, member 4 | DNAJA4 | Hs.513053 | 1554334_a_at |
| DnaJ (Hsp40) homolog, subfamily B, member 1 | DNAJB1 | Hs.515210 | 200666_s_at |
| DnaJ (Hsp40) homolog, subfamily B, member 4 | DNAJB4 | Hs.380282 | 203811_s_at |
| DnaJ (Hsp40) homolog, subfamily B, member 6 | DNAJB6 | Hs.490745 | 208810_at |
| DnaJ (Hsp40) homolog, subfamily B, member 6 | DNAJB6 | Hs.490745 | 208811_s_at |
| DnaJ (Hsp40) homolog, subfamily B, member 6 | DNAJB6 | Hs.490745 | 209015_s_at |
| DnaJ (Hsp40) homolog, subfamily B, member 8 | DNAJB8 | Hs.518241 | 237284_at |
| DnaJ (Hsp40) homolog, subfamily B, member 9 | DNAJB9 | Hs.6790 | 202842_s_at |
| DnaJ (Hsp40) homolog, subfamily C, member 1 | DNAJC1 | Hs.499000 | 222620_s_at |
| DnaJ (Hsp40) homolog, subfamily C, member 1 | DNAJC1 | Hs.499000 | 222621_at |
| DnaJ (Hsp40) homolog, subfamily C, member 1 | DNAJC1 | Hs.499000 | 242216_at |
| DnaJ (Hsp40) homolog, subfamily C, member 12 | DNAJC12 | Hs.260720 | 223722_at |
| DnaJ (Hsp40) homolog, subfamily C, member 4 | DNAJC4 | Hs.172847 | 213919_at |
| DnaJ (Hsp40) homolog, subfamily C, member 9 | DNAJC9 | Hs.523037 | 213088_s_at |
| DnaJ homology subfamily A member 5 | DNAJA5 | Hs.131887 | 238337_s_at |
| docking protein 5 | DOK5 | Hs.473133 | 214844_s_at |
| dolichyl-diphosphooligosaccharide-protein | DDOST | Hs.523145 | 208674_x_at |
| glycosyltransferase | | | |
| dolichyl-phosphate (UDP-N-acetylglucosamine) N- | DPAGT1 | Hs.524081 | 209509_s_at |
| acetylglucosaminephosphotransferase 1 (GlcNAc- | | | |
| 1-P transferase) | | | |
| dolichyl-phosphate mannosyltransferase | DPM1 | Hs.549080 | 202673_at |
| polypeptide 1, catalytic subunit | | | |
| Dolichyl-phosphate mannosyltransferase | DPM1 | Hs.549080 | 237213_at |
| polypeptide 1, catalytic subunit | | | |
| dom-3 homolog Z (C. elegans) | DOM3Z | Hs.153299 | 38157_at |
| dopachrome tautomerase (dopachrome delta- | DCT | Hs.301865 | 205338_s_at |
| isomerase, tyrosine-related protein 2) | | | |
| dopamine receptor D5 | DRD5 | Hs.380681 | 208486_at |
| doublecortin and CaM kinase-like 1 | DCAMKL1 | Hs.507755 | 205399_at |
| Down syndrome critical region gene 1 | DSCR1 | Hs.282326 | 208370_s_at |
| Down syndrome critical region gene 2 | DSCR2 | Hs.473838 | 203405_at |
| Down syndrome critical region gene 3 | DSCR3 | Hs.369488 | 233108_at |
| Down syndrome critical region gene 8 | DSCR8 | Hs.192371 | 241224_x_at |
| down-regulated by Ctnnb1, a | DRCTNNB1A | Hs.85603 | 231396_s_at |
| downregulated in ovarian cancer 1 | DOC1 | Hs.104672 | 1554965_at |
| downregulated in ovarian cancer 1 | DOC1 | Hs.104672 | 204135_at |
| down-regulator of transcription 1, TBP-binding | DR1 | Hs.348418 | 209188_x_at |
| (negative cofactor 2) | | | |
| DPH2-like 1 (S. cerevisiae) /// candidate tumor | DPH2L1 | Hs.513856 | 222041_at |
| suppressor in ovarian cancer 2 | /// | | |
| | OVCA2 | | |
| DRE1 protein | DRE1 | Hs.407709 | 206551_x_at |
| DRE1 protein | DRE1 | Hs.407709 | 221985_at |
| dual adaptor of phosphotyrosine and 3- | DAPP1 | Hs.436271 | 219290_x_at |
| phosphoinositides | | | |
| dual specificity phosphatase 1 | DUSP1 | Hs.171695 | 201041_s_at |
| dual specificity phosphatase 10 | DUSP10 | Hs.497822 | 215501_s_at |
| dual specificity phosphatase 10 | DUSP10 | Hs.497822 | 221563_at |
| dual specificity phosphatase 19 | DUSP19 | Hs.132237 | 1555598_a_at |
| dual specificity phosphatase 21 | DUSP21 | Hs.534478 | 220515_at |
| Dual specificity phosphatase 22 | DUSP22 | Hs.29106 | 226440_at |
| dual specificity phosphatase 6 | DUSP6 | Hs.298654 | 208892_s_at |
| dUTP pyrophosphatase | DUT | Hs.527980 | 208955_at |
| dUTP pyrophosphatase | DUT | Hs.527980 | 208956_x_at |
| dUTP pyrophosphatase | DUT | Hs.527980 | 209932_s_at |
| dymeclin | DYM | Hs.162996 | 223171_at |
| Dynamin 1-like /// CGI-04 protein | DNM1L | Hs.505231 /// | 226154_at |
| | /// CGI04 | Hs.550499 | |
| dynamin 3 | DNM3 | Hs.494751 | 209839_at |
| Dynein light chain 2 | Dlc2 | Hs.514368 | 225494_at |
| dynein light chain 2 | Dlc2 | Hs.514368 | 229106_at |
| dynein, axonemal, heavy polypeptide 7 | DNAH7 | Hs.97403 | 214222_at |
| dynein, axonemal, heavy polypeptide 7 | DNAH7 | Hs.97403 | 234476_at |
| dynein, axonemal, intermediate polypeptide 1 | DNAI1 | Hs.112667 | 233195_at |
| dynein, cytoplasmic, intermediate polypeptide 1 | DNCI1 | Hs.440364 | 205348_s_at |
| dynein, cytoplasmic, intermediate polypeptide 2 /// | DNCI2 | Hs.546250 | 211684_s_at |
| dynein, cytoplasmic, intermediate polypeptide 2 | | | |
| dynein, cytoplasmic, light intermediate polypeptide 2 | DNCLI2 | Hs.369068 | 224616_at |
| dynein, cytoplasmic, light polypeptide 1 | DNCL1 | Hs.5120 | 200703_at |
| dynein, cytoplasmic, light polypeptide 2A | DNCL2A | Hs.531876 | 217917_s_at |
| dynein, cytoplasmic, light polypeptide 2A | DNCL2A | Hs.531876 | 217918_at |
| Dystonin | DST | Hs.485616 | 215810_x_at |
| dystrobrevin, alpha | DTNA | Hs.58919 | 205741_s_at |
| Dystrobrevin, alpha | DTNA | Hs.58919 | 240125_at |
| E1A binding protein p300 | EP300 | Hs.517517 | 202221_s_at |
| E2a-Pbx1-associated protein | EB-1 | Hs.506458 | 227439_at |
| E2F transcription factor 2 | E2F2 | Hs.194333 | 228361_at |
| E3 identified by differential display | EDD | Hs.492445 | 208882_s_at |
| E3 identified by differential display | EDD | Hs.492445 | 232264_at |
| E74-like factor 2 (ets domain transcription factor) | ELF2 | Hs.260903 | 203822_s_at |
| Early B-cell factor | EBF | Hs.308048 | 227646_at |
| Early endosome antigen 1, 162 kD | EEA1 | Hs.506309 | 1564798_at |
| early growth response 1 | EGR1 | Hs.326035 | 201694_s_at |
| Early growth response 1 | EGR1 | Hs.326035 | 227404_s_at |
| early growth response 3 | EGR3 | Hs.534313 | 206115_at |
| EBNA1 binding protein 2 | EBNA1BP2 | Hs.346868 | 201323_at |
| echinoderm microtubule associated protein like 2 | EML2 | Hs.24178 | 204398_s_at |
| Echinoderm microtubule associated protein like 5 | EML5 | Hs.549263 | 242443_at |
| ecotropic viral integration site 1 | EVI1 | Hs.165830 | 226420_at |
| ectodysplasin A receptor | EDAR | Hs.171971 | 220048_at |
| ectonucleoside triphosphate diphosphohydrolase 1 | ENTPD1 | Hs.550467 | 209473_at |
| ectonucleoside triphosphate diphosphohydrolase 4 | ENTPD4 | Hs.444389 | 204077_x_at |
| ectonucleotide | ENPP2 | Hs.190977 | 209392_at |
| pyrophosphatase/phosphodiesterase 2 (autotaxin) | | | |
| EF hand domain family, member D2 | EFHD2 | Hs.465374 | 222483_at |
| EF-hand domain (C-terminal) containing 1 | EFHC1 | Hs.403171 | 225656_at |
| EF-hand domain (C-terminal) containing 2 | EFHC2 | Hs.521953 | 220591_s_at |
| EH domain binding protein 1 | EHBP1 | Hs.271667 | 212650_at |
| EH-domain containing 1 | EHD1 | Hs.523774 | 209037_s_at |
| ELAV (embryonic lethal, abnormal vision, | ELAVL2 | Hs.166109 | 228260_at |
| Drosophila)-like 2 (Hu antigen B) | | | |
| ELAV (embryonic lethal, abnormal vision, | ELAVL3 | Hs.1701 | 227612_at |
| Drosophila)-like 3 (Hu antigen C) | | | |
| ELAV (embryonic lethal, abnormal vision, | ELAVL4 | Hs.213050 | 206051_at |
| Drosophila)-like 4 (Hu antigen D) | | | |
| ELAV (embryonic lethal, abnormal vision, | ELAVL4 | Hs.213050 | 241424_at |
| Drosophila)-like 4 (Hu antigen D) | | | |
| ELG protein | HSA277841 | Hs.120963 | 218896_s_at |
| ELISC-1 | — | Hs.128434 | 215287_at |
| ELISC-1 | — | Hs.128434 | 244354_at |
| ELL associated factor 1 | EAF1 | Hs.474479 | 226952_at |
| ELMO domain containing 2 | ELMOD2 | Hs.450105 | 226502_at |
| ELMO domain containing 2 | ELMOD2 | Hs.450105 | 233912_x_at |
| elongation factor RNA polymerase II-like 3 | ELL3 | Hs.171466 | 219518_s_at |
| elongation factor Tu GTP binding domain | EFTUD1 | Hs.459114 | 218973_at |
| containing 1 | | | |
| elongation factor, RNA polymerase II, 2 | ELL2 | Hs.192221 | 226099_at |
| Elongation factor, RNA polymerase II, 2 | ELL2 | Hs.192221 | 240038_at |
| ELOVL family member 5, elongation of long chain | ELOVL5 | Hs.520189 | 1566079_at |
| fatty acids (FEN1/Elo2, SUR4/Elo3-like, yeast) | | | |
| ELOVL family member 5, elongation of long chain | ELOVL5 | Hs.520189 | 214153_at |
| fatty acids (FEN1/Elo2, SUR4/Elo3-like, yeast) | | | |
| ELOVL family member 5, elongation of long chain | ELOVL5 | Hs.520189 | 215082_at |
| fatty acids (FEN1/Elo2, SUR4/Elo3-like, yeast) | | | |
| ELYS transcription factor-like protein TMBS62 | ELYS | Hs.300887 | 226115_at |
| endo-beta-N-acetylglucosaminidase | FLJ21865 | Hs.29288 | 65635_at |
| Endothelial PAS domain protein 1 | EPAS1 | Hs.468410 | 242868_at |
| endothelin receptor type B | EDNRB | Hs.82002 | 204271_s_at |
| Enhancer of polycomb homolog 1 (Drosophila) | EPC1 | Hs.167805 | 216437_at |
| enhancer of polycomb homolog 1 (Drosophila) | EPC1 | Hs.167805 | 225417_at |
| enhancer of polycomb homolog 1 (Drosophila) | EPC1 | Hs.167805 | 234969_s_at |
| Enhancer of zeste homolog 2 (Drosophila) | EZH2 | Hs.444082 | 215006_at |
| enolase 1, (alpha) | ENO1 | Hs.517145 | 201231_s_at |
| enoyl Coenzyme A hydratase domain containing 1 | ECHDC1 | Hs.486410 | 233124_s_at |
| Enoyl Coenzyme A hydratase domain containing 1 | ECHDC1 | Hs.486410 | 243303_at |
| EPH receptor A10 | EPHA10 | Hs.129435 | 243717_at |
| EPH receptor A5 | EPHA5 | Hs.479853 | 1557697_at |
| EPH receptor A5 | EPHA5 | Hs.479853 | 215664_s_at |
| epidermal growth factor receptor (erythroblastic | EGFR | Hs.488293 | 211551_at |
| leukemia viral (v-erb-b) oncogene homolog, avian) | | | |
| epidermal growth factor receptor pathway | EPS15 | Hs.83722 | 217887_s_at |
| substrate 15 | | | |
| epidermal growth factor receptor pathway | EPS15L1 | Hs.292072 | 222113_s_at |
| substrate 15-like 1 | | | |
| epididymal sperm binding protein 1 | ELSPBP1 | Hs.104894 | 220366_at |
| epithelial protein lost in neoplasm beta | EPLIN | Hs.525419 | 217892_s_at |
| Epithelial stromal interaction 1 (breast) | EPSTI1 | Hs.546467 | 1556297_a_at |
| epithelial stromal interaction 1 (breast) | EPSTI1 | Hs.546467 | 227609_at |
| EPM2A (laforin) interacting protein 1 | EPM2AIP1 | Hs.28020 | 202909_at |
| EPM2A (laforin) interacting protein 1 | EPM2AIP1 | Hs.28020 | 227847_at |
| Era G-protein-like 1 (E. coli) | ERAL1 | Hs.3426 | 212087_s_at |
| ERO1-like (S. cerevisiae) | ERO1L | Hs.525339 | 222646_s_at |
| Erythrocyte membrane protein band 4.1-like 2 | EPB41L2 | Hs.486470 | 242652_at |
| erythrocyte membrane protein band 4.1-like 3 | EPB41L3 | Hs.213394 | 212681_at |
| erythrocyte membrane protein band 4.1-like 3 /// | EPB41L3 | Hs.213394 | 211776_s_at |
| erythrocyte membrane protein band 4.1-like 3 | | | |
| esophageal cancer related gene 4 protein | ECRG4 | Hs.43125 | 223623_at |
| Espin | ESPN | Hs.147953 | 242327_x_at |
| essential meiotic endonuclease 1 homolog 1 (S. pombe) | EME1 | Hs.514330 | 234465_at |
| esterase D/formylglutathione hydrolase | ESD | Hs.432491 | 215096_s_at |
| estrogen receptor binding protein | ERBP | Hs.85769 | 202776_at |
| estrogen receptor binding site associated, antigen, 9 | EBAG9 | Hs.409368 | 204278_s_at |
| estrogen-related receptor beta like 1 | ESRRBL1 | Hs.412196 | 218100_s_at |
| ethanolamine kinase 1 | ETNK1 | Hs.240056 | 219017_at |
| ethanolamine kinase 2 | ETNK2 | Hs.497469 | 219268_at |
| etoposide induced 2.4 mRNA | EI24 | Hs.343911 | 216396_s_at |
| ets variant gene 1 | ETV1 | Hs.22634 | 206501_x_at |
| ets variant gene 6 (TEL oncogene) | ETV6 | Hs.504765 | 225764_at |
| euchromatic histone methyltransferase 1 | EHMT1 | Hs.495511 | 225461_at |
| eukaryotic elongation factor-2 kinase | EEF2K | Hs.549151 | 225545_at |
| eukaryotic elongation factor-2 kinase | EEF2K | Hs.549151 | 225546_at |
| eukaryotic translation elongation factor 1 alpha 1 | EEF1A1 | Hs.439552 | 204892_x_at |
| eukaryotic translation elongation factor 1 alpha 1 | EEF1A1 | Hs.439552 | 206559_x_at |
| eukaryotic translation elongation factor 1 alpha 1 | EEF1A1 | Hs.439552 | 213477_x_at |
| eukaryotic translation elongation factor 1 alpha 1 | EEF1A1 | Hs.439552 | 213614_x_at |
| eukaryotic translation elongation factor 1 beta 2 | EEF1B2 | Hs.421608 | 200705_s_at |
| eukaryotic translation elongation factor 1 delta | EEF1D | Hs.333388 | 203113_s_at |
| (guanine nucleotide exchange protein) | | | |
| Eukaryotic translation elongation factor 1 delta | EEF1D | Hs.333388 | 214395_x_at |
| (guanine nucleotide exchange protein) | | | |
| eukaryotic translation elongation factor 1 epsilon 1 | EEF1E1 | Hs.88977 | 204905_s_at |
| Eukaryotic translation elongation factor 1 epsilon 1 | EEF1E1 | Hs.88977 | 213907_at |
| eukaryotic translation initiation factor 1A, X-linked | EIF1AX | Hs.522590 | 201019_s_at |
| eukaryotic translation initiation factor 2, subunit 1 | EIF2S1 | Hs.151777 | 201143_s_at |
| alpha, 35 kDa | | | |
| eukaryotic translation initiation factor 2, subunit 2 | EIF2S2 | Hs.429180 | 208726_s_at |
| beta, 38 kDa | | | |
| Eukaryotic translation initiation factor 2, subunit 3 | EIF2S3 | Hs.539684 | 224935_at |
| gamma, 52 kDa | | | |
| eukaryotic translation initiation factor 2, subunit 3 | EIF2S3 | Hs.539684 | 224936_at |
| gamma, 52 kDa | | | |
| eukaryotic translation initiation factor 2-alpha | EIF2AK1 | Hs.520205 | 217736_s_at |
| kinase 1 | | | |
| eukaryotic translation initiation factor 2B, subunit 2 | EIF2B2 | Hs.409137 | 202461_at |
| beta, 39 kDa | | | |
| eukaryotic translation initiation factor 2C, 3 | EIF2C3 | Hs.530333 | 219426_at |
| eukaryotic translation initiation factor 3, subunit 10 | EIF3S10 | Hs.523299 | 200595_s_at |
| theta, 150/170 kDa | | | |
| eukaryotic translation initiation factor 3 subunit 10 | EIF3S10 | Hs.523299 | 200596_s_at |
| theta, 150/170 kDa | | | |
| eukaryotic translation initiation factor 3, subunit 6 | EIF3S6 | Hs.405590 | 208697_s_at |
| 48 kDa | | | |
| Eukaryotic translation initiation factor 3, subunit 6 | EIF3S6 | Hs.405590 | 235429_at |
| 48 kDa | | | |
| Eukaryotic translation initiation factor 3, subunit 6 | EIF3S6 | Hs.405590 | 236989_at |
| 48 kDa | | | |
| eukaryotic translation initiation factor 3, subunit 6 | EIF3S6IP | Hs.446852 | 217719_at |
| interacting protein | | | |
| eukaryotic translation initiation factor 3, subunit 9 | EIF3S9 | Hs.371001 | 203462_x_at |
| eta, 116 kDa | | | |
| eukaryotic translation initiation factor 3, subunit 9 | EIF3S9 | Hs.371001 | 208688_x_at |
| eta, 116 kDa | | | |
| eukaryotic translation initiation factor 4 gamma, 1 | EIF4G1 | Hs.433750 | 208624_s_at |
| eukaryotic translation initiation factor 4 gamma, 1 | EIF4G1 | Hs.433750 | 208625_s_at |
| eukaryotic translation initiation factor 4 gamma, 2 | EIF4G2 | Hs.183684 | 200004_at |
| /// eukaryotic translation initiation factor 4 gamma, 2 | | | |
| eukaryotic translation initiation factor 4 gamma, 3 | EIF4G3 | Hs.467084 | 1554309_at |
| eukaryotic translation initiation factor 4 gamma, 3 | EIF4G3 | Hs.467084 | 201935_s_at |
| Eukaryotic translation initiation factor 4 gamma, 3 | EIF4G3 | Hs.467084 | 243149_at |
| eukaryotic translation initiation factor 4A, isoform 1 | EIF4A1 | Hs.129673 | 201530_x_at |
| Eukaryotic translation initiation factor 4A, isoform 1 | EIF4A1 | Hs.129673 | 214805_at |
| eukaryotic translation initiation factor 4A, isoform 2 | EIF4A2 | Hs.478553 | 1556350_a_at |
| eukaryotic translation initiation factor 4A, isoform 2 | EIF4A2 | Hs.478553 | 200912_s_at |
| eukaryotic translation initiation factor 4B | EIF4B | Hs.512629 | 211938_at |
| eukaryotic translation initiation factor 4E | EIF4E | Hs.249718 | 201435_s_at |
| eukaryotic translation initiation factor 4E | EIF4E | Hs.249718 | 201437_s_at |
| eukaryotic translation initiation factor 4E | EIF4E | Hs.249718 | 237718_at |
| eukaryotic translation initiation factor 4E binding | EIF4EBP2 | Hs.522995 | 208770_s_at |
| protein 2 | | | |
| Eukaryotic translation initiation factor 4E binding | EIF4EBP2 | Hs.522995 | 224653_at |
| protein 2 | | | |
| eukaryotic translation initiation factor 4E member 2 | EIF4E2 | Hs.292026 | 213571_s_at |
| Eukaryotic translation initiation factor 4E member 2 | EIF4E2 | Hs.292026 | 226734_at |
| eukaryotic translation initiation factor 5 | EIF5 | Hs.433702 | 208290_s_at |
| eukaryotic translation initiation factor 5 | EIF5 | Hs.433702 | 208705_s_at |
| eukaryotic translation initiation factor 5 | EIF5 | Hs.433702 | 208706_s_at |
| eukaryotic translation initiation factor 5 | EIF5 | Hs.433702 | 208707_at |
| eukaryotic translation initiation factor 5 | EIF5 | Hs.433702 | 208708_x_at |
| eukaryotic translation initiation factor 5A | EIF5A | Hs.534314 | 201123_s_at |
| eukaryotic translation initiation factor 5A2 | EIF5A2 | Hs.164144 | 235296_at |
| Ewing sarcoma breakpoint region 1 | EWSR1 | Hs.374477 | 209214_s_at |
| Ewing sarcoma breakpoint region 1 | EWSR1 | Hs.374477 | 210011_s_at |
| Ewing sarcoma breakpoint region 1 | EWSR1 | Hs.374477 | 229966_at |
| excision repair cross-complementing rodent repair | ERCC5 | Hs.258429 | 202414_at |
| deficiency, complementation group 5 (xeroderma | | | |
| pigmentosum, complementation group G | | | |
| (Cockayne syndrome)) | | | |
| excision repair cross-complementing rodent repair | ERCC6 | — | 207347_at |
| deficiency, complementation group 6 | | | |
| exosome component 3 | EXOSC3 | Hs.549154 | 227916_x_at |
| exosome component 3 | EXOSC3 | Hs.549154 | 233495_at |
| exosome component 8 | EXOSC8 | Hs.294041 | 215136_s_at |
| Exostoses (multiple) 1 | EXT1 | Hs.492618 | 15643781_a_at |
| Exostoses (multiple) 1 | EXT1 | Hs.492618 | 215206_at |
| Exostoses (multiple) 1 | EXT1 | Hs.492618 | 232174_at |
| Exostoses (multiple) 1 | EXT1 | Hs.492618 | 234634_at |
| Exostoses (multiple) 1 | EXT1 | Hs.492618 | 239227_at |
| exostoses (multiple)-like 2 | EXTL2 | Hs.357637 | 209537_at |
| Exportin 7 | XPO7 | Hs.172685 | 235796_at |
| exportin, tRNA (nuclear export receptor for tRNAs) | XPOT | Hs.85951 | 212160_at |
| F11 receptor | F11R | Hs.517293 | 223000_s_at |
| Factor for adipocyte differentiation 158 | FAD158 | Hs.412836 | 1570007_at |
| factor for adipocyte differentiation 158 | FAD158 | Hs.412836 | 223533_at |
| family with sequence similarity 13, member A1 | FAM13A1 | Hs.97270 | 202973_x_at |
| family with sequence similarity 18, member B | FAM18B | Hs.87295 | 218446_s_at |
| family with sequence similarity 20, member A | FAM20A | Hs.268874 | 241981_at |
| family with sequence similarity 20, member A | FAM20A | Hs.268874 | 242945_at |
| Family with sequence similarity 20, member C | FAM20C | Hs.370354 | 244457_at |
| family with sequence similarity 21, member C /// | FAM21C | Hs.365286 | 211068_x_at |
| family with sequence similarity 21, member C | | | |
| family with sequence similarity 26, member C | FAM26C | Hs.530398 | 1554367_at |
| family with sequence similarity 3, member C | FAM3C | Hs.434053 | 201889_at |
| family with sequence similarity 32, member A | FAM32A | Hs.4766 | 201863_at |
| family with sequence similarity 33, member A | FAM33A | Hs.463607 | 225684_at |
| family with sequence similarity 35, member A | FAM35A | Hs.500419 | 220547_s_at |
| family with sequence similarity 36, member A | FAM36A | Hs.411490 | 224824_at |
| family with sequence similarity 40, member A | FAM40A | Hs.169577 | 225226_at |
| Family with sequence similarity 40, member A | FAM40A | Hs.169577 | 228944_at |
| family with sequence similarity 41, member C /// | FAM41C | Hs.449006 | 232899_at |
| similar to RPL23AP7 protein | /// | | |
| | MGC70863 | | |
| family with sequence similarity 44, member C | FAM44C | Hs.127882 | 231368_at |
| family with sequence similarity 45, member B /// | FAM45B | Hs.434241 | 221804_s_at |
| family with sequence similarity 45, member A | /// | | |
| | FAM45A | | |
| family with sequence similarity 46, member C | FAM46C | Hs.356216 | 220306_at |
| family with sequence similarity 46, member C | FAM46C | Hs.356216 | 226811_at |
| family with sequence similarity 48, member A | FAM48A | Hs.435815 | 216682_s_at |
| family with sequence similarity 48, member A | FAM48A | Hs.435815 | 220408_x_at |
| family with sequence similarity 48, member A | FAM48A | Hs.435815 | 221774_x_at |
| family with sequence similarity 49, member B | FAM49B | Hs.492869 | 217916_s_at |
| family with sequence similarity 53, member C | FAM53C | Hs.54056 | 218023_s_at |
| family with sequence similarity 54, member A | FAM54A | Hs.121536 | 228069_at |
| family with sequence similarity 59, member A | FAM59A | Hs.444314 | 219377_at |
| family with sequence similarity 60, member A | FAM60A | Hs.505154 | 220147_s_at |
| family with sequence similarity 60, member A | FAM60A | Hs.505154 | 223038_s_at |
| family with sequence similarity 61, member A | FAM61A | Hs.407368 | 212132_at |
| family with sequence similarity 61, member A | FAM61A | Hs.407368 | 222099_s_at |
| family with sequence similarity 8, member A1 | FAM8A1 | Hs.95260 | 203420_at |
| Fanconi anemia, complementation group C | FANCC | Hs.494529 | 205189_s_at |
| Fanconi anemia, complementation group D2 | FANCD2 | Hs.208388 | 1568889_at |
| Fanconi anemia, complementation group F | FANCF | Hs.523543 | 222713_s_at |
| farnesyl diphosphate synthase (farnesyl | FDPS | Hs.335918 | 201275_at |
| pyrophosphate synthetase, | | | |
| dimethylallyltranstransferase, | | | |
| geranyltranstransferase) | | | |
| farnesyl-diphosphate farnesyltransferase 1 | FDFT1 | Hs.546253 | 208647_at |
| Farnesyl-diphosphate farnesyltransferase 1 | FDFT1 | Hs.546253 | 238666_at |
| Farnesyl-diphosphate farnesyltransferase 1 | FDFT1 | Hs.546253 | 241954_at |
| farnesyltransferase, CAAX box, alpha /// | FNTA | Hs.370312 | 200090_at |
| farnesyltransferase, CAAX box, alpha | | | |
| Fas (TNF receptor superfamily, member 6) | FAS | Hs.244139 | 204780_s_at |
| fasciculation and elongation protein zeta 1 (zygin I) | FEZ1 | Hs.224008 | 203562_at |
| fascin homolog 3, actin-bundling protein, testicular | FSCN3 | Hs.128402 | 220379_at |
| (Strongylocentrotus purpuratus) | | | |
| fasting-inducible integral membrane protein TM6P1 | FLJ90024 | Hs.356187 | 226239_at |
| FAT tumor suppressor homolog 1 (Drosophila) | FAT | Hs.481371 | 201579_at |
| FAT tumor suppressor homolog 3 (Drosophila) | FAT3 | Hs.98523 | 236029_at |
| fatty acid 2-hydroxylase | FA2H | Hs.461329 | 219429_at |
| fatty acid binding protein 5 (psoriasis-associated) | FABP5 | Hs.408061 | 202345_s_at |
| Fatty acid binding protein 5 (psoriasis-associated) | FABP5 | Hs.408061 | 236271_at |
| F-box and leucine-rich repeat protein 11 | FBXL11 | Hs.124147 | 208988_at |
| F-box and leucine-rich repeat protein 12 | FBXL12 | Hs.12439 | 220127_s_at |
| F-box and leucine-rich repeat protein 13 | FBXL13 | Hs.434284 | 1553798_a_at |
| F-box and leucine-rich repeat protein 14 | FBXL14 | Hs.367956 | 213145_at |
| F-box and leucine-rich repeat protein 17 | FBXL17 | Hs.112143 | 227203_at |
| F-box and leucine-rich repeat protein 20 | FBXL20 | Hs.462946 | 235089_at |
| F-box and WD-40 domain protein 12 | FBXW12 | Hs.288793 | 215600_x_at |
| F-box and WD-40 domain protein 2 | FBXW2 | Hs.494985 | 218941_at |
| F-box and WD-40 domain protein 9 | FBXW9 | Hs.515154 | 223419_at |
| F-box protein 10 | FBXO10 | Hs.130774 | 227222_at |
| F-box protein 11 | FBXO11 | Hs.549201 | 203255_at |
| F-box protein 15 | FBXO15 | Hs.465411 | 231472_at |
| F-box protein 21 | FBXO21 | Hs.159699 | 212229_s_at |
| F-box protein 28 | FBXO28 | Hs.64691 | 1555971_s_at |
| F-box protein 33 | FBXO33 | Hs.324342 | 226970_at |
| F-box protein 34 | FBXO34 | Hs.525348 | 218539_at |
| F-box protein 38 /// F-box protein 38 | FBXO38 | Hs.483772 | 221257_x_at |
| F-box protein 45 | FBXO45 | Hs.518526 | 225099_at |
| F-box protein 45 | FBXO45 | Hs.518526 | 225100_at |
| F-box protein 7 | FBXO7 | Hs.5912 | 1554423_a_at |
| F-box protein 7 | FBXO7 | Hs.5912 | 201178_at |
| F-box protein 8 | FBXO8 | Hs.76917 | 1554806_a_at |
| F-box protein 9 | FBXO9 | Hs.216653 | 1566509_s_at |
| F-box protein 9 | FBXO9 | Hs.216653 | 210638_s_at |
| F-box protein 9 | FBXO9 | Hs.216653 | 212991_at |
| Fc fragment of IgE, high affinity I, receptor for; | FCER1G | Hs.433300 | 204232_at |
| gamma polypeptide | | | |
| Fc receptor-like protein 3 | FCRH3 | Hs.292449 | 231093_at |
| FCH and double SH3 domains 2 | FCHSD2 | Hs.445748 | 242563_at |
| FCH domain only 2 | FCHO2 | Hs.165762 | 228220_at |
| fer (fps/fes related) tyrosine kinase | FER | Hs.221472 | 206412_at |
| (phosphoprotein NCP94) | | | |
| fer-1-like 3, myoferlin (C. elegans) | FER1L3 | Hs.500572 | 201798_s_at |
| FERM and PDZ domain containing 2 | FRMPD2 | Hs.532437 | 239275_at |
| FERM domain containing 3 | FRMD3 | Hs.127535 | 229893_at |
| FERM domain containing 3 | FRMD3 | Hs.127535 | 230645_at |
| FERM domain containing 4A | FRMD4A | Hs.552599 | 1554034_a_at |
| FERM domain containing 4A | FRMD4A | Hs.552599 | 225167_at |
| FERM, RhoGEF (ARHGEF) and pleckstrin domain | FARP1 | Hs.403917 | 239246_at |
| protein 1 (chondrocyte-derived) | | | |
| ferredoxin 1 | FDX1 | Hs.744 | 203647_s_at |
| ferritin, heavy polypeptide 1 | FTH1 | Hs.446345 | 200748_s_at |
| ferritin, heavy polypeptide pseudogene 1 /// ferritin, | FTHP1 | Hs.453583 | 211628_x_at |
| heavy polypeptide pseudogene 1 | | | |
| fetal Alzheimer antigen | FALZ | Hs.444200 | 207186_s_at |
| fetal Alzheimer antigen | FALZ | Hs.444200 | 231953_at |
| fetal Alzheimer antigen | FALZ | Hs.444200 | 232909_s_at |
| Fetuin B | FETUB | Hs.81073 | 214417_s_at |
| FGFR1 oncogene partner | FGFR1OP | Hs.487175 | 1568678_s_at |
| FGFR1 oncogene partner | FGFR1OP | Hs.487175 | 205588_s_at |
| FGFR1 oncogene partner 2 | FGFR1OP2 | Hs.406184 | 1556283_s_at |
| fibrillarin /// fibrillarin | FBL | Hs.299002 | 211623_s_at |
| fibrinogen-like 1 | FGL1 | Hs.491143 | 205305_at |
| Fibroblast growth factor 12 | FGF12 | Hs.185577 | 1562794_at |
| fibroblast growth factor 13 | FGF13 | Hs.6540 | 205110_s_at |
| Fibroblast growth factor 18 | FGF18 | Hs.87191 | 231382_at |
| fibroblast growth factor 9 (glia-activating factor) | FGF9 | Hs.111 | 206404_at |
| fibroblast growth factor receptor 1 (fms-related | FGFR1 | Hs.549034 | 215404_x_at |
| tyrosine kinase 2, Pfeiffer syndrome) | | | |
| Fibroblast growth factor receptor 1 (fms-related | FGFR1 | Hs.549034 | 241724_x_at |
| tyrosine kinase 2, Pfeiffer syndrome) | | | |
| fibromodulin | FMOD | Hs.519168 | 202709_at |
| fibronectin type III and ankyrin repeat domains 1 | FANK1 | Hs.352591 | 232968_at |
| Fibronectin type III domain containing 3A | FNDC3A | Hs.508010 | 228534_s_at |
| fibronectin type III domain containing 3B | FNDC3B | Hs.159430 | 218618_s_at |
| Fibronectin type III domain containing 3B | FNDC3B | Hs.159430 | 244022_at |
| fibronectin type III domain containing 5 | FNDC5 | Hs.524234 | 226097_at |
| filamin C, gamma (actin binding protein 280) | FLNC | Hs.58414 | 207876_s_at |
| Finkel-Biskis-Reilly murine sarcoma virus (FBR- | FAU | Hs.387208 | 200019_s_at |
| MuSV) ubiquitously expressed (fox derived); | | | |
| ribosomal protein S30 /// Finkel-Biskis-Reilly | | | |
| murine sarcoma virus (FBR-MuSV) ubiquitously | | | |
| expressed (fox derived); ribosomal protein S30 | | | |
| FIS | FIS | Hs.8373 | 239380_at |
| FK506 binding protein 1A, 12 kDa | FKBP1A | Hs.471933 | 200709_at |
| FK506 binding protein 1A, 12 kDa | FKBP1A | Hs.471933 | 210187_at |
| FK506 binding protein 3, 25 kDa | FKBP3 | Hs.509226 | 218003_s_at |
| FK506 binding protein 5 | FKBP5 | Hs.407190 | 224840_at |
| FK506 binding protein 7 | FKBP7 | Hs.410378 | 224002_s_at |
| FKSG17 | FKSG17 | Hs.458400 | 211445_x_at |
| FKSG44 gene | FKSG44 | Hs.362974 | 227964_at |
| flavin containing monooxygenase 5 | FMO5 | Hs.303476 | 205776_at |
| FLJ00290 protein | FLJ00290 | Hs.535044 | 236467_at |
| FLJ10378 protein | FLJ10378 | Hs.480712 | 1554038_at |
| FLJ10378 protein | FLJ10378 | Hs.480712 | 231011_at |
| FLJ10378 protein | FLJ10378 | Hs.480712 | 231824_at |
| FLJ10378 protein | FLJ10378 | Hs.480712 | 242153_at |
| FLJ16287 protein | FLJ16287 | Hs.529141 | 1569077_x_at |
| FLJ20160 protein | FLJ20160 | Hs.418581 | 225325_at |
| FLJ20259 protein | FLJ20259 | Hs.297389 | 209174_s_at |
| FLJ20859 gene | FLJ20859 | Hs.512693 | 218366_x_at |
| FLJ20859 gene | FLJ20859 | Hs.512693 | 223528_s_at |
| FLJ20859 gene | FLJ20859 | Hs.512693 | 223988_x_at |
| FLJ25323 protein | FLJ25323 | Hs.534649 | 239977_at |
| FLJ27505 protein | FLJ27505 | Hs.177983 | 237428_at |
| FLJ30851 protein | FLJ30851 | Hs.467868 | 230435_at |
| FLJ34306 protein | FLJ34306 | Hs.500495 | 229962_at |
| FLJ34870 protein | FLJ34870 | Hs.270083 | 1561135_at |
| FLJ37099 protein | FLJ37099 | Hs.127350 | 233294_at |
| FLJ41131 protein | FLJ41131 | Hs.59839 | 222052_at |
| FLJ41603 protein | FLJ41603 | Hs.256206 | 227717_at |
| FLJ44216 protein | FLJ44216 | Hs.348516 | 228805_at |
| FLJ45235 protein | FLJ45235 | Hs.130054 | 236421_at |
| FLJ45803 protein | FLJ45803 | Hs.125166 | 238701_x_at |
| FLJ46072 protein | FLJ46072 | Hs.67776 | 226129_at |
| FLN29 gene product | FLN29 | Hs.5148 | 35254_at |
| folate receptor 1 (adult) /// folate receptor 1 (adult) | FOLR1 | Hs.73769 | 211074_at |
| follicular lymphoma variant translocation 1 | FVT1 | Hs.74050 | 202419_at |
| Follistatin-like 4 | FSTL4 | Hs.483390 | 1565909_at |
| Forkhead box C1 | FOXC1 | Hs.348883 | 213260_at |
| Forkhead box C2 (MFH-1, mesenchyme forkhead | FOXC2 | Hs.436448 | 239058_at |
| 1) | | | |
| forkhead box J3 | FOXJ3 | Hs.26023 | 217310_s_at |
| forkhead box K2 | FOXK2 | Hs.132161 | 203064_s_at |
| Forkhead box O3A | FOXO3A | Hs.220950 | 1569477_at |
| forkhead box O3A | FOXO3A | Hs.220950 | 204131_s_at |
| forkhead box O3A | FOXO3A | Hs.220950 | 224891_at |
| Forkhead box O3A | FOXO3A | Hs.220950 | 242320_at |
| Forkhead box P1 | FOXP1 | Hs.431498 | 215221_at |
| forkhead box P1 | FOXP1 | Hs.431498 | 223937_at |
| forkhead box P1 | FOXP1 | Hs.431498 | 224837_at |
| forkhead box P1 | FOXP1 | Hs.431498 | 224838_at |
| Forkhead box P1 | FOXP1 | Hs.431498 | 244845_at |
| forkhead box protein O6 | FOXO6 | — | 239657_x_at |
| forkhead box Q1 | FOXQ1 | Hs.297452 | 227475_at |
| formin binding protein 1-like | FNBP1L | Hs.134060 | 215017_s_at |
| Formin binding protein 1-like | FNBP1L | Hs.134060 | 242310_at |
| formin binding protein 3 | FNBP3 | Hs.298735 | 213729_at |
| formin binding protein 3 | FNBP3 | Hs.298735 | 218053_at |
| Formin-like 3 | FMNL3 | Hs.179838 | 230850_at |
| four and a half LIM domains 1 | FHL1 | Hs.435369 | 214505_s_at |
| four and a half LIM domains 2 | FHL2 | Hs.443687 | 202949_s_at |
| four and a half LIM domains 5 | FHL5 | Hs.283689 | 1555191_a_at |
| FP6778 | — | Hs.191482 | 225935_at |
| FP6778 | — | Hs.191482 | 227069_at |
| fracture callus 1 homolog (rat) | FXC1 | Hs.54943 | 217981_s_at |
| fracture callus 1 homolog (rat) | FXC1 | Hs.54943 | 222481_at |
| fragile X mental retardation, autosomal homolog 1 | FXR1 | Hs.478407 | 201635_s_at |
| fragile X mental retardation, autosomal homolog 1 | FXR1 | Hs.478407 | 201637_s_at |
| frequently rearranged in advanced T-cell | FRAT1 | Hs.126057 | 219889_at |
| lymphomas | | | |
| fucosyltransferase 10 (alpha (1,3) | FUT10 | Hs.458713 | 235472_at |
| fucosyltransferase) | | | |
| fucosyltransferase 11 (alpha (1,3) | FUT11 | Hs.500082 | 238551_at |
| fucosyltransferase) | | | |
| fucosyltransferase 2 (secretor status included) | FUT2 | Hs.443160 | 210608_s_at |
| fucosyltransferase 3 (galactoside 3(4)-L- | FUT3 | Hs.169238 | 214088_s_at |
| fucosyltransferase, Lewis blood group included) | | | |
| fucosyltransferase 8 (alpha (1,6) | FUT8 | Hs.118722 | 242889_x_at |
| fucosyltransferase) | | | |
| Full length insert cDNA clone YS02G11 | — | Hs.535341 | 242841_at |
| Full length insert cDNA clone YT8SD04 | — | Hs.384648 | 1561926_at |
| Full length insert cDNA clone ZB55F04 | — | Hs.142505 | 1557149_at |
| Full length insert cDNA clone ZD69D05 | — | Hs.379253 | 229994_at |
| Full length insert cDNA clone ZD73H04 | — | Hs.126397 | 240539_at |
| Full length insert cDNA clone ZD79H10 | — | Hs.22380 | 226272_at |
| Full length insert cDNA YU10E03 | — | Hs.508829 | 229026_at |
| Full open reading frame cDNA clone | — | Hs.55468 | 230795_at |
| RZPDo834C0824D for gene HIST2H4, histone 2, | | | |
| H4; complete cds, incl. stopcodon | | | |
| Full-length cDNA clone CS0DC001YL20 of | — | Hs.416013 | 238833_at |
| Neuroblastoma Cot 25-normalized of Homo | | | |
| sapiens (human) | | | |
| Full-length cDNA clone CS0DE002YJ16 of | — | Hs.287759 | 232513_x_at |
| Placenta of Homo sapiens (human) | | | |
| Full-length oDNA clone CS0DF027YF17 of Fetal | — | Hs.389452 | 242323_at |
| brain of Homo sapiens (human) | | | |
| Full-length cDNA clone CS0DF038YJ21 of Fetal | — | Hs.551805 | 222361_at |
| brain of Homo sapiens (human) | | | |
| Full-length cDNA clone CS0DI053YO09 of | — | Hs.151692 | 239017_at |
| Placenta Cot 25-normalized of Homo sapiens | | | |
| (human) | | | |
| Full-length cDNA clone CS0DI062YF05 of | — | Hs.536889 | 235829_at |
| Placenta Cot 25-normalized of Homo sapiens | | | |
| (human) | | | |
| Full-length cDNA clone CS0DM002YA18 of Fetal | — | Hs.436379 | 228240_at |
| liver of Homo sapiens (human) | | | |
| fumarylacetoacetate hydrolase domain containing 1 | FAHD1 | Hs.513265 | 226767_s_at |
| fumarylacetoacetate hydrolase domain containing 1 | FAHD1 | Hs.513265 | 227960_s_at |
| FUN14 domain containing 2 | FUNDC2 | Hs.356050 | 223042_s_at |
| FUS interacting protein (serine-arginine rich) 1 | FUSIP1 | Hs.3530 | 213594_x_at |
| fused toes homolog (mouse) | FTS | Hs.380897 | 223894_s_at |
| fusion (involved in t(12; 16) in malignant | FUS | Hs.513522 | 200959_at |
| liposarcoma) | | | |
| FXYD domain containing ion transport regulator 3 | FXYD3 | Hs.301350 | 202488_s_at |
| FYN oncogene related to SRC, FGR, YES | FYN | Hs.390567 | 210105_s_at |
| FYN oncogene related to SRC, FGR, YES | FYN | Hs.390567 | 243006_at |
| FYVE and coiled-coil domain containing 1 | FYCO1 | Hs.200227 | 244145_at |
| FYVE, RhoGEF and PH domain containing 4 | FGD4 | Hs.117835 | 227948_at |
| G kinase anchoring protein 1 | GKAP1 | Hs.522255 | 229311_at |
| G kinase anchoring protein 1 | GKAP1 | Hs.522255 | 229312_s_at |
| G kinase anchoring protein 1 | GKAP1 | Hs.522255 | 234192_s_at |
| G patch domain containing 2 | GPATC2 | Hs.420757 | 1556126_s_at |
| G protein-coupled receptor 18 | GPR18 | Hs.549036 | 210279_at |
| G protein-coupled receptor 56 | GPR56 | Hs.513633 | 212070_at |
| G protein-coupled receptor 61 | GPR61 | Hs.514690 | 244618_at |
| G protein-coupled receptor 89 | GPR89 | Hs.319301 | 220642_x_at |
| G protein-coupled receptor kinase 4 | GRK4 | Hs.32959 | 210600_s_at |
| G1 to S phase transition 1 | GSPT1 | Hs.528780 | 240452_at |
| G1 to S phase transition 2 /// G1 to S phase | GSPT2 | Hs.59523 | 205541_s_at |
| transition 2 | | | |
| G-2 and S-phase expressed 1 /// G-2 and S-phase | GTSE1 | Hs.386189 | 211040_x_at |
| expressed 1 | | | |
| GA binding protein transcription factor, alpha | GABPA | Hs.473470 | 227428_at |
| subunit 60 kDa | | | |
| GABA(A) receptor-associated protein | GABARAP | HS.84359 | 200645_at |
| GABA(A) receptor-associated protein like 1 | GABARAPL1 | Hs.524250 | 208868_s_at |
| GABA(A) receptor-associated protein like 1 | GABARAPL1 | Hs.524250 | 208869_s_at |
| GABA(A) receptor-associated protein-like 2 | GABARAPL2 | Hs.461379 | 209046_s_at |
| galactosamine (N-acetyl)-6-sulfate sulfatase | GALNS | Hs.271383 | 206335_at |
| (Morquio syndrome, mucopolysaccharidosis type | | | |
| IVA) | | | |
| galactosylceramidase (Krabbe disease) | GALC | Hs.513439 | 211810_s_at |
| gamma tubulin ring complex protein (76p gene) | 76P | Hs.549135 | 211337_s_at |
| gamma-aminobutyric acid (GABA) A receptor, beta 3 | GABRB3 | Hs.302352 | 229724_at |
| gamma-glutamyl hydrolase (conjugase, | GGH | Hs.78619 | 203560_at |
| folylpolygammaglutamyl hydrolase) | | | |
| gamma-glutamyltransferase 1 | GGT1 | Hs.287505 | 207131_x_at |
| gamma-glutamyltransferase 1 | GGT1 | Hs.287505 | 208284_x_at |
| gamma-glutamyltransferase-like activity 4 | GGTLA4 | Hs.355394 | 211416_x_at |
| GATA binding protein 3 | GATA3 | Hs.524134 | 209604_s_at |
| Gem (nuclear organelle) associated protein 4 | GEMIN4 | Hs.499620 | 223783_s_at |
| Gene differentially expressed in prostate | GDEP | Hs.104215 | 1554591_at |
| gene model 83 | Gm83 | Hs.532265 | 225676_s_at |
| gene model 83 | Gm83 | Hs.532265 | 231784_s_at |
| general transcription factor II, i /// general | GTF2I /// | Hs.520459 | 201065_s_at |
| transcription factor II, i, pseudogene 1 | GTF2IP1 | | |
| general transcription factor II, i /// general | GTF2I /// | Hs.520459 | 210891_s_at |
| transcription factor II, i, pseudogene 1 | GTF2IP1 | | |
| General transcription factor IIA, 1, 19/37 kDa | GTF2A1 | Hs.510068 | 1559009_at |
| general transcription factor IIA, 2, 12 kDa | GTF2A2 | Hs.512934 | 202678_at |
| General transcription factor IIA, 2, 12 kDa | GTF2A2 | Hs.512934 | 230376_at |
| General transcription factor IIA, 2, 12 kDa | GTF2A2 | Hs.512934 | 243985_at |
| general transcription factor IIE, polypeptide 1, | GTF2E1 | Hs.445272 | 205930_at |
| alpha 56 kDa | | | |
| general transcription factor IIE, polypeptide 2, beta | GTF2E2 | Hs.77100 | 202680_at |
| 34 kDa | | | |
| general transcription factor IIF, polypeptide 1, | GTF2F1 | Hs.68257 | 202356_s_at |
| 74 kDa | | | |
| general transcription factor IIH, polypeptide 1, | GTF2H1 | Hs.523510 | 202451_at |
| 62 kDa | | | |
| general transcription factor IIH, polypeptide 2, | GTF2H2 | Hs.191356 | 221540_x_at |
| 44 kDa | | | |
| general transcription factor IIH, polypeptide 5 | GTF2H5 | Hs.356224 | 213357_at |
| geranylgeranyl diphosphate synthase 1 | GGPS1 | Hs.55498 | 202321_at |
| geranylgeranyl diphosphate synthase 1 | GGPS1 | Hs.55498 | 202322_s_at |
| germ cell associated 1 | GSG1 | Hs.240053 | 241553_at |
| germ cell associated 1 /// germ cell associated 1 | GSG1 | Hs.240053 | 221252_s_at |
| germ cell associated 2 (haspin) | GSG2 | Hs.534059 | 223759_s_at |
| germ cell-less homolog 1 (Drosophila) /// germ cell- | GCL /// | Hs.484313 | 234488_s_at |
| less homolog 1 (Drosophila)-like | GMCL1L | | |
| GLI-Kruppel family member HKR1 | HKR1 | Hs.129165 | 227603_at |
| Glioma amplified sequence 64 | — | Hs.416155 | 238853_at |
| glioma tumor suppressor candidate region gene 2 | GLTSCR2 | Hs.421907 | 217807_s_at |
| glucocorticoid induced transcript 1 | GLCCI1 | Hs.131673 | 225706_at |
| glucosamine (UDP-N-acetyl)-2-epimerase/N- | GNE | Hs.5920 | 205042_at |
| acetylmannosamine kinase | | | |
| glucosamine-phosphate N-acetyltransferase 1 | GNPNAT1 | Hs.478025 | 225853_at |
| glucosaminyl (N-acetyl) transferase 3, mucin type | GCNT3 | Hs.194710 | 219508_at |
| glucose phosphate isomerase | GPI | Hs.466471 | 208308_s_at |
| glucosidase, alpha; neutral AB | GANAB | Hs.76847 | 214626_s_at |
| Glucosidase, beta (bile acid) 2 | GBA2 | Hs.443134 | 228292_at |
| glutamate dehydrogenase 2 | GLUD2 | Hs.368538 | 215794_x_at |
| glutamate receptor, ionotropic, N-methyl D- | GRINL1A | Hs.437256 | 212241_at |
| aspartate-like 1A | | | |
| glutamate receptor, metabotropic 6 | GRM6 | Hs.248131 | 208035_at |
| glutamate-ammonia ligase (glutamine synthase) | GLUL | Hs.518525 | 200648_s_at |
| glutamate-ammonia ligase (glutamine synthase) | GLUL | Hs.518525 | 215001_s_at |
| glutamate-ammonia ligase (glutamine synthase) | GLUL | Hs.518525 | 217202_s_at |
| glutamate-cysteine ligase, catalytic subunit | GCLC | Hs.271264 | 202922_at |
| glutaminase | GLS | HS.116448 | 203159_at |
| Glutamine-fructose-6-phosphate transaminase 1 | GFPT1 | Hs.468864 | 1560596_at |
| glutaminyl-tRNA synthetase | QARS | Hs.79322 | 217846_at |
| glutamyl-prolyl-tRNA synthetase | EPRS | Hs.497788 | 200842_s_at |
| glutaredoxin 2 | GLRX2 | Hs.458283 | 219933_at |
| glutathione peroxidase 4 (phospholipid | GPX4 | Hs.433951 | 201106_at |
| hydroperoxidase) | | | |
| Glutathione S-transferase A1 | GSTA1 | Hs.446309 | 215766_at |
| glutathione S-transferase kappa 1 | GSTK1 | Hs.390667 | 217751_at |
| glutathione S-transferase M1 | GSTM1 | Hs.301961 | 215333_x_at |
| glutathione S-transferase M2 (muscle) | GSTM2 | Hs.279837 | 204418_x_at |
| glutathione S-transferase M3 (brain) | GSTM3 | Hs.2006 | 202554_s_at |
| glutathione S-transferase M3 (brain) | GSTM3 | Hs.2006 | 235867_at |
| glutathione S-transferase M4 | GSTM4 | Hs.348387 | 210912_x_at |
| glutathione S-transferase M5 | GSTM5 | Hs.75652 | 205752_s_at |
| glutathione S-transferase omega 1 | GSTO1 | Hs.190028 | 201470_at |
| glutathione synthetase | GSS | Hs.82327 | 201415_at |
| glutathione synthetase /// glutathione synthetase | GSS | Hs.82327 | 211630_s_at |
| glycerol kinase 2 | GK2 | Hs.98008 | 215430_at |
| glycerol-3-phosphate dehydrogenase 2 | GPD2 | Hs.148266 | 210007_s_at |
| (mitochondrial) | | | |
| Glycerol-3-phosphate dehydrogenase 2 | GPD2 | Hs.148266 | 243598_at |
| (mitochondrial) | | | |
| glycerophosphodiester phosphodiesterase domain | GDPD1 | Hs.153485 | 1555606_a_at |
| containing 1 | | | |
| glycerophosphodiester phosphodiesterase domain | GDPD3 | Hs.289015 | 219722_s_at |
| containing 3 | | | |
| glycine cleavage system protein H (aminomethyl | GCSH | Hs.546256 | 213129_s_at |
| carrier) | | | |
| glycine cleavage system protein H (aminomethyl | GCSH | Hs.546256 | 213133_s_at |
| carrier) | | | |
| Glycine cleavage system protein H (aminomethyl | IQWD1 | Hs.435741 | 235613_at |
| carrier) | | | |
| glycogen synthase kinase 3 alpha | GSK3A | Hs.466828 | 632_at |
| Glycogen synthase kinase 3 beta | GSK3B | Hs.445733 | 226191_at |
| glycogenin | GYG | Hs.477892 | 211275_s_at |
| glycolipid transfer protein | GLTP | Hs.381256 | 226177_at |
| glycoprotein (transmembrane) nmb | GPNMB | Hs.190495 | 201141_at |
| glycoprotein VI (platelet) | GP6 | Hs.272216 | 220336_s_at |
| glycosyltransferase 25 domain containing 2 | GLT25D2 | Hs.387995 | 209883_at |
| glycosyltransferase-like domain containing 1 | GTDC1 | Hs.44780 | 220853_at |
| glypican 2 (cerebroglycan) | GPC2 | Hs.211701 | 239422_at |
| GNAS complex locus | GNAS | Hs.125898 | 200780_x_at |
| GNAS complex locus | GNAS | Hs.125898 | 200981_x_at |
| GNAS complex locus | GNAS | Hs.125898 | 211858_x_at |
| GNAS complex locus | GNAS | Hs.125898 | 212273_x_at |
| GNAS complex locus | GNAS | Hs.125898 | 214157_at |
| GNAS complex locus | GNAS | Hs.125898 | 228173_at |
| golgi apparatus protein 1 | GLG1 | Hs.201712 | 214730_s_at |
| golgi associated PDZ and coiled-coil motif | GOPC | Hs.191539 | 227214_at |
| containing | | | |
| Golgi associated PDZ and coiled-coil motif | GOPC | Hs.191539 | 236862_at |
| containing | | | |
| golgi associated, gamma adaptin ear containing, | GGA1 | Hs.499158 | 45572_s_at |
| ARF binding protein 1 | | | |
| golgi associated, gamma adaptin ear containing, | GGA1 | Hs.499158 | 50277_at |
| ARF binding protein 1 | | | |
| golgi associated, gamma adaptin ear containing, | GGA2 | Hs.460336 | 208913_at |
| ARF binding protein 2 | | | |
| golgi associated, gamma adaptin ear containing, | GGA2 | Hs.460336 | 210658_s_at |
| ARF binding protein 2 | | | |
| golgi associated, gamma adaptin ear containing, | GGA3 | Hs.87726 | 211815_s_at |
| ARF binding protein 3 | | | |
| golgi autoantigen, golgin subfamily a, 3 | GOLGA3 | Hs.507333 | 1554987_at |
| golgi autoantigen, golgin subfamily a, 5 | GOLGA5 | Hs.104320 | 218241_at |
| golgi autoantigen, golgin subfamily a, member 6 /// | GOLGA6 | Hs.326276 | 207575_at |
| similar to Golgi autoantigen, golgin subfamily A | /// | | |
| member 6 (Golgin linked to PML) (Golgin-like | LOC388137 | | |
| protein) /// similar to COMM domain containing 4 | /// | | |
| | LOC440292 | | |
| golgi autoantigen, golgin subfamily b, macrogolgin | GOLGB1 | Hs.213389 | 201057_s_at |
| (with transmembrane signal), 1 | | | |
| golgi phosphoprotein 3 (coat-protein) | GOLPH3 | Hs.408909 | 217803_at |
| golgi phosphoprotein 4 | GOLPH4 | Hs.143600 | 238002_at |
| golgi reassembly stacking protein 2, 55 kDa | GORASP2 | Hs.431317 | 207812_s_at |
| golgi reassembly stacking protein 2, 55 kDa | GORASP2 | Hs.431317 | 208842_s_at |
| golgi reassembly stacking protein 2, 55 kDa | GORASP2 | Hs.431317 | 208843_s_at |
| golgi SNAP receptor complex member 1 | GOSR1 | Hs.462680 | 204630_s_at |
| golgi SNAP receptor complex member 2 | GOSR2 | Hs.463278 | 213180_s_at |
| golgin-67 | GOLGIN- | Hs.182982 | 208797_s_at |
| | 67 | | |
| golgin-67 | GOLGIN- | Hs.182982 | 208798_x_at |
| | 67 | | |
| golgin-67 | GOLGIN- | Hs.182982 | 213650_at |
| | 67 | | |
| GPI deacylase | PGAP1 | Hs.229988 | 220576_at |
| GPI7 protein | GPI7 | Hs.7099 | 218652_s_at |
| grancalcin, EF-hand calcium binding protein /// | GCA | Hs.377894 | 203765_at |
| grancalcin, EF-hand calcium binding protein | | | |
| GRINL1A complex upstream protein | Gup1 | Hs.50841 | 228568_at |
| GRIP and coiled-coil domain containing 1 | GCC1 | Hs.521168 | 218912_at |
| growth arrest-specific 5 | GAS5 | Hs.531856 | 228238_at |
| growth factor receptor-bound protein 10 | GRB10 | Hs.164060 | 209409_at |
| growth factor receptor-bound protein 10 | GRB10 | Hs.164060 | 210999_s_at |
| growth hormone inducible transmembrane protein | GHITM | Hs.352656 | 1554510_s_at |
| Growth hormone inducible transmembrane protein | GHITM | Hs.352656 | 1564148_at |
| growth hormone inducible transmembrane protein | GHITM | Hs.352656 | 209249_s_at |
| GTF2I repeat domain containing 2 | GTF2IRD2 | Hs.270962 | 228765_at |
| GTP binding protein 4 | GTPBP4 | Hs.215766 | 218239_s_at |
| GTPase activating protein and VPS9 domains 1 | GAPVD1 | Hs.495134 | 212802_s_at |
| GTPase activating protein and VPS9 domains 1 | GAPVD1 | Hs.495134 | 214869_x_at |
| GTPase activating Rap/RanGAP domain-like 1 | GARNL1 | Hs.113150 | 234923_at |
| GTP-binding protein PTD004 | PTD004 | Hs.157351 | 219293_s_at |
| guanine monphosphate synthetase | GMPS | Hs.546324 | 214431_at |
| guanine nucleotide binding protein (G protein) | GNA12 | Hs.487341 | 224681_at |
| alpha 12 | | | |
| guanine nucleotide binding protein (G protein), | GNA11 | Hs.515056 | 213766_x_at |
| alpha 11 (Gq class) | | | |
| guanine nucleotide binding protein (G protein), | GNA11 | Hs.515056 | 213944_x_at |
| alpha 11 (Gq class) | | | |
| guanine nucleotide binding protein (G protein), | GNA11 | Hs.515056 | 40562_at |
| alpha 11 (Gq class) | | | |
| guanine nucleotide binding protein (G protein), | GNA13 | Hs.515018 | 224761_at |
| alpha 13 | | | |
| guanine nucleotide binding protein (G protein), | GNB1 | Hs.430425 | 200745_s_at |
| beta polypeptide 1 | | | |
| Guanine nucleotide binding protein (G protein), | GNB1 | Hs.430425 | 241865_at |
| beta polypeptide 1 | | | |
| Guanine nucleotide binding protein (G protein), | GNG10 | Hs.501879 | 240326_at |
| gamma 10 | | | |
| Guanine nucleotide binding protein (G protein), | GNG2 | Hs.187772 | 243819_at |
| gamma 2 | | | |
| guanine nucleotide binding protein (G protein), | GNG3 | Hs.179915 | 222005_s_at |
| gamma 3 | | | |
| Guanine nucleotide binding protein (G protein), | GNG4 | Hs.159711 | 1566512_at |
| gamma 4 | | | |
| guanine nucleotide binding protein-like 1 | GNL1 | Hs.83147 | 203307_at |
| Guanine nucleotide binding protein-like 1 | GNL1 | Hs.83147 | 233544_at |
| guanylate binding protein 2, interferon-inducible | GBP2 | Hs.386567 | 242907_at |
| guanylate cyclase activator 1A (retina) | GUCA1A | Hs.92858 | 1255_g_at |
| guanylate cyclase activator 1A (retina) | GUCA1A | Hs.92858 | 206062_at |
| GULP, engulfment adaptor PTB domain containing 1 | GULP1 | Hs.470887 | 204235_s_at |
| H19, imprinted maternally expressed untranslated | H19 | Hs.551588 | 224997_x_at |
| mRNA | | | |
| H2A histone family, member X | H2AFX | Hs.477879 | 213344_s_at |
| H2A histone family, member Z | H2AFZ | Hs.119192 | 213911_s_at |
| H3 histone, family 3A | H3F3A | Hs.533624 | 208755_x_at |
| H3 histone, family 3A | H3F3A | Hs.533624 | 211940_x_at |
| H3 histone, family 3A | H3F3A | Hs.533624 | 213828_x_at |
| H3 histone, family 3A /// H3 histone, family 3A | H3F3A | Hs.533624 | 200080_s_at |
| H3 histone, family 3B (H3.3B) | H3F3B | Hs.180877 | 209069_s_at |
| H3 histone, family 3B (H3.3B) | H3F3B | Hs.180877 | 211997_x_at |
| H3 histone, family 3B (H3.3B) | H3F3B | Hs.180877 | 211998_at |
| H3 histone, family 3B (H3.3B) | H3F3B | Hs.180877 | 211999_at |
| hairy/enhancer-of-split related with YRPW motif- | HEYL | Hs.472566 | 226828_s_at |
| like | | | |
| HBS1-like (S. cerevisiae) | HBS1L | Hs.378532 | 209316_s_at |
| headcase homolog (Drosophila) | HECA | Hs.197644 | 218603_at |
| heat shock 10 kDa protein 1 (chaperonin 10) | HSPE1 | Hs.1197 | 205133_s_at |
| heat shock 60 kDa protein 1 (chaperonin) | HSPD1 | Hs.113684 | 200807_s_at |
| heat shock 70 kDa protein 1A | HSPA1A | Hs.520028 | 200799_at |
| heat shock 70 kDa protein 1B | HSPA1B | Hs.274402 | 202581_at |
| heat shock 70 kDa protein 1-like | HSPA1L | Hs.80288 | 210189_at |
| heat shock 70 kDa protein 4-like | HSPA4L | Hs.135554 | 205543_at |
| heat shock 70 kDa protein 8 | HSPA8 | Hs.180414 | 221891_x_at |
| heat shock 90 kDa protein 1, alpha | HSPCA | Hs.523560 | 210211_s_at |
| heat shock 90 kDa protein 1, alpha | HSPCA | Hs.523560 | 211968_s_at |
| heat shock 90 kDa protein 1, alpha | HSPCA | Hs.523560 | 211969_at |
| heat shock 90 kDa protein 1, alpha | HSPCA | Hs.523560 | 214328_s_at |
| heat shock factor binding protein 1 | HSBP1 | Hs.250899 | 200941_at |
| heat shock transcription factor 2 binding protein | HSF2BP | Hs.406157 | 207020_at |
| heat shock transcription factor, Y-linked 1 /// heat | HSFY1 | Hs.448418 | 224007_at |
| shock transcription factor, Y linked 2 | /// | | |
| | HSFY2 | | |
| hect (homologous to the E6-AP (UBE3A) carboxyl | HERC1 | Hs.210385 | 218306_s_at |
| terminus) domain and RCC1 (CHC1)-like domain | | | |
| (RLD) 1 | | | |
| hect domain and RLD 4 | HERC4 | Hs.51891 | 225988_at |
| hect domain and RLD 5 | HERC5 | Hs.26663 | 219863_at |
| HECT domain containing 2 | HECTD2 | Hs.66378 | 227568_at |
| HECT, UBA and WWE domain containing 1 | HUWE1 | Hs.136905 | 208598_s_at |
| hematopoietic stem/progenitor cells 176 | HSPC176 | Hs.461722 | 218354_at |
| hemochromatosis | HFE | Hs.233325 | 206087_x_at |
| hemochromatosis | HFE | Hs.233325 | 211328_x_at |
| hemogen | HEMGN | Hs.176626 | 223670_s_at |
| heparan sulfate (glucosamine) 3-O- | HS3ST2 | Hs.115830 | 219697_at |
| sulfotransferase 2 | | | |
| hepatitis B virus x associated protein | HBXAP | Hs.420229 | 218166_s_at |
| hepatitis B virus x associated protein | HBXAP | Hs.420229 | 222540_s_at |
| hepatitis B virus x interacting protein | HBXIP | Hs.439815 | 202299_s_at |
| hepatitis B virus x interacting protein | HBXIP | Hs.439815 | 202300_at |
| hepatocellularcarcinoma-associated antigen | DKFZP586D0919 | Hs.528351 | 213861_s_at |
| HCA557a | | | |
| hepatocyte growth factor-regulated tyrosine kinase. | HGS | Hs.514590 | 210428_s_at |
| substrate | | | |
| hepatoma derived growth factor-like 1 | HDGFL1 | Hs.97124 | 232680_at |
| hepatoma-derived growth factor (high-mobility | HDGF | Hs.506748 | 200896_x_at |
| group protein 1-like) | | | |
| hepatoma-derived growth factor, related protein 3 | HDGFRP3 | Hs.513954 | 209524_at |
| hepatoma-derived growth factor, related protein 3 | HDGFRP3 | Hs.513954 | 209526_s_at |
| hepatoma-derived growth factor, related protein 3 | HDGFRP3 | Hs.513954 | 216693_x_at |
| Hepatoma-derived growth factor-related protein 2 | HDGF2 | Hs.43071 | 207730_x_at |
| Hermansky-Pudlak syndrome 5 | HPS5 | Hs.437599 | 204544_at |
| HERV-H LTR-associating 2 | HHLA2 | Hs.252351 | 234624_at |
| HERV-H LTR-associating 3 | HHLA3 | Hs.142245 | 234665_x_at |
| HESB like domain containing 2 | HBLD2 | Hs.449291 | 209273_s_at |
| HESB like domain containing 2 | HBLD2 | Hs.449291 | 209274_s_at |
| HESB like domain containing 2 /// HESB like | HBLD2 | Hs.449291 | 221425_s_at |
| domain containing 2 | | | |
| heterogeneous nuclear ribonucleoprotein A/B | HNRPAB | Hs.248746 | 201277_s_at |
| heterogeneous nuclear ribonucleoprotein A0 | HNRPA0 | Hs.96996 | 201055_s_at |
| Heterogeneous nuclear ribonucleoprotein A0 | HNRPA0 | Hs.96996 | 229083_at |
| heterogeneous nuclear ribonucleoprotein A1 | HNRPA1 | Hs.546261 | 214280_x_at |
| heterogeneous nuclear ribonucleoprotein A1 /// | HNRPA1 | Hs.546261 | 200016_x_at |
| heterogeneous nuclear ribonucleoprotein A1 | | | |
| heterogeneous nuclear ribonucleoprotein A1 /// | HNRPA1 | Hs.534833 | 213356_x_at |
| similar to Heterogeneous nuclear ribonucleoprotein | /// | | |
| A1 (Helix-destabilizing protein) (Single-strand | LOC344741 | | |
| binding protein) (hnRNP core protein A1) (HDP) /// | /// | | |
| similar to Heterogeneous nuclear ribonucleoprotein | LOC389674 | | |
| A1 (Helix-destabilizing protein) (Single-strand | /// | | |
| binding protein) (hnRNP core protein A1) (HDP-1) | LOC391670 | | |
| (Topoisomerase-inhibitor suppressed) /// similar to | /// | | |
| Heterogeneous nuclear ribonucleoprotein A1 | LOC402112 | | |
| (Helix-destabilizing protein) (Single-strand binding | /// | | |
| protein) (hnRNP core protein A1) (HDP) /// similar | LOC402562 | | |
| to Heterogeneous nuclear ribonucleoprotein A1 | /// | | |
| (Helix-destabilizing protein) (Single-strand binding | LOC439963 | | |
| protein) (hnRNP core protein A1) (HDP) /// similar | /// | | |
| to Heterogeneous nuclear ribonucleoprotein A1 | LOC440125 | | |
| (Helix-destabilizing protein) (Single-strand binding | /// | | |
| protein) (hnRNP core protein A1) (HDP-1) | LOC441507 | | |
| (Topoisomerase-inhibitor suppressed) /// similar to | | | |
| heterogeneous nuclear ribonucleoprotein A1 /// | | | |
| similar to Heterogeneous nuclear ribonucleoprotein | | | |
| A1 (Helix-destabilizing protein) (Single-strand | | | |
| binding protein) (hnRNP core protein A1) (HDP) /// | | | |
| similar to Heterogeneous nuclear ribonucleoprotein | | | |
| A1 (Helix-destabilizing protein) (Single-strand | | | |
| binding protein) (hnRNP core protein A1) (HDP) | | | |
| heterogeneous nuclear ribonucleoprotein A1 /// | HNRPA1 | Hs.534833 | 216559_x_at |
| similar to Hnrpa1 protein /// similar to | /// | | |
| Heterogeneous nuclear ribonucleoprotein A1 | LOC120364 | | |
| (Helix-destabilizing protein) (Single-strand binding | /// | | |
| protein) (hnRNP core protein A1) (HDP) /// similar | LOC344741 | | |
| to Heterogeneous nuclear ribonucleoprotein A1 | /// | | |
| (Helix-destabilizing protein) (Single-strand binding | LOC389674 | | |
| protein) (hnRNP core protein A1) (HDP-1) | /// | | |
| (Topoisomerase-inhibitor suppressed) /// similar to | LOC391670 | | |
| Heterogeneous nuclear ribonucleoprotein A1 | /// | | |
| (Helix-destabilizing protein) (Single-strand binding | LOC402112 | | |
| protein) (hnRNP core protein A1) (HDP) /// similar | /// | | |
| to Heterogeneous nuclear ribonucleoprotein A1 | LOC402562 | | |
| (Helix-destabilizing protein) (Single-strand binding | /// | | |
| protein) (hnRNP core protein A1) (HDP) /// similar | LOC439963 | | |
| to Heterogeneous nuclear ribonucleoprotein A1 | /// | | |
| (Helix-destabilizing protein) (Single-strand binding | LOC440125 | | |
| protein) (hnRNP core protein A1) (HDP-1) | | | |
| (Topoisomerase-inhibitor suppressed) /// similar to | | | |
| heterogeneous nuclear ribonucleoprotein A1 /// | | | |
| similar to Heterogeneous nuclear ribonucleoprotein | | | |
| A1 (Helix-destabilizing protein) (Single-strand | | | |
| binding protein) (hnRNP core protein A1) (HDP) | | | |
| heterogeneous nuclear ribonucleoprotein A1 /// | HNRPA1 | Hs.534833 | 216497_at |
| similar to Hnrpa1 protein /// similar to | /// | | |
| Heterogeneous nuclear ribonucleoprotein A1 | LOC120364 | | |
| (Helix-destabilizing protein) (Single-strand binding | /// | | |
| protein) (hnRNP core protein A1) (HDP) /// similar | LOC344741 | | |
| to Heterogeneous nuclear ribonucleoprotein A1 | /// | | |
| (Helix-destabilizing protein) (Single-strand binding | LOC389674 | | |
| protein) (hnRNP core protein A1) (HDP-1) | /// | | |
| (Topoisomerase-inhibitor suppressed) /// similar to | LOC391670 | | |
| Heterogeneous nuclear ribonucleoprotein A1 | /// | | |
| (Helix-destabilizing protein) (Single-strand binding | LOC402112 | | |
| protein) (hnRNP core protein A1) (HDP) /// similar | /// | | |
| to Heterogeneous nuclear ribonucleoprotein A1 | LOC402562 | | |
| (Helix-destabilizing protein) (Single-strand binding | /// | | |
| protein) (hnRNP core protein A1) (HDP) /// similar | LOC439963 | | |
| to Heterogeneous nuclear ribonucleoprotein A1 | /// | | |
| (Helix-destabilizing protein) (Single-strand binding | LOC440125 | | |
| protein) (hnRNP core protein A1) (HDP-1) | /// | | |
| (Topoisomerase-inhibitor suppressed) /// similar to | LOC441507 | | |
| heterogeneous nuclear ribonucleoprotein A1 /// | | | |
| similar to Heterogeneous nuclear ribonucleoprotein | | | |
| A1 (Helix-destabilizing protein) (Single-strand | | | |
| binding protein) (hnRNP core protein A1) (HDP) /// | | | |
| similar to Heterogeneous nuclear ribonucleoprotein | | | |
| A1 (Helix-destabilizing protein) (Single-strand | | | |
| binding protein) (hnRNP core protein A1) (HDP) | | | |
| heterogeneous nuclear ribonucleoprotein A2/B1 | HNRPA2B1 | Hs.487774 | 205292_s_at |
| Heterogeneous nuclear ribonucleoprotein A2/B1 | HNRPA2B1 | Hs.487774 | 225932_s_at |
| heterogeneous nuclear ribonucleoprotein A3 | HNRPA3 | Hs.516539 | 211932_at |
| heterogeneous nuclear ribonucleoprotein A3 | HNRPA3P1 | Hs.524276 | 211931_s_at |
| pseudogene 1 /// heterogeneous nuclear | /// | | |
| ribonucleoprotein A3 | HNRPA3 | | |
| heterogeneous nuclear ribonucleoprotein C | HNRPC | Hs.449114 | 212626_x_at |
| (C1/C2) | | | |
| heterogeneous nuclear ribonucleoprotein C | HNRPC | Hs.449114 | 214737_x_at |
| (C1/C2) | | | |
| heterogeneous nuclear ribonucleoprotein C | HNRPC | Hs.449114 | 200014_s_at |
| (C1/C2) /// heterogeneous nuclear | | | |
| ribonucleoprotein C (C1/C2) | | | |
| heterogeneous nuclear ribonucleoprotein D-like | HNRPDL | Hs.527105 | 201993_x_at |
| heterogeneous nuclear ribonucleoprotein D-like | HNRPDL | Hs.527105 | 209067_s_at |
| Heterogeneous nuclear ribonucleoprotein D-like | HNRPDL | Hs.527105 | 212454_x_at |
| heterogeneous nuclear ribonucleoprotein H1 (H) | HNRPH1 | Hs.202166 | 201031_s_at |
| heterogeneous nuclear ribonucleoprotein H3 (2H9) | HNRPH3 | Hs.499891 | 207127_s_at |
| heterogeneous nuclear ribonucleoprotein H3 (2H9) | HNRPH3 | Hs.499891 | 208990_s_at |
| heterogeneous nuclear ribonucleoprotein H3 (2H9) | HNRPH3 | Hs.499891 | 210110_x_at |
| heterogeneous nuclear ribonucleoprotein H3 (2H9) | HNRPH3 | Hs.499891 | 210588_x_at |
| heterogeneous nuclear ribonucleoprotein K /// | HNRPK | Hs.522257 | 200097_s_at |
| heterogeneous nuclear ribonucleoprotein K | | | |
| heterogeneous nuclear ribonucleoprotein L | HNRPL | Hs.446623 | 35201_at |
| Heterogeneous nuclear ribonucleoprotein M | HNRPM | Hs.465808 | 1555843_at |
| heterogeneous nuclear ribonucleoprotein M | HNRPM | Hs.465808 | 1556084_at |
| heterogeneous nuclear ribonucleoprotein R | HNRPR | Hs.373763 | 208766_s_at |
| heterogeneous nuclear ribonucleoprotein U | HNRPU | Hs.166463 | 200593_s_at |
| (scaffold attachment factor A) | | | |
| heterogeneous nuclear ribonucleoprotein U | HNRPU | Hs.166463 | 200594_x_at |
| (scaffold attachment factor A) | | | |
| hexokinase 2 | HK2 | Hs.406266 | 202934_at |
| high density lipoprotein binding protein (vigilin) | HDLBP | Hs.471851 | 221767_x_at |
| high mobility group nucleosomal binding domain 3 | HMGN3 | Hs.77558 | 209377_s_at |
| high-mobility group box 1 | HMGB1 | Hs.434102 | 200680_x_at |
| high-mobility group box 1 | HMGB1 | Hs.434102 | 214938_x_at |
| high-mobility group box 1 | HMGB1 | Hs.434102 | 224731_at |
| high-mobility group nucleosomal binding domain 2 | HMGN2 | Hs.181163 | 208668_x_at |
| high-mobility group nucleosome binding domain 1 | HMGN1 | Hs.356285 | 200943_at |
| high-mobility group nucleosome binding domain 1 | HMGN1 | Hs.356285 | 200944_s_at |
| hippocalcin-like 1 | HPCAL1 | Hs.467696 | 212552_at |
| HIR histone cell cycle regulation defective homolog | HIRA | Hs.474206 | 240451_at |
| A (S. cerevisiae) | | | |
| HIRA interacting protein 5 | HIRIP5 | Hs.430439 | 218946_at |
| histamine receptor H3 | HRH3 | Hs.251399 | 1555715_a_at |
| histidine triad nucleotide binding protein 1 | HINT1 | Hs.483305 | 1555961_a_at |
| histidine triad nucleotide binding protein 2 /// | HINT2 | Hs.70573 | 224415_s_at |
| histidine triad nucleotide binding protein 2 | | | |
| histone 1, H2bc | HIST1H2BC | Hs.546314 | 236193_at |
| histone 1, H2bd | HIST1H2BD | Hs.130853 | 209911_x_at |
| histone 1, H4d | HIST1H4D | Hs.248179 | 208076_at |
| histone 2, H2aa | HIST2H2AA | Hs.530461 | 218280_x_at |
| histone 2, H2be | HIST2H2BE | Hs.2178 | 202708_s_at |
| histone acetyltransferase 1 | HAT1 | Hs.470611 | 203138_at |
| histone deacetylase 2 | HDAC2 | Hs.3352 | 201833_at |
| histone deacetylase 3 | HDAC3 | Hs.519632 | 216326_s_at |
| Histone deacetylase 3 | HDAC3 | Hs.519632 | 240482_at |
| histone deacetylase 9 | HDAC9 | Hs.196054 | 234393_at |
| HIV TAT specific factor 1 | HTATSF1 | Hs.204475 | 202602_s_at |
| HIV-1 induced protein HIN-1 | HSHIN1 | Hs.270851 | 203480_s_at |
| HIV-1 Rev binding protein | HRB | Hs.443507 | 218091_at |
| HIV-1 Rev binding protein | HRB | Hs.443507 | 218092_s_at |
| HIV-1 rev binding protein 2 | HRB2 | Hs.269857 | 232441_at |
| HIV-1 Tat interactive protein 2, 30 kDa | HTATIP2 | Hs.90753 | 209448_at |
| HIV-1 Tat interactive protein 2, 30 kDa | HTATIP2 | Hs.90753 | 210253_at |
| HLA class II region expressed gene KE2 | HKE2 | Hs.446374 | 242048_at |
| HLA complex group 18 | HCG18 | Hs.301985 | 242812_at |
| HLA complex group 2 pseudogene 7 | HCG2P7 | Hs.512758 | 216229_x_at |
| HLA complex group 8 | HCG8 | — | 215985_at |
| HLA-B associated transcript 3 | BAT3 | Hs.440900 | 210208_x_at |
| HLA-G histocompatibility antigen, class I, G | HLA-G | Hs.512152 | 211528_x_at |
| HMG2 like | LOC127540 | Hs.409307 | 230473_s_at |
| HMT1 hnRNP methyltransferase-like 4 (S. cerevisiae) | HRMT1L4 | Hs.504530 | 230839_at |
| Holocarboxylase synthetase (biotin-[proprionyl- | HLCS | Hs.371350 | 241726_at |
| Coenzyme A-carboxylase (ATP-hydrolysing)] | | | |
| ligase) | | | |
| Homeo box A13 | HOXA13 | Hs.414467 | 238571_at |
| homeo box A3 | HOXA3 | Hs.533357 | 235521_at |
| homeo box B9 | HOXB9 | Hs.463350 | 226461_at |
| homeo box C4 /// hypothetical gene supported by | HOXC4 | Hs.350378 | 206194_at |
| AK022887; AK056417 | /// | | |
| | FLJ12825 | | |
| homeo box C6 | HOXC6 | Hs.820 | 206858_s_at |
| Homeo box D8 | HOXD8 | Hs.301963 | 231906_at |
| Homeobox C14 | LOC360030 | Hs.484779 | 232744_x_at |
| homeodomain interacting protein kinase 1 | HIPK1 | Hs.532363 | 1552515_at |
| homeodomain interacting protein kinase 1 | HIPK1 | Hs.532363 | 212291_at |
| Homeodomain interacting protein kinase 2 | HIPK2 | Hs.397465 | 225116_at |
| homeodomain interacting protein kinase 3 | HIPK3 | Hs.201918 | 207764_s_at |
| homer homolog 1 (Drosophila) | HOMER1 | Hs.129051 | 226651_at |
| Homo sapiens, clone IMAGE: 2900578, mRNA, | — | Hs.375766 | 227710_s_at |
| partial cds | | | |
| Homo sapiens, clone IMAGE: 3604678, mRNA | — | Hs.527697 | 227484_at |
| Homo sapiens, clone IMAGE: 3930408, mRNA | — | Hs.34068 | 235010_at |
| Homo sapiens, clone IMAGE: 4044872, mRNA | — | Hs.205745 | 1569344_a_at |
| Homo sapiens, clone IMAGE: 4096273, mRNA | — | Hs.4055 | 1555832_s_at |
| Homo sapiens, clone IMAGE: 4096273, mRNA | — | Hs.4055 | 224606_at |
| Homo sapiens, clone IMAGE: 4214313, mRNA | — | Hs.288478 | 237203_at |
| Homo sapiens, clone IMAGE: 4214654, mRNA | — | Hs.16193 | 213156_at |
| Homo sapiens, clone IMAGE: 4214654, mRNA | — | Hs.16193 | 213158_at |
| Homo sapiens, clone IMAGE: 4246459, mRNA | — | Hs.516739 | 1564701_at |
| Homo sapiens, clone IMAGE: 4401608, mRNA | — | Hs.162105 | 233142_at |
| Homo sapiens, clone IMAGE: 4523945, mRNA | — | Hs.128770 | 238139_at |
| Homo sapiens, clone IMAGE: 4700331, mRNA | — | Hs.541578 | 1570269_at |
| Homo sapiens, clone IMAGE: 4723407, mRNA | — | Hs.374729 | 1563138_at |
| partial cds | | | |
| Homo sapiens, clone IMAGE: 4747087, mRNA | — | Hs.487393 | 226715_at |
| Homo sapiens, clone IMAGE: 4798168, mRNA | — | Hs.550817 | 1568799_at |
| Homo sapiens, clone IMAGE: 4819956, mRNA | — | Hs.127043 | 1563367_at |
| partial cds | | | |
| Homo sapiens, clone IMAGE: 4822139, mRNA | — | Hs.456065 | 1556263_s_at |
| Homo sapiens, clone IMAGE: 4822266, mRNA | — | Hs.385810 | 1568887_at |
| Homo sapiens, clone IMAGE: 4822684, mRNA | — | Hs.506258 | 1568915_at |
| Homo sapiens, clone IMAGE: 4822830, mRNA | — | Hs.330376 | 1560135_at |
| Homo sapiens, clone IMAGE: 4822875, mRNA | — | Hs.535410 | 215504_x_at |
| Homo sapiens, clone IMAGE: 4823092, mRNA | — | Hs.145221 | 1557838_at |
| Homo sapiens, clone IMAGE: 4823221, mRNA | — | Hs.99503 | 1568872_at |
| Homo sapiens, clone IMAGE: 4823340, mRNA | — | Hs.144492 | 1563103_at |
| Homo sapiens, clone IMAGE: 4825762, mRNA | — | Hs.275746 | 1561877_at |
| Homo sapiens, clone IMAGE: 4825783, mRNA | — | Hs.553019 | 1561618_at |
| Homo sapiens, clone IMAGE: 4826526, mRNA | — | Hs.191814 | 1557645_at |
| Homo sapiens, clone IMAGE: 4827386, mRNA | — | Hs.527876 | 1570372_at |
| Homo sapiens, clone IMAGE: 4828652, mRNA | — | Hs.319053 | 1569646_a_at |
| Homo sapiens, clone IMAGE: 4828738, mRNA | — | Hs.519887 | 1560070_at |
| Homo sapiens, clone IMAGE: 4829423, mRNA | — | Hs.122199 | 240685_at |
| Homo sapiens, clone IMAGE: 4829846, mRNA | — | Hs.156898 | 1568711_a_at |
| Homo sapiens, clone IMAGE: 4830945, mRNA | — | Hs.401404 | 1556850_at |
| Homo sapiens, clone IMAGE: 4831083, mRNA | — | Hs.385630 | 1569786_at |
| Homo sapiens, clone IMAGE: 4837645, mRNA, | — | Hs.149762 | 1560634_a_at |
| partial cds | | | |
| Homo sapiens, clone IMAGE: 4837650, mRNA, | — | Hs.363526 | 235696_at |
| partial cds | | | |
| Homo sapiens, clone IMAGE: 4837974, mRNA | — | Hs.407594 | 1561598_at |
| Homo sapiens, clone IMAGE: 4838968, mRNA | — | Hs.549478 | 237260_at |
| Homo sapiens, clone IMAGE: 4839037, mRNA | — | Hs.515075 | 1562276_at |
| Homo sapiens, clone IMAGE: 5019307, mRNA | — | Hs.445239 | 228642_at |
| Homo sapiens, clone IMAGE: 5103171, mRNA, | — | Hs.408584 | 240371_at |
| partial cds | | | |
| Homo sapiens, clone IMAGE: 5165176, mRNA | — | Hs.504540 | 1562674_at |
| Homo sapiens, clone IMAGE: 5166236, mRNA | — | Hs.479288 | 241432_at |
| Homo sapiens, clone IMAGE: 5166543, mRNA | — | Hs.407621 | 1559806_at |
| Homo sapiens, clone IMAGE: 5167446, mRNA | — | Hs.105323 | 1556183_at |
| Homo sapiens, clone IMAGE: 5167600, mRNA | — | Hs.178144 | 235736_at |
| Homo sapiens, clone IMAGE: 5168221, mRNA | — | Hs.254230 | 1564685_a_at |
| Homo sapiens, clone IMAGE: 5169759, mRNA | — | Hs.525499 | 1570234_at |
| Homo sapiens, clone IMAGE: 5170410, mRNA | — | Hs.434359 | 1563039_at |
| Homo sapiens, clone IMAGE: 5170855, mRNA | — | Hs.257807 | 1563115_at |
| Homo sapiens, clone IMAGE: 5171618, mRNA | — | Hs.112065 | 1560282_at |
| Homo sapiens, clone IMAGE: 5215917, mRNA | — | Hs.549543 | 228826_at |
| Homo sapiens, clone IMAGE: 5218705, mRNA | — | Hs.334269 | 1559220_at |
| Homo sapiens, clone IMAGE: 5222445, mRNA | — | Hs.434728 | 1560517_s_at |
| Homo sapiens, clone IMAGE: 5225645, mRNA | — | Hs.535921 | 1559870_at |
| Homo sapiens, clone IMAGE: 5244076, mRNA | — | Hs.528230 | 1560225_at |
| Homo sapiens, clone IMAGE: 5260583, mRNA /// | TOP1 | Hs.311402 /// | 1558354_s_at |
| Topoisomerase (DNA) I | | Hs.472737 | |
| Homo sapiens, clone IMAGE: 5261213, mRNA | — | Hs.371609 | 228315_at |
| Homo sapiens, clone IMAGE: 5261865, mRNA | — | Hs.547136 | 1560131_at |
| Homo sapiens, clone IMAGE: 5265638, mRNA | — | Hs.129283 | 1569996_at |
| Homo sapiens, clone IMAGE: 5266257, mRNA | — | Hs.146285 | 1568791_s_at |
| Homo sapiens, clone IMAGE: 5266772, mRNA | — | Hs.126005 | 1568663_a_at |
| Homo sapiens, clone IMAGE: 5267277, mRNA | — | Hs.407613 | 1557875_at |
| Homo sapiens, clone IMAGE: 5267398, mRNA | — | Hs.40289 | 225856_at |
| Homo sapiens, clone IMAGE: 5267398, mRNA | — | Hs.40289 | 227531_at |
| Homo sapiens, clone IMAGE: 5267453, mRNA | — | Hs.88594 | 1558440_at |
| Homo sapiens, clone IMAGE: 5267606, mRNA | — | Hs.371732 | 1556518_at |
| Homo sapiens, clone IMAGE: 5268616, mRNA | — | Hs.125503 | 1559344_at |
| Homo sapiens, clone IMAGE: 5268696, mRNA | — | Hs.90286 | 226546_at |
| Homo sapiens, clone IMAGE: 5269228, mRNA | — | Hs.125983 | 1556617_a_at |
| Homo sapiens, clone IMAGE: 5269899, mRNA | — | Hs.21965 | 237365_at |
| Homo sapiens, clone IMAGE: 5270538, mRNA | — | Hs.292105 | 1557491_at |
| Homo sapiens, clone IMAGE: 5271876, mRNA | — | Hs.125923 | 1559540_at |
| Homo sapiens, clone IMAGE: 5272062, mRNA | — | Hs.202419 | 1556913_a_at |
| Homo sapiens, clone IMAGE: 5272174, mRNA | — | Hs.409982 | 1568784_at |
| Homo sapiens, clone IMAGE: 5272174, mRNA | — | Hs.409982 | 1568785_a_at |
| Homo sapiens, clone IMAGE: 5272626, mRNA | — | Hs.511837 | 1561705_at |
| Homo sapiens, clone IMAGE: 5273537, mRNA | — | Hs.97740 | 244325_at |
| Homo sapiens, clone IMAGE: 5273707, mRNA | — | Hs.541333 | 1566689_at |
| Homo sapiens, clone IMAGE: 5274897, mRNA | — | Hs.351680 | 225225_at |
| Homo sapiens, clone IMAGE: 5276804, mRNA | — | Hs.535008 | 242305_at |
| Homo sapiens, clone IMAGE: 5278284, mRNA | — | Hs.438623 | 231992_x_at |
| Homo sapiens, clone IMAGE: 5278517, mRNA | — | Hs.517821 | 224644_at |
| Homo sapiens, clone IMAGE: 5285282, mRNA /// | — | Hs.370049 /// | 227917_at |
| MRNA; cDNA DKFZp434N2116 (from clone | | Hs.448067 /// | |
| DKFZp434N2116) /// CDNA FLJ39459 fis, clone | | Hs.491069 | |
| PROST2011439. | | | |
| Homo sapiens, clone IMAGE: 5288537, mRNA | — | Hs.451353 | 226137_at |
| Homo sapiens, clone IMAGE: 5288946, mRNA | — | Hs.375804 | 1561560_at |
| Homo sapiens, clone IMAGE: 5295612, mRNA | — | Hs.552762 | 1568615_a_at |
| Homo sapiens, clone IMAGE: 5296329, mRNA | — | Hs.178067 | 231191_at |
| Homo sapiens, clone IMAGE: 5297356, mRNA | — | Hs.382014 | 240828_at |
| Homo sapiens, clone IMAGE: 5298702, mRNA | — | Hs.407545 | 1568908_at |
| Homo sapiens, clone IMAGE: 5312078, mRNA | — | Hs.327170 | 1569416_at |
| Homo sapiens, clone IMAGE: 5480271, mRNA | — | Hs.427902 | 1560290_at |
| Homo sapiens, clone IMAGE: 5742085, mRNA | — | Hs.407533 | 1562943_at |
| Homo sapiens, clone IMAGE: 5742629, mRNA | — | Hs.546998 | 1562052_at |
| Homo sapiens, clone IMAGE: 5760143, mRNA | — | Hs.545915 | 1560794_at |
| Homo sapiens, clone IMAGE: 6152133, mRNA | — | Hs.547728 | 232975_at |
| Homo sapiens, Similar to carnitine deficiency- | — | Hs.128454 | 1569453_a_at |
| associated gene expressed in ventricle 1, clone | | | |
| IMAGE: 4837775, mRNA | | | |
| Homo sapiens, Similar to deafness, autosomal | — | Hs.87734 | 235365_at |
| dominant 5 homolog (human), clone | | | |
| IMAGE: 4551670, mRNA | | | |
| Homo sapiens, Similar to diaphanous homolog 3 | — | Hs.444351 | 229097_at |
| (Drosophila), clone IMAGE: 5277415, mRNA | | | |
| Homo sapiens, Similar to hypothetical protein | — | Hs.406810 | 1569482_at |
| FLJ20378, clone IMAGE: 5262617, mRNA | | | |
| Homo sapiens, Similar to hypothetical protein | — | Hs.252866 | 1568876_a_at |
| FLJ21394, clone IMAGE: 4820924, mRNA | | | |
| Homo sapiens, Similar to hypothetical protein | — | Hs.545353 | 1569680_at |
| FLJ21463, clone IMAGE: 4288701, mRNA | | | |
| Homo sapiens, Similar to hypothetical protein | — | Hs.200022 | 224752_at |
| MGC10526, clone IMAGE: 4133906, mRNA | | | |
| Homo sapiens, Similar to likely ortholog of yeast | — | Hs.397886 | 1570503_at |
| ARV1, clone IMAGE: 4576306, mRNA | | | |
| Homo sapiens, Similar to likely ortholog of yeast | — | Hs.28540 | 213929_at |
| ARV1, clone IMAGE: 4733238, mRNA | | | |
| Homo sapiens, Similar to Rho-associated, coiled- | — | Hs.17820 | 213044_at |
| coil containing protein kinase 1, clone | | | |
| IMAGE: 5269982, mRNA | | | |
| Homo sapiens, Similar to unnamed HERV-H | — | Hs.515223 | 227919_at |
| protein, clone IMAGE: 3996038, mRNA | | | |
| homocysteine-inducible, endoplasmic reticulum | HERPUD1 | Hs.146393 | 217168_s_at |
| stress-inducible, ubiquitin-like domain member 1 | | | |
| homolog of yeast INO80 | INO80 | Hs.292949 | 229356_x_at |
| homolog of yeast TIM14 | TIM14 | Hs.230601 | 225358_at |
| homolog of yeast TIM14 | TIM14 | Hs.230601 | 225359_at |
| HOM-TES-103 tumor antigen-like | HOM- | Hs.15243 | 36030_at |
| | TES-103 | | |
| hook homolog 1 (Drosophila) | HOOK1 | Hs.378836 | 219976_at |
| hook homolog 3 (Drosophila) | HOOK3 | Hs.162852 | 235114_x_at |
| hook homolog 3 (Drosophila) /// hook homolog 3 | HOOK3 | Hs.162852 | 224359_s_at |
| (Drosophila) | | | |
| host cell factor C2 | HCFC2 | Hs.506558 | 219484_at |
| HP1-BP74 | HP1- | Hs.142442 | 224592_x_at |
| | BP74 | | |
| HRAS-like suppressor | HRASLS | Hs.36761 | 219983_at |
| H-rev107-like protein 5 | HRLP5 | Hs.410316 | 231050_at |
| H-rev107-like protein 5 | HRLP5 | Hs.410316 | 231452_at |
| HS1 binding protein | HAX1 | Hs.199625 | 201145_at |
| HSPC009 protein | HSPC009 | Hs.16059 | 218026_at |
| HSPC038 protein | LOC51123 | Hs.374485 | 218059_at |
| HSPC049 protein | HSPC049 | Hs.371722 | 230888_at |
| HSPC103 | — | Hs.317051 | 1557817_a_at |
| HSPC103 | — | Hs.317051 | 1557818_x_at |
| HSPC128 protein | HSPC128 | Hs.90527 | 218936_s_at |
| HSPC128 protein | HSPC128 | Hs.90527 | 222792_s_at |
| HSPC135 protein | HSPC135 | Hs.127496 | 221046_s_at |
| HSPC163 protein | HSPC163 | Hs.445890 | 223993_s_at |
| HSPC182 protein | HSPC182 | Hs.30026 | 223051_at |
| human immunodeficiency virus type I enhancer | HIVEP1 | Hs.549037 | 204512_at |
| binding protein 1 | | | |
| Human immunodeficiency virus type I enhancer | HIVEP3 | Hs.325890 | 244764_at |
| binding protein 3 | | | |
| huntingtin interacting protein 2 | HIP2 | Hs.50308 | 202347_s_at |
| huntingtin interacting protein B | HYPB | Hs.517941 | 215038_s_at |
| Huntingtin interacting protein B | HYPB | Hs.517941 | 241458_at |
| Huntingtin interacting protein E | HYPE | Hs.549110 | 219910_at |
| Huntingtin interacting protein K | HYPK | Hs.511978 | 218680_x_at |
| hyaluronan and proteoglycan link protein 3 | HAPLN3 | Hs.447530 | 227262_at |
| hyaluronan binding protein 4 | HABP4 | Hs.494567 | 209818_s_at |
| hyaluronan synthase 2 | HAS2 | Hs.159226 | 206432_at |
| Hyaluronan-mediated motility receptor (RHAMM) | HMMR | Hs.72550 | 1557029_at |
| hyaluronan-mediated motility receptor (RHAMM) | HMMR | Hs.72550 | 207165_at |
| hydroxyacyl-Coenzyme A dehydrogenase/3- | HADHB | Hs.515848 | 201007_at |
| ketoacyl-Coenzyme A thiolase/enoyl-Coenzyme A | | | |
| hydratase (trifunctional protein), beta subunit | | | |
| hydroxysteroid (17-beta) dehydrogenase 12 | HSD17B12 | Hs.132513 | 217869_at |
| hypermethylated in cancer 2 | HIC2 | Hs.517434 | 212964_at |
| Hyperparathyroidism 2 (with jaw tumor) | HRPT2 | Hs.378996 | 222320_at |
| Hypothetical 55.1 kDa protein F09G8.5 in | LOC220074 | Hs.317243 | 217706_at |
| chromosome III | | | |
| Hypothetical 55.1 kDa protein F09G8.5 in | LOC220074 | Hs.317243 | 226717_at |
| chromosome III | | | |
| hypothetical BC042079 locus | LOC474358 | — | 237425_at |
| Hypothetical gene LOC130063 | — | Hs.437426 | 244728_at |
| hypothetical gene LOC132870 | LOC132870 | Hs.122053 | 237769_at |
| Hypothetical gene LOC133874 | — | Hs.551577 | 1569417_at |
| hypothetical gene LOC133874 | LOC133874 | Hs.551577 | 237956_s_at |
| Hypothetical gene supported by AF086559; | — | Hs.367856 | 233399_x_at |
| BC065734 | | | |
| Hypothetical gene supported by AF275804 | — | Hs.237396 | 238022_at |
| hypothetical gene supported by AK055666; | FLJ31104 | Hs.482141 | 1557466_at |
| BC039324 | | | |
| hypothetical gene supported by AK075484; | LOC339804 | Hs.140617 | 1568658_at |
| BC014578 | | | |
| hypothetical gene supported by AK075564; | LOC400451 | Hs.27373 | 51158_at |
| BC060873 | | | |
| Hypothetical gene supported by AK091527 | — | Hs.407087 | 242853_at |
| Hypothetical gene supported by AK091718 | — | Hs.446271 | 226635_at |
| Hypothetical gene supported by AK092637 | — | Hs.220931 | 237484_at |
| hypothetical gene supported by AK093266 | LOC400238 | Hs.131037 | 1557657_a_at |
| Hypothetical gene supported by AK093334; | LOC255326 | Hs.314437 /// | 239151_at |
| AL833330; BC020871; BC032492 /// Homo | | Hs.499534 /// | |
| sapiens, Similar to ARF GTPase-activating protein, | | Hs.531181 | |
| clone IMAGE: 5245118, mRNA /// Hypothetical | | | |
| protein LOC255326 | | | |
| hypothetical gene supported by AK094796 | LOC400764 | Hs.297988 | 230433_at |
| Hypothetical gene supported by AK094957 | — | Hs.131718 | 244564_at |
| Hypothetical gene supported by AK094963 | — | Hs.381996 | 1561389_at |
| hypothetical gene supported by AK097381; | LOC400680 | Hs.531627 | 1562348_at |
| BC040866 | | | |
| hypothetical gene supported by AK123449; | LOC401155 | Hs.494023 | 244395_at |
| BX641014 /// hypothetical gene supported by | /// | | |
| AK123449; BX641014 | FLJ41455 | | |
| hypothetical gene supported by AK124699 | FLJ42709 | Hs.171132 | 229014_at |
| hypothetical gene supported by AK128660 | LOC400566 | Hs.499607 | 229238_at |
| hypothetical gene supported by AL449243 | LOC91689 | Hs.306083 | 225794_s_at |
| Hypothetical gene supported by AL713796 | — | Hs.389638 | 225046_at |
| Hypothetical gene supported by BC013438 | LOC375295 | Hs.126007 | 234826_at |
| hypothetical gene supported by BC015790; | LOC400573 | Hs.306721 | 236001_at |
| BC041634 | | | |
| Hypothetical gene supported by BC017229 | — | Hs.102276 | 235938_at |
| Hypothetical gene supported by BC028186 | — | Hs.385650 | 1557754_at |
| hypothetical gene supported by BC029568 | MGC39584 | Hs.130535 | 240814_at |
| Hypothetical gene supported by BC029568 | MGC39584 | Hs.130535 | 242822_at |
| hypothetical gene supported by BC032913; | LOC389023 | Hs.97540 | 236351_at |
| BC048425 | | | |
| hypothetical gene supported by BC032955 | LOC401530 | Hs.521524 | 1570388_a_at |
| hypothetical gene supported by BC032955 /// | LOC401530 | Hs.521524 | 1556378_a_at |
| hypothetical gene supported by AK127288; | /// | | |
| AY343901 | LOC440896 | | |
| hypothetical gene supported by BC033316 /// | LOC400742 | Hs.465825 | 1556195_a_at |
| hypothetical gene supported by BC033316 | /// | | |
| | LOC440572 | | |
| hypothetical gene supported by BC037858 | LOC440117 | Hs.127100 | 240268_at |
| hypothetical gene supported by BC039313 | LOC284861 | Hs.546531 | 233338_at |
| hypothetical gene supported by BC040576 | LOC400920 | Hs.434698 | 239558_at |
| Hypothetical gene supported by BC042734 | — | Hs.434230 | 1568660_a_at |
| hypothetical gene supported by BC044741 | LOC387872 | Hs.434392 | 244429_at |
| hypothetical gene supported by BX248251 | LOC387978 | Hs.92556 | 243061_at |
| Hypothetical gene supported by BX647608 | — | Hs.411391 | 232113_at |
| hypothetical gene supported by NM_173604 | LOC439919 | Hs.166754 | 1553614_a_at |
| hypothetical gene supported by NM_173604 | LOC439919 | Hs.166754 | 241953_at |
| Hypothetical LOC 441046 | — | Hs.518857 | 213251_at |
| hypothetical LOC129881 | LOC129881 | Hs.370111 | 236909_at |
| hypothetical LOC131076 | LOC131076 | Hs.220594 | 241943_at |
| hypothetical LOC149018 | LOC149018 | Hs.125785 | 231363_at |
| Hypothetical LOC151363 | — | Hs.350698 | 237982_at |
| hypothetical LOC158813 | LOC158813 | Hs.98197 | 231159_at |
| Hypothetical LOC203413 | LOC203413 | Hs.97892 | 1559258_a_at |
| Hypothetical LOC253662 | — | Hs.190267 | 237343_at |
| hypothetical LOC283677 | LOC283677 | Hs.97610 | 240689_at |
| hypothetical LOC283768 | LOC283768 | Hs.169639 | 222149_x_at |
| hypothetical LOC283768 /// golgi autoantigen, | LOC283768 | Hs.169639 | 213737_x_at |
| golgin family member /// similar to hypothetical | /// | | |
| protein | LOC390535 | | |
| | /// | | |
| | LOC440234 | | |
| hypothetical LOC284067 | LOC284067 | Hs.252707 | 231453_at |
| Hypothetical LOC387905 | — | Hs.147563 | 1561221_x_at |
| Hypothetical LOC387937 | — | Hs.458461 | 217332_at |
| Hypothetical LOC388134 | — | Hs.33333 | 225270_at |
| hypothetical LOC388381 | LOC388381 | Hs.250182 | 244316_at |
| Hypothetical LOC388397 | — | Hs.528473 | 227227_at |
| Hypothetical LOC388572 | — | Hs.534673 | 231886_at |
| Hypothetical LOC388650 | LOC388650 | Hs.180946 | 213689_x_at |
| hypothetical LOC388650 | LOC388650 | Hs.180946 | 216044_x_at |
| hypothetical LOC388684 | LOC388684 | Hs.263424 | 244843_x_at |
| Hypothetical LOC388727 | — | Hs.23459 | 1556274_at |
| hypothetical LOC388727 | LOC388727 | Hs.23459 | 231233_at |
| hypothetical LOC388882 | LOC388882 | Hs.126178 | 1554940_a_at |
| hypothetical LOC389048 | LOC389048 | Hs.546579 | 242881_x_at |
| hypothetical LOC389177 | FLJ23172 | Hs.306885 | 217016_x_at |
| Hypothetical LOC389232 | — | Hs.126428 | 237758_at |
| hypothetical LOC389440 | LOC389440 | Hs.32804 | 230487_at |
| hypothetical LOC389624 | LOC389624 | Hs.129800 | 244135_at |
| Hypothetical LOC389634 | — | Hs.491073 | 1560001_at |
| hypothetical LOC389715 | LOC389715 | Hs.522093 | 216084_at |
| hypothetical LOC389715 | LOC389715 | Hs.522093 | 241690_at |
| Hypothetical LOC399933 | — | Hs.503429 | 228612_at |
| hypothetical LOC400236 | LOC400236 | — | 227887_at |
| Hypothetical LOC400590 | LOC400590 | Hs.406166 | 241910_x_at |
| hypothetical LOC400843 | LOC400843 | Hs.5215 | 225860_at |
| hypothetical LOC401022 | LOC401022 | Hs.98661 | 242042_s_at |
| hypothetical LOC401074 | LOC401074 | Hs.528540 | 1559826_a_at |
| Hypothetical LOC401131 | — | Hs.458285 | 1566144_at |
| Hypothetical LOC401131 | — | Hs.458285 | 1566146_x_at |
| Hypothetical LOC402532 | — | Hs.418231 | 243009_at |
| Hypothetical LOC402563 | — | Hs.489028 | 236282_at |
| hypothetical LOC79954 | FLJ14075 | Hs.222494 | 222684_s_at |
| hypothetical LOC83640 | MGC2560 | Hs.80624 | 225210_s_at |
| hypothetical locus FLJ25758 | FLJ25758 | Hs.367930 | 1554286_at |
| hypothetical locus FLJ35487 | FLJ35487 | Hs.407054 | 1563743_at |
| hypothetical locus LOC494558 | LOC494558 | Hs.130722 | 236071_at |
| Hypothetical protein | — | Hs.369455 | 1560818_at |
| Hypothetical protein | — | Hs.369455 | 1560819_a_at |
| Hypothetical protein | — | Hs.369455 | 231440_at |
| hypothetical protein A-211C6.1 | LOC57149 | Hs.185489 | 203897_at |
| Hypothetical protein BC001096 | LOC92689 | Hs.476517 | 213455_at |
| hypothetical protein BC004360 | LOC87769 | Hs.350868 | 232422_at |
| hypothetical protein BC007899 | LOC148898 | Hs.61884 | 229086_at |
| hypothetical protein BC008207 | LOC92345 | Hs.332844 | 244828_x_at |
| hypothetical protein BC008322 | MGC15763 | Hs.475602 | 227686_at |
| hypothetical protein BC009467 | LOC158960 | Hs.111407 | 1558685_a_at |
| hypothetical protein BC009514 | LOC127253 | Hs.348411 | 227141_at |
| hypothetical protein BC009518 | LOC90799 | Hs.515009 | 225705_at |
| hypothetical protein BC011880 | LOC113444 | Hs.27160 | 225404_at |
| hypothetical protein BC011880 | LOC113444 | Hs.27160 | 239510_at |
| hypothetical protein BC015148 | LOC93081 | Hs.398111 | 213346_at |
| hypothetical protein BC017169 | LOC91137 | Hs.75639 | 212833_at |
| hypothetical protein BC019238 | LOC120379 | Hs.420662 | 235062_at |
| hypothetical protein C8orf9 | LOC157740 | — | 239877_at |
| hypothetical protein DJ328E19.C1.1 /// | DJ328E1 | Hs.515947 | 212854_x_at |
| hypothetical protein FLJ20719 /// AG1 protein | 9.C1.1 /// | | |
| | FLJ20719 | | |
| | /// AG1 | | |
| hypothetical protein DJ328E19.C1.1 /// | DJ328E1 | Hs.546664 | 226740_x_at |
| hypothetical protein FLJ20719 /// hypothetical | 9.C1.1 /// | | |
| protein LOC200030 /// hypothetical protein | FLJ20719 | | |
| MGC8902 /// AG1 protein /// hypothetical | /// | | |
| LOC440675 | LOC200030 | | |
| | /// | | |
| | MGC8902 | | |
| | /// AG1 | | |
| | /// | | |
| | LOC440675 | | |
| hypothetical protein DJ971N18.2 | DJ971N18.2 | Hs.169358 | 201580_s_at |
| hypothetical protein DKFZp313A2432 | DKFZp313A2432 | Hs.349096 | 226278_at |
| hypothetical protein DKFZp434F142 | DKFZp434F142 | Hs.307081 | 224123_at |
| hypothetical protein DKFZp434G156 | NAG6 | Hs.521178 | 226972_s_at |
| hypothetical protein DKFZp434H0115 | DKFZP434H0115 | Hs.201134 | 223924_at |
| hypothetical protein DKFZP434I0714 | DKFZP434I0714 | Hs.142307 | 231954_at |
| hypothetical protein DKFZp434K191 | DKFZp434K191 | Hs.318898 | 1557633_at |
| hypothetical protein DKFZp434L142 | DKFZp434L142 | Hs.323583 | 223204_at |
| hypothetical protein DKFZp434M202 | DKFZp434M202 | Hs.387671 | 231371_at |
| hypothetical protein DKFZp434O0320 | DKFZp434O0320 | Hs.194995 | 234925_at |
| hypothetical protein DKFZp434P055 | DKFZp434P055 | Hs.132519 | 232398_at |
| Hypothetical protein DKFZp547A023 | DKFZp547A023 | Hs.485899 | 226000_at |
| Hypothetical protein DKFZp547B1713 | DKFZp547B1713 | Hs.549239 | 240803_at |
| hypothetical protein DKFZp547E052 | DKFZp547E052 | Hs.471514 | 233164_x_at |
| Hypothetical protein DKFZp547K1113 | DKFZp547K1113 | Hs.459311 | 213658_at |
| hypothetical protein DKFZp564D172 | DKFZP564D172 | Hs.127788 | 212936_at |
| Hypothetical protein DKFZp564I0422 | DKFZP564I0422 | Hs.245798 | 230380_at |
| hypothetical protein DKFZp564K0822 /// | DKFZP564K0822 | Hs.488307 | 208091_s_at |
| hypothetical protein DKFZp564K0822 | | | |
| hypothetical protein DKFZp566M1046 | DKFZP566M1046 | Hs.501793 | 223637_s_at |
| Hypothetical protein DKFZp566M114 | DKFZP566M114 | Hs.146162 | 235143_at |
| Hypothetical protein DKFZp566N034 | DKFZP566N034 | Hs.369471 | 217713_x_at |
| Hypothetical protein DKFZp667M2411 | DKFZp667M2411 | Hs.462684 | 230099_at |
| hypothetical protein DKFZp727G131 | DKFZp727G131 | Hs.546520 | 238609_at |
| hypothetical protein DKFZp761I2123 | DKFZp761I2123 | Hs.77978 | 54970_at |
| hypothetical protein DKFZp762F0713 | DKFZp762F0713 | Hs.416214 | 242592_at |
| hypothetical protein DKFZp779O175 | DKFZp779O175 | Hs.124047 | 238952_x_at |
| hypothetical protein ET | ET | Hs.464166 | 221192_x_at |
| hypothetical protein ET | ET | Hs.464166 | 223242_s_at |
| hypothetical protein FLJ10081 | FLJ10081 | Hs.516341 | 224318_s_at |
| hypothetical protein FLJ10094 | FLJ10094 | Hs.128258 | 219501_at |
| Hypothetical protein FLJ10156 | FLJ10156 | Hs.404323 | 230076_at |
| hypothetical protein FLJ10159 | FLJ10159 | Hs.445244 | 218974_at |
| hypothetical protein FLJ10199 | FLJ10199 | Hs.465305 | 218815_s_at |
| hypothetical protein FLJ10276 | FLJ10276 | Hs.353454 | 218004_at |
| hypothetical protein FLJ10357 | FLJ10357 | Hs.35125 | 241627_x_at |
| hypothetical protein FLJ10385 | FLJ10385 | Hs.437460 | 44563_at |
| hypothetical protein FLJ10534 | FLJ10534 | Hs.388170 | 218155_x_at |
| hypothetical protein FLJ10534 | FLJ10534 | Hs.388170 | 218156_s_at |
| hypothetical protein FLJ10534 | FLJ10534 | Hs.388170 | 221987_s_at |
| Hypothetical protein FLJ10618 | FLJ10618 | Hs.144130 | 201918_at |
| Hypothetical protein FLJ10618 | FLJ10618 | Hs.144130 | 201919_at |
| hypothetical protein FLJ10652 | FLJ10652 | Hs.445129 | 218614_at |
| Hypothetical protein FLJ10656 | P15RS | Hs.464912 | 228566_at |
| hypothetical protein FLJ10700 | FLJ10700 | Hs.232197 | 218464_s_at |
| hypothetical protein FLJ10726 | FLJ10726 | Hs.195060 | 218314_s_at |
| hypothetical protein FLJ10769 | FLJ10769 | Hs.408324 | 217940_s_at |
| hypothetical protein FLJ10808 | FLJ10808 | Hs.212774 | 222600_s_at |
| hypothetical protein FLJ10826 | FLJ10826 | Hs.231883 | 225106_s_at |
| hypothetical protein FLJ10901 | FLJ10901 | Hs.518997 | 219010_at |
| hypothetical protein FLJ10979 /// hypothetical | FLJ10979 | Hs.461819 | 221707_s_at |
| protein FLJ10979 | | | |
| hypothetical protein FLJ10996 | FLJ10996 | Hs.107845 | 219774_at |
| Hypothetical protein FLJ11000 | FLJ11000 | Hs.521215 | 243465_at |
| hypothetical protein FLJ11011 | FLJ11011 | Hs.492031 | 1562458_at |
| Hypothetical protein FLJ11016 | FLJ11016 | Hs.139053 | 243673_at |
| hypothetical protein FLJ11029 | FLJ11029 | Hs.274448 | 219392_x_at |
| hypothetical protein FLJ11029 | FLJ11029 | Hs.274448 | 232215_x_at |
| Hypothetical protein FLJ11155 | FLJ11155 | Hs.176227 | 1562608_at |
| hypothetical protein FLJ11236 | FLJ11236 | — | 228433_at |
| hypothetical protein FLJ11280 | FLJ11280 | Hs.3346 | 226062_x_at |
| hypothetical protein FLJ11301 | FLJ11301 | Hs.518505 | 221536_s_at |
| hypothetical protein FLJ11305 | FLJ11305 | Hs.508769 | 219940_s_at |
| hypothetical protein FLJ11795 | FLJ11795 | Hs.436214 | 220112_at |
| Hypothetical protein FLJ11795 | FLJ11795 | Hs.436214 | 240456_at |
| hypothetical protein FLJ11800 | FLJ11800 | Hs.287456 | 220575_at |
| hypothetical protein FLJ11848 | FLJ11848 | Hs.525017 | 218957_s_at |
| hypothetical protein FLJ11850 | FLJ11850 | Hs.7966 | 222941_at |
| Hypothetical protein FLJ11996 | FLJ11996 | Hs.131173 | 242664_at |
| hypothetical protein FLJ12118 | FLJ12118 | Hs.508725 | 218153_at |
| hypothetical protein FLJ12118 | FLJ12118 | Hs.508725 | 231023_at |
| hypothetical protein FLJ12151 | FLJ12151 | — | 215373_x_at |
| hypothetical protein FLJ12355 | FLJ12355 | Hs.287521 | 220465_at |
| hypothetical protein FLJ12476 | FLJ12476 | Hs.444535 | 239813_at |
| hypothetical protein FLJ12505 | FLJ12505 | Hs.96885 | 1555092_at |
| hypothetical protein FLJ12666 | FLJ12666 | Hs.293563 | 222458_s_at |
| hypothetical protein FLJ12671 /// hypothetical | FLJ12671 | Hs.301904 | 208114_s_at |
| protein FLJ12671 | | | |
| hypothetical protein FLJ13089 | FLJ13089 | Hs.530941 | 236816_at |
| hypothetical protein FLJ13236 | FLJ13236 | Hs.170298 | 244193_at |
| hypothetical protein FLJ13273 | FLJ13273 | Hs.161429 | 1554518_at |
| hypothetical protein FLJ13456 | FLJ13456 | Hs.476280 | 225350_s_at |
| hypothetical protein FLJ13511 | 7h3 | Hs.528701 | 44702_at |
| Hypothetical protein FLJ13611 | FLJ13611 | Hs.482301 | 234788_x_at |
| Hypothetical protein FLJ13710 | FLJ13710 | Hs.387057 | 222835_at |
| hypothetical protein FLJ13840 | FLJ13840 | Hs.123515 | 220283_at |
| hypothetical protein FLJ13984 | FLJ13984 | Hs.135146 | 1554667_s_at |
| hypothetical protein FLJ14001 | FLJ14001 | Hs.479612 | 238983_at |
| hypothetical protein FLJ14154 | FLJ14154 | Hs.513296 | 218661_at |
| hypothetical protein FLJ14154 | FLJ14154 | Hs.513296 | 45526_g_at |
| hypothetical protein FLJ14346 | FLJ14346 | Hs.469925 | 220720_x_at |
| hypothetical protein FLJ14451 | FLJ14451 | Hs.528696 | 227430_at |
| hypothetical protein FLJ14624 | FLJ14624 | Hs.190983 | 225666_at |
| hypothetical protein FLJ14721 | FLJ14721 | Hs.144655 | 226487_at |
| hypothetical protein FLJ14775 | FLJ14775 | Hs.103555 | 225319_s_at |
| hypothetical protein FLJ14904 | FLJ14904 | Hs.180191 | 229475_at |
| hypothetical protein FLJ14966 | FLJ14966 | Hs.128196 | 243611_at |
| hypothetical protein FLJ20152 | FLJ20152 | Hs.481704 | 218532_s_at |
| hypothetical protein FLJ20232 | FLJ20232 | Hs.148677 | 204593_s_at |
| hypothetical protein FLJ20232 | FLJ20232 | Hs.148677 | 221516_s_at |
| hypothetical protein FLJ20291 | FLJ20291 | Hs.406223 | 218655_s_at |
| hypothetical protein FLJ20294 | FLJ20294 | Hs.546404 | 52731_at |
| hypothetical protein FLJ20308 | FLJ20308 | Hs.462392 | 1553101_a_at |
| hypothetical protein FLJ20308 | FLJ20308 | Hs.462392 | 234302_s_at |
| Hypothetical protein FLJ20309 | FLJ20309 | Hs.471205 | 227837_at |
| hypothetical protein FLJ20323 /// hypothetical | FLJ20323 | Hs.520215 | 211724_x_at |
| protein FLJ20323 | | | |
| hypothetical protein FLJ20340 | LAX | Hs.272794 | 207734_at |
| hypothetical protein FLJ20345 | FLJ20345 | Hs.408843 | 1555820_a_at |
| hypothetical protein FLJ20366 | FLJ20366 | Hs.390738 | 218692_at |
| hypothetical protein FLJ20399 | FLJ20399 | Hs.534460 | 47105_at |
| hypothetical protein FLJ20425 | LYAR | Hs.425427 | 223413_s_at |
| Hypothetical protein FLJ20444 | FLJ20444 | Hs.459704 | 1557584_at |
| hypothetical protein FLJ20449 | FLJ20449 | Hs.124519 | 220129_at |
| hypothetical protein FLJ20531 | FLJ20531 | Hs.377705 | 220661_s_at |
| Hypothetical protein FLJ20533 | FLJ20533 | Hs.106650 | 226083_at |
| hypothetical protein FLJ20558 | FLJ20558 | Hs.413123 | 219128_at |
| hypothetical protein FLJ20604 | KS | Hs.55781 | 219133_at |
| hypothetical protein FLJ20618 | FLJ20618 | Hs.120446 | 222244_s_at |
| hypothetical protein FLJ20674 | FLJ20674 | Hs.187624 | 220137_at |
| Hypothetical protein FLJ20701 | FLJ20701 | Hs.409352 | 1559910_at |
| hypothetical protein FLJ20719 /// hypothetical | FLJ20719 | Hs.546664 | 201103_x_at |
| protein LOC200030 /// hypothetical protein | /// | | |
| MGC8902 /// AG1 protein /// hypothetical | LOC200030 | | |
| LOC440675 | /// | | |
| | MGC8902 | | |
| | /// AG1 | | |
| | /// | | |
| | LOC440675 | | |
| hypothetical protein FLJ20719 /// hypothetical | FLJ20719 | Hs.546664 | 213612_x_at |
| protein MGC8902 /// AG1 protein /// hypothetical | /// | | |
| LOC440675 | MGC8902 | | |
| | /// AG1 | | |
| | /// | | |
| | LOC440675 | | |
| hypothetical protein FLJ20772 | FLJ20772 | Hs.9925 | 219299_at |
| hypothetical protein FLJ21106 | FLJ21106 | Hs.445817 | 219980_at |
| hypothetical protein FLJ21168 | FLJ21168 | Hs.24808 | 204665_at |
| hypothetical protein FLJ21272 | FLJ21272 | — | 220467_at |
| hypothetical protein FLJ21415 | FLJ21415 | Hs.528681 | 218867_s_at |
| hypothetical protein FLJ21616 | FLJ21616 | Hs.194225 | 219269_at |
| Hypothetical protein FLJ21742 | FLJ21742 | Hs.306809 | 232730_at |
| hypothetical protein FLJ21918 | FLJ21918 | Hs.436585 | 219395_at |
| Hypothetical protein FLJ21924 | FLJ21924 | Hs.369368 | 244188_at |
| hypothetical protein FLJ22028 | FLJ22028 | Hs.546437 | 219802_at |
| hypothetical protein FLJ22318 | FLJ22318 | Hs.519804 | 218262_at |
| hypothetical protein FLJ22531 | FLJ22531 | Hs.292088 | 238593_at |
| hypothetical protein FLJ22639 | FLJ22639 | Hs.351445 | 220399_at |
| hypothetical protein FLJ22662 | FLJ22662 | Hs.131933 | 218454_at |
| Hypothetical protein FLJ22662 | FLJ22662 | Hs.131933 | 236422_at |
| hypothetical protein FLJ23129 | FLJ23129 | Hs.49421 | 220769_s_at |
| hypothetical protein FLJ23191 | FLJ23191 | Hs.16026 | 219747_at |
| hypothetical protein FLJ23323 | FLJ23323 | Hs.59425 | 1554082_a_at |
| hypothetical protein FLJ23323 | FLJ23323 | Hs.59425 | 218754_at |
| hypothetical protein FLJ23441 | FLJ23441 | Hs.503389 | 219217_at |
| hypothetical protein FLJ23518 | FLJ23518 | Hs.525088 | 223300_s_at |
| hypothetical protein FLJ23598 | FLJ23598 | Hs.147377 | 220390_at |
| hypothetical protein FLJ23790 | FLJ23790 | Hs.549261 | 226294_x_at |
| hypothetical protein FLJ23825 | FLJ23825 | Hs.444743 | 1555866_a_at |
| hypothetical protein FLJ23861 | FLJ23861 | Hs.122934 | 1554791_a_at |
| hypothetical protein FLJ23861 | FLJ23861 | Hs.122934 | 215046_at |
| hypothetical protein FLJ23861 | FLJ23861 | Hs.122934 | 231252_at |
| hypothetical protein FLJ23878 | FLJ23878 | Hs.194609 | 1553315_at |
| hypothetical protein FLJ23878 | FLJ23878 | Hs.194609 | 1554074_s_at |
| Hypothetical protein FLJ25006 | FLJ25006 | Hs.351173 | 244547_at |
| hypothetical protein FLJ25059 | FLJ25059 | Hs.502223 | 229025_s_at |
| hypothetical protein FLJ25168 | FLJ25168 | Hs.128813 | 1553400_a_at |
| hypothetical protein FLJ25333 | FLJ25333 | Hs.276287 | 240065_at |
| hypothetical protein FLJ25371 | FLJ25371 | Hs.415576 | 1553838_at |
| hypothetical protein FLJ25555 | FLJ25555 | Hs.334715 | 227720_at |
| hypothetical protein FLJ25660 | FLJ25660 | Hs.128069 | 242259_at |
| hypothetical protein FLJ25770 | FLJ25770 | Hs.529680 | 243875_at |
| hypothetical protein FLJ25955 | FLJ25955 | Hs.424594 | 242162_at |
| hypothetical protein FLJ30430 | FLJ30430 | — | 1552942_at |
| hypothetical protein FLJ30525 | FLJ30525 | Hs.7962 | 225841_at |
| Hypothetical protein FLJ31153 | FLJ31153 | Hs.514179 | 240529_at |
| hypothetical protein FLJ31295 | FLJ31295 | Hs.23492 | 226509_at |
| hypothetical protein FLJ31340 | FLJ31340 | Hs.208093 | 244317_at |
| Hypothetical protein FLJ31810 | FLJ31810 | Hs.145642 | 232720_at |
| Hypothetical protein FLJ31951 | FLJ31951 | Hs.349306 | 236322_at |
| hypothetical protein FLJ31978 | FLJ31978 | Hs.12381 | 229770_at |
| hypothetical protein FLJ32115 | FLJ32115 | Hs.162143 | 227450_at |
| hypothetical protein FLJ32440 | FLJ32440 | Hs.388297 | 226536_at |
| hypothetical protein FLJ32499 | FLJ32499 | Hs.27475 | 226833_at |
| hypothetical protein FLJ32658 | FLJ32658 | Hs.511847 | 240950_s_at |
| hypothetical protein FLJ32786 | FLJ32786 | Hs.149774 | 244710_at |
| hypothetical protein FLJ32796 | FLJ32796 | Hs.129293 | 1552384_a_at |
| hypothetical protein FLJ32915 | FLJ32915 | Hs.98133 | 227687_at |
| hypothetical protein FLJ32926 | FLJ32926 | Hs.112645 | 233157_x_at |
| hypothetical protein FLJ32940 | DKFZp686H1423 | Hs.496400 | 1552870_s_at |
| hypothetical protein FLJ32942 | FLJ32942 | Hs.524476 | 227711_at |
| hypothetical protein FLJ32949 | FLJ32949 | Hs.533644 | 238784_at |
| hypothetical protein FLJ32954 | FLJ32954 | Hs.532901 | 1553232_at |
| hypothetical protein FLJ32978 | FLJ32978 | Hs.97527 | 1553373_at |
| Hypothetical protein FLJ33708 | FLJ33708 | Hs.170081 | 242399_at |
| hypothetical protein FLJ34443 | FLJ34443 | Hs.26410 | 228318_s_at |
| hypothetical protein FLJ34658 | FLJ34658 | Hs.254699 | 235904_at |
| hypothetical protein FLJ34969 | FLJ34969 | Hs.91085 | 226965_at |
| hypothetical protein FLJ35382 | FLJ35382 | Hs.520192 | 244103_at |
| hypothetical protein FLJ35390 | FLJ35390 | Hs.448456 | 1564207_at |
| hypothetical protein FLJ35630 | FLJ35630 | Hs.400698 | 229603_at |
| hypothetical protein FLJ35802 | FLJ35802 | Hs.246284 | 1552587_at |
| hypothetical protein FLJ35867 | FLJ35867 | Hs.418287 | 226562_at |
| hypothetical protein FLJ36144 | FLJ36144 | Hs.372394 | 1560790_at |
| Hypothetical protein FLJ36166 | FLJ36166 | Hs.148768 | 215143_at |
| hypothetical protein FLJ36754 | FLJ36754 | Hs.519343 | 1553581_s_at |
| hypothetical protein FLJ38281 | FLJ38281 | Hs.306478 | 1570366_x_at |
| hypothetical protein FLJ38288 | FLJ38288 | Hs.109540 | 228652_at |
| Hypothetical protein FLJ38348 | FLJ38348 | Hs.546523 | 213294_at |
| Hypothetical protein FLJ38374 | FLJ38374 | Hs.449065 | 216053_x_at |
| hypothetical protein FLJ38964 | FLJ38964 | Hs.444911 | 235699_at |
| hypothetical protein FLJ38973 | FLJ38973 | Hs.471040 | 227973_at |
| hypothetical protein FLJ38973 | FLJ38973 | Hs.471040 | 238974_at |
| hypothetical protein FLJ38984 | FLJ38984 | Hs.112023 | 212791_at |
| hypothetical protein FLJ39599 | FLJ39599 | Hs.401798 | 1559167_x_at |
| hypothetical protein FLJ40176 | FLJ40176 | Hs.368382 | 1561846_s_at |
| hypothetical protein FLJ40201 | FLJ40201 | Hs.376018 | 1557016_a_at |
| hypothetical protein FLJ40235 | FLJ40235 | Hs.381087 | 1553887_at |
| hypothetical protein FLJ40243 | FLJ40243 | Hs.97714 | 240411_at |
| hypothetical protein FLJ40244 | FLJ40244 | Hs.434123 | 233462_at |
| hypothetical protein FLJ40342 | FLJ40342 | Hs.463303 | 1553626_a_at |
| hypothetical protein FLJ40342 | FLJ40342 | Hs.463303 | 1553627_s_at |
| hypothetical protein FLJ40342 | FLJ40342 | Hs.463303 | 1554772_at |
| Hypothetical protein FLJ40342 | FLJ40342 | Hs.463303 | 1558854_a_at |
| Hypothetical protein FLJ40342 | FLJ40342 | Hs.463303 | 231651_at |
| hypothetical protein FLJ40427 | FLJ40427 | Hs.201378 | 243802_at |
| hypothetical protein FLJ40432 | FLJ40432 | Hs.471234 | 228810_at |
| hypothetical protein FLJ40473 | FLJ40473 | — | 1558987_at |
| hypothetical protein FLJ90575 | FLJ90575 | Hs.381181 | 238682_at |
| hypothetical protein FLJ90709 | FLJ90709 | Hs.128696 | 243709_at |
| hypothetical protein H41 | H41 | Hs.518265 | 212010_s_at |
| hypothetical protein H41 | H41 | Hs.518265 | 213548_s_at |
| Hypothetical protein H41 | H41 | Hs.518265 | 228746_s_at |
| hypothetical protein HS747E2A | HS747E2A | Hs.50891 | 206839_at |
| hypothetical protein HSPC016 | HSPC016 | Hs.546394 | 221791_s_at |
| hypothetical protein HSPC129 | HSPC129 | Hs.497967 | 1555106_a_at |
| hypothetical protein HSPC148 | HSPC148 | Hs.503597 | 223067_at |
| hypothetical protein HSPC196 | HSPC196 | Hs.406530 | 223113_at |
| hypothetical protein HSPC268 | HSPC268 | Hs.370475 | 226780_s_at |
| hypothetical protein KIAA1434 | KIAA1434 | Hs.472040 | 224826_at |
| hypothetical protein LOC115749 | LOC115749 | Hs.28700 | 236840_at |
| hypothetical protein LOC120376 | LOC120376 | Hs.31409 | 228338_at |
| Hypothetical protein LOC123722 | LOC123722 | Hs.513091 | 228930_at |
| Hypothetical protein LOC124512 | LOC124512 | Hs.74655 | 225808_at |
| hypothetical protein LOC128344 | LOC128344 | Hs.172510 | 228100_at |
| hypothetical protein LOC144438 | LOC144438 | Hs.92308 | 225493_at |
| hypothetical protein LOC144571 | LOC144571 | — | 1564139_at |
| hypothetical protein LOC144997 | LOC144997 | — | 227289_at |
| Hypothetical protein LOC146174 | LOC146174 | Hs.549248 | 230296_at |
| hypothetical protein LOC147004 | LOC147004 | Hs.434122 | 1564203_at |
| hypothetical protein LOC148145 | LOC148145 | Hs.112651 | 1557550_at |
| hypothetical protein LOC148189 | LOC148189 | Hs.190090 | 235191_at |
| hypothetical protein LOC148203 | LOC148203 | Hs.359535 | 229700_at |
| Hypothetical protein LOC149401 | LOC149401 | Hs.191475 | 242232_at |
| hypothetical protein LOC149832 | LOC149832 | Hs.126857 | 228456_s_at |
| hypothetical protein LOC149837 | LOC149837 | Hs.123609 | 229992_at |
| hypothetical protein LOC150271 | LOC150271 | — | 228658_at |
| hypothetical protein LOC150297 | LOC150297 | Hs.426551 | 1555990_at |
| hypothetical protein LOC150297 | LOC150297 | Hs.426551 | 1555991_s_at |
| hypothetical protein LOC151121 | LOC151121 | Hs.531687 | 1560750_at |
| hypothetical protein LOC151300 | LOC151300 | Hs.420716 | 239506_s_at |
| hypothetical protein LOC151300 | LOC151300 | Hs.420716 | 239507_at |
| hypothetical protein LOC151658 | LOC151658 | Hs.159043 | 238283_at |
| Hypothetical protein LOC152485 | LOC152485 | Hs.133916 | 228046_at |
| Hypothetical protein LOC152485 | LOC152485 | Hs.133916 | 231902_at |
| Hypothetical protein LOC152485 | LOC152485 | Hs.133916 | 243618_s_at |
| Hypothetical protein LOC152519 | LOC152519 | Hs.134190 | 1562792_at |
| hypothetical protein LOC153546 | LOC153546 | — | 236124_at |
| hypothetical protein LOC153561 | LOC153561 | Hs.545578 | 213089_at |
| hypothetical protein LOC153561 | LOC153561 | Hs.545578 | 232889_at |
| hypothetical protein LOC157693 | LOC157693 | Hs.521342 | 1559140_at |
| hypothetical protein LOC157697 | LOC157697 | Hs.128130 | 1563315_s_at |
| hypothetical protein LOC157697 | LOC157697 | Hs.128130 | 227017_at |
| Hypothetical protein LOC158160 | LOC158160 | Hs.380900 | 244075_at |
| hypothetical protein LOC161527 | LOC161527 | Hs.534573 | 213212_x_at |
| hypothetical protein LOC162427 | LOC162427 | Hs.463079 | 212697_at |
| hypothetical protein LOC169355 | LOC169355 | Hs.122077 | 1568638_a_at |
| hypothetical protein LOC196913 | LOC196913 | Hs.406934 | 1564785_at |
| hypothetical protein LOC199899 | LOC199899 | Hs.116210 | 242284_at |
| hypothetical protein LOC200030 | LOC200030 | Hs.515837 | 229447_x_at |
| hypothetical protein LOC200030 /// hypothetical | LOC200030 | Hs.515947 | 242191_at |
| LOC400781 /// AG1 protein | /// | | |
| | LOC400781 | | |
| | /// | | |
| | AG1 | | |
| hypothetical protein LOC200205 | LOC200205 | Hs.237017 | 231983_at |
| hypothetical protein LOC201243 | LOC201243 | Hs.380704 | 240082_s_at |
| hypothetical protein LOC201895 | LOC201895 | Hs.205952 | 224990_at |
| hypothetical protein LOC202051 | LOC202051 | Hs.373612 | 1558641_at |
| hypothetical protein LOC202051 | LOC202051 | Hs.373612 | 238027_at |
| hypothetical protein LOC220930 | LOC220930 | Hs.372654 | 229090_at |
| hypothetical protein LOC221122 | LOC221122 | Hs.551656 | 1560279_a_at |
| hypothetical protein LOC221442 | LOC221442 | Hs.374076 | 236832_at |
| hypothetical protein LOC221810 | LOC221810 | Hs.363292 | 221910_at |
| hypothetical protein LOC222070 | LOC222070 | — | 51774_s_at |
| hypothetical protein LOC222070 | LOC222070 | — | 58900_at |
| hypothetical protein LOC222967 /// hypothetical | LOC222967 | Hs.510465 | 1557417_s_at |
| protein LOC285927 | /// | | |
| | LOC285927 | | |
| Hypothetical protein LOC254128 | LOC254128 | Hs.535621 | 1557059_at |
| hypothetical protein LOC254128 | LOC254128 | Hs.535621 | 235132_at |
| hypothetical protein LOC255326 | LOC255326 | Hs.314437 | 231106_at |
| hypothetical protein LOC255330 | LOC255330 | Hs.145435 | 244681_at |
| hypothetical protein LOC255480 | LOC255480 | Hs.363081 | 239919_at |
| hypothetical protein LOC255783 | LOC255783 | Hs.128690 | 227325_at |
| Hypothetical protein LOC256021 | LOC256021 | Hs.209203 | 243509_at |
| hypothetical protein LOC256273 | LOC256273 | Hs.99626 | 229466_at |
| hypothetical protein LOC257407 | LOC257407 | Hs.526596 | 1556464_a_at |
| hypothetical protein LOC282980 | LOC282980 | — | 1564209_at |
| hypothetical protein LOC283377 | LOC283377 | Hs.128676 | 225616_at |
| hypothetical protein LOC283403 | LOC283403 | Hs.142880 | 1560277_a_at |
| hypothetical protein LOC283404 | LOC283404 | Hs.550633 | 1558195_at |
| hypothetical protein LOC283480 | LOC283480 | Hs.549300 | 237392_at |
| hypothetical protein LOC283481 | LOC283481 | — | 230351_at |
| hypothetical protein LOC283666 | LOC283666 | Hs.512943 | 226682_at |
| hypothetical protein LOC283680 | LOC283680 | Hs.220864 | 225077_at |
| hypothetical protein LOC283854 | LOC283854 | Hs.266768 | 1556954_at |
| hypothetical protein LOC283914 | LOC283914 | Hs.385772 | 1561518_at |
| Hypothetical protein LOC283970 | LOC283970 | Hs.165328 | 221992_at |
| Hypothetical protein LOC284009 | LOC284009 | Hs.224187 | 238931_at |
| hypothetical protein LOC284058 | LOC284058 | Hs.405514 | 225117_at |
| hypothetical protein LOC284058 | LOC284058 | Hs.405514 | 243589_at |
| hypothetical protein LOC284242 | LOC284242 | — | 1560250_s_at |
| hypothetical protein LOC284267 | LOC284267 | Hs.514843 | 228453_at |
| hypothetical protein LOC284356 | LOC284356 | Hs.547530 | 1555842_at |
| Hypothetical protein LOC284371 | LOC284371 | Hs.178471 | 229279_at |
| hypothetical protein LOC284591 | LOC284591 | Hs.133183 | 1557055_s_at |
| Hypothetical protein LOC284701 /// CDNA | LOC284701 | Hs.512176 /// | 231821_x_at |
| FLJ41452 fis, clone BRSTN2010363 /// | | Hs.530543 /// | |
| Hypothetical gene supported by AK128780 | | Hs.546653 | |
| hypothetical protein LOC284701 /// hypothetical | LOC284701 | Hs.546654 | 225899_x_at |
| gene supported by AK128780 /// FLJ45445 protein | /// | | |
| /// hypothetical gene supported by AK093729; | LOC388312 | | |
| BX647918 /// similar to hypothetical protein | /// | | |
| LOC349114 /// hypothetical gene supported by | FLJ45445 | | |
| AK097080; AL117642; BC047304; BC054485 | /// | | |
| | LOC440470 | | |
| | /// | | |
| | LOC440740 | | |
| | /// | | |
| | LOC441000 | | |
| hypothetical protein LOC284701 /// hypothetical | LOC284701 | Hs.546653 | 227129_x_at |
| gene supported by AK128780 /// FLJ45445 protein | /// | | |
| /// hypothetical gene supported by AK097080; | LOC388312 | | |
| AL117642; BC047304; BC054485 | /// | | |
| | FLJ45445 | | |
| | /// | | |
| | LOC441000 | | |
| hypothetical protein LOC284757 | LOC284757 | Hs.126778 | 236846_at |
| hypothetical protein LOC284837 | LOC284837 | Hs.313689 | 1563088_a_at |
| Hypothetical protein LOC284958 | LOC284958 | Hs.434250 | 1560204_at |
| hypothetical protein LOC284958 | LOC284958 | Hs.434250 | 1562020_s_at |
| hypothetical protein LOC285147 | LOC285147 | Hs.467627 | 236166_at |
| hypothetical protein LOC285148 | LOC285148 | Hs.509314 | 225908_at |
| hypothetical protein LOC285148 | LOC285148 | Hs.509314 | 230621_at |
| hypothetical protein LOC285194 | LOC285194 | Hs.120364 | 1558602_a_at |
| hypothetical protein LOC285331 | LOC285331 | Hs.476399 | 228122_at |
| Hypothetical protein LOC285389 | LOC285389 | Hs.157102 | 244668_at |
| hypothetical protein LOC285429 | LOC285429 | Hs.213307 | 1562209_at |
| Hypothetical protein LOC285505 | LOC285505 | Hs.535739 | 231557_at |
| hypothetical protein LOC285550 | LOC285550 | Hs.399980 | 227466_at |
| hypothetical protein LOC285636 | LOC285636 | Hs.29899 | 226159_at |
| hypothetical protein LOC285638 | LOC285638 | Hs.532104 | 1563489_at |
| hypothetical protein LOC285735 | LOC285735 | Hs.407549 | 1561691_at |
| hypothetical protein LOC285740 | LOC285740 | Hs.432656 | 237614_at |
| hypothetical protein LOC286009 | LOC286009 | — | 1562664_at |
| hypothetical protein LOC286052 | LOC286052 | — | 241370_at |
| hypothetical protein LOC286109 | LOC286109 | — | 243304_at |
| hypothetical protein LOC286114 | LOC286114 | Hs.357242 | 244255_at |
| Hypothetical protein LOC286144 | LOC286144 | Hs.440643 | 225600_at |
| hypothetical protein LOC286254 | LOC286254 | Hs.170035 | 1559765_a_at |
| hypothetical protein LOC338579 | LOC338579 | Hs.434365 | 1561225_at |
| hypothetical protein LOC338653 | LOC338653 | — | 1562960_at |
| hypothetical protein LOC338758 | LOC338758 | Hs.136888 | 238893_at |
| hypothetical protein LOC339287 | LOC339287 | Hs.532786 | 224765_at |
| hypothetical protein LOC339448 | LOC339448 | Hs.103939 | 222000_at |
| hypothetical protein LOC339448 | LOC339448 | Hs.103939 | 238010_at |
| Hypothetical protein LOC339524 | LOC339524 | Hs.306423 | 215039_at |
| hypothetical protein LOC339524 | LOC339524 | Hs.306423 | 235870_at |
| hypothetical protein LOC339666 | LOC339666 | Hs.531913 | 234385_at |
| hypothetical protein LOC339803 | LOC339803 | Hs.252433 | 227941_at |
| hypothetical protein LOC339834 | LOC339834 | Hs.383008 | 1569690_at |
| hypothetical protein LOC339894 | LOC339894 | Hs.478050 | 1561723_at |
| hypothetical protein LOC339903 | LOC339903 | Hs.146346 | 228721_at |
| hypothetical protein LOC339988 | LOC339988 | Hs.434746 | 1562697_at |
| hypothetical protein LOC339988 | LOC339988 | Hs.434746 | 1562698_x_at |
| hypothetical protein LOC340085 | LOC340085 | — | 232455_x_at |
| hypothetical protein LOC340239 | LOC340239 | Hs.520877 | 1557664_at |
| hypothetical protein LOC340529 | LOC340529 | Hs.450320 | 1557203_at |
| hypothetical protein LOC346653 | LOC346653 | Hs.445236 | 237320_at |
| hypothetical protein LOC348801 | LOC348801 | Hs.550566 | 227181_at |
| hypothetical protein LOC374768 | LOC374768 | Hs.499707 | 240878_at |
| hypothetical protein LOC375035 | LOC375035 | Hs.546557 | 214838_at |
| hypothetical protein LOC51057 | LOC51057 | Hs.414952 | 224180_x_at |
| hypothetical protein LOC51057 | LOC51057 | Hs.414952 | 231770_x_at |
| hypothetical protein LOC51233 | LOC51233 | Hs.517466 | 221108_at |
| hypothetical protein LOC51233 | LOC51233 | Hs.517466 | 237325_at |
| Hypothetical protein LOC51234 | LOC51234 | Hs.250905 | 226741_at |
| hypothetical protein LOC51321 | LOC51321 | Hs.268122 | 218167_at |
| hypothetical protein LOC56757 | LOC56757 | — | 225509_at |
| hypothetical protein LOC57821 | LOC57821 | Hs.130746 | 1555112_a_at |
| hypothetical protein LOC57821 | LOC57821 | Hs.130746 | 206721_at |
| hypothetical protein LOC90110 | LOC90110 | Hs.547488 | 226842_at |
| hypothetical protein LOC90321 | LOC90321 | Hs.439662 | 227284_at |
| hypothetical protein LOC90520 | LOC90520 | Hs.552781 | 233342_at |
| hypothetical protein LOC91548 | LOC91548 | Hs.367839 | 234341_x_at |
| hypothetical protein LOC92482 | LOC92482 | Hs.192249 | 213220_at |
| hypothetical protein LOC92912 | LOC92912 | Hs.23033 | 224747_at |
| hypothetical protein MAC30 | MAC30 | Hs.199695 | 212279_at |
| hypothetical protein MAC30 | MAC30 | Hs.199695 | 212281_s_at |
| hypothetical protein MAC30 | MAC30 | Hs.199695 | 212282_at |
| hypothetical protein MDS025 | MDS025 | Hs.368866 | 218288_s_at |
| Hypothetical protein MGC10067 | MGC10067 | Hs.190447 | 239489_at |
| hypothetical protein MGC10067 | MGC10067 | Hs.190447 | 243916_x_at |
| hypothetical protein MGC10198 | MGC10198 | Hs.133337 | 225574_at |
| hypothetical protein MGC10850 | MGC10850 | — | 223707_at |
| hypothetical protein MGC10986 | MGC10986 | Hs.514402 | 242117_at |
| hypothetical protein MGC11324 /// hypothetical | MGC11324 | Hs.99196 | 224480_s_at |
| protein MGC11324 | | | |
| Hypothetical protein MGC12458 | MGC12458 | Hs.134857 | 1557674_s_at |
| hypothetical protein MGC12760 | MGC12760 | Hs.522876 | 1569293_x_at |
| hypothetical protein MGC12966 /// hypothetical | MGC12966 | Hs.7980 | 224452_s_at |
| protein MGC12966 | | | |
| hypothetical protein MGC12981 | MGC12981 | Hs.104203 | 224946_s_at |
| hypothetical protein MGC13008 | MGC13008 | — | 224132_at |
| Hypothetical protein MGC13057 | MGC13057 | Hs.389311 | 232773_at |
| hypothetical protein MGC14128 | BJ-TSA-9 | Hs.379821 | 239586_at |
| hypothetical protein MGC14151 | MGC14151 | Hs.333414 | 225441_x_at |
| hypothetical protein MGC14151 /// hypothetical | MGC14151 | Hs.333414 | 224512_s_at |
| protein MGC14151 | | | |
| hypothetical protein MGC14156 | MGC14156 | Hs.26136 | 224660_at |
| Hypothetical protein MGC14161 | MGC14161 | Hs.368399 | 240502_at |
| hypothetical protein MGC14425 | MGC14425 | — | 1552665_at |
| hypothetical protein MGC14436 | MGC14436 | Hs.334525 | 1553811_at |
| hypothetical protein MGC14801 /// hypothetical | MGC14801 | Hs.523932 | 224443_at |
| protein MGC14801 | | | |
| hypothetical protein MGC14801 /// hypothetical | MGC14801 | Hs.523932 | 224444_s_at |
| protein MGC14801 | | | |
| hypothetical protein MGC15882 | MGC15882 | Hs.194610 | 231514_at |
| hypothetical protein MGC16169 | MGC16169 | Hs.292986 | 226126_at |
| hypothetical protein MGC16207 | MGC16207 | — | 225211_at |
| hypothetical protein MGC17791 | MGC17791 | Hs.465643 | 227420_at |
| Hypothetical protein MGC19780 | MGC19780 | Hs.408927 | 1560533_at |
| hypothetical protein MGC20481 | MGC20481 | Hs.146520 | 1558648_at |
| hypothetical protein MGC20481 | MGC20481 | Hs.146520 | 225397_at |
| hypothetical protein MGC21416 | MGC21416 | Hs.82719 | 212340_at |
| hypothetical protein MGC21416 | MGC21416 | Hs.82719 | 212341_at |
| hypothetical protein MGC21518 | MGC21518 | Hs.353163 | 226565_at |
| hypothetical protein MGC22014 | MGC22014 | Hs.516107 | 235542_at |
| Hypothetical protein MGC22265 | MGC22265 | Hs.374448 | 1556742_at |
| Hypothetical protein MGC22265 /// Similar to | MGC22265 | Hs.374448 /// | 213605_s_at |
| bA110H4.2 (similar to membrane protein) | | Hs.482414 | |
| hypothetical protein MGC22773 | MGC22773 | Hs.151193 | 1553674_at |
| hypothetical protein MGC23401 | MGC23401 | Hs.527874 | 213065_at |
| hypothetical protein MGC23909 | MGC23909 | Hs.355606 | 226276_at |
| hypothetical protein MGC24103 | MGC24103 | — | 232568_at |
| hypothetical protein MGC24132 | MGC24132 | Hs.149487 | 1553689_s_at |
| hypothetical protein MGC2574 | MGC2574 | Hs.4253 | 203119_at |
| hypothetical protein MGC2654 | MGC2654 | Hs.306380 | 218945_at |
| hypothetical protein MGC26594 | MGC26594 | Hs.97335 | 1554545_at |
| hypothetical protein MGC26733 | MGC26733 | Hs.269977 | 1555298_a_at |
| hypothetical protein MGC26733 | MGC26733 | Hs.269977 | 1562371_s_at |
| hypothetical protein MGC26856 | MGC26856 | Hs.551586 | 238296_at |
| hypothetical protein MGC26989 | MGC26989 | Hs.514662 | 1558834_s_at |
| hypothetical protein MGC27069 | LOC151649 | Hs.552611 | 1552747_a_at |
| hypothetical protein MGC27277 | MGC27277 | Hs.133977 | 242283_at |
| hypothetical protein MGC2744 | MGC2744 | Hs.317403 | 222064_s_at |
| hypothetical protein MGC2744 | MGC2744 | Hs.317403 | 238740_at |
| hypothetical protein MGC2747 | MGC2747 | Hs.356467 | 224712_x_at |
| hypothetical protein MGC29463 | MGC29463 | Hs.483537 | 227166_at |
| hypothetical protein MGC2963 /// hypothetical | MGC2963 | Hs.30011 | 221255_s_at |
| protein MGC2963 | | | |
| hypothetical protein MGC29875 | MGC29875 | Hs.194754 | 220251_at |
| hypothetical protein MGC29898 | MGC29898 | Hs.479298 | 235259_at |
| hypothetical protein MGC29898 | MGC29898 | Hs.479298 | 235260_s_at |
| hypothetical protein MGC30208 | MGC30208 | Hs.135215 | 229736_at |
| hypothetical protein MGC3123 | MGC3123 | Hs.181391 | 218419_s_at |
| hypothetical protein MGC3234 | MGC3234 | Hs.144468 | 223061_at |
| hypothetical protein MGC33212 | MGC33212 | — | 228606_at |
| hypothetical protein MGC3329 | MGC3329 | Hs.513862 | 226744_at |
| hypothetical protein MGC33371 | MGC33371 | Hs.288304 | 232048_at |
| hypothetical protein MGC33407 | MGC33407 | Hs.209206 | 243208_x_at |
| hypothetical protein MGC33648 | MGC33648 | Hs.85950 | 238465_at |
| hypothetical protein MGC33839 | MGC33839 | Hs.348531 | 231162_at |
| hypothetical protein MGC33867 | MGC33867 | Hs.520094 | 224708_at |
| hypothetical protein MGC33887 | MGC33887 | Hs.408676 | 213644_at |
| hypothetical protein MGC33948 | MGC33948 | Hs.367437 | 1552988_at |
| hypothetical protein MGC34034 | MGC34034 | Hs.126165 | 1552900_a_at |
| hypothetical protein MGC34732 | MGC34732 | Hs.129284 | 237160_at |
| Hypothetical protein MGC35048 | MGC35048 | Hs.460217 | 216135_at |
| hypothetical protein MGC35097 | MGC35097 | Hs.13781 | 225755_at |
| hypothetical protein MGC35118 | MGC35118 | Hs.179646 | 1560184_at |
| hypothetical protein MGC35274 | MGC35274 | Hs.134045 | 226748_at |
| Hypothetical protein MGC39325 | MGC39325 | Hs.154652 | 232668_at |
| hypothetical protein MGC39389 | HEXIM2 | Hs.56382 | 238547_at |
| hypothetical protein MGC39518 | MGC39518 | Hs.482656 | 1554178_a_at |
| hypothetical protein MGC39520 | MGC39520 | Hs.549268 | 1552526_at |
| hypothetical protein MGC39606 | MGC39606 | Hs.374414 | 227909_at |
| hypothetical protein MGC39633 | MGC39633 | Hs.436121 | 235208_at |
| hypothetical protein MGC40178 | MGC40178 | Hs.124463 | 243884_at |
| Hypothetical protein MGC40222 | MGC40222 | Hs.173337 | 244829_at |
| hypothetical protein MGC40405 | MGC40405 | Hs.18564 | 226332_at |
| Hypothetical protein MGC40405 | MGC40405 | Hs.18564 | 233204_at |
| Hypothetical protein MGC40405 | MGC40405 | Hs.18564 | 233867_at |
| hypothetical protein MGC42174 | MGC42174 | Hs.471637 | 238602_at |
| hypothetical protein MGC42630 | MGC42630 | Hs.31240 | 227563_at |
| hypothetical protein MGC4268 | MGC4268 | Hs.546449 | 223297_at |
| hypothetical protein MGC42718 | MGC42718 | Hs.98660 | 231266_at |
| hypothetical protein MGC4278 | MGC4278 | — | 220352_x_at |
| hypothetical protein MGC4308 /// hypothetical | MGC4308 | Hs.551567 | 224523_s_at |
| protein MGC4308 | | | |
| hypothetical protein MGC43690 | MGC43690 | Hs.131903 | 235298_at |
| hypothetical protein MGC45477 | MGC45477 | Hs.511015 | 1554250_s_at |
| hypothetical protein MGC45731 | MGC45731 | Hs.381105 | 227080_at |
| hypothetical protein MGC46336 | MGC46336 | Hs.381893 | 1555078_at |
| hypothetical protein MGC46336 | MGC46336 | Hs.381893 | 1564362_x_at |
| Hypothetical protein MGC4692 | MGC4692 | Hs.442634 | 222622_at |
| hypothetical protein MGC4771 | MGC4771 | — | 210723_x_at |
| hypothetical protein MGC48972 | MGC48972 | Hs.101007 | 239648_at |
| hypothetical protein MGC5139 | MGC5139 | Hs.127610 | 202365_at |
| Hypothetical protein MGC5178 | MGC5178 | Hs.460558 | 209836_x_at |
| hypothetical protein MGC52057 | MGC52057 | Hs.21929 | 239028_at |
| hypothetical protein MGC5242 | MGC5242 | Hs.521213 | 220949_s_at |
| hypothetical protein MGC5306 | MGC5306 | Hs.355750 | 218750_at |
| hypothetical protein MGC5306 | MGC5306 | Hs.355750 | 222728_s_at |
| hypothetical protein MGC5590 | MGC5590 | — | 220931_at |
| hypothetical protein MGC8721 | MGC8721 | Hs.521487 | 200847_s_at |
| hypothetical protein MGC9913 | MGC9913 | Hs.23133 | 244740_at |
| hypothetical protein MGC9913 | MGC9913 | Hs.23133 | 244741_s_at |
| hypothetical protein PRO1855 | PRO1855 | Hs.370927 | 222231_s_at |
| Hypothetical protein PRO2730 | PRO2730 | Hs.194110 | 201934_at |
| hypothetical protein PRO2852 | PRO2852 | — | 223797_at |
| hypothetical protein RP4-622L5 | RP4- | Hs.272299 | 223656_s_at |
| | 622L5 | | |
| hypothetical protein TI-227H | TI-227H | — | 212337_at |
| hypothetical protein TI-227H | TI-227H | — | 212725_s_at |
| hypoxia-inducible factor 1, alpha subunit (basic | HIF1A | Hs.509554 | 200989_at |
| helix-loop-helix transcription factor) | | | |
| hypoxia-inducible factor prolyl 4-hydroxylase | PH-4 | Hs.271224 | 222125_s_at |
| IBR domain containing 2 | IBRDC2 | Hs.148741 | 228153_at |
| IGF-II mRNA-binding protein 2 | IMP-2 | Hs.35354 | 218847_at |
| IGF-II mRNA-binding protein 3 | IMP-3 | Hs.432616 | 203819_s_at |
| IGF-II mRNA-binding protein 3 | IMP-3 | Hs.432616 | 203820_s_at |
| IL2-inducible T-cell kinase | ITK | Hs.483938 | 211339_s_at |
| IMAGE: 729984 clone, mRNA | — | Hs.553160 | 1560920_s_at |
| immediate early response 2 | IER2 | Hs.501629 | 202081_at |
| immediate early response 3 | IER3 | Hs.76095 | 201631_s_at |
| immediate early response 5 | IER5 | Hs.15725 | 218611_at |
| Immunoglobulin (CD79A) binding protein 1 | IGBP1 | Hs.496267 | 242337_at |
| immunoglobulin superfamily, member 3 | IGSF3 | Hs.171057 | 202421_at |
| immunoglobulin superfamily, member 4 | IGSF4 | Hs.370510 | 209030_s_at |
| Immunoglobulin superfamily, member 4 | IGSF4 | Hs.370510 | 232793_at |
| importin 11 | IPO11 | Hs.482269 | 234304_s_at |
| importin 7 | IPO7 | Hs.523470 | 200992_at |
| importin 8 | IPO8 | Hs.505136 | 225528_at |
| importin 9 | IPO9 | Hs.497384 | 217885_at |
| importin 9 | IPO9 | Hs.497384 | 225728_at |
| influenza virus NS1A binding protein | IVNS1ABP | Hs.497183 | 206245_s_at |
| Inhibin, alpha | INHA | Hs.407506 | 1569409_x_at |
| inhibin, beta C | INHBC | Hs.374664 | 207688_s_at |
| inhibitor of DNA binding 2, dominant negative | ID2 | Hs.180919 | 201565_s_at |
| helix-loop-helix protein | | | |
| inhibitor of DNA binding 2, dominant negative | ID2 /// | Hs.180919 | 201566_x_at |
| helix-loop-helix protein /// inhibitor of DNA binding | ID2B | | |
| 2B, dominant negative helix-loop-helix protein | | | |
| inhibitor of DNA binding 2, dominant negative | ID2 /// | Hs.180919 | 213931_at |
| helix-loop-helix protein /// inhibitor of DNA binding | ID2B | | |
| 2B, dominant negative helix-loop-helix protein | | | |
| inhibitor of DNA binding 4, dominant negative | ID4 | Hs.519601 | 209291_at |
| helix-loop-helix protein | | | |
| inhibitor of DNA binding 4, dominant negative | ID4 | Hs.519601 | 209293_x_at |
| helix-loop-helix protein | | | |
| inhibitor of growth family, member 3 | ING3 | Hs.489811 | 205070_at |
| inner membrane protein, mitochondrial (mitofilin) | IMMT | Hs.148559 | 200955_at |
| Inner membrane protein, mitochondrial (mitofilin) | IMMT | Hs.148559 | 242361_at |
| inorganic pyrophosphatase 2 | PPA2 | Hs.480452 | 220741_s_at |
| inositol hexaphosphate kinase 2 | IHPK2 | Hs.476221 | 223165_s_at |
| inositol polyphosphate-1-phosphatase | INPP1 | Hs.32309 | 202794_at |
| inositol polyphosphate-4-phosphatase, type II, | INPP4B | Hs.480837 | 205376_at |
| 105 kDa | | | |
| inositol polyphosphate-4-phosphatase, type II, | INPP4B | Hs.480837 | 223878_at |
| 105 kDa | | | |
| inositol polyphosphate-5-phosphatase, 40 kDa | INPP5A | Hs.523360 | 203006_at |
| insulin receptor | INSR | Hs.465744 | 213792_s_at |
| Insulin receptor | INSR | Hs.465744 | 227432_s_at |
| insulin receptor substrate 2 | IRS2 | Hs.442344 | 209184_s_at |
| insulin-degrading enzyme | IDE | Hs.500546 | 203328_x_at |
| integral membrane glycoprotein-like /// similar to | LOC166994 | Hs.531427 | 1558011_at |
| bA110H4.2 (similar to membrane protein) /// similar | /// | | |
| to bA110H4.2 (similar to membrane protein) /// | LOC340089 | | |
| similar to bA110H4.2 (similar to membrane protein) | /// | | |
| | LOC441081 | | |
| | /// | | |
| | LOC441098 | | |
| integral membrane protein 2B | ITM2B | Hs.446450 | 217732_s_at |
| integrin beta 3 binding protein (beta3-endonexin) | ITGB3BP | Hs.166539 | 205176_s_at |
| integrin beta 4 binding protein | ITGB4BP | Hs.534326 | 210213_s_at |
| integrin, alpha 10 | ITGA10 | Hs.158237 | 206766_at |
| integrin, alpha 2b (platelet glycoprotein IIb of | ITGA2B | Hs.411312 | 206493_at |
| IIb/IIIa complex, antigen CD41B) | | | |
| integrin, beta 1 (fibronectin receptor, beta | ITGB1 | Hs.429052 | 211945_s_at |
| polypeptide, antigen CD29 includes MDF2, | | | |
| MSK12) | | | |
| integrin, beta 3 (platelet glycoprotein IIIa, antigen | ITGB3 | Hs.218040 | 216261_at |
| CD61) | | | |
| Integrin, beta-like 1 (with EGF-like repeat domains) | ITGBL1 | Hs.508597 | 233185_at |
| inter-alpha (globulin) inhibitor H3 | ITIH3 | Hs.76716 | 205755_at |
| inter-alpha (globulin) inhibitor H4 (plasma | ITIH4 | Hs.518000 | 242720_at |
| Kallikrein-sensitive glycoprotein) | | | |
| interferon induced transmembrane protein 1 (9-27) | IFITM1 | Hs.458414 | 214022_s_at |
| interferon regulatory factor 1 | IRF1 | Hs.436061 | 202531_at |
| interferon regulatory factor 6 | IRF6 | Hs.355827 | 202597_at |
| interferon-induced protein with tetratricopeptide | IFIT3 | Hs.549041 | 229450_at |
| repeats 3 | | | |
| interleukin 1 receptor antagonist | IL1RN | Hs.81134 | 212657_s_at |
| interleukin 13 receptor, alpha 2 | IL13RA2 | Hs.336046 | 206172_at |
| Interleukin 24 | IL24 | Hs.411311 | 239122_at |
| Interleukin 4 receptor | IL4R | Hs.513457 | 242743_at |
| interleukin 6 receptor | IL6R | Hs.135087 | 226333_at |
| interleukin 6 receptor /// interleukin 6 receptor | IL6R | Hs.135087 | 205945_at |
| interleukin 6 signal transducer (gp130, oncostatin | IL6ST | Hs.532082 | 204864_s_at |
| M receptor) | | | |
| interleukin 7 | IL7 | Hs.536926 | 206693_at |
| interleukin enhancer binding factor 3, 90 kDa | ILF3 | Hs.465885 | 211375_s_at |
| intersectin 1 (SH3 domain protein) | ITSN1 | Hs.160324 | 35776_at |
| intersectin 2 | ITSN2 | Hs.432562 | 209898_x_at |
| intracisternal A particle-promoted polypeptide | IPP | Hs.157180 | 1554740_a_at |
| IQ motif and WD repeats 1 | IQWD1 | Hs.435741 | 217908_s_at |
| IQ motif and WD repeats 1 | IQWD1 | Hs.435741 | 232349_x_at |
| IQ motif containing F1 | IQCF1 | Hs.381854 | 1552997_a_at |
| IQ motif containing F3 | IQCF3 | Hs.254808 | 1555235_s_at |
| IQ motif containing F3 | IQCF3 | Hs.254808 | 236871_s_at |
| IQ motif containing G | IQCG | Hs.518536 | 221185_s_at |
| IQ motif containing GTPase activating protein 1 | IQGAP1 | Hs.430551 | 210840_s_at |
| IQ motif containing GTPase activating protein 3 | IQGAP3 | Hs.133294 | 229538_s_at |
| IQ motif containing with AAA domain | IQCA | Hs.129174 | 238584_at |
| ISL1 transcription factor, LIM/homeodomain, (islet- | ISL1 | Hs.505 | 206104_at |
| 1) | | | |
| islet cell autoantigen 1, 69 kDa /// islet cell | ICA1 | Hs.487561 | 211740_at |
| autoantigen 1, 69 kDa | | | |
| isocitrate dehydrogenase 1 (NADP+), soluble | IDH1 | Hs.11223 | 1555037_a_at |
| isocitrate dehydrogenase 3 (NAD+) alpha | IDH3A | Hs.250616 | 202069_s_at |
| isoleucine-tRNA synthetase | IARS | Hs.445403 | 204744_s_at |
| Isoleucine-tRNA synthetase | IARS | Hs.445403 | 239794_at |
| Isopentenyl-diphosphate delta isomerase | IDI1 | Hs.283652 | 242255_at |
| itchy homolog E3 ubiquitin protein ligase (mouse) | ITCH | Hs.472509 | 209744_x_at |
| itchy homolog E3 ubiquitin protein ligase (mouse) | ITCH | Hs.472509 | 217094_s_at |
| Janus kinase 1 (a protein tyrosine kinase) | JAK1 | Hs.551505 | 201648_at |
| jumonji domain containing 1A | JMJD1A | Hs.531819 | 212689_s_at |
| jumonji domain containing 1C | JMJD1C | Hs.413416 | 224933_s_at |
| jumonji domain containing 1C | JMJD1C | Hs.413416 | 228793_at |
| jumonji domain containing 2A | JMJD2A | Hs.155983 | 203204_s_at |
| jumonji domain containing 2C | JMJD2C | Hs.157106 | 214861_at |
| Jumonji domain containing 2C | JMJD2C | Hs.157106 | 239285_at |
| Jumonji, AT rich interactive domain 1A (RBBP2- | JARID1A | Hs.76272 | 202040_s_at |
| like) | | | |
| Jumonji, AT rich interactive domain 1B (RBP2-like) | JARID1B | Hs.443650 | 201548_s_at |
| Jumonji, AT rich interactive domain 1B (RBP2-like) | JARID1B | Hs.443650 | 201549_x_at |
| Jumonji, AT rich interactive domain 1B (RBP2-like) | JARID1B | Hs.443650 | 211202_s_at |
| Jumonji, AT rich interactive domain 2 | JARID2 | Hs.269059 | 203297_s_at |
| jumping translocation breakpoint | JTB | Hs.6396 | 210434_x_at |
| jumping translocation breakpoint | JTB | Hs.6396 | 210927_x_at |
| jun D proto-oncogene | JUND | Hs.2780 | 203752_s_at |
| junction-mediating and regulatory protein | JMY | Hs.482605 | 241985_at |
| junctophilin 1 | JPH1 | Hs.160574 | 1553533_at |
| KARP-1-binding protein | KAB | Hs.533635 | 207719_x_at |
| KARP-1-binding protein /// hypothetical protein | KAB /// | Hs.533635 | 1552717_s_at |
| MGC26143 | MGC26143 | | |
| karyopherin (importin) beta 1 | KPNB1 | Hs.532793 | 208974_x_at |
| karyopherin (importin) beta 1 | KPNB1 | Hs.532793 | 213507_s_at |
| Karyopherin (importin) beta 1 | KPNB1 | Hs.532793 | 213574_s_at |
| Karyopherin (importin) beta 1 | KPNB1 | Hs.532793 | 213803_at |
| karyopherin alpha 1 (importin alpha 5) | KPNA1 | Hs.161008 | 213741_s_at |
| karyopherin alpha 2 (RAG cohort 1, importin alpha | KPNA2 | Hs.159557 | 201088_at |
| 1) | | | |
| karyopherin alpha 2 (RAG cohort 1, importin alpha | KPNA2 | Hs.159557 | 211762_s_at |
| 1) /// karyopherin alpha 2 (RAG cohort 1, importin | | | |
| alpha 1) | | | |
| karyopherin alpha 3 (importin alpha 4) | KPNA3 | Hs.527919 | 221502_at |
| karyopherin alpha 3 (importin alpha 4) | KPNA3 | Hs.527919 | 221503_s_at |
| Karyopherin alpha 3 (importin alpha 4) | KPNA3 | Hs.527919 | 233224_at |
| karyopherin alpha 4 (importin alpha 3) | KPNA4 | Hs.288193 | 225268_at |
| Karyopherin alpha 5 (importin alpha 6) | KPNA5 | Hs.182971 | 227934_at |
| karyopherin alpha 6 (importin alpha 7) | KPNA6 | Hs.470588 | 212101_at |
| karyopherin alpha 6 (importin alpha 7) | KPNA6 | Hs.470588 | 212102_s_at |
| Karyopherin alpha 6 (importin alpha 7) | KPNA6 | Hs.470588 | 226976_at |
| katanin p60 (ATPase-containing) subunit A 1 | KATNA1 | Hs.131838 | 205526_s_at |
| katanin p60 subunit A-like 1 | KATNAL1 | Hs.119672 | 223790_at |
| Katanin p60 subunit A-like 1 | KATNAL1 | Hs.119672 | 243828_at |
| katanin p80 (WD repeat containing) subunit B 1 | KATNB1 | Hs.275675 | 203162_s_at |
| KDEL (Lys-Asp-Glu-Leu) endoplasmic reticulum | KDELR2 | Hs.520210 | 200698_at |
| protein retention receptor 2 | | | |
| KDEL (Lys-Asp-Glu-Leu) endoplasmic reticulum | KDELR2 | Hs.520210 | 200700_s_at |
| protein retention receptor 2 | | | |
| kelch domain containing 3 | KLHDC3 | Hs.412468 | 214383_x_at |
| kelch domain containing 4 | KLHDC4 | Hs.461688 | 232171_x_at |
| kelch domain containing 5 | KLHDC5 | Hs.505104 | 225961_at |
| kelch repeat and BTB (POZ) domain containing 2 | KBTBD2 | Hs.372541 | 212447_at |
| kelch repeat and BTB (POZ) domain containing 2 | KBTBD2 | Hs.372541 | 223584_s_at |
| kelch repeat and BTB (POZ) domain containing 3 | KBTBD3 | Hs.101949 | 228777_at |
| kelch repeat and BTB (POZ) domain containing 4 | KBTBD4 | Hs.440695 | 218570_at |
| kelch repeat and BTB (POZ) domain containing 7 | KBTBD7 | Hs.63841 | 223412_at |
| kelch/ankyrin repeat containing cyclin A1 | KARCA1 | Hs.507290 | 227582_at |
| interacting protein | | | |
| kelch-like 10 (Drosophila) | KLHL10 | Hs.127510 | 1552425_a_at |
| Kelch-like 11 (Drosophila) | KLHL11 | Hs.13268 | 226883_at |
| kelch-like 12 (Drosophila) | KLHL12 | Hs.282878 | 225068_at |
| kelch-like 18 (Drosophila) | KLHL18 | Hs.517946 | 212882_at |
| kelch-like 20 (Drosophila) | KLHL20 | Hs.495035 | 204177_s_at |
| kelch-like 7 (Drosophila) | KLHL7 | Hs.385861 | 220238_s_at |
| kelch-like 7 (Drosophila) | KLHL7 | Hs.385861 | 220239_at |
| kelch-like 7 (Drosophila) | KLHL7 | Hs.385861 | 223250_at |
| kelch-like 8 (Drosophila) | KLHL8 | Hs.106601 | 226874_at |
| keratin 10 (epidermolytic hyperkeratosis; keratosis | KRT10 | Hs.99936 | 207023_x_at |
| palmaris et plantaris) | | | |
| keratin 10 (epidermolytic hyperkeratosis; keratosis | KRT10 | Hs.99936 | 210633_x_at |
| palmaris et plantaris) | | | |
| keratin 10 (epidermolytic hyperkeratosis; keratosis | KRT10 | Hs.99936 | 213287_s_at |
| palmaris et plantaris) | | | |
| keratin 25A | KRT25A | Hs.55412 | 237905_at |
| keratin associated protein 4-9 | KRTAP4-9 | Hs.307018 | 234685_x_at |
| keratin, hair, basic, 5 | KRTHB5 | Hs.182507 | 207670_at |
| keratinocyte associated protein 2 | KRTCAP2 | Hs.516671 | 224885_s_at |
| KH domain containing, RNA binding, signal | KHDRBS3 | Hs.444558 | 209781_s_at |
| transduction associated 3 | | | |
| KIAA0097 gene product | ch-TOG | Hs.201253 | 212832_s_at |
| KIAA0101 | KIAA0101 | Hs.81892 | 202503_s_at |
| KIAA0101 /// KIAA0101 | KIAA0101 | Hs.81892 | 211713_x_at |
| KIAA0117 protein | KIAA0117 | Hs.535224 | 212591_at |
| KIAA0153 protein | KIAA0153 | Hs.517670 | 216251_s_at |
| KIAA0182 protein | KIAA0182 | Hs.461647 | 212057_at |
| KIAA0186 gene product | KIAA0186 | Hs.360033 | 206102_at |
| KIAA0195 gene product | KIAA0195 | Hs.514474 | 202650_s_at |
| KIAA0217 | KIAA0217 | Hs.498455 | 208952_s_at |
| KIAA0217 | KIAA0217 | Hs.498455 | 228196_s_at |
| KIAA0217 | KIAA0217 | Hs.498455 | 241965_at |
| KIAA0226 | KIAA0226 | Hs.478868 | 212735_at |
| KIAA0232 gene product | KIAA0232 | Hs.79276 | 212441_at |
| KIAA0247 | KIAA0247 | Hs.440025 | 202181_at |
| KIAA0251 protein | KIAA0251 | Hs.370781 | 212053_at |
| KIAA0251 protein | KIAA0251 | Hs.370781 | 232288_at |
| KIAA0252 | KIAA0252 | Hs.511096 | 212302_at |
| KIAA0261 | KIAA0261 | Hs.203099 | 1554441_a_at |
| KIAA0261 | KIAA0261 | Hs.203099 | 212264_s_at |
| KIAA0265 protein | KIAA0265 | Hs.520710 | 210111_s_at |
| KIAA0265 protein | KIAA0265 | Hs.520710 | 238000_at |
| KIAA0286 protein | KIAA0286 | Hs.533787 | 212619_at |
| KIAA0329 | KIAA0329 | Hs.195667 | 204308_s_at |
| KIAA0352 gene product | KIAA0352 | Hs.131212 | 205256_at |
| KIAA0368 | KIAA0368 | Hs.368255 | 214356_s_at |
| KIAA0368 | KIAA0368 | Hs.368255 | 236368_at |
| KIAA0391 | KIAA0391 | Hs.458487 | 202713_s_at |
| KIAA0420 gene product | KIAA0420 | Hs.512856 | 210169_at |
| KIAA0423 | KIAA0423 | Hs.371078 | 213304_at |
| KIAA0431 protein | KIAA0431 | Hs.16349 | 201854_s_at |
| KIAA0446 gene product | KIAA0446 | Hs.532375 | 32091_at |
| KIAA0467 protein | KIAA0467 | Hs.301943 | 203900_at |
| KIAA0478 gene product | KIAA0478 | Hs.528723 | 203958_s_at |
| KIAA0492 protein | KIAA0492 | Hs.531342 | 215109_at |
| KIAA0494 gene product | KIAA0494 | Hs.100874 | 201778_s_at |
| KIAA0495 | KIAA0495 | Hs.49658 | 213340_s_at |
| KIAA0563-related gene | LOC114659 | Hs.514071 | 229231_at |
| KIAA0564 protein | KIAA0564 | Hs.368282 | 214231_s_at |
| KIAA0663 gene product | KIAA0663 | Hs.532399 | 205788_s_at |
| KIAA0674 | KIAA0674 | Hs.522351 | 237868_x_at |
| KIAA0685 | KIAA0685 | Hs.449098 | 1570210_x_at |
| KIAA0746 protein | KIAA0746 | Hs.479384 | 212311_at |
| KIAA0794 protein | KIAA0794 | Hs.518524 | 212840_at |
| KIAA0794 protein | KIAA0794 | Hs.518524 | 217100_s_at |
| KIAA0802 | KIAA0802 | Hs.464563 | 213358_at |
| KIAA0804 | KIAA0804 | Hs.269263 | 1561467_at |
| KIAA0804 | KIAA0804 | Hs.269263 | 236937_at |
| KIAA0804 | KIAA0804 | Hs.269263 | 239917_at |
| KIAA0828 protein | KIAA0828 | Hs.195058 | 212814_at |
| KIAA0828 protein | KIAA0828 | Hs.195058 | 215672_s_at |
| KIAA0830 protein | KIAA0830 | Hs.167115 | 212570_at |
| KIAA0830 protein | KIAA0830 | Hs.167115 | 212573_at |
| KIAA0831 | KIAA0831 | Hs.414809 | 233984_at |
| KIAA0841 | KIAA0841 | Hs.7426 | 36888_at |
| KIAA0863 protein | KIAA0863 | Hs.131915 | 203321_s_at |
| KIAA0877 protein | KIAA0877 | Hs.408623 | 1560915_at |
| KIAA0877 protein | KIAA0877 | Hs.408623 | 1560916_a_at |
| KIAA0892 | KIAA0892 | Hs.112751 | 212505_s_at |
| KIAA0907 protein | KIAA0907 | Hs.24656 | 202220_at |
| KIAA0912 protein | Cep152 | Hs.443005 | 215170_s_at |
| KIAA0912 protein | Cep152 | Hs.443005 | 237129_at |
| KIAA0934 | KIAA0934 | Hs.432397 | 1560763_at |
| KIAA0980 protein | KIAA0980 | Hs.472347 | 207705_s_at |
| KIAA0998 | KIAA0998 | Hs.200747 | 214672_at |
| KIAA0998 | KIAA0998 | Hs.200747 | 215898_at |
| KIAA0998 | KIAA0998 | Hs.200747 | 243988_at |
| KIAA0998 /// KIAA0998 | KIAA0998 | Hs.200747 | 208099_x_at |
| KIAA0999 protein | KIAA0999 | Hs.167451 | 1554929_at |
| KIAA0999 protein | KIAA0999 | Hs.167451 | 1563455_at |
| KIAA0999 protein | KIAA0999 | Hs.167451 | 204157_s_at |
| KIAA0999 protein | KIAA0999 | Hs.167451 | 242920_at |
| KIAA1002 protein | KIAA1002 | Hs.443673 | 203831_at |
| KIAA1008 | KIAA1008 | Hs.531029 | 222607_s_at |
| KIAA1012 | KIAA1012 | Hs.202001 | 207305_s_at |
| KIAA1026 protein | KIAA1026 | Hs.368823 | 213478_at |
| KIAA1043 protein | KIAA1043 | Hs.387856 | 215146_s_at |
| KIAA1049 protein | KIAA1049 | Hs.415342 | 213311_s_at |
| KIAA1052 protein | Cep164 | Hs.504009 | 1558953_s_at |
| KIAA1076 protein | KIAA1076 | Hs.507122 | 213153_at |
| KIAA1102 protein | KIAA1102 | Hs.335163 | 212325_at |
| KIAA1102 protein | KIAA1102 | Hs.335163 | 212327_at |
| KIAA1102 protein | KIAA1102 | Hs.335163 | 212328_at |
| KIAA1109 | KIAA1109 | Hs.408142 | 212779_at |
| KIAA1109 | KIAA1109 | Hs.408142 | 216294_s_at |
| KIAA1115 | KIAA1115 | Hs.515610 | 209229_s_at |
| KIAA1126 protein | KIAA1126 | Hs.372492 | 225598_at |
| KIAA1128 | KIAA1128 | Hs.461988 | 209378_s_at |
| KIAA1128 | KIAA1128 | Hs.461988 | 240499_at |
| KIAA1143 | KIAA1143 | Hs.476082 | 226816_s_at |
| KIAA1191 protein /// KIAA1191 protein | KIAA1191 | Hs.519783 | 224502_s_at |
| KIAA1193 | KIAA1193 | Hs.101891 | 44822_s_at |
| KIAA1211 protein | KIAA1211 | Hs.479783 | 1564828_at |
| KIAA1211 protein | KIAA1211 | Hs.479783 | 227230_s_at |
| KIAA1212 | KIAA1212 | Hs.292925 | 239233_at |
| KIAA1217 | KIAA1217 | Hs.445885 | 231807_at |
| KIAA1219 protein | KIAA1219 | Hs.436705 | 224678_at |
| KIAA1219 protein | KIAA1219 | Hs.436705 | 242233_at |
| KIAA1238 protein | KIAA1238 | Hs.504670 | 225999_at |
| KIAA1238 protein | KIAA1238 | Hs.504670 | 226164_x_at |
| KIAA1238 protein | KIAA1238 | Hs.504670 | 229344_x_at |
| KIAA1238 protein | KIAA1238 | Hs.504670 | 242870_at |
| KIAA1257 | KIAA1257 | Hs.518247 | 1554852_a_at |
| KIAA1272 protein | KIAA1272 | Hs.472285 | 232500_at |
| KIAA1327 protein | KIAA1327 | Hs.106204 | 225821_s_at |
| KIAA1333 | KIAA1333 | Hs.509008 | 223254_s_at |
| KIAA1333 | KIAA1333 | Hs.509008 | 223256_at |
| KIAA1344 | KIAA1344 | Hs.532609 | 226747_at |
| KIAA1345 protein | KIAA1345 | Hs.303820 | 234936_s_at |
| KIAA1377 protein | KIAA1377 | Hs.156352 | 235956_at |
| KIAA1387 protein | KIAA1387 | Hs.516182 | 222270_at |
| KIAA1393 | KIAA1393 | Hs.380159 | 221952_x_at |
| KIAA1393 | KIAA1393 | Hs.380159 | 227653_at |
| KIAA1430 | KIAA1430 | Hs.535734 | 226254_s_at |
| KIAA1432 | KIAA1432 | Hs.211520 | 226222_at |
| KIAA1447 protein | KIAA1447 | Hs.514580 | 1559048_at |
| KIAA1450 protein | KIAA1450 | Hs.377588 | 225924_at |
| KIAA1450 protein | KIAA1450 | Hs.377588 | 226460_at |
| KIAA1458 protein | KIAA1458 | Hs.479677 | 224844_at |
| KIAA1458 protein | KIAA1458 | Hs.479677 | 224845_s_at |
| KIAA1530 protein | KIAA1530 | Hs.380475 | 233893_s_at |
| KIAA1536 protein | KIAA1536 | Hs.156667 | 209002_s_at |
| KIAA1596 | KIAA1596 | Hs.509229 | 234733_s_at |
| KIAA1596 | KIAA1596 | Hs.509229 | 242711_x_at |
| KIAA1600 | KIAA1600 | Hs.192619 | 226155_at |
| KIAA1608 | KIAA1608 | Hs.435064 | 226849_at |
| KIAA1632 | KIAA1632 | Hs.546416 | 227638_at |
| KIAA1632 | KIAA1632 | Hs.546416 | 232030_at |
| KIAA1641 | KIAA1641 | Hs.516348 | 220940_at |
| KIAA1666 protein | KIAA1666 | Hs.115429 | 230062_at |
| KIAA1698 protein | KIAA1698 | Hs.458390 | 53968_at |
| KIAA1704 | KIAA1704 | Hs.507922 | 229078_s_at |
| KIAA1706 protein | KIAA1706 | Hs.487994 | 1563621_at |
| KIAA1712 | KIAA1712 | Hs.551562 | 241426_at |
| KIAA1713 | KIAA1713 | Hs.464876 | 233536_at |
| KIAA1718 protein | KIAA1718 | Hs.308710 | 221778_at |
| KIAA1727 protein | KIAA1727 | Hs.132629 | 226599_at |
| KIAA1731 protein | KIAA1731 | Hs.458418 | 1569302_at |
| KIAA1754-like | KIAA1754L | Hs.65009 | 240037_at |
| KIAA1772 | KIAA1772 | Hs.54838 | 233977_at |
| KIAA1815 | KIAA1815 | Hs.87128 | 222603_at |
| KIAA1856 protein | KIAA1856 | Hs.520638 | 229257_at |
| KIAA1904 protein | KIAA1904 | Hs.250726 | 1559072_a_at |
| KIAA1935 protein | KIAA1935 | Hs.438851 | 226720_at |
| KIAA1949 | KIAA1949 | Hs.101150 | 224927_at |
| KIAA1950 protein | KIAA1950 | Hs.42221 | 1561179_s_at |
| KIAA1958 | KIAA1958 | Hs.533491 | 235112_at |
| KIAA1961 gene | KIAA1961 | Hs.483329 | 239646_at |
| KIAA1972 protein | KIAA1972 | Hs.460885 | 225770_at |
| KIAA1972 protein | KIAA1972 | Hs.460885 | 225773_at |
| KIAA2010 | KIAA2010 | Hs.533887 | 220368_s_at |
| KIAA2026 | KIAA2026 | Hs.255430 | 228446_at |
| kinectin 1 (kinesin receptor) | KTN1 | Hs.509414 | 200914_x_at |
| kinectin 1 (kinesin receptor) /// protein disulfide | KTN1 /// | Hs.509414 | 200915_x_at |
| isomerase-associated 6 | PDIA6 | | |
| kinectin 1 (kinesin receptor) /// protein disulfide | KTN1 /// | Hs.509414 | 214709_s_at |
| isomerase-associated 6 | PDIA6 | | |
| Kinesin 2 60/70 kDa | KNS2 | Hs.20107 | 216187_x_at |
| kinesin family member 14 | KIF14 | Hs.3104 | 206364_at |
| kinesin family member 15 | KIF15 | Hs.307529 | 219306_at |
| kinesin family member 18A /// kinesin family | KIF18A | Hs.301052 | 221258_s_at |
| member 18A | | | |
| Kinesin family member 1B | KIF1B | Hs.97858 | 225878_at |
| kinesin family member 1B | KIF1B | Hs.97858 | 226968_at |
| Kinesin family member 27 | KIF27 | Hs.546403 | 240547_at |
| kinesin family member 2C | KIF2C | Hs.69360 | 209408_at |
| kinesin family member 2C | KIF2C | Hs.69360 | 211519_s_at |
| kinesin family member 3A | KIF3A | Hs.43670 | 213623_at |
| kinesin family member 3B | KIF3B | Hs.369670 | 203943_at |
| kinesin family member 5B | KIF5B | Hs.327736 | 201991_s_at |
| kinesin family member 5B | KIF5B | Hs.327736 | 201992_s_at |
| kinesin family member 5B | KIF5B | Hs.327736 | 224662_at |
| Kruppel-like factor 10 | KLF10 | Hs.435001 | 202393_s_at |
| Kruppel-like factor 11 | KLF11 | Hs.12229 | 1553137_s_at |
| Kruppel-like factor 4 (gut) | KLF4 | Hs.376206 | 221841_s_at |
| Kruppel-like factor 5 (intestinal) | KLF5 | Hs.508234 | 209212_s_at |
| Kruppel-like factor 7 (ubiquitous) | KLF7 | Hs.471221 | 1555420_a_at |
| Kruppel-like factor 7 (ubiquitous) | KLF7 | Hs.471221 | 204334_at |
| Kruppel-like factor 9 | KLF9 | Hs.150557 | 230636_s_at |
| Ku70-binding protein 3 | KUB3 | Hs.61188 | 227678_at |
| kynureninase (L-kynurenine hydrolase) | KYNU | Hs.470126 | 217388_s_at |
| kynurenine 3-monooxygenase (kynurenine 3- | KMO | Hs.409081 | 205306_x_at |
| hydroxylase) | | | |
| kynurenine 3-monooxygenase (kynurenine 3- | KMO | Hs.409081 | 205307_s_at |
| hydroxylase) | | | |
| lactamase, beta | LACTB | Hs.410388 | 226354_at |
| lactamase, beta 2 | LACTB2 | Hs.118554 | 222714_s_at |
| lactate dehydrogenase A | LDHA | Hs.2795 | 200650_s_at |
| lactate dehydrogenase B | LDHB | Hs.446149 | 201030_x_at |
| lactate dehydrogenase C | LDHC | Hs.99881 | 207022_s_at |
| LAG1 longevity assurance homolog 3 (S. cerevisiae) | LASS3 | Hs.416099 | 1554253_a_at |
| LAG1 longevity assurance homolog 6 (S. cerevisiae) | LASS6 | Hs.506829 | 235463_s_at |
| lamina-associated polypeptide 1B | LAP1B | Hs.496459 | 212409_s_at |
| laminin, alpha 4 | LAMA4 | Hs.213861 | 202202_s_at |
| Laminin, beta 1 | LAMB1 | Hs.489646 | 236437_at |
| laminin, beta 4 | LAMB4 | Hs.62022 | 215516_at |
| LanC lantibiotic synthetase component C-like 2 | LANCL2 | Hs.224282 | 218219_s_at |
| (bacterial) | | | |
| LAT1-3TM protein | LAT1- | Hs.444600 | 210396_s_at |
| | 3TM | | |
| latent transforming growth factor beta binding | LTBP1 | Hs.49787 | 202729_s_at |
| protein 1 | | | |
| latent transforming growth factor beta binding | LTBP2 | Hs.512776 | 204682_at |
| protein 2 | | | |
| LATS, large tumor suppressor, homolog 2 | LATS2 | Hs.78960 | 223380_s_at |
| (Drosophila) | | | |
| LCHN protein | LCHN | Hs.233044 | 223162_s_at |
| lectin, galactoside-binding, soluble, 3 (galectin 3) /// | LGALS3 | Hs.531081 | 208949_s_at |
| galectin-3 internal gene | /// | | |
| | GALIG | | |
| lectin, galactoside-binding, soluble, 8 (galectin 8) | LGALS8 | Hs.4082 | 208934_s_at |
| LEM domain containing 1 | LEMD1 | Hs.181245 | 229927_at |
| leptin receptor overlapping transcript | LEPROT | — | 202378_s_at |
| leptin receptor overlapping transcript-like 1 | LEPROTL1 | Hs.146585 | 202594_at |
| leptin receptor overlapping transcript-like 1 | LEPROTL1 | Hs.146585 | 202595_s_at |
| leucine proline-enriched proteoglycan (leprecan) 1 | LEPRE1 | Hs.437656 | 220750_s_at |
| leucine rich repeat (in FLII) interacting protein 1 | LRRFIP1 | Hs.471779 | 201861_s_at |
| leucine rich repeat (in FLII) interacting protein 1 | LRRFIP1 | Hs.471779 | 211452_x_at |
| leucine rich repeat (in FLII) interacting protein 1 | LRRFIP1 | Hs.471779 | 223492_s_at |
| leucine rich repeat (in FLII) interacting protein 2 | LRRFIP2 | Hs.475319 | 220610_s_at |
| Leucine rich repeat (in FLII) interacting protein 2 | LRRFIP2 | Hs.475319 | 230082_at |
| Leucine rich repeat containing 16 | LRRC16 | Hs.116470 | 230793_at |
| leucine rich repeat containing 27 | LRRC27 | Hs.119897 | 228886_at |
| leucine rich repeat containing 6 | LRRC6 | Hs.179238 | 206483_at |
| Leucine rich repeat containing 7 | LRRC7 | Hs.479658 | 242050_at |
| Leucine rich repeat neuronal 3 | LRRN3 | Hs.3781 | 243512_x_at |
| leucine zipper protein 1 | LUZP1 | Hs.257900 | 1558173_a_at |
| leucine zipper protein 1 | LUZP1 | Hs.257900 | 225831_at |
| leucine zipper, down-regulated in cancer 1 | LDOC1 | Hs.45231 | 204454_at |
| leucine zipper-EF-hand containing transmembrane | LETM2 | Hs.264887 | 1552546_a_at |
| protein 2 | | | |
| leucine-rich PPR-motif containing | LRPPRC | Hs.368084 | 211971_s_at |
| leucine-rich repeats and immunoglobulin-like | LRIG2 | Hs.448972 | 205953_at |
| domains 2 | | | |
| leucyl-tRNA synthetase | LARS | Hs.432674 | 222427_s_at |
| leukocyte receptor cluster (LRC) member 10 | LENG10 | — | 1564776_at |
| leukocyte receptor cluster (LRC) member 8 | LENG8 | Hs.502378 | 224673_at |
| Leukotriene B4 12-hydroxydehydrogenase | LTB4DH | Hs.546348 | 242775_at |
| leupaxin | LPXN | Hs.125474 | 216250_s_at |
| leupaxin | LPXN | Hs.125474 | 242778_at |
| likely ortholog of chicken chondrocyte protein with | CHPPR | Hs.521608 | 203208_s_at |
| a poly-proline region | | | |
| likely ortholog of mouse gene trap locus 3 | GTL3 | Hs.532755 | 217957_at |
| likely ortholog of mouse hypoxia induced gene 1 | HIG1 | Hs.7917 | 217845_x_at |
| likely ortholog of mouse immediate early response, | LEREPO4 | Hs.368598 | 201593_s_at |
| erythropoietin 4 | | | |
| likely ortholog of mouse immediate early response, | LEREPO4 | Hs.368598 | 201595_s_at |
| erythropoietin 4 | | | |
| likely ortholog of mouse la related protein | LARP | Hs.292078 | 212137_at |
| likely ortholog of mouse limb-bud and heart gene | LBH | Hs.468044 | 221011_s_at |
| /// likely ortholog of mouse limb-bud and heart | | | |
| gene | | | |
| likely ortholog of mouse neighbor of Punc E11 | NOPE | Hs.458607 | 227870_at |
| likely ortholog of mouse Sds3 | SDS3 | Hs.416630 | 224974_at |
| likely ortholog of mouse spermatogenic Zip 1 | SPZ1 | Hs.519403 | 224167_at |
| likely ortholog of mouse TORC2-specific protein | AVO3 | Hs.407926 | 228248_at |
| AVO3 (S. cerevisiae) | | | |
| LIM and senescent cell antigen-like domains 3 | LIMS3 | Hs.469633 | 223800_s_at |
| LIM and SH3 protein 1 | LASP1 | Hs.334851 | 200618_at |
| LIM domain binding 3 | LDB3 | Hs.49998 | 213371_at |
| LIM domain containing preferred translocation | LPP | Hs.444362 | 202822_at |
| partner in lipoma | | | |
| LIM domains containing 1 | LIMD1 | Hs.193370 | 234687_x_at |
| LIM homeobox 2 | LHX2 | Hs.445265 | 206140_at |
| LIM homeobox 4 | LHX4 | Hs.496545 | 1553157_at |
| Limkain beta 2 | FLJ22471 | Hs.114111 | 1556624_at |
| limkain beta 2 | FLJ22471 | Hs.114111 | 218175_at |
| lin-28 homolog (C. elegans) | LIN28 | Hs.86154 | 219823_at |
| lipin 1 | LPIN1 | Hs.467740 | 212272_at |
| lipin 1 | LPIN1 | Hs.467740 | 212274_at |
| lipin 1 | LPIN1 | Hs.467740 | 212276_at |
| lipin 2 | LPIN2 | Hs.268581 | 202459_s_at |
| lipocalin 6 | LCN6 | Hs.98132 | 238071_at |
| Lipoma HMGIC fusion partner | LHFP | Hs.507798 | 232935_at |
| LOC132671 | LOC132671 | Hs.527090 | 229331_at |
| LOC132671 | LOC132671 | Hs.527090 | 230723_at |
| LOC136263 | LOC136263 | Hs.351816 | 1553329_at |
| LOC150368 protein | LOC150368 | Hs.368312 | 226504_at |
| LOC399491 protein | LOC399491 | — | 214035_x_at |
| LOC439987 | — | Hs.164298 | 229318_at |
| LOC439987 | — | Hs.164298 | 232601_at |
| LOC440135 | — | Hs.549373 | 234056_at |
| LOC440135 /// Homo sapiens, clone | — | Hs.375766 /// | 228913_at |
| IMAGE: 2900578, mRNA, partial cds | | Hs.549373 | |
| LOC440286 | LOC440286 | Hs.419760 | 237017_s_at |
| LOC440309 | — | Hs.459403 | 225951_s_at |
| LOC440441 | — | Hs.463231 | 230388_s_at |
| LOC440448 | — | Hs.528952 | 224806_at |
| LOC440460 | SH3GLP3 | Hs.293560 | 227567_at |
| LOC440570 | — | Hs.308060 | 241835_at |
| LOC440570 | LOC440570 | Hs.308060 | 242884_at |
| LOC440667 | LOC440667 | Hs.549388 | 229872_s_at |
| LOC440701 | LOC440701 | Hs.550597 | 225722_at |
| LOC440806 | — | Hs.474303 | 224621_at |
| LOC440918 | — | Hs.74921 | 230203_at |
| LOC441297 | LOC441297 | Hs.535040 | 228899_at |
| LOC441423 | LOC441423 | Hs.535066 | 241803_s_at |
| LOC441434 | — | Hs.351215 | 1554516_at |
| LOC441434 | — | Hs.351215 | 1554517_x_at |
| LOC441699 | — | Hs.535102 | 1566887_x_at |
| loss of heterozygosity, 3, chromosomal region 2, | LOH3CR2A | Hs.433151 | 220244_at |
| gene A | | | |
| low molecular mass ubiquinone-binding protein | QP-C | Hs.146602 | 201568_at |
| (9.5 kD) | | | |
| LPS-responsive vesicle trafficking, beach and | LRBA | Hs.480938 | 212692_s_at |
| anchor containing | | | |
| LPS-responsive vesicle trafficking, beach and | LRBA | Hs.480938 | 214109_at |
| anchor containing | | | |
| LUC7-like (S. cerevisiae) | LUC7L | Hs.16803 | 223295_s_at |
| LUC7-like 2 (S. cerevisiae) | LUC7L2 | Hs.530118 | 220099_s_at |
| lung cancer-related protein 8 | HLC-8 | Hs.12929 | 223352_s_at |
| lymphocyte adaptor protein | LNK | Hs.506784 | 203320_at |
| lymphocyte antigen 6 complex, locus G5C | LY6G5C | Hs.25738 | 219860_at |
| lymphocyte antigen 6 complex, locus G6C | LY6G6C | Hs.241586 | 207114_at |
| lymphoid enhancer-binding factor 1 | LEF1 | Hs.125132 | 243362_s_at |
| lymphoid enhancer-binding factor 1 | LEF1 | Hs.125132 | 243363_at |
| LYRIC/3D3 | LYRIC | Hs.377155 | 212250_at |
| LYRIC/3D3 | LYRIC | Hs.377155 | 212251_at |
| lysophosphatidylglycerol acyltransferase 1 | LPGAT1 | Hs.497674 | 1555058_a_at |
| lysophospholipase I | LYPLA1 | Hs.435850 | 203007_x_at |
| lysophospholipase II /// similar to Acyl-protein | LYPLA2 | Hs.534075 | 202292_x_at |
| thioesterase 2 (Lysophospholipase II) (LPL-I) | /// | | |
| | LOC388499 | | |
| lysosomal associated protein transmembrane 4 | LAPTM4B | Hs.492314 | 214039_s_at |
| beta | | | |
| lysosomal trafficking regulator | LYST | Hs.532411 | 210943_s_at |
| lysosomal-associated membrane protein 1 | LAMP1 | Hs.494419 | 201552_at |
| lysosomal-associated membrane protein 1 | LAMP1 | Hs.494419 | 201553_s_at |
| lysosomal-associated membrane protein 3 | LAMP3 | Hs.518448 | 205569_at |
| lysosomal-associated protein transmembrane 4 | LAPTM4A | Hs.467807 | 200673_at |
| alpha | | | |
| lysyl-tRNA synthetase | KARS | Hs.3100 | 200840_at |
| M8 protein | LOC149830 | Hs.126516 | 233975_at |
| macrophage erythroblast attacher | MAEA | Hs.139896 | 207922_s_at |
| MAD1 mitotic arrest deficient-like 1 (yeast) | MAD1L1 | Hs.209128 | 233921_s_at |
| MADS box transcription enhancer factor 2, | MEF2A | Hs.268675 | 239571_at |
| polypeptide A (myocyte enhancer factor 2A) | | | |
| Major histocompatibility complex, class I, A | HLA-A | Hs.549038 | 213932_x_at |
| major histocompatibility complex, class II, DM | HLA- | Hs.351279 | 217478_s_at |
| alpha | DMA | | |
| major histocompatibility complex, class II, DM beta | HLA- | Hs.1162 | 203932_at |
| /// major histocompatibility complex, class II, DM | DMB | | |
| beta | | | |
| major histocompatibility complex, class II, DR beta | HLA- | Hs.520049 | 209312_x_at |
| 1 /// major histocompatibility complex, class II, DR | DRB1 | | |
| beta 1 | | | |
| Major histocompatibility complex, class II, DR beta 4 | HLA- | Hs.520049 | 208306_x_at |
| | DRB1 | | |
| Mak3 homolog (S. cerevisiae) | MAK3 | Hs.269528 | 231199_at |
| Mak3 homolog (S. cerevisiae) | MAK3 | Hs.269528 | 244341_at |
| makorin, ring finger protein, 1 | MKRN1 | Hs.490347 | 209845_at |
| makorin, ring finger protein, 1 /// makorin, ring | MKRN1 | Hs.490347 | 201285_at |
| finger protein, 1 | | | |
| makorin, ring finger protein, 2 | MKRN2 | Hs.279474 | 222510_s_at |
| mal, T-cell differentiation protein | MAL | Hs.80395 | 204777_s_at |
| malic enzyme 1, NADP(+)-dependent, cytosolic | ME1 | Hs.21160 | 204059_s_at |
| Mannose receptor, C type 2 | MRC2 | Hs.7835 | 244788_at |
| MAP kinase interacting serine/threonine kinase 1 | MKNK1 | Hs.371594 | 209467_s_at |
| MAP kinase interacting serine/threonine kinase 1 | MKNK1 | Hs.371594 | 243256_at |
| MAP kinase interacting serine/threonine kinase 2 | MKNK2 | Hs.515032 | 218205_s_at |
| MAP/microtubule affinity-regulating kinase 1 | MARK1 | Hs.497806 | 221047_s_at |
| MAP/microtubule affinity-regulating kinase 1 | MARK1 | Hs.497806 | 226653_at |
| MARCKS-like 1 | MARCKSL1 | Hs.75061 | 200644_at |
| Mariner-like element-containing mRNA, clone | — | Hs.546722 | 239409_at |
| pcHMT2. | | | |
| mastermind-like 2 (Drosophila) | MAML2 | Hs.428214 | 235457_at |
| Mastermind-like 2 (Drosophila) | MAML2 | Hs.428214 | 244414_at |
| Mastermind-like 3 (Drosophila) | MAML3 | Hs.444627 | 242794_at |
| maternally expressed 3 | MEG3 | Hs.525589 | 212732_at |
| matrin 3 | MATR3 | Hs.268939 | 214363_s_at |
| matrix metalloproteinase 12 (macrophage | MMP12 | Hs.1695 | 204580_at |
| elastase) | | | |
| matrix metalloproteinase 19 | MMP19 | Hs.154057 | 204574_s_at |
| matrix metalloproteinase 9 (gelatinase B, 92 kDa | MMP9 | Hs.297413 | 203936_s_at |
| gelatinase, 92 kDa type IV collagenase) | | | |
| MAX dimerization protein 1 | MAD | Hs.468908 | 226275_at |
| MAX-like protein X | MLX | Hs.383019 | 213708_s_at |
| MBC3205 | UNQ501 | Hs.8036 | 225003_at |
| Mbt domain containing 1 | MBTD1 | Hs.369586 | 226797_at |
| MCF.2 cell line derived transforming sequence-like | MCF2L | Hs.170422 | 243983_at |
| McKusick-Kaufman syndrome | MKKS | Hs.472119 | 222530_s_at |
| MCM3 minichromosome maintenance deficient 3 | MCM3APAS | Hs.517368 | 220459_at |
| (S. cerevisiae) associated protein antisense | | | |
| Mdm4, transformed 3T3 cell double minute 1, p53 | MDM1 | Hs.171265 | 213761_at |
| binding protein (mouse) | | | |
| Mdm4, transformed 3T3 cell double minute 4, p53 | MDM4 | Hs.497492 | 225740_x_at |
| binding protein (mouse) | | | |
| Mdm4, transformed 3T3 cell double minute 4, p53 | MDM4 | Hs.497492 | 235589_s_at |
| binding protein (mouse) | | | |
| mediator of DNA damage checkpoint 1 | MDC1 | Hs.433653 | 203062_s_at |
| mediator of RNA polymerase II transcription, | MED19 | Hs.43619 | 226293_at |
| subunit 19 homolog (yeast) | | | |
| mediator of RNA polymerase II transcription, | MED19 | Hs.43619 | 226300_at |
| subunit 19 homolog (yeast) | | | |
| Mediator of RNA polymerase II transcription, | MED31 | Hs.43566 | 236241_at |
| subunit 31 homolog (yeast) | | | |
| mediator of RNA polymerase II transcription, | MED9 | Hs.244595 | 218372_at |
| subunit 9 homolog (yeast) | | | |
| meiosis-specific nuclear structural protein 1 | MNS1 | Hs.444483 | 219703_at |
| Meis1, myeloid ecotropic viral integration site 1 | MEIS1 | Hs.526754 | 204069_at |
| homolog (mouse) | | | |
| Meis1, myeloid ecotropic viral integration site 1 | MEIS2 | Hs.510989 | 207480_s_at |
| homolog 2 (mouse) | | | |
| mel transforming oncogene-like 1 | MELL1 | Hs.546429 | 1552930_at |
| melanoma antigen family B, 2 | MAGEB2 | Hs.113824 | 206218_at |
| melanoma associated antigen (mutated) 1 | MUM1 | Hs.515016 | 221290_s_at |
| melanoma associated antigen (mutated) 1 | MUM1 | Hs.515016 | 223347_at |
| Melanoma associated gene | D2S448 | Hs.332197 | 212012_at |
| melanophilin | MLPH | Hs.102406 | 218211_s_at |
| membrane cofactor protein (CD46, trophoblast- | MCP | Hs.510402 | 207549_x_at |
| lymphocyte cross-reactive antigen) | | | |
| membrane component, chromosome 11, surface | M11S1 | Hs.471818 | 200722_s_at |
| marker 1 | | | |
| Membrane component, chromosome 11, surface | M11S1 | Hs.471818 | 226990_at |
| marker 1 | | | |
| Membrane component, chromosome 11, surface | M11S1 | Hs.471818 | 244778_x_at |
| marker 1 | | | |
| membrane frizzled-related protein | MFRP | Hs.512769 | 224286_at |
| membrane progestin receptor alpha | MPRA | Hs.523652 | 242123_at |
| membrane-associated guanylate kinase-related | MAGI-3 | Hs.486189 | 226770_at |
| (MAGI-3) | | | |
| membrane-associated ring finger (C3HC4) 5 | 5-Mar | Hs.549165 | 218582_at |
| membrane-associated ring finger (C3HC4) 6 | 6-Mar | Hs.432862 | 201737_s_at |
| membrane-associated ring finger (C3HC4) 7 | 7-Mar | Hs.529272 | 202654_x_at |
| membrane-associated ring finger (C3HC4) 8 | 8-Mar | Hs.499489 | 231933_at |
| membrane-spanning 4-domains, subfamily A, | MS4A6E | Hs.126580 | 231335_at |
| member 6E | | | |
| meningioma expressed antigen 5 (hyaluronidase) | MGEA5 | Hs.500842 | 200898_s_at |
| meningioma expressed antigen 5 (hyaluronidase) | MGEA5 | Hs.500842 | 223494_at |
| mesenchymal stem cell protein DSC92 | NEUGRIN | Hs.513145 | 217722_s_at |
| Mesenchymal stem cell protein DSC92 | NEUGRIN | Hs.513145 | 228541_x_at |
| mesoderm development candidate 2 | MESDC2 | Hs.513070 | 224675_at |
| mesoderm posterior 2 | MESP2 | Hs.37311 | 1556015_a_at |
| metallothionein-like 5, testis-specific (tesmin) | MTL5 | Hs.145932 | 219786_at |
| metal-regulatory transcription factor 1 | MTF1 | Hs.471991 | 205323_s_at |
| metastasis associated 1 /// metastasis associated 1 | MTA1 | Hs.525629 | 211783_s_at |
| metastasis associated lung adenocarcinoma | MALAT1 | Hs.187199 | 1558678_s_at |
| transcript 1 (non-coding RNA) | | | |
| metastasis associated lung adenocarcinoma | MALAT1 | Hs.187199 | 224558_s_at |
| transcript 1 (non-coding RNA) | | | |
| Metastasis associated lung adenocarcinoma | MALAT1 | Hs.187199 | 228582_x_at |
| transcript 1 (non-coding RNA) | | | |
| methionine adenosyltransferase II, alpha | MAT2A | Hs.516157 | 200768_s_at |
| methionine adenosyltransferase II, alpha | MAT2A | Hs.516157 | 200769_s_at |
| Methionine adenosyltransferase II, alpha | MAT2A | Hs.516157 | 213705_at |
| methionine adenosyltransferase II, beta | MAT2B | Hs.54642 | 217993_s_at |
| Methionine sulfoxide reductase A | MSRA | Hs.490981 | 237798_at |
| methionyl aminopeptidase 2 | METAP2 | Hs.444986 | 213899_at |
| Methionyl aminopeptidase 2 | METAP2 | Hs.444986 | 244185_at |
| methyl-CpG binding domain protein 4 | MBD4 | Hs.35947 | 209579_s_at |
| methyl-CpG binding domain protein 5 | MBD5 | Hs.458312 | 220195_at |
| Methylcrotonoyl-Coenzyme A carboxylase 2 (beta) | MCCC2 | Hs.167531 | 1556345_s_at |
| methylcrotonoyl-Coenzyme A carboxylase 2 (beta) | MCCC2 | Hs.167531 | 209623_at |
| methylenetetrahydrofolate dehydrogenase | MTHFD1L | Hs.268698 | 225520_at |
| (NADP+ dependent) 1-like | | | |
| Methylmalonic aciduria (cobalamin deficiency) type A | MMAA | Hs.117747 | 242750_at |
| Methylosome protein 50 | MEP50 | Hs.204773 | 242398_x_at |
| methylthioadenosine phosphorylase | MTAP | Hs.193268 | 211364_at |
| Methyltransferase 5 domain containing 1 | FLJ33997 | Hs.243326 | 243736_at |
| methyltransferase like 4 | METTL4 | Hs.126888 | 219698_s_at |
| methyltransferase like 5 | METTL5 | Hs.470553 | 221570_s_at |
| mevalonate kinase (mevalonic aciduria) | MVK | Hs.130607 | 36907_at |
| MHC class II transactivator | MHC2TA | Hs.126714 | 241520_x_at |
| MICAL-like 1 | MICAL- | Hs.517610 | 55081_at |
| | L1 | | |
| microfibrillar-associated protein 1 | MFAP1 | Hs.61418 | 203406_at |
| microfibrillar-associated protein 3-like | MFAP3L | Hs.178121 | 210493_s_at |
| microfibrillar-associated protein 3-like | MFAP3L | Hs.178121 | 210843_s_at |
| microseminoprotein, beta- | MSMB | Hs.255462 | 210297_s_at |
| microtubule associated monoxygenase, calponin | MICAL2 | Hs.501928 | 212473_s_at |
| and LIM domain containing 2 | | | |
| microtubule associated monoxygenase, calponin | MICAL3 | Hs.165551 | 229474_at |
| and LIM domain containing 3 | | | |
| microtubule associated serine/threonine kinase | MAST4 | Hs.133539 | 225613_at |
| family member 4 | | | |
| microtubule-associated protein 1 light chain 3 | MAP1LC3A | Hs.472513 | 227219_x_at |
| alpha | | | |
| microtubule-associated protein 1 light chain 3 beta | MAP1LC3B | Hs.356061 | 208785_s_at |
| Microtubule-associated protein 1B | MAP1B | Hs.535786 | 212233_at |
| microtubule-associated protein 4 | MAP4 | Hs.517949 | 212566_at |
| microtubule-associated protein 4 | MAP4 | Hs.517949 | 243_g_at |
| microtubule-associated protein, RP/EB family, | MAPRE1 | Hs.472437 | 200712_s_at |
| member 1 | | | |
| microtubule-associated protein, RP/EB family, | MAPRE1 | Hs.472437 | 200713_s_at |
| member 1 | | | |
| microtubule-associated protein, RP/EB family, | MAPRE2 | Hs.532824 | 202501_at |
| member 2 | | | |
| midline 1 (Opitz/BBB syndrome) | MID1 | Hs.27695 | 203637_s_at |
| midline 2 | MID2 | Hs.12256 | 208384_s_at |
| misato | MST | Hs.279763 | 218296_x_at |
| misato | MST | Hs.279763 | 224233_s_at |
| mitochondrial capsule selenoprotein | MCSP | Hs.111850 | 206406_at |
| mitochondrial carrier homolog 1 (C. elegans) | MTCH1 | Hs.485262 | 221619_s_at |
| mitochondrial carrier homolog 2 (C. elegans) | MTCH2 | Hs.269944 | 222403_at |
| mitochondrial carrier protein | MGC4399 | Hs.443826 | 223296_at |
| mitochondrial carrier triple repeat 1 | MCART1 | Hs.46791 | 232092_at |
| mitochondrial ribosomal protein 63 | MRP63 | Hs.458367 | 204386_s_at |
| mitochondrial ribosomal protein 63 | MRP63 | Hs.458367 | 204387_x_at |
| mitochondrial ribosomal protein L15 | MRPL15 | Hs.18349 | 218027_at |
| mitochondrial ribosomal protein L3 | MRPL3 | Hs.205163 | 208787_at |
| mitochondrial ribosomal protein L30 | MRPL30 | Hs.346736 | 224971_at |
| Mitochondrial ribosomal protein L30 | MRPL30 | Hs.346736 | 243801_x_at |
| mitochondrial ribosomal protein L32 | MRPL32 | Hs.50252 | 225260_s_at |
| mitochondrial ribosomal protein L33 | MRPL33 | Hs.515879 | 203781_at |
| mitochondrial ribosomal protein L36 /// | MRPL36 | Hs.32196 | 224331_s_at |
| mitochondrial ribosomal protein L36 | | | |
| mitochondrial ribosomal protein L39 | MRPL39 | Hs.420696 | 218558_s_at |
| Mitochondrial ribosomal protein L43 | MRPL43 | Hs.421848 | 1556078_at |
| mitochondrial ribosomal protein L51 | MRPL51 | Hs.55847 | 223086_x_at |
| mitochondrial ribosomal protein L9 | MRPL9 | Hs.288936 | 209609_s_at |
| mitochondrial ribosomal protein S10 | MRPS10 | Hs.380887 | 218106_s_at |
| mitochondrial ribosomal protein S14 | MRPS14 | Hs.247324 | 203800_s_at |
| mitochondrial ribosomal protein S17 | MRPS17 | Hs.44298 | 218982_s_at |
| mitochondrial ribosomal protein S18B | MRPS18B | Hs.274417 | 217408_at |
| mitochondrial ribosomal protein S21 | MRPS21 | Hs.405880 | 222997_s_at |
| mitochondrial ribosomal protein S24 | MRPS24 | Hs.284286 | 224948_at |
| mitochondrial ribosomal protein S28 | MRPS28 | Hs.521124 | 219819_s_at |
| Mitochondrial ribosomal protein S6 | MRPS6 | Hs.302742 | 212944_at |
| mitochondrial ribosomal protein S6 | MRPS6 | Hs.302742 | 224919_at |
| mitochondrial ribosomal protein S7 | MRPS7 | Hs.71787 | 217932_at |
| mitochondrial ribosome recycling factor | MRRF | Hs.368011 | 225126_at |
| mitochondrial solute carrier protein | MSCP | Hs.122514 | 218136_s_at |
| Mitochondrial solute carrier protein | MSCP | Hs.122514 | 221155_x_at |
| mitochondrial solute carrier protein | MSCP | Hs.122514 | 222529_at |
| Mitochondrial solute carrier protein | MSCP | Hs.122514 | 237568_at |
| mitochondrial translation optimization 1 homolog | MTO1 | Hs.347614 | 218716_x_at |
| (S. cerevisiae) | | | |
| mitochondrial translation optimization 1 homolog | MTO1 | Hs.347614 | 233665_x_at |
| (S. cerevisiae) | | | |
| mitochondrial translational initiation factor 2 | MTIF2 | Hs.149894 | 203095_at |
| mitochondrial translational initiation factor 3 | MTIF3 | Hs.534582 | 223356_s_at |
| mitofusin 2 | MFN2 | Hs.376681 | 216205_s_at |
| mitogen-activated protein kinase 10 | MAPK10 | Hs.25209 | 204813_at |
| mitogen-activated protein kinase 13 | MAPK13 | Hs.178695 | 210058_at |
| mitogen-activated protein kinase 13 | MAPK13 | Hs.178695 | 210059_s_at |
| mitogen-activated protein kinase 8 | MAPK8 | Hs.522924 | 226048_at |
| Mitogen-activated protein kinase 8 | MAPK8 | Hs.522924 | 229664_at |
| mitogen-activated protein kinase kinase 4 | MAP2K4 | Hs.514681 | 203265_s_at |
| mitogen-activated protein kinase kinase 4 | MAP2K4 | Hs.514681 | 203266_s_at |
| Mitogen-activated protein kinase kinase 5 | MAP2K5 | Hs.114198 | 235601_at |
| mitogen-activated protein kinase kinase kinase 1 | MAP3K1 | Hs.508461 | 225927_at |
| mitogen-activated protein kinase kinase kinase 2 | MAP3K2 | Hs.145605 | 226979_at |
| mitogen-activated protein kinase kinase kinase | MAP4K4 | Hs.431550 | 206571_s_at |
| kinase 4 | | | |
| Mitogen-activated protein kinase kinase kinase | MAP4K4 | Hs.431550 | 244846_at |
| kinase 4 | | | |
| mitogen-activated protein kinase kinase kinase | MAP4K5 | Hs.130491 | 203553_s_at |
| kinase 5 | | | |
| mixed lineage kinase 4 | KIAA1804 | Hs.546455 | 228565_at |
| MLF1 interacting protein | MLF1IP | Hs.481307 | 218883_s_at |
| MLF1 interacting protein | MLF1IP | Hs.481307 | 229305_at |
| Mlx interactor | MONDOA | Hs.437153 | 225157_at |
| MOB1, Mps One Binder kinase activator-like 2B | MOBKL2B | Hs.369022 | 226844_at |
| (yeast) | | | |
| Mof4 family associated protein 1 | MRFAP1 | Hs.406590 | 226091_s_at |
| molybdenum cofactor synthesis 1 /// molybdenum | MOCS1 | Hs.357128 | 211673_s_at |
| cofactor synthesis 1 | | | |
| molybdenum cofactor synthesis 3 | MOCS3 | Hs.159410 | 206141_at |
| monoamine oxidase A | MAOA | Hs.183109 | 204388_s_at |
| monoamine oxidase A | MAOA | Hs.183109 | 204389_at |
| Monoamine oxidase A | MAOA | Hs.183109 | 234534_at |
| monoglyceride lipase /// monoglyceride lipase | MGLL | Hs.277035 | 211026_s_at |
| monooxygenase, DBH-like 1 | MOXD1 | Hs.6909 | 1554474_a_at |
| more than blood homolog | MTB | Hs.18616 | 219588_s_at |
| morn | LOC283385 | Hs.434154 | 237211_x_at |
| mortality factor 4 like 1 | MORF4L1 | Hs.374503 | 217982_s_at |
| mortality factor 4 like 1 | MORF4L1 | Hs.374503 | 224561_s_at |
| mortality factor 4 like 1 /// mortality factor 4 | MORF4L1 | Hs.534391 | 221381_s_at |
| | /// | | |
| | MORF4 | | |
| mortality factor 4 like 2 | MORF4L2 | Hs.326387 | 201994_at |
| motile sperm domain containing 1 | MOSPD1 | Hs.378505 | 218853_s_at |
| Mouse Mammary Turmor Virus Receptor homolog 1 | MTVR1 | Hs.25723 | 32209_at |
| Mov10l1, Moloney leukemia virus 10-like 1, | MOV10L1 | Hs.62880 | 239257_at |
| homolog (mouse) | | | |
| M-phase phosphoprotein, mpp8 | HSMPP8 | Hs.549166 | 225041_at |
| MRNA (clone ICRFp507I1077) | — | Hs.43744 | 226773_at |
| MRNA from chromosome 5q21-22, clone: 357Ex. | — | Hs.26968 | 214202_at |
| MRNA full length insert cDNA clone EUROIMAGE | — | Hs.388742 | 213386_at |
| 1585492 | | | |
| MRNA full length insert cDNA clone EUROIMAGE | — | Hs.284310 | 215572_at |
| 254679 | | | |
| MRNA of muscle specific gene M1. | — | Hs.58049 | 231051_at |
| MRNA similar to major histocompatibility complex, | — | Hs.199142 | 239975_at |
| class II, DP beta 1 (cDNA clone MGC: 24119 | | | |
| IMAGE: 4663904), complete cds | | | |
| MRNA, chromosome 1 specific transcript | — | Hs.127406 | 239784_at |
| KIAA0504. | | | |
| MRNA; cDNA DKFZp434A202 (from clone | — | Hs.189242 | 215322_at |
| DKFZp434A202) | | | |
| MRNA; cDNA DKFZp434B1023 (from clone | — | Hs.535406 | 233194_at |
| DKFZp434B1023) | | | |
| MRNA; cDNA DKFZp434D1322 (from clone | — | Hs.518292 | 234443_at |
| DKFZp434D1322) | | | |
| MRNA; cDNA DKFZp434E1812 (from clone | — | Hs.537604 | 234578_at |
| DKFZp434E1812) | | | |
| MRNA; cDNA DKFZp434F1626 (from clone | — | Hs.383800 | 1558262_at |
| DKFZp434F1626) | | | |
| MRNA; cDNA DKFZp434F1626 (from clone | — | Hs.383800 | 231404_at |
| DKFZp434F1626) | | | |
| MRNA; cDNA DKFZp434K0610 (from clone | — | Hs.409816 | 216499_at |
| DKFZp434K0610) | | | |
| MRNA; cDNA DKFZp434M054 (from clone | — | Hs.548646 | 217446_x_at |
| DKFZp434M054) | | | |
| MRNA; cDNA DKFZp434N2419 (from clone | — | Hs.383791 | 1560559_at |
| DKFZp434N2419) | | | |
| MRNA; cDNA DKFZp451D1716 (from clone | — | Hs.22005 | 237761_at |
| DKFZp451D1716) | | | |
| MRNA; cDNA DKFZp564B206 (from clone | — | Hs.543875 | 234509_at |
| DKFZp564B206) | | | |
| MRNA; cDNA DKFZp564E233 (from clone | — | Hs.518003 | 216524_x_at |
| DKFZp564E233) | | | |
| MRNA; cDNA DKFZp566E213 (from clone | — | Hs.444231 | 234603_at |
| DKFZp566E213) | | | |
| MRNA; cDNA DKFZp566P1546 (from clone | — | Hs.383807 | 242510_at |
| DKFZp566P1546) | | | |
| MRNA; cDNA DKFZp586F0922 (from clone | — | Hs.194678 | 225812_at |
| DKFZp586F0922) | | | |
| MRNA; cDNA DKFZp586O1318 (from clone | — | Hs.22689 | 215528_at |
| DKFZp586O1318) | | | |
| MRNA; cDNA DKFZp686D22106 (from clone | — | Hs.445036 | 227931_at |
| DKFZp686D22106) | | | |
| MRNA; cDNA DKFZp686J0929 (from clone | — | Hs.376849 | 1566032_at |
| DKFZp686J0929) | | | |
| MRNA; cDNA DKFZp686N1136 (from clone | — | Hs.548588 | 1557589_a_at |
| DKFZp686N1136) | | | |
| MRNA; cDNA DKFZp761B0221 (from clone | — | Hs.541755 | 1563715_at |
| DKFZp761B0221) | | | |
| MRNA; cDNA DKFZp761I2317 (from clone | — | Hs.442983 | 1558142_at |
| DKFZp761I2317) | | | |
| MRNA; cDNA DKFZp761I2317 (from clone | — | Hs.442983 | 238468_at |
| DKFZp761I2317) | | | |
| MRNA; cDNA DKFZp762C074 (from clone | — | Hs.546985 | 1560661_x_at |
| DKFZp762C074) | | | |
| mSin3A-associated protein 130 | SAP130 | Hs.32995 | 220367_s_at |
| mucin 17 | MUC17 | Hs.271819 | 232321_at |
| mucin 19 | MUC19 | Hs.244017 | 1553436_at |
| mucin 3A, intestinal | MUC3A | Hs.533959 | 217117_x_at |
| mucin 4, tracheobronchial | MUC4 | Hs.369646 | 217109_at |
| mucosa associated lymphoid tissue lymphoma | MALT1 | Hs.180566 | 210017_at |
| translocation gene 1 | | | |
| multimerin 2 | MMRN2 | Hs.524479 | 219091_s_at |
| multiple C2-domains with two transmembrane | MCTP2 | Hs.33368 | 220603_s_at |
| regions 2 | | | |
| Multiple C2-domains with two transmembrane | MCTP2 | Hs.33368 | 239893_at |
| regions 2 | | | |
| Multiple C2-domains with two transmembrane | MCTP2 | Hs.33368 | 243109_at |
| regions 2 | | | |
| multiple PDZ domain protein | MPDZ | Hs.169378 | 205079_s_at |
| MUS81 endonuclease homolog (yeast) | MUS81 | Hs.288798 | 218463_s_at |
| musashi homolog 2 (Drosophila) | MSI2 | Hs.134470 | 225240_s_at |
| Muscleblind-like (Drosophila) | MBNL1 | Hs.478000 | 1556658_a_at |
| Muscleblind-like (Drosophila) | MBNL1 | Hs.478000 | 235879_at |
| Muscleblind-like (Drosophila) | MBNL1 | Hs.478000 | 241681_at |
| Muscleblind-like 2 (Drosophila) | MBNL2 | Hs.125715 | 232138_at |
| muted homolog (mouse) | MUTED | — | 226544_x_at |
| MYC associated factor X | MAX | Hs.285354 | 209332_s_at |
| MYC induced nuclear antigen | MINA | Hs.476972 | 213188_s_at |
| myelin expression factor 2 | MYEF2 | Hs.552594 | 222771_s_at |
| myelin expression factor 2 | MYEF2 | Hs.552594 | 232676_x_at |
| myelin protein zero-like 1 | MPZL1 | Hs.493919 | 210210_at |
| myelin transcription factor 1 | MYT1 | Hs.279562 | 1556269_at |
| myelin-associated oligodendrocyte basic protein | MOBP | Hs.121333 | 207659_s_at |
| myelin-associated oligodendrocyte basic protein | MOBP | Hs.121333 | 242765_at |
| myeloid cell leukemia sequence 1 (BCL2-related) | MCL1 | Hs.532826 | 200797_s_at |
| myeloid cell leukemia sequence 1 (BCL2-related) | MCL1 | Hs.532826 | 200798_x_at |
| Myeloid cell leukemia sequence 1 (BCL2-related) | MCL1 | Hs.532826 | 214057_at |
| myeloid differentiation primary response gene (88) | MYD88 | Hs.82116 | 209124_at |
| myeloid leukemia factor 1 | MLF1 | Hs.85195 | 204784_s_at |
| myeloid/lymphoid or mixed-lineage leukemia | MLL /// | Hs.258855 | 1565254_s_at |
| (trithorax homolog, Drosophila) /// elongation factor | ELL | | |
| RNA polymerase II | | | |
| myeloid/lymphoid or mixed-lineage leukemia | MLLT10 | Hs.30385 | 1563321_s_at |
| (trithorax homolog, Drosophila); translocated to, 10 | | | |
| Myeloid/lymphoid or mixed-lineage leukemia | MLLT10 | Hs.30385 | 237600_at |
| (trithorax homolog, Drosophila); translocated to, 10 | | | |
| Myeloid/lymphoid or mixed-lineage leukemia | MLLT10 | Hs.30385 | 243003_at |
| (trithorax homolog, Drosophila); translocated to, 10 | | | |
| myeloid/lymphoid or mixed-lineage leukemia | MLLT2 | Hs.480190 | 201924_at |
| (trithorax homolog, Drosophila); translocated to, 2 | | | |
| Myeloid/lymphoid or mixed-lineage leukemia | MLLT2 | Hs.480190 | 237006_at |
| (trithorax homolog, Drosophila); translocated to, 2 | | | |
| myeloid/lymphoid or mixed-lineage leukemia 3 | MLL3 | Hs.270492 | 222413_s_at |
| myeloid/lymphoid or mixed-lineage leukemia 5 | MLL5 | Hs.551548 | 223190_s_at |
| (trithorax homolog, Drosophila) | | | |
| myeloid-associated differentiation marker | MYADM | Hs.380906 | 225673_at |
| myoglobin | MB | Hs.517586 | 204179_at |
| myo-inositol monophosphatase A3 | IMPA3 | Hs.438689 | 224743_at |
| Myomesin family, member 3 | MYOM3 | Hs.523413 | 238124_at |
| myoneurin | MYNN | Hs.507025 | 224206_x_at |
| myosin head domain containing 1 | MYOHD1 | Hs.302051 | 219320_at |
| myosin head domain containing 1 | MYOHD1 | Hs.302051 | 225947_at |
| myosin IG | MYO1G | Hs.37617 | 244654_at |
| myosin IIIA | MYO3A | Hs.131367 | 244364_at |
| myosin IXA | MYO9A | Hs.546268 | 219027_s_at |
| Myosin IXA | MYO9A | Hs.546268 | 242512_at |
| myosin light chain 1 slow a | MLC1SA | Hs.524500 | 204173_at |
| myosin phosphatase-Rho interacting protein | M-RIP | Hs.462341 | 214771_x_at |
| myosin regulatory light chain MRCL3 /// myosin | MRCL3 | Hs.464472 | 201318_s_at |
| regulatory light chain MRLC2 | /// | | |
| | MRLC2 | | |
| myosin regulatory light chain MRLC2 | MRLC2 | Hs.464472 | 221474_at |
| myosin VB | MYO5B | Hs.550481 | 225299_at |
| myosin XVA | MYO15A | Hs.462390 | 220288_at |
| myosin, heavy polypeptide 10, non-muscle | MYH10 | Hs.16355 | 212372_at |
| myosin, light polypeptide 4, alkali; atrial, embryonic | MYL4 | Hs.463300 | 216054_x_at |
| myosin, light polypeptide 6, alkali, smooth muscle | MYL6 | Hs.505705 | 212082_s_at |
| and non-muscle | | | |
| myosin, light polypeptide kinase | MYLK | Hs.477375 | 224823_at |
| myotubularin related protein 12 | MTMR12 | Hs.481836 | 220953_s_at |
| myotubularin related protein 2 | MTMR2 | Hs.181326 | 203211_s_at |
| myotubularin related protein 2 | MTMR2 | Hs.181326 | 214649_s_at |
| Myotubularin related protein 3 | MTMR3 | Hs.474536 | 240271_at |
| myotubularin related protein 4 | MTMR4 | Hs.514373 | 214268_s_at |
| myotubularin related protein 9 | MTMR9 | Hs.528673 | 204837_at |
| myotubularin related protein 9 | MTMR9 | Hs.528673 | 213278_at |
| myristoylated alanine-rich protein kinase C | MARCKS | Hs.519909 | 201669_s_at |
| substrate | | | |
| Myristoylated alanine-rich protein kinase C | MARCKS | Hs.519909 | 213002_at |
| substrate | | | |
| MYST histone acetyltransferase (monocytic | MYST3 | Hs.491577 | 202423_at |
| leukemia) 3 | | | |
| MYST histone acetyltransferase 2 /// MYST histone | MYST2 | Hs.21907 | 200049_at |
| acetyltransferase 2 | | | |
| N-acetylated alpha-linked acidic dipeptidase 2 | NAALAD2 | Hs.503560 | 233815_at |
| N-acetylated alpha-linked acidic dipeptidase 2 | NAALADL2 | Hs.86024 | 236818_at |
| N-acetylgalactosaminidase, alpha- | NAGA | Hs.75372 | 202943_s_at |
| N-acetylneuraminate pyruvate lyase | NPL | Hs.496969 | 240440_at |
| (dihydrodipicolinate synthase) | | | |
| N-acetylneuraminic acid synthase (sialic acid | NANS | Hs.522310 | 218189_s_at |
| synthase) | | | |
| N-acetyltransferase 1 (arylamine N- | NAT1 | Hs.155956 | 214440_at |
| acetyltransferase) | | | |
| N-acetyltransferase 5 (ARD1 homolog, S. cerevisiae) | NAT5 | Hs.368783 | 223040_at |
| N-acetyltransferase-like protein | FLJ10774 | Hs.523598 | 217884_at |
| NACHT, leucine rich repeat and PYD (pyrin | NALP1 | Hs.104305 /// | 1562256_at |
| domain) containing 1 /// NACHT, leucine rich | | Hs.513902 | |
| repeat and PYD (pyrin domain) containing 1 | | | |
| NACHT, leucine rich repeat and PYD (pyrin | NALP1 | Hs.104305 /// | 1562257_x_at |
| domain) containing 1 /// NACHT, leucine rich | | Hs.513902 | |
| repeat and PYD (pyrin domain) containing 1 | | | |
| N-acyl-phosphatidylethanolamine-hydrolyzing | NAPEPLD | Hs.324271 | 233539_at |
| phospholipase D | | | |
| N-acyl-phosphatidylethanolamine-hydrolyzing | NAPEPLD | Hs.324271 | 238722_x_at |
| phospholipase D | | | |
| N-acyl-phosphatidylethanolamine-hydrolyzing | NAPEPLD | Hs.324271 | 242635_s_at |
| phospholipase D | | | |
| N-acylsphingosine amidohydrolase (acid | ASAH1 | Hs.527412 | 210980_s_at |
| ceramidase) 1 | | | |
| N-acylsphingosine amidohydrolase (acid | ASAH1 | Hs.527412 | 213702_x_at |
| ceramidase) 1 | | | |
| N-acylsphingosine amidohydrolase (acid | ASAH1 | Hs.527412 | 213902_at |
| ceramidase) 1 | | | |
| N-acylsphingosine amidohydrolase (acid | ASAHL | Hs.437365 | 215178_x_at |
| ceramidase)-like | | | |
| NAD kinase | FLJ13052 | Hs.456572 | 208918_s_at |
| NADH dehydrogenase (ubiquinone) 1 alpha | NDUFA10 | Hs.277677 | 217860_at |
| subcomplex, 10, 42 kDa | | | |
| NADH dehydrogenase (ubiquinone) 1 alpha | NDUFA5 | Hs.83916 | 201304_at |
| subcomplex, 5, 13 kDa | | | |
| NADH dehydrogenase (ubiquinone) 1 alpha | NDUFA9 | Hs.75227 | 208969_at |
| subcomplex, 9, 39 kDa | | | |
| NADH dehydrogenase (ubiquinone) 1 beta | NDUFB1 | Hs.183435 | 206790_s_at |
| subcomplex, 1, 7 kDa | | | |
| NADH dehydrogenase (ubiquinone) 1 beta | NDUFB2 | Hs.324250 | 218200_s_at |
| subcomplex, 2, 8 kDa | | | |
| NADH dehydrogenase (ubiquinone) 1 beta | NDUFB2 | Hs.324250 | 218201_at |
| subcomplex, 2, 8 kDa | | | |
| NADH dehydrogenase (ubiquinone) 1 beta | NDUFB6 | Hs.493668 | 1559042_at |
| subcomplex, 6, 17 kDa | | | |
| NADH dehydrogenase (ubiquinone) 1 beta | NDUFB6 | Hs.493668 | 203613_s_at |
| subcomplex, 6, 17 kDa | | | |
| NADH dehydrogenase (ubiquinone) 1 beta | NDUFB8 | Hs.523215 | 201227_s_at |
| subcomplex, 8, 19 kDa | | | |
| NADH dehydrogenase (ubiquinone) 1 beta | NDUFB9 | Hs.15977 | 222992_s_at |
| subcomplex, 9, 22 kDa | | | |
| NADH dehydrogenase (ubiquinone) Fe—S protein | NDUFS6 | Hs.408257 | 203606_at |
| 6, 13 kDa (NADH-coenzyme Q reductase) | | | |
| NADH dehydrogenase (ubiquinone) Fe—S protein | NDUFS8 | Hs.90443 | 232169_x_at |
| 8, 23 kDa (NADH-coenzyme Q reductase) | | | |
| NADPH cytochrome B5 oxidoreductase | NCB5OR | Hs.5741 | 219079_at |
| napsin B aspartic peptidase pseudogene | NAPSB | Hs.322854 | 228056_s_at |
| nardilysin (N-arginine dibasic convertase) | NRD1 | Hs.551509 | 208709_s_at |
| Nardilysin (N-arginine dibasic convertase) | NRD1 | Hs.551509 | 229422_at |
| Nardilysin (N-arginine dibasic convertase) | NRD1 | Hs.551509 | 242235_x_at |
| nasal embryonic LHRH factor | NELF | Hs.455336 | 221214_s_at |
| nascent-polypeptide-associated complex alpha | NACA | Hs.505735 | 200735_x_at |
| polypeptide | | | |
| nascent-polypeptide-associated complex alpha | NACA | Hs.505735 | 208635_x_at |
| polypeptide | | | |
| natural killer-tumor recognition sequence | NKTR | Hs.529509 | 1557737_s_at |
| NCK adaptor protein 1 | NCK1 | Hs.477693 | 229895_s_at |
| NCK adaptor protein 1 | NCK1 | Hs.477693 | 233274_at |
| NCK adaptor protein 1 /// NCK adaptor protein 1 | NCK1 | Hs.477693 | 211063_s_at |
| Nedd4 binding protein 1 | N4BP1 | Hs.511839 | 48612_at |
| Nedd4 family interacting protein 2 | NDFIP2 | Hs.525093 | 224799_at |
| Nedd4 family interacting protein 2 | NDFIP2 | Hs.525093 | 224801_at |
| Neighbor of BRCA1 gene 1 | M17S2 | Hs.550589 | 1568857_a_at |
| neighbor of BRCA1 gene 1 | NBR1 | Hs.277721 | 201383_s_at |
| Nemo like kinase | NLK | Hs.208759 | 1556568_a_at |
| nemo like kinase | NLK | Hs.208759 | 222589_at |
| Nemo like kinase | NLK | Hs.208759 | 238624_at |
| nephrosis 2, idiopathic, steroid-resistant (podocin) | NPHS2 | Hs.412710 | 220424_at |
| N-ethylmaleimide-sensitive factor | NSF | Hs.431279 | 1566785_x_at |
| Neural cell adhesion molecule 1 | NCAM1 | Hs.503878 | 227394_at |
| neural precursor cell expressed, developmentally | NEDD1 | Hs.270084 | 1560116_a_at |
| down-regulated 1 | | | |
| neural precursor cell expressed, developmentally | NEDD4L | Hs.185677 | 212445_s_at |
| down-regulated 4-like | | | |
| Neural precursor cell expressed, developmentally | NEDD4L | Hs.185677 | 226974_at |
| down-regulated 4-like | | | |
| neurobeachin | NBEA | Hs.491172 | 221207_s_at |
| neurobeachin | NBEA | Hs.491172 | 226439_s_at |
| neuroepithelial cell transforming gene 1 | NET1 | Hs.25155 | 201829_at |
| neurofibromin 1 (neurofibromatosis, von | NF1 | Hs.435956 | 204325_s_at |
| Recklinghausen disease, Watson disease) | | | |
| neurofilament 3 (150 kDa medium) | NEF3 | Hs.458657 | 205113_at |
| neurofilament, heavy polypeptide 200 kDa | NEFH | Hs.198760 | 33767_at |
| neuroligin 2 | NLGN2 | Hs.26229 | 1554428_s_at |
| Neurolysin (metallopeptidase M3 family) | NLN | Hs.247460 | 234762_x_at |
| neuron navigator 1 | NAV1 | Hs.497369 | 224772_at |
| neuron navigator 2 | NAV2 | Hs.502116 | 1556606_at |
| Neuronal PAS domain protein 3 | NPAS3 | Hs.509113 | 239611_at |
| neuro-oncological ventral antigen 1 | NOVA1 | Hs.211225 | 205794_s_at |
| neuropeptide Y | NPY | Hs.1832 | 206001_at |
| neuropeptide Y receptor Y5 | NPY5R | Hs.519058 | 207400_at |
| neurotrophic tyrosine kinase, receptor, type 3 | NTRK3 | Hs.410969 | 206462_s_at |
| neurotrophic tyrosine kinase, receptor, type 3 | NTRK3 | Hs.410969 | 217033_x_at |
| neurotrophic tyrosine kinase, receptor, type 3 | NTRK3 | Hs.410969 | 217377_x_at |
| Neutral sphingomyelinase (N-SMase) activation | NSMAF | Hs.372000 | 232148_at |
| associated factor | | | |
| Nexilin (F actin binding protein) | NEXN | Hs.22370 | 212847_at |
| NFS1 nitrogen fixation 1 (S. cerevisiae) | NES1 | Hs.194692 | 218455_at |
| NGFI-A binding protein 1 (EGR1 binding protein 1) | NAB1 | Hs.107474 | 211139_s_at |
| Ngg1 interacting factor 3 like 1 binding protein 1 | NIF3L1BP1 | Hs.288151 | 218334_at |
| N-glycanase 1 | NGLY1 | Hs.368960 | 220742_s_at |
| NHP2 non-histone chromosome protein 2-like 1 (S. cerevisiae) | NHP2L1 | Hs.182255 | 201077_s_at |
| nicotinamide nucleotide adenylyltransferase 2 | NMNAT2 | Hs.497123 | 1552712_a_at |
| nicotinamide nucleotide adenylyltransferase 2 | NMNAT2 | Hs.497123 | 1556029_s_at |
| nicotinamide nucleotide transhydrogenase | NNT | Hs.482043 | 238530_at |
| NifU-like N-terminal domain containing | NIFUN | Hs.350702 | 209075_s_at |
| Nijmegen breakage syndrome 1 (nibrin) | NBS1 | Hs.492208 | 202907_s_at |
| NIMA (never in mitosis gene a)-related kinase 11 | NEK11 | Hs.200813 | 219542_at |
| NIMA (never in mitosis gene a)-related kinase 1 | NEK1 | Hs.481181 | 1564093_at |
| NIMA (never in mitosis gene a)-related kinase 1 | NEK1 | Hs.481181 | 213328_at |
| NIMA (never in mitosis gene a)-related kinase 2 | NEK2 | Hs.153704 | 204641_at |
| NIMA (never in mitosis gene a)-related kinase 2 /// | NEK2 | Hs.153704 | 211080_s_at |
| NIMA (never in mitosis gene a)-related kinase 2 | | | |
| NIMA (never in mitosis gene a)-related kinase 3 /// | NEK3 | Hs.409989 | 211089_s_at |
| NIMA (never in mitosis gene a)-related kinase 3 | | | |
| NIMA (never in mitosis gene a)-related kinase 6 | NEK6 | Hs.197071 | 223159_s_at |
| Nipped-B homolog (Drosophila) | NIPBL | Hs.481927 | 213918_s_at |
| nischarin | NISCH | Hs.435290 | 201591_s_at |
| nitric oxide synthase 3 antisense | NOS3AS | Hs.546535 | 229252_at |
| nitric oxide synthase interacting protein | NOSIP | Hs.7236 | 217950_at |
| NK3 transcription factor related, locus 1 | NKX3-1 | Hs.55999 | 209706_at |
| (Drosophila) | | | |
| NMD3 homolog (S. cerevisiae) | NMD3 | Hs.492805 | 218036_x_at |
| NMD3 homolog (S. cerevisiae) | NMD3 | Hs.492805 | 222497_x_at |
| N-myc downstream regulated gene 1 | NDRG1 | Hs.372914 | 200632_s_at |
| NODAL modulator 2 | NOMO2 | Hs.227823 | 217225_x_at |
| NOL1/NOP2/Sun domain family, member 3 | NSUN3 | Hs.401062 | 219458_s_at |
| NOL1/NOP2/Sun domain family, member 4 | NSUN4 | Hs.163424 | 226351_at |
| NOL1/NOP2/Sun domain family, member 4 | NSUN4 | Hs.163424 | 236247_at |
| NOL1/NOP2/Sun domain family, member 4 | NSUN4 | Hs.163424 | 242741_x_at |
| NOL1/NOP2/Sun domain family, member 6 | NSUN6 | Hs.396175 | 230200_at |
| NOL1/NOP2/Sun domain family, member 6 | NSUN6 | Hs.396175 | 242239_at |
| non-metastatic cells 5, protein expressed in | NME5 | Hs.519602 | 206197_at |
| (nucleoside-diphosphate kinase) | | | |
| normal mucosa of esophagus specific 1 | NMES1 | Hs.112242 | 223484_at |
| Notch homolog 2 (Drosophila) | NOTCH2 | Hs.549056 | 202443_x_at |
| Notch homolog 2 (Drosophila) N-terminal like | NOTCH2NL | Hs.502564 | 214722_at |
| NSE1 | NSE1 | Hs.260855 | 234331_s_at |
| N-terminal asparagine amidase | NTAN1 | Hs.351573 | 213061_s_at |
| nuclear autoantigenic sperm protein (histone- | NASP | Hs.319334 | 201969_at |
| binding) | | | |
| nuclear cap binding protein subunit 2, 20 kDa | NCBP2 | Hs.240770 | 201517_at |
| nuclear cap binding protein subunit 2, 20 kDa | NCBP2 | Hs.240770 | 201521_s_at |
| nuclear distribution gene C homolog (A. nidulans) | NUDC | Hs.263812 | 210574_s_at |
| nuclear domain 10 protein | NDP52 | Hs.514920 | 1560631_at |
| nuclear domain 10 protein | NDP52 | Hs.514920 | 238560_at |
| nuclear factor (erythroid-derived 2)-like 1 | NFE2L1 | Hs.514284 | 200759_x_at |
| nuclear factor (erythroid-derived 2)-like 2 | NFE2L2 | Hs.155396 | 201146_at |
| nuclear factor (erythroid-derived 2)-like 3 | NFE2L3 | Hs.404741 | 204702_s_at |
| Nuclear factor I/B | NFIB | Hs.370359 | 209289_at |
| nuclear factor I/B | NFIB | Hs.370359 | 209290_s_at |
| Nuclear factor I/B | NFIB | Hs.370359 | 230791_at |
| Nuclear factor I/C (CCAAT-binding transcription | NFIC | Hs.170131 | 226895_at |
| factor) | | | |
| nuclear factor of activated T-cells, cytoplasmic, | NFATC3 | Hs.341716 | 210556_at |
| calcineurin-dependent 3 | | | |
| Nuclear factor of kappa light polypeptide gene | NFKB1 | Hs.431926 | 239876_at |
| enhancer in B-cells 1 (p105) | | | |
| nuclear factor of kappa light polypeptide gene | NFKBIZ | Hs.319171 | 223217_s_at |
| enhancer in B-cells inhibitor, zeta | | | |
| nuclear factor of kappa light polypeptide gene | NFKBIZ | Hs.319171 | 223218_s_at |
| enhancer in B-cells inhibitor, zeta | | | |
| nuclear factor, interleukin 3 regulated | NFIL3 | Hs.79334 | 203574_at |
| nuclear fragile X mental retardation protein | NUFIP1 | Hs.525006 | 205134_s_at |
| interacting protein 1 | | | |
| nuclear phosphoprotein similar to S. cerevisiae | PWP1 | Hs.506652 | 201606_s_at |
| PWP1 | | | |
| nuclear pore complex interacting protein /// | NPIP /// | Hs.528782 | 204538_x_at |
| hypothetical protein LOC339047 /// similar to | LOC339047 | | |
| hypothetical protein LOC339047 | /// | | |
| | LOC440341 | | |
| nuclear pore complex interacting protein /// | NPIP /// | Hs.528782 | 214870_x_at |
| hypothetical protein LOC339047 /// similar to | LOC339047 | | |
| hypothetical protein LOC339047 | /// | | |
| | LOC440341 | | |
| nuclear protein UKp68 | FLJ11806 | Hs.325846 | 204216_s_at |
| nuclear protein UKp68 | FLJ11806 | Hs.325846 | 213064_at |
| Nuclear receptor coactivator 2 | NCOA2 | Hs.446678 | 242369_x_at |
| Nuclear receptor coactivator 2 /// Nuclear receptor | NCOA2 | Hs.446678 | 212867_at |
| coactivator 2 | | | |
| nuclear receptor coactivator 3 | NCOA3 | Hs.382168 | 207700_s_at |
| nuclear receptor coactivator 4 | NCOA4 | Hs.522932 | 210774_s_at |
| nuclear receptor coactivator 6 | NCOA6 | Hs.368971 | 208979_at |
| nuclear receptor coactivator 6 interacting protein | NCOA6IP | Hs.335068 | 238346_s_at |
| nuclear receptor coactivator 7 | NCOA7 | Hs.171426 | 225344_at |
| nuclear receptor co-repressor 1 | NCOR1 | Hs.462323 | 200854_at |
| nuclear receptor interacting protein 1 | NRIP1 | Hs.155017 | 202600_s_at |
| nuclear receptor interacting protein 3 | NRIP3 | Hs.523467 | 219557_s_at |
| nuclear receptor subfamily 2, group C, member 2 | NR2C2 | Hs.549074 | 206038_s_at |
| nuclear receptor subfamily 3, group C, member 1 | NR3C1 | Hs.122926 | 201865_x_at |
| (glucocorticoid receptor) | | | |
| Nuclear receptor subfamily 3, group C, member 1 | NR3C1 | Hs.122926 | 232431_at |
| (glucocorticoid receptor) | | | |
| nuclear receptor subfamily 3, group C, member 1 | NR3C1 | Hs.122926 | 211671_s_at |
| (glucocorticoid receptor) /// nuclear receptor | | | |
| subfamily 3, group C, member 1 (glucocorticoid | | | |
| receptor) | | | |
| Nuclear receptor subfamily 3, group C, member 2 | NR3C2 | Hs.163924 | 239673_at |
| Nuclear receptor subfamily 4, group A, member 2 | NR4A2 | Hs.165258 | 235739_at |
| nuclear RNA export factor 1 | NXF1 | Hs.523739 | 208922_s_at |
| nuclear RNA export factor 2 | NXF2 | Hs.530358 | 220981_x_at |
| nuclear transcription factor Y, alpha | NFYA | Hs.10441 | 204109_s_at |
| Nuclear transcription factor Y, gamma | NFYC | Hs.233458 | 241630_at |
| nuclear transport factor 2 | NUTF2 | Hs.356630 | 202397_at |
| nuclear ubiquitous casein kinase and cyclin- | NUCKS | Hs.213061 | 217802_s_at |
| dependent kinase substrate | | | |
| nuclear ubiquitous casein kinase and cyclin- | NUCKS | Hs.213061 | 229353_s_at |
| dependent kinase substrate | | | |
| nucleobindin 2 | NUCB2 | Hs.128686 | 203675_at |
| nucleolar and coiled-body phosphoprotein 1 | NOLC1 | Hs.523238 | 205895_s_at |
| nucleolar and coiled-body phosphoprotein 1 | NOLC1 | Hs.523238 | 211951_at |
| nucleolar and spindle associated protein 1 | NUSAP1 | Hs.511093 | 218039_at |
| Nucleolar protein 4 | NOL4 | Hs.546322 | 238605_at |
| nucleolin | NCL | Hs.79110 | 200610_s_at |
| nucleophosmin (nucleolar phosphoprotein B23, | NPM1 | Hs.519452 | 200063_s_at |
| numatrin) /// nucleophosmin (nucleolar | | | |
| phosphoprotein B23, numatrin) | | | |
| nucleophosmin (nucleolar phosphoprotein B23, | NPM1 | Hs.519452 | 221691_x_at |
| numatrin) /// nucleophosmin (nucleolar | | | |
| phosphoprotein B23, numatrin) | | | |
| nucleoporin 133 kDa | NUP133 | Hs.12457 | 202184_s_at |
| nucleoporin 153 kDa | NUP153 | Hs.121088 | 1559064_at |
| nucleoporin 153 kDa | NUP153 | Hs.121088 | 202097_at |
| nucleoporin 155 kDa | NUP155 | Hs.481942 | 206550_s_at |
| nucleoporin 210 kDa-like | NUP210L | Hs.67639 | 232598_at |
| nucleoporin 214 kDa | NUP214 | Hs.461860 | 202155_s_at |
| nucleoporin 35 kDa | NUP35 | Hs.180591 | 225470_at |
| nucleoporin 50 kDa | NUP50 | Hs.475103 | 213682_at |
| nucleoporin 50 kDa | NUP50 | Hs.475103 | 218294_s_at |
| nucleoporin 98 kDa | NUP98 | Hs.524750 | 203194_s_at |
| nucleoporin like 2 | NUPL2 | Hs.408241 | 204003_s_at |
| nucleoredoxin | NXN | Hs.527989 | 219489_s_at |
| nucleosome assembly protein 1-like 1 | NAP1L1 | Hs.524599 | 204528_s_at |
| nucleosome assembly protein 1-like 1 | NAP1L1 | Hs.524599 | 208752_x_at |
| nucleosome assembly protein 1-like 1 | NAP1L1 | Hs.524599 | 208753_s_at |
| nucleosome assembly protein 1-like 1 | NAP1L1 | Hs.524599 | 208754_s_at |
| nucleosome assembly protein 1-like 1 | NAP1L1 | Hs.524599 | 212967_x_at |
| nucleosome assembly protein 1-like 1 | NAP1L1 | Hs.524599 | 213864_s_at |
| NudC domain containing 3 | NUDCD3 | Hs.488171 | 201270_x_at |
| NudC domain containing 3 | KIAA1068 | Hs.488171 | 242696_at |
| nudE nuclear distribution gene E homolog like 1 | NDEL1 | Hs.372123 | 208093_s_at |
| (A. nidulans) /// nudE nuclear distribution gene E | | | |
| homolog like 1 (A. nidulans) | | | |
| nudix (nucleoside diphosphate linked moiety X)- | NUDT10 | Hs.375178 | 241596_at |
| type motif 10 | | | |
| nudix (nucleoside diphosphate linked moiety X)- | NUDT4 | Hs.506325 | 206302_s_at |
| type motif 4 | | | |
| nudix (nucleoside diphosphate linked moiety X)- | NUDT4 | Hs.506325 | 212181_s_at |
| type motif 4 | | | |
| Numb homolog (Drosophila)-like | NUMBL | Hs.326953 | 242195_x_at |
| nurim (nuclear envelope membrane protein) | NRM | Hs.519993 | 225592_at |
| NYD-SP11 protein | NYD- | Hs.326745 | 224135_at |
| | SP11 | | |
| NY-REN-58 antigen | NY- | Hs.279209 | 213241_at |
| | REN-58 | | |
| OCIA domain containing 1 | OCIAD1 | Hs.518750 | 232644_x_at |
| OCIA domain containing 1 | OCIAD1 | Hs.518750 | 239748_x_at |
| olfactomedin-like 2B | OLFML2B | Hs.507515 | 213125_at |
| olfactory receptor, family 2, subfamily H, member 1 | OR2H1 | Hs.434715 | 1556986_at |
| olfactory receptor, family 2, subfamily H, member 1 | OR2H1 | Hs.434715 | 1556987_s_at |
| Olfactory receptor, family 2, subfamily H, member 1 | OR2H1 | Hs.434715 | 1567656_at |
| olfactory receptor, family 2, subfamily H, member 1 | OR2H1 | Hs.434715 | 1567657_at |
| olfactory receptor, family 4, subfamily N, member 4 | OR4N4 | Hs.525666 | 1560735_s_at |
| Olfactory receptor, family 4, subfamily N, member 4 | OR4N4 | Hs.525666 | 1564855_at |
| Olfactory receptor, family 4, subfamily N, member 4 | OR4N4 | Hs.525666 | 1564856_s_at |
| olfactory receptor, family 4, subfamily N, member 4 | OR4N4 | Hs.525666 | 1560734_at |
| /// olfactory receptor, family 4, subfamily Q, | /// | | |
| member 3 | OR4Q3 | | |
| olfactory receptor, family 7, subfamily E, member | OR7E156P | Hs.363054 | 222327_x_at |
| 156 pseudogene | | | |
| olfactory receptor, family 7, subfamily E, member | OR7E47P | Hs.404129 | 216698_x_at |
| 47 pseudogene /// olfactory receptor, family 7, | /// | | |
| subfamily E, member 18 pseudogene /// olfactory | OR7E18P | | |
| receptor, family 7, subfamily E, member 35 | /// | | |
| pseudogene /// similar to olfactory receptor, family | OR7E35P | | |
| 7, subfamily A, member 17 | /// | | |
| | LOC441453 | | |
| Oligonucleotide/oligosaccharide-binding fold | OBFC1 | Hs.134491 | 240824_at |
| containing 1 | | | |
| oligophrenin 1 | OPHN1 | Hs.128824 | 206323_x_at |
| OMA1 homolog, zinc metallopeptidase (S. cerevisiae) | OMA1 | Hs.425769 | 226019_at |
| opsin 3 (encephalopsin, panopsin) | OPN3 | Hs.534399 | 219032_x_at |
| opsin 5 | OPN5 | Hs.213717 | 233657_at |
| optic atrophy 1 (autosomal dominant) | OPA1 | Hs.478708 | 212213_x_at |
| optineurin | OPTN | Hs.332706 | 202074_s_at |
| organic solute transporter alpha | OSTalpha | Hs.155932 | 229230_at |
| origin recognition complex, subunit 6 homolog-like | ORC6L | Hs.49760 | 219105_x_at |
| (yeast) | | | |
| ORM1-like 1 (S. cerevisiae) | ORMDL1 | Hs.512600 | 223187_s_at |
| ornithine aminotransferase (gyrate atrophy) | OAT | Hs.523332 | 201599_at |
| ornithine decarboxylase 1 | ODC1 | Hs.467701 | 200790_at |
| ornithine decarboxylase antizyme 3 | OAZ3 | Hs.407882 | 222075_s_at |
| Ornithine decarboxylase antizyme inhibitor | OAZIN | Hs.459106 | 240231_at |
| osteopetrosis associated transmembrane protein 1 | OSTM1 | Hs.226780 | 218196_at |
| OTU domain, ubiquitin aldehyde binding 1 | OTUB1 | Hs.473788 | 201245_s_at |
| OTU domain, ubiquitin aldehyde binding 1 | OTUB1 | Hs.473788 | 38710_at |
| outer dense fiber of sperm tails 1 | ODF1 | Hs.159274 | 214485_at |
| outer dense fiber of sperm tails 2 | ODF2 | Hs.129055 | 210415_s_at |
| ovostatin 2 | OVOS2 | Hs.524331 | 228245_s_at |
| oxidation resistance 1 | OXR1 | Hs.148778 | 218197_s_at |
| oxidation resistance 1 | OXR1 | Hs.148778 | 223879_s_at |
| oxidised low density lipoprotein (lectin-like) | OLR1 | Hs.412484 | 210004_at |
| receptor 1 | | | |
| oxysterol binding protein | OSBP | Hs.502688 | 1563051_at |
| oxysterol binding protein | OSBP | Hs.502688 | 201800_s_at |
| Oxysterol binding protein 2 | OSBP2 | Hs.517546 | 239872_at |
| oxysterol binding protein-like 8 | OSBPL8 | Hs.430849 | 212582_at |
| oxysterol binding protein-like 9 | OSBPL9 | Hs.21938 | 218047_at |
| oxytocin receptor | OXTR | Hs.2820 | 206825_at |
| p10-binding protein | Cep70 | Hs.531962 | 224150_s_at |
| p21 (CDKN1A)-activated kinase 2 | PAK2 | Hs.518530 | 244268_x_at |
| p21/Cdc42/Rac1-activated kinase 1 (STE20 | PAK1 | Hs.435714 | 230100_x_at |
| homolog, yeast) | | | |
| p53-inducible cell-survival factor | P53CSV | Hs.69499 | 218403_at |
| p8 protein (candidate of metastasis 1) | P8 | Hs.513463 | 209230_s_at |
| PAI-1 mRNA-binding protein | PAI- | Hs.530412 | 209669_s_at |
| | RBP1 | | |
| PAI-1 mRNA-binding protein | PAI- | Hs.530412 | 217724_at |
| | RBP1 | | |
| paired box gene 2 | PAX2 | Hs.155644 | 206228_at |
| paired box gene 8 | PAX8 | Hs.469728 | 121_at |
| palladin | KIAA0992 | Hs.151220 | 200897_s_at |
| Palladin | KIAA0992 | Hs.151220 | 234135_x_at |
| pallidin homolog (mouse) | PLDN | Hs.7037 | 224892_at |
| Pantothenate kinase 1 | PANK1 | Hs.465933 | 228717_at |
| PAP associated domain containing 1 | PAPD1 | Hs.173946 | 233873_x_at |
| pappalysin 2 /// pappalysin 2 | PAPPA2 | Hs.187284 | 211918_x_at |
| Par-3 partitioning defective 3 homolog (C. elegans) | PARD3 | Hs.131489 | 1556105_at |
| par-6 partitioning defective 6 homolog alpha | PARD6A | Hs.112933 | 205245_at |
| (C. elegans) | | | |
| paraneoplastic antigen MA1 | PNMA1 | Hs.194709 | 218224_at |
| paraspeckle component 1 | PSPC1 | Hs.213198 | 218371_s_at |
| PARK2 co-regulated | PACRG | Hs.25791 | 243122_at |
| Parkinson disease (autosomal recessive, early | PARK7 | Hs.419640 | 200006_at |
| onset) 7 /// Parkinson disease (autosomal | | | |
| recessive, early onset) 7 | | | |
| patched homolog (Drosophila) | PTCH | Hs.494538 | 1555520_at |
| paternally expressed 3 | PEG3 | Hs.201776 | 209242_at |
| PAX interacting (with transcription-activation | PAXIP1 | Hs.443881 | 212825_at |
| domain) protein 1 | | | |
| PBX/knotted 1 homeobox 2 | PKNOX2 | Hs.278564 | 63305_at |
| PCTAIRE protein kinase 2 | PCTK2 | Hs.506415 | 243993_at |
| PDZ and LIM domain 1 (elfin) | PDLIM1 | Hs.368525 | 208690_s_at |
| PDZ and LIM domain 5 | PDLIM5 | Hs.480311 | 212412_at |
| PDZ binding kinase | PBK | Hs.104741 | 219148_at |
| PDZ domain containing 3 | PDZK3 | Hs.481819 | 209493_at |
| PDZ domain containing 8 | PDZK8 | Hs.501149 | 225829_at |
| PDZ domain containing 8 | PDZK8 | Hs.501149 | 225830_at |
| pecanex homolog (Drosophila) | PCNX | Hs.158722 | 215175_at |
| PEF protein with a long N-terminal hydrophobic | PEFLIN | Hs.470417 | 217923_at |
| domain | | | |
| Pellino homolog 1 (Drosophila) | PELI1 | Hs.7886 | 232213_at |
| peptidylprolyl isomerase (cyclophilin)-like 1 | PPIL1 | Hs.27693 | 222500_at |
| peptidylprolyl isomerase (cyclophilin)-like 5 | PPIL5 | Hs.451090 | 235113_at |
| peptidylprolyl isomerase A (cyclophilin A) | PPIA | Hs.356331 | 201293_x_at |
| peptidylprolyl isomerase A (cyclophilin A) | PPIA | Hs.356331 | 211378_x_at |
| peptidylprolyl isomerase A (cyclophilin A) | PPIA | Hs.356331 | 211978_x_at |
| peptidylprolyl isomerase A (cyclophilin A) | PPIA | Hs.356331 | 212661_x_at |
| peptidylprolyl isomerase A (cyclophilin A) /// | PPIA | Hs.356331 | 211765_x_at |
| peptidylprolyl isomerase A (cyclophilin A) | | | |
| peptidylprolyl isomerase C (cyclophilin C) | PPIC | Hs.110364 | 204517_at |
| peptidylprolyl isomerase D (cyclophilin D) | PPID | Hs.183958 | 204185_x_at |
| pericentriolar material 1 | PCM1 | Hs.491148 | 209997_x_at |
| Pericentriolar material 1 | PCM1 | Hs.491148 | 233270_x_at |
| period homolog 1 (Drosophila) | PER1 | Hs.445534 | 36829_at |
| period homolog 2 (Drosophila) | PER2 | Hs.58756 | 205251_at |
| peroxiredoxin 2 | PRDX2 | Hs.432121 | 215067_x_at |
| peroxiredoxin 3 | PRDX3 | Hs.523302 | 209766_at |
| peroxiredoxin 4 | PRDX4 | Hs.83383 | 201923_at |
| peroxiredoxin 6 | PRDX6 | Hs.120 | 200845_s_at |
| Peroxisomal acyl-CoA thioesterase | PTE1 | Hs.444776 | 236514_at |
| peroxisomal biogenesis factor 11B | PEX11B | Hs.504284 | 202658_at |
| peroxisomal biogenesis factor 16 | PEX16 | Hs.100915 | 49878_at |
| peroxisomal biogenesis factor 3 | PEX3 | Hs.7277 | 203972_s_at |
| peroxisomal biogenesis factor 5 | PEX5 | Hs.479944 | 203244_at |
| peroxisomal biogenesis factor 7 /// peroxisomal | PEX7 | Hs.280932 | 211033_s_at |
| biogenesis factor 7 | | | |
| peroxisomal D3,D2-enoyl-CoA isomerase | PECI | Hs.15250 | 218025_s_at |
| Peroxisomal Ion protease | LONP | Hs.551565 | 221834_at |
| peroxisomal trans-2-enoyl-CoA reductase | PECR | Hs.281680 | 223619_x_at |
| peroxisomal, testis specific 1 | PXT1 | Hs.520097 | 1552599_at |
| peroxisome biogenesis factor 13 | PEX13 | Hs.336219 | 205246_at |
| PERP, TP53 apoptosis effector | PERP | Hs.520421 | 222392_x_at |
| PFTAIRE protein kinase 1 | PFTK1 | Hs.430742 | 204604_at |
| PFTAIRE protein kinase 1 | PFTK1 | Hs.430742 | 211502_s_at |
| PHD finger protein 1 | PHF1 | Hs.166204 | 40446_at |
| PHD finger protein 10 | PHF10 | Hs.435933 | 219126_at |
| PHD finger protein 10 | PHF10 | Hs.435933 | 221786_at |
| PHD finger protein 13 | PHF13 | Hs.516079 | 225005_at |
| PHD finger protein 2 | PHF2 | Hs.211441 | 212726_at |
| PHD finger protein 20 | PHF20 | Hs.517044 | 206567_s_at |
| PHD finger protein 20-like 1 | PHF20L1 | Hs.304362 | 222133_s_at |
| PHD finger protein 20-like 1 | PHF20L1 | Hs.304362 | 227523_s_at |
| PHD finger protein 7 | PHF7 | Hs.372719 | 215622_x_at |
| PHD finger protein 7 | PHF7 | Hs.372719 | 221611_s_at |
| PHD finger protein 7 | PHF7 | Hs.372719 | 221930_at |
| phosducin-like 3 /// hypothetical protein FLJ12205 | PDCL3 | Hs.449206 | 219043_s_at |
| | /// | | |
| | LOC285359 | | |
| phosphatase and actin regulator 2 | PHACTR2 | Hs.102471 | 204049_s_at |
| Phosphatase and actin regulator 4 | PHACTR4 | Hs.225641 | 233319_x_at |
| phosphatase and tensin homolog (mutated in | PTEN | Hs.500466 | 204053_x_at |
| multiple advanced cancers 1) | | | |
| Phosphatase and tensin homolog (mutated in | PTEN | Hs.500466 | 225363_at |
| multiple advanced cancers 1) | | | |
| phosphatase and tensin homolog (mutated in | PTENP1 | Hs.493716 | 217492_s_at |
| multiple advanced cancers 1), pseudogene 1 | | | |
| phosphatidic acid phosphatase type 2 domain | LOC403313 | Hs.107510 | 227385_at |
| containing 2 | | | |
| phosphatidic acid phosphatase type 2A | PPAP2A | Hs.435122 | 210946_at |
| Phosphatidic acid phosphatase type 2B | PPAP2B | Hs.405156 | 232324_x_at |
| phosphatidylinositol 4-kinase, catalytic, alpha | PIK4CA | Hs.448225 | 213408_s_at |
| polypeptide /// hypothetical protein LOC220686 | /// | | |
| | LOC220686 | | |
| Phosphatidylinositol binding clathrin assembly | PICALM | Hs.163893 | 239102_s_at |
| protein | | | |
| phosphatidylinositol glycan, class A (paroxysmal | PIGA | Hs.137154 | 205281_s_at |
| nocturnal hemoglobinuria) /// phosphatidylinositol | | | |
| glycan, class A (paroxysmal nocturnal | | | |
| hemoglobinuria) | | | |
| phosphatidylinositol glycan, class B | PIGB | Hs.126115 | 205452_at |
| phosphatidylinositol glycan, class B | PIGB | Hs.126115 | 214152_at |
| phosphatidylinositol glycan, class C | PIGC | Hs.188456 | 202846_s_at |
| phosphatidylinositol glycan, class C | PIGC | Hs.188456 | 216593_s_at |
| phosphatidylinositol glycan, class F | PIGF | Hs.468415 | 205077_s_at |
| phosphatidylinositol glycan, class F | PIGF | Hs.468415 | 205078_at |
| phosphatidylinositol glycan, class L | PIGL | Hs.167559 | 205873_at |
| phosphatidylinositol glycan, class V | PIGV | Hs.259605 | 51146_at |
| phosphatidylinositol transfer protein, alpha | PITPNA | Hs.429819 | 201192_s_at |
| phosphatidylinositol transfer protein, beta | PITPNB | Hs.7370 | 202522_at |
| Phosphatidylinositol transfer protein, beta | PITPNB | Hs.7370 | 244826_at |
| phosphatidylinositol-4-phosphate 5-kinase, type I, | PIP5K1B | Hs.534371 | 205632_s_at |
| beta | | | |
| phosphatidylinositol-4-phosphate 5-kinase, type II, | PIP5K2A | Hs.499029 | 205570_at |
| alpha | | | |
| phosphatidylserine receptor | PTDSR | Hs.514505 | 212723_at |
| phosphatidylserine synthase 1 | PTDSS1 | Hs.292579 | 201433_s_at |
| phosphodiesterase 10A | PDE10A | Hs.487129 | 211171_s_at |
| phosphodiesterase 1A, calmodulin-dependent | PDE1A | Hs.416061 | 208396_s_at |
| phosphodiesterase 1C, calmodulin-dependent | PDE1C | Hs.487897 | 207303_at |
| 70 kDa | | | |
| phosphodiesterase 4B, cAMP-specific | PDE4B | Hs.198072 | 211302_s_at |
| (phosphodiesterase E4 dunce homolog, | | | |
| Drosophila) | | | |
| phosphodiesterase 4C, cAMP-specific | PDE4C | Hs.437211 | 206792_x_at |
| (phosphodiesterase E1 dunce homolog, | | | |
| Drosophila) | | | |
| Phosphodiesterase 4D interacting protein | PDE4DIP | Hs.487925 | 244511_at |
| (myomegalin) | | | |
| phosphofructokinase, muscle | PFKM | Hs.75160 | 210976_s_at |
| phosphofructokinase, platelet | PFKP | Hs.26010 | 201037_at |
| phosphofurin acidic cluster sorting protein 1 | PACS1 | Hs.536275 | 224658_x_at |
| phosphoglucomutase 2 | PGM2 | Hs.23363 | 225366_at |
| phosphoglucomutase 2-like 1 | PGM2L1 | Hs.26612 | 229553_at |
| phosphoglycerate kinase 1 | PGK1 | Hs.78771 | 200738_s_at |
| phosphoglycerate kinase 2 | PGK2 | Hs.367727 | 217009_at |
| phosphoglycerate mutase 1 (brain) | PGAM1 | Hs.447492 | 200886_s_at |
| phosphohistidine phosphatase 1 | PHPT1 | Hs.409834 | 223207_x_at |
| Phosphoinositide-3-kinase, class 2, alpha | PIK3C2A | Hs.175343 | 241905_at |
| polypeptide | | | |
| phosphoinositide-3-kinase, regulatory subunit 1 | PIK3R1 | Hs.132225 | 212239_at |
| (p85 alpha) | | | |
| phosphoinositide-3-kinase, regulatory subunit 1 | PIK3R1 | Hs.132225 | 212240_s_at |
| (p85 alpha) | | | |
| phosphoinositide-3-kinase, regulatory subunit 5, | PIK3R5 | Hs.278901 | 220566_at |
| p101 | | | |
| phospholamban | PLN | Hs.170839 | 204938_s_at |
| phospholipase A2, group VI (cytosolic, calcium- | PLA2G6 | Hs.170479 | 210647_x_at |
| independent) | | | |
| Phospholipase C, beta 1 (phosphoinositide- | PLCB1 | Hs.310537 | 244726_at |
| specific) | | | |
| phospholipase C, beta 4 | PLCB4 | Hs.472101 | 203895_at |
| Phospholipase C, beta 4 | PLCB4 | Hs.472101 | 240728_at |
| phospholipase C, gamma 2 (phosphatidylinositol- | PLCG2 | Hs.413111 | 204613_at |
| specific) | | | |
| phospholipase C, zeta 1 | PLCZ1 | Hs.97542 | 231425_at |
| phospholipid scramblase 2 | PLSCR2 | Hs.147305 | 207374_at |
| Phosphonoformate immuno-associated protein 5 | PFAAP5 | Hs.507680 | 221899_at |
| phosphoprotein enriched in astrocytes 15 | PEA15 | Hs.517216 | 200787_s_at |
| phosphoprotein enriched in astrocytes 15 | PEA15 | Hs.517216 | 200788_s_at |
| phosphoribosyl pyrophosphate synthetase 1 | PRPS1 | Hs.56 | 209440_at |
| phosphoribosyl pyrophosphate synthetase- | PRPSAP1 | Hs.77498 | 202529_at |
| associated protein 1 | | | |
| Phosphoribosylglycinamide formyltransferase, | GART | Hs.473648 | 230097_at |
| phosphoribosylglycinamide synthetase, | | | |
| phosphoribosylaminoimidazole synthetase | | | |
| phosphorylase kinase, beta | PHKB | Hs.78060 | 202738_s_at |
| phosphoserine aminotransferase 1 | PSAT1 | Hs.494261 | 223062_s_at |
| phytanoyl-CoA hydroxylase (Refsum disease) | PHYH | Hs.498732 | 203335_at |
| phytanoyl-CoA hydroxylase interacting protein-like | PHYHIPL | Hs.499704 | 226623_at |
| phytoceramidase, alkaline | PHCA | Hs.23862 | 222688_at |
| PI-3-kinase-related kinase SMG-1 /// PI-3-kinase- | SMG1 /// | Hs.460179 | 244766_at |
| related kinase SMG-1-like /// hLAT1-3TM /// similar | KIAA0220 | | |
| to PI-3-kinase-related kinase SMG-1 isoform 1; | /// | | |
| lambda/iota protein kinase C-interacting protein; | IMAA /// | | |
| phosphatidylinositol 3-kinase-related protein | LOC440345 | | |
| kinase /// hypothetica protein LOC440354 | /// | | |
| | LOC440354 | | |
| PI-3-kinase-related kinase SMG-1-like | KIAA0220 | Hs.528462 | 211996_s_at |
| pinin, desmosome associated protein | PNN | Hs.409965 | 1567213_at |
| pinin, desmosome associated protein | PNN | Hs.409965 | 1567214_a_at |
| piwi-like 1 (Drosophila) | PIWIL1 | Hs.405659 | 214868_at |
| PLAC8-like 1 | PLAC8L1 | Hs.162369 | 237783_at |
| Placental growth factor, vascular endothelial | PGF | Hs.252820 | 215179_x_at |
| growth factor-related protein | | | |
| plakophilin 4 | PKP4 | Hs.407580 | 201928_at |
| Plakophilin 4 | PKP4 | Hs.407580 | 236752_at |
| plasminogen-like B1 | PLGLB1 | Hs.424184 | 205871_at |
| platelet-activating factor acetylhydrolase, isoform | PAFAH1B1 | Hs.77318 | 200815_s_at |
| Ib, alpha subunit 45 kDa | | | |
| platelet-activating factor acetylhydrolase, isoform | PAFAH1B2 | Hs.188501 | 224777_s_at |
| Ib, beta subunit 30 kDa | | | |
| pleckstrin and Sec7 domain containing 3 | PSD3 | Hs.434255 | 203355_s_at |
| pleckstrin homology domain containing, family A | PLEKHA3 | Hs.41086 | 223370_at |
| (phosphoinositide binding specific) member 3 | | | |
| pleckstrin homology domain containing, family A | PLEKHA3 | Hs.41086 | 227658_s_at |
| (phosphoinositide binding specific) member 3 | | | |
| pleckstrin homology domain containing, family A | PLEKHA5 | Hs.188614 | 220952_s_at |
| member 5 | | | |
| Pleckstrin homology domain containing, family A | PLEKHA5 | Hs.188614 | 237483_at |
| member 5 | | | |
| Pleckstrin homology domain containing, family A | PLEKHA6 | Hs.253146 | 238134_at |
| member 6 | | | |
| pleckstrin homology domain containing, family B | PLEKHB1 | Hs.445489 | 209504_s_at |
| (evectins) member 1 | | | |
| Pleckstrin homology domain containing, family B | PLEKHB2 | Hs.469944 | 201410_at |
| (evectins) member 2 | | | |
| pleckstrin homology domain containing, family J | PLEKHJ1 | Hs.501353 | 218290_at |
| member 1 | | | |
| pleckstrin homology, Sec7 and coiled-coil domains | PSCD2 | Hs.8517 | 209158_s_at |
| 2 (cytohesin-2) | | | |
| pleckstrin homology-like domain, family A, member 2 | PHLDA2 | Hs.14036 | 209802_at |
| pleckstrin homology-like domain, family A, member 2 | PHLDA2 | Hs.154036 | 209803_s_at |
| pleiomorphic adenoma gene-like 2 | PLAGL2 | Hs.154104 | 202924_s_at |
| PMS1 postmeiotic segregation increased 1 (S. cerevisiae) | PMS1 | Hs.111749 | 213677_s_at |
| PNAS-13 | LOC441212 | Hs.292057 | 223945_x_at |
| Poliovirus receptor-related 3 | PVRL3 | Hs.293917 | 222167_at |
| Poliovirus receptor-related 3 | PVRL3 | Hs.293917 | 241970_at |
| poly (ADP-ribose) polymerase family, member 1 | PARP1 | Hs.177766 | 208644_at |
| poly (ADP-ribose) polymerase family, member 2 | PARP2 | Hs.409412 | 215773_x_at |
| poly (ADP-ribose) polymerase family, member 6 | PARP6 | Hs.270244 | 219639_x_at |
| Poly (ADP-ribose) polymerase family, member 9 | PARP9 | Hs.518200 | 227807_at |
| poly(A) binding protein interacting protein 2 | PAIP2 | Hs.396644 | 222983_s_at |
| poly(A) binding protein, cytoplasmic 1 | PABPC1 | Hs.387804 | 215157_x_at |
| poly(A) binding protein, cytoplasmic 3 /// poly(A) | PABPC3 | Hs.387804 | 215823_x_at |
| binding protein, cytoplasmic 1 /// similar to | /// | | |
| Polyadenylate-binding protein 1 (Poly(A)-binding | PABPC1 | | |
| protein 1) (PABP 1) | /// | | |
| | LOC341315 | | |
| poly(A) binding protein, cytoplasmic 3 /// poly(A) | PABPC3 | Hs.458280 | 208113_x_at |
| binding protein, cytoplasmic 3 | | | |
| poly(A) binding protein, nuclear 1 | PABPN1 | Hs.117176 | 201544_x_at |
| poly(A) binding protein, nuclear 1 | PABPN1 | Hs.117176 | 213046_at |
| poly(A) polymerase alpha | PAPOLA | Hs.253726 | 209388_at |
| poly(A) polymerase alpha | PAPOLA | Hs.253726 | 212718_at |
| poly(A) polymerase alpha | PAPOLA | Hs.253726 | 228569_at |
| poly(A) polymerase beta (testis specific) | PAPOLB | Hs.487409 | 242158_at |
| poly(A) polymerase gamma | PAPOLG | Hs.146123 | 222839_s_at |
| poly(A)-specific ribonuclease (deadenylation | PARN | Hs.253197 | 203905_at |
| nuclease) | | | |
| Poly(rC) binding protein 2 | PCBP2 | Hs.546271 | 213517_at |
| polyamine oxidase (exo-N4-amino) | PAOX | Hs.532469 | 221941_at |
| polyamine oxidase (exo-N4-amino) | PAOX | Hs.532469 | 50400_at |
| polybromo 1 | PB1 | Hs.189920 | 220355_s_at |
| Polycomb group ring finger 3 | PCGF3 | Hs.144309 | 243329_at |
| polycomb group ring finger 4 | PCGF4 | Hs.380403 | 202265_at |
| polycomb group ring finger 5 | PCGF5 | Hs.500512 | 227935_s_at |
| polycomb group ring finger 6 /// polycomb group | PCGF6 | Hs.335808 | 224326_s_at |
| ring finger 6 | | | |
| Polycystic kidney disease 1 (autosomal dominant) | PKD1 | Hs.75813 | 241090_at |
| polyhomeotic-like 1 (Drosophila) | PHC1 | Hs.305985 | 225958_at |
| polyhomeotic-like 2 (Drosophila) | PHC2 | Hs.524271 | 200919_at |
| polymerase (DNA directed) sigma | POLS | Hs.481542 | 202466_at |
| polymerase (DNA directed), beta | POLB | Hs.521563 | 203616_at |
| Polymerase (DNA directed), epsilon | POLE | Hs.524871 | 1560509_at |
| polymerase (DNA directed), epsilon 3 (p17 | POLE3 | Hs.108112 | 208828_at |
| subunit) | | | |
| Polymerase (DNA directed), eta | POLH | Hs.439153 | 1557700_at |
| polymerase (DNA directed), eta | POLH | Hs.439153 | 219380_x_at |
| polymerase (DNA-directed), delta interacting | POLDIP2 | Hs.241543 | 217806_s_at |
| protein 2 | | | |
| polymerase (RNA) I polypeptide B, 128 kDa | POLR1B | Hs.86337 | 220113_x_at |
| polymerase (RNA) I polypeptide B, 128 kDa | POLR1B | Hs.86337 | 223403_s_at |
| polymerase (RNA) I polypeptide B, 128 kDa | POLR1B | Hs.86337 | 233341_s_at |
| polymerase (RNA) I polypeptide C, 30 kDa | POLR1C | Hs.520146 | 209317_at |
| polymerase (RNA) II (DNA directed) polypeptide B, | POLR2B | Hs.479814 | 1555837_s_at |
| 140 kDa | | | |
| polymerase (RNA) II (DNA directed) polypeptide B, | POLR2B | Hs.479814 | 201803_at |
| 140 kDa | | | |
| polymerase (RNA) II (DNA directed) polypeptide C, | POLR2C | Hs.79402 | 216282_x_at |
| 33 kDa | | | |
| polymerase (RNA) II (DNA directed) polypeptide D | POLR2D | Hs.444887 | 203664_s_at |
| polymerase (RNA) II (DNA directed) polypeptide H | POLR2H | Hs.432574 | 209302_at |
| polymerase (RNA) III (DNA directed) polypeptide C | POLR3C | Hs.550501 | 210573_s_at |
| (62 kD) | | | |
| polymerase (RNA) III (DNA directed) polypeptide E | POLR3E | Hs.460298 | 218016_s_at |
| (80 kD) | | | |
| polymerase (RNA) III (DNA directed) polypeptide G | POLR3GL | Hs.9088 | 223269_at |
| (32 kD) like | | | |
| Polypyrimidine tract binding protein 2 | PTBP2 | Hs.269895 | 1560271_at |
| POM121 membrane glycoprotein (rat) /// nuclear | POM121 | Hs.488624 | 212178_s_at |
| envelope pore membrane LOC340318 | /// | | |
| | LOC340318 | | |
| POM121-like protein | DKFZP434P211 | Hs.328458 | 222253_s_at |
| postmeiotic segregation increased 2-like 1 | PMS2L1 | Hs.520575 | 214756_x_at |
| postmeiotic segregation increased 2-like 1 | PMS2L1 | Hs.520575 | 216843_x_at |
| postmeiotic segregation increased 2-like 1 | PMS2L1 | Hs.520575 | 217485_x_at |
| postmeiotic segregation increased 2-like 2 | PMS2L2 | Hs.292996 | 215412_x_at |
| Postmeiotic segregation increased 2-like 3 | PMS2L3 | Hs.549057 | 214473_x_at |
| Postmeiotic segregation increased 2-like 3 | PMS2L3 | Hs.549057 | 216525_x_at |
| postmeiotic segregation increased 2-like 5 | PMS2L5 | Hs.397073 | 213893_x_at |
| potassium channel modulatory factor 1 | KCMF1 | Hs.345694 | 217938_s_at |
| potassium channel regulator | KCNRG | Hs.508035 | 240288_at |
| potassium channel tetramerisation domain | KCTD10 | Hs.524731 | 223208_at |
| containing 10 | | | |
| Potassium channel tetramerisation domain | KCTD13 | Hs.534590 | 238142_at |
| containing 13 | | | |
| potassium channel, subfamily K, member 4 | KCNK4 | Hs.97174 | 219883_at |
| potassium channel, subfamily V, member 2 | KCNV2 | Hs.131130 | 217522_at |
| potassium inwardly-rectifying channel, subfamily J, | KCNJ14 | Hs.144011 | 220776_at |
| member 14 | | | |
| potassium large conductance calcium-activated | KCNMB4 | Hs.525529 | 219287_at |
| channel, subfamily M, beta member 4 | | | |
| potassium voltage-gated channel, subfamily H | KCNH7 | Hs.470445 | 224099_at |
| (eag-related), member 7 | | | |
| POU domain, class 3, transcription factor 3 | POU3F3 | Hs.248158 | 228780_at |
| PPAR binding protein | PPARBP | Hs.462956 | 203497_at |
| PQ loop repeat containing 1 | PQLC1 | Hs.288284 | 218208_at |
| PR domain containing 10 | PRDM10 | Hs.275086 | 219515_at |
| PR domain containing 2, with ZNF domain | PRDM2 | Hs.371823 | 205277_at |
| PR domain containing 4 | PRDM4 | Hs.506655 | 49485_at |
| PR domain containing 5 | PRDM5 | Hs.132593 | 220792_at |
| Prader-Willi/Angelman region-1 | PAR1 | Hs.546847 | 226587_at |
| praja 1 | PJA1 | Hs.522679 | 218667_at |
| praja 2, RING-H2 motif containing | PJA2 | Hs.483036 | 201133_s_at |
| pre-B-cell colony enhancing factor 1 | PBEF1 | Hs.489615 | 217738_at |
| pre-B-cell colony enhancing factor 1 | PBEF1 | Hs.489615 | 217739_s_at |
| Pre-B-cell leukemia transcription factor 1 | PBX1 | Hs.493096 | 1562235_s_at |
| Pre-B-cell leukemia transcription factor 1 | PBX1 | Hs.493096 | 212151_at |
| pre-B-cell leukemia transcription factor 3 | PBX3 | Hs.428027 | 204082_at |
| pre-B-cell leukemia transcription factor interacting | PBXIP1 | Hs.505806 | 212259_s_at |
| protein 1 | | | |
| PREDICTED: Homo sapiens olfactory receptor, | — | Hs.507861 | 217499_x_at |
| family 7, subfamily E, member 31 pseudogene | | | |
| (OR7E31P), mRNA | | | |
| pregnancy specific beta-1-glycoprotein 3 /// | PSG3 | Hs.466843 | 211741_x_at |
| pregnancy specific beta-1-glycoprotein 3 | | | |
| pregnancy specific beta-1-glycoprotein 6 | PSG6 | Hs.466849 | 208106_x_at |
| pregnancy-associated plasma protein A, | PAPPA | Hs.494928 | 224940_s_at |
| pappalysin 1 | | | |
| pregnancy-associated plasma protein A, | PAPPA | Hs.494928 | 228128_x_at |
| pappalysin 1 | | | |
| pregnancy-zone protein | PZP | Hs.480143 | 207330_at |
| pre-mRNA cleavage complex II protein Pcf11 | PCF11 | Hs.128959 | 227622_at |
| presenilin 1 (Alzheimer disease 3) | PSEN1 | Hs.3260 | 203460_s_at |
| prickle-like 1 (Drosophila) | PRICKLE1 | Hs.524348 | 226065_at |
| PRO0149 protein | PRO0149 | Hs.221497 | 1568954_s_at |
| PRO0149 protein | PRO0149 | Hs.221497 | 225183_at |
| PRO0149 protein | PRO0149 | Hs.221497 | 225197_at |
| PRO0611 protein | PUM1 | Hs.281707 | 241079_at |
| PRO1073 protein | PRO1073 | — | 223577_x_at |
| PRO1073 protein | PRO1073 | — | 231735_s_at |
| PRO1550 | — | Hs.371588 | 1556543_at |
| PRO1550 | — | Hs.371588 | 230332_at |
| PRO1550 | — | Hs.371588 | 234032_at |
| procollagen (type III) N-endopeptidase | PCOLN3 | Hs.461777 | 201933_at |
| procollagen-lysine, 2-oxoglutarate 5-dioxygenase 3 | PLOD3 | Hs.153357 | 202185_at |
| profilin 2 | PFN2 | Hs.91747 | 204992_s_at |
| Profilin 2 | PFN2 | Hs.91747 | 237116_at |
| profilin family, member 4 | PFN4 | Hs.442718 | 235627_at |
| progestin and adipoQ receptor family member III | PAQR3 | Hs.368305 | 1557576_at |
| progestin and adipoQ receptor family member III | PAQR3 | Hs.368305 | 213372_at |
| programmed cell death 10 | PDCD10 | Hs.478150 | 210907_s_at |
| programmed cell death 2 | PDCD2 | Hs.367900 | 228420_at |
| programmed cell death 4 (neoplastic | PDCD4 | Hs.232543 | 202731_at |
| transformation inhibitor) | | | |
| programmed cell death 6 /// aryl-hydrocarbon | PDCD6 | Hs.50823 | 229354_at |
| receptor repressor | /// AHRR | | |
| programmed cell death 6 /// hypothetical gene | PDCD6 | Hs.518410 | 1568763_s_at |
| supported by AK055127; BC053586; BC067863 | /// | | |
| | LOC440997 | | |
| programmed cell death 6 interacting protein | PDCD6IP | Hs.475896 | 217746_s_at |
| projection protein PF6 | PF6 | Hs.528821 | 233516_s_at |
| prokineticin 1 | PROK1 | Hs.514793 | 229124_at |
| prolactin | PRL | Hs.1905 | 205445_at |
| proliferating cell nuclear antigen | PCNA | Hs.147433 | 201202_at |
| Proline rich Gla (G-carboxyglutamic acid) 4 | PRRG4 | Hs.471695 | 238513_at |
| (transmembrane) | | | |
| proline-rich cyclin A1-interacting protein | PROCA1 | Hs.207471 | 232647_at |
| proline-rich nuclear receptor coactivator 2 | PNRC2 | Hs.7862 | 217779_s_at |
| proline-rich protein BstNI subfamily 1 /// proline-rich | PRB1 /// | Hs.283658 | 210597_x_at |
| protein BstNI subfamily 2 | PRB2 | | |
| pro-melanin-concentrating hormone-like 1 /// pro- | PMCHL1 | Hs.247975 | 224418_x_at |
| melanin-concentrating hormone-like 1 | | | |
| pro-melanin-concentrating hormone-like 1 /// pro- | PMCHL1 | Hs.247975 | 224419_x_at |
| melanin-concentrating hormone-like 1 | | | |
| prominin 1 | PROM1 | Hs.479220 | 204304_s_at |
| pro-oncosis receptor inducing membrane injury | PORIMIN | Hs.503709 | 211967_at |
| gene | | | |
| propionyl Coenzyme A carboxylase, beta | PCCB | Hs.63788 | 212694_s_at |
| polypeptide | | | |
| proprotein convertase subtilisin/kexin type 5 | PCSK5 | Hs.368542 | 205559_s_at |
| Prostate tumor overexpressed gene 1 | PTOV1 | Hs.515540 | 240615_at |
| prostatic binding protein | PBP | Hs.433863 | 210825_s_at |
| protamine 1 | PRM1 | Hs.2909 | 206358_at |
| protamine 2 | PRM2 | Hs.2324 | 210122_at |
| protease, serine, 22 | PRSS22 | Hs.459709 | 205847_at |
| proteasome (prosome, macropain) 26S subunit, | PSMC2 | Hs.437366 | 201068_s_at |
| ATPase, 2 | | | |
| proteasome (prosome, macropain) 26S subunit, | PSMD1 | Hs.3887 | 201198_s_at |
| non-ATPase, 1 | | | |
| proteasome (prosome, macropain) 26S subunit, | PSMD12 | Hs.4295 | 202352_s_at |
| non-ATPase, 12 | | | |
| proteasome (prosome, macropain) activator | PSME4 | Hs.413801 | 212220_at |
| subunit 4 | | | |
| proteasome (prosome, macropain) activator | PSME4 | Hs.413801 | 212222_at |
| subunit 4 | | | |
| Proteasome (prosome, macropain) activator | PSME4 | Hs.413801 | 237180_at |
| subunit 4 | | | |
| proteasome (prosome, macropain) inhibitor subunit | PSMF1 | Hs.471917 | 201053_s_at |
| 1 (PI31) | | | |
| Proteasome (prosome, macropain) inhibitor | PSMF1 | Hs.471917 | 235993_at |
| subunit 1 (PI31) | | | |
| proteasome (prosome, macropain) subunit, alpha | PSMA4 | Hs.251531 | 203396_at |
| type, 4 | | | |
| proteasome (prosome, macropain) subunit, alpha | PSMA6 | Hs.446260 | 208805_at |
| type, 6 | | | |
| proteasome (prosome, macropain) subunit, beta | PSMB1 | Hs.352768 | 214288_s_at |
| type, 1 | | | |
| proteasome (prosome, macropain) subunit, beta | PSMB5 | Hs.422990 | 208799_at |
| type, 5 | | | |
| proteasome regulatory particle subunit p44S10 | p44S10 | Hs.152536 | 202753_at |
| protein (peptidyl-prolyl cis/trans isomerase) NIMA- | PIN4 | Hs.118076 | 204571_x_at |
| interacting, 4 (parvulin) | | | |
| protein (peptidyl-prolyl cis/trans isomerase) NIMA- | PIN4 | Hs.118076 | 204572_s_at |
| interacting, 4 (parvulin) | | | |
| protein (peptidyl-prolyl cis/trans isomerase) NIMA- | PIN4 | Hs.118076 | 214224_s_at |
| interacting, 4 (parvulin) | | | |
| protein 7 transactivated by hepatitis B virus X | XTP7 | Hs.337557 | 1552889_a_at |
| antigen (HBxAg) | | | |
| protein disulfide isomerase-associated 5 | PDIA5 | Hs.477352 | 203857_s_at |
| protein disulfide isomerase-associated 6 | PDIA6 | Hs.212102 | 207668_x_at |
| protein disulfide isomerase-associated 6 | PDIA6 | Hs.212102 | 208639_x_at |
| Protein geranylgeranyltransferase type I, beta | PGGT1B | Hs.254006 | 242844_at |
| subunit | | | |
| Protein inhibitor of activated STAT, 1 | PIAS1 | Hs.162458 | 222371_at |
| protein inhibitor of activated STAT, 2 | PIAS2 | Hs.514846 | 1555514_a_at |
| Protein inhibitor of activated STAT, 2 | PIAS2 | Hs.514846 | 244633_at |
| protein kinase C and casein kinase substrate in | PACSIN2 | Hs.162877 | 1554691_a_at |
| neurons 2 | | | |
| protein kinase C binding protein 1 | PRKCBP1 | Hs.446240 | 230533_at |
| protein kinase C, beta 1 | PRKCB1 | Hs.460355 | 209685_s_at |
| Protein kinase C, eta | PRKCH | Hs.333907 | 1559425_at |
| protein kinase C, iota | PRKCI | Hs.399873 | 209678_s_at |
| protein kinase D3 /// protein kinase D3 | PRKD3 | Hs.173536 | 211084_x_at |
| protein kinase LYK5 | LYK5 | Hs.549229 | 52169_at |
| protein kinase N2 | PKN2 | Hs.440833 | 212629_s_at |
| protein kinase NYD-SP25 | NYD- | Hs.351815 | 231554_at |
| | SP25 | | |
| protein kinase NYD-SP25 | NYD- | Hs.351815 | 240051_at |
| | SP25 | | |
| protein kinase, AMP-activated, gamma 2 non- | PRKAG2 | Hs.549162 | 218292_s_at |
| catalytic subunit | | | |
| protein kinase, AMP-activated, gamma 2 non- | PRKAG2 | Hs.549162 | 233748_x_at |
| catalytic subunit | | | |
| protein kinase, cAMP-dependent, regulatory, type | PRKAR1A | Hs.280342 | 200604_s_at |
| I, alpha (tissue specific extinguisher 1) | | | |
| protein kinase, cAMP-dependent, regulatory, type | PRKAR1A | Hs.280342 | 200605_s_at |
| I, alpha (tissue specific extinguisher 1) | | | |
| protein kinase, cAMP-dependent, regulatory, type | PRKAR2A | Hs.517841 | 204842_x_at |
| II, alpha | | | |
| Protein kinase, cAMP-dependent, regulatory, type | PRKAR2A | Hs.517841 | 225011_at |
| II, alpha | | | |
| Protein kinase, interferon-inducible double | PRKRA | Hs.405537 | 244047_at |
| stranded RNA dependent activator | | | |
| protein O-fucosyltransferase 2 | POFUT2 | Hs.517345 | 209578_s_at |
| protein phosphatase 1, catalytic subunit, beta | PPP1CB | Hs.468018 | 201408_at |
| isoform | | | |
| protein phosphatase 1, catalytic subunit, gamma | PPP1CC | Hs.79081 | 200726_at |
| isoform | | | |
| protein phosphatase 1, regulatory (inhibitor) | PPP1R11 | Hs.82887 | 201500_s_at |
| subunit 11 | | | |
| protein phosphatase 1, regulatory (inhibitor) | PPP1R2 | Hs.184840 | 202165_at |
| subunit 2 | | | |
| protein phosphatase 1, regulatory (inhibitor) | PPP1R2 | Hs.184840 | 202166_s_at |
| subunit 2 | | | |
| protein phosphatase 1, regulatory (inhibitor) | PPP1R2P9 | Hs.127689 | 207377_at |
| subunit 2 pseudogene 9 | | | |
| protein phosphatase 1, regulatory (inhibitor) | PPP1R9A | Hs.489160 | 221088_s_at |
| subunit 9A | | | |
| Protein phosphatase 1, regulatory subunit 7 | PPP1R7 | Hs.36587 | 1559054_a_at |
| protein phosphatase 1, regulatory subunit 7 | PPP1R7 | Hs.36587 | 201214_s_at |
| Protein phosphatase 1A (formerly 2C), | PPM1A | Hs.130036 | 231370_at |
| magnesium-dependent, alpha isoform | | | |
| protein phosphatase 1A (formerly 2C), | PPM1A | Hs.130036 | 203966_s_at |
| magnesium-dependent, alpha isoform /// protein | | | |
| phosphatase 1A (formerly 2C), magnesium- | | | |
| dependent, alpha isoform | | | |
| protein phosphatase 1B (formerly 2C), | PPM1B | Hs.416769 | 209296_at |
| magnesium-dependent, beta isoform | | | |
| protein phosphatase 1G (formerly 2C), | PPM1G | Hs.17883 | 200913_at |
| magnesium-dependent, gamma isoform | | | |
| protein phosphatase 1J (PP2C domain containing) | PPM1J | Hs.179735 | 229484_at |
| Protein phosphatase 2 (formerly 2A), catalytic | PPP2CA | Hs.483408 | 215628_x_at |
| subunit, alpha isoform | | | |
| protein phosphatase 2 (formerly 2A), regulatory | PPP2R2A | Hs.146339 | 202313_at |
| subunit B (PR 52), alpha isoform | | | |
| protein phosphatase 2 (formerly 2A), regulatory | PPP2R2A | Hs.146339 | 236492_at |
| subunit B (PR 52), alpha isoform | | | |
| protein phosphatase 2 (formerly 2A), regulatory | PPP2R2B | Hs.193825 | 213849_s_at |
| subunit B (PR 52), beta isoform | | | |
| protein phosphatase 2 (formerly 2A), regulatory | PPP2R3A | Hs.518155 | 207749_s_at |
| subunit B″, alpha | | | |
| protein phosphatase 2, regulatory subunit B (B56), | PPP2R5D | Hs.533308 | 202513_s_at |
| delta isoform | | | |
| protein phosphatase 2, regulatory subunit B (B56), | PPP2R5D | Hs.533308 | 211159_s_at |
| delta isoform | | | |
| protein phosphatase 2, regulatory subunit B (B56), | PPP2R5E | Hs.334868 | 203338_at |
| epsilon isoform | | | |
| Protein phosphatase 2, regulatory subunit B (B56), | PPP2R5E | Hs.334868 | 228070_at |
| epsilon isoform | | | |
| protein phosphatase 2, regulatory subunit B (B56), | PPP2R5E | Hs.334868 | 229322_at |
| epsilon isoform | | | |
| protein phosphatase 2, regulatory subunit B (B56), | PPP2R5C | Hs.368264 | 1557718_at |
| gamma isoform | | | |
| protein phosphatase 2, regulatory subunit B (B56), | PPP2R5C | Hs.368264 | 201877_s_at |
| gamma isoform | | | |
| Protein phosphatase 3 (formerly 2B), catalytic | PPP3CA | Hs.435512 | 229606_at |
| subunit, alpha isoform (calcineurin A alpha) | | | |
| protein phosphatase 3 (formerly 2B), regulatory | PPP3R1 | Hs.280604 | 204506_at |
| subunit B, 19 kDa, alpha isoform (calcineurin B, | | | |
| type I) | | | |
| Protein phosphatase 3 (formerly 2B), regulatory | PPP3R1 | Hs.280604 | 241786_at |
| subunit B, 19 kDa, alpha isoform (calcineurin B, | | | |
| type I) | | | |
| protein phosphatase 3 (formerly 2B), regulatory | PPP3R2 | Hs.151167 | 231304_at |
| subunit B, 19 kDa, beta isoform (calcineurin B, type | | | |
| II) | | | |
| protein phosphatase 4, regulatory subunit 1 | PPP4R1 | Hs.464595 | 201594_s_at |
| protein phosphatase 6, catalytic subunit | PPP6C | Hs.495128 | 203529_at |
| protein predicted by clone 23733 | HSU79274 | Hs.436618 | 204521_at |
| protein regulator of cytokinesis 1 | PRC1 | Hs.459362 | 218009_s_at |
| Protein tyrosine phosphatase type IVA, member 1 | PTP4A1 | Hs.227777 | 1559322_at |
| protein tyrosine phosphatase type IVA, member 1 | PTP4A1 | Hs.227777 | 200730_s_at |
| protein tyrosine phosphatase type IVA, member 1 | PTP4A1 | Hs.227777 | 200733_s_at |
| protein tyrosine phosphatase type IVA, member 2 | PTP4A2 | Hs.470477 | 208616_s_at |
| protein tyrosine phosphatase type IVA, member 2 | PTP4A2 | Hs.470477 | 208617_s_at |
| protein tyrosine phosphatase type IVA, member 3 | PTP4A3 | Hs.43666 | 209695_at |
| protein tyrosine phosphatase, mitochondrial 1 | PTPMT1 | Hs.429506 | 223808_s_at |
| protein tyrosine phosphatase, non-receptor type 1 | PTPN1 | Hs.417549 | 202716_at |
| protein tyrosine phosphatase, non-receptor type 11 | PTPN11 | Hs.506852 | 205868_s_at |
| (Noonan syndrome 1) | | | |
| Protein tyrosine phosphatase, non-receptor type | PTPN13 | Hs.436142 | 243792_x_at |
| 13 (APO-1/CD95 (Fas)-associated phosphatase) | | | |
| protein tyrosine phosphatase, non-receptor type 6 | PTPN6 | Hs.63489 | 206687_s_at |
| protein tyrosine phosphatase, non-receptor type | PTPNS1L2 | Hs.339789 | 232891_at |
| substrate 1-like 2 | | | |
| Protein tyrosine phosphatase, receptor type, A | VPS16 | Hs.269577 | 233313_at |
| protein tyrosine phosphatase, receptor type, f | PPFIA1 | Hs.530749 | 210235_s_at |
| polypeptide (PTPRF), interacting protein (liprin), | | | |
| alpha 1 | | | |
| protein tyrosine phosphatase, receptor type, f | PPFIA1 | Hs.530749 | 210236_at |
| polypeptide (PTPRF), interacting protein (liprin), | | | |
| alpha 1 | | | |
| protein tyrosine phosphatase, receptor type, M | PTPRM | Hs.49774 | 1555579_s_at |
| proteoglycan 1, secretory granule | PRG1 | Hs.1908 | 201859_at |
| prothymosin, alpha (gene sequence 28) | PTMA | Hs.459927 | 200772_x_at |
| prothymosin, alpha (gene sequence 28) /// | PTMA /// | Hs.459925 | 216515_x_at |
| hypothetical gene supported by BC013859 | LOC441454 | | |
| prothymosin, alpha (gene sequence 28) /// similar | PTMA /// | Hs.459925 | 200773_x_at |
| to prothymosin alpha /// hypothetical gene | LOC440085 | | |
| supported by BC013859 | /// | | |
| | LOC441454 | | |
| protocadherin 17 | PCDH17 | Hs.106511 | 205656_at |
| protocadherin 18 | PCDH18 | Hs.518989 | 225975_at |
| protocadherin alpha 9 | PCDHA9 | — | 208205_at |
| protocadherin gamma subfamily A, 4 | PCDHGA4 | — | 1552735_at |
| protocadherin LKC | PC-LKC | Hs.4205 | 220186_s_at |
| protoporphyrinogen oxidase | PPOX | Hs.517373 | 238117_at |
| PRP18 pre-mRNA processing factor 18 homolog | PRPF18 | Hs.161181 | 223685_s_at |
| (yeast) | | | |
| PRP18 pre-mRNA processing factor 18 homolog | PRPF18 | Hs.161181 | 232473_at |
| (yeast) | | | |
| PRP4 pre-mRNA processing factor 4 homolog B | PRPF4B | Hs.159014 | 202127_at |
| (yeast) | | | |
| prune homolog (Drosophila) | PRUNE | Hs.78524 | 209586_s_at |
| prune homolog (Drosophila) | PRUNE | Hs.78524 | 210988_s_at |
| PTD012 protein | PTD012 | Hs.8360 | 229851_s_at |
| PTD015 protein | PTD015 | Hs.503357 | 221600_s_at |
| PTEN induced putative kinase 1 | PINK1 | Hs.389171 | 209019_s_at |
| PTK2 protein tyrosine kinase 2 | PTK2 | Hs.395482 | 1559529_at |
| PTK2 protein tyrosine kinase 2 | PTK2 | Hs.395482 | 241453_at |
| PTK2 protein tyrosine kinase 2 | PTK2 | Hs.395482 | 242240_at |
| PTK7 protein tyrosine kinase 7 | PTK7 | Hs.90572 | 207011_s_at |
| PTK9 protein tyrosine kinase 9 | PTK9 | Hs.189075 | 201745_at |
| PTPRF interacting protein, binding protein 1 (liprin | PPFIBP1 | Hs.172445 | 1569464_at |
| beta 1) | | | |
| PTX1 protein | PTX1 | Hs.339453 | 226422_at |
| pumilio homolog 1 (Drosophila) | PUM1 | Hs.281707 | 201166_s_at |
| pumilio homolog 2 (Drosophila) | PUM2 | Hs.467824 | 216221_s_at |
| purinergic receptor P2Y, G-protein coupled, 5 | P2RY5 | Hs.123464 | 218589_at |
| purine-rich element binding protein A | PURA | Hs.443121 | 204020_at |
| purine-rich element binding protein B | PURB | Hs.349150 | 226762_at |
| purine-rich element binding protein B | PURB | Hs.349150 | 227718_at |
| purine-rich element binding protein G | PURG | Hs.373778 | 235634_at |
| putative acyl-CoA dehydrogenase | FLJ12592 | Hs.441378 | 225573_at |
| putative homeodomain transcription factor 1 | PHTF1 | Hs.486246 | 205702_at |
| putative homeodomain transcription factor 1 | PHTF1 | Hs.486246 | 210191_s_at |
| putative MAPK activating protein | MGC3794 | Hs.209431 | 1553677_a_at |
| putative membrane protein | LOC54499 | Hs.93832 | 208716_s_at |
| putative neuronal cell adhesion molecule | PUNC | Hs.512847 | 232037_at |
| putative nuclear protein ORF1-FL49 | ORF1- | Hs.483561 | 224707_at |
| | FL49 | | |
| Putative olfactory receptor (clone ht2) | — | Hs.129832 | 215463_at |
| putative small membrane protein NID67 | NID67 | Hs.29444 | 223276_at |
| putative translation initiation factor | SUI1 | Hs.150580 | 211956_s_at |
| pyridoxal (pyridoxine, vitamin B6) kinase | PDXK | Hs.284491 | 202671_s_at |
| pyridoxal (pyridoxine, vitamin B6) kinase | PDXK | Hs.284491 | 218018_at |
| pyridoxine 5′-phosphate oxidase | PNPO | Hs.514278 | 218511_s_at |
| pyruvate dehydrogenase kinase, isoenzyme 1 | PDK1 | Hs.470633 | 226452_at |
| Pyruvate dehydrogenase kinase, isoenzyme 3 | PDK3 | Hs.444422 | 221957_at |
| pyruvate dehydrogenase phosphatase regulatory | PDPR | Hs.461183 | 220236_at |
| subunit | | | |
| pyruvate dehydrogenase phosphatase regulatory | PDPR | Hs.461183 | 224902_at |
| subunit | | | |
| Pyruvate kinase, muscle | PKM2 | Hs.198281 | 213700_s_at |
| QIL1 protein | QIL1 | Hs.356626 | 225823_at |
| Quaking homolog, KH domain RNA binding | QKI | Hs.510324 | 241938_at |
| (mouse) | | | |
| quiescin Q6 | QSCN6 | Hs.518374 | 201482_at |
| Quinolinate phosphoribosyltransferase (nicotinate- | QPRT | Hs.513484 | 1564129_a_at |
| nucleotide pyrophosphorylase (carboxylating)) | | | |
| R3H domain (binds single-stranded nucleic acids) | R3HDM | Hs.412462 | 202754_at |
| containing | | | |
| Rab geranylgeranyltransferase, beta subunit | RABGGTB | Hs.78948 | 209180_at |
| RAB GTPase activating protein 1 | RABGAP1 | Hs.271341 | 204028_s_at |
| RAB GTPase activating protein 1 | RABGAP1 | Hs.271341 | 213313_at |
| RAB GTPase activating protein 1-like | RABGAP1L | Hs.495391 | 213982_s_at |
| RAB guanine nucleotide exchange factor (GEF) 1 | RABGEF1 | Hs.530053 | 218310_at |
| RAB interacting factor | RABIF | Hs.90875 | 204478_s_at |
| RAB11 family interacting protein 1 (class I) | RAB11FIP1 | Hs.191179 | 225177_at |
| RAB11 family interacting protein 2 (class I) | RAB11FIP2 | Hs.173656 | 203883_s_at |
| RAB11 family interacting protein 3 (class II) | RAB11FIP3 | Hs.531642 | 228613_at |
| RAB11 family interacting protein 5 (class I) | RAB11FIP5 | Hs.24557 | 210879_s_at |
| RAB13, member RAS oncogene family | RAB13 | Hs.151536 | 202252_at |
| RAB14, member RAS oncogene family | RAB14 | Hs.371563 | 211503_s_at |
| RAB18, member RAS oncogene family | RAB18 | Hs.406799 | 229398_at |
| RAB18, member RAS oncogene family /// RAB18, | RAB18 | Hs.406799 | 224377_s_at |
| member RAS oncogene family | | | |
| RAB1A, member RAS oncogene family | RAB1A | Hs.310645 | 207791_s_at |
| RAB1A, member RAS oncogene family | RAB1A | Hs.310645 | 208724_s_at |
| RAB1A, member RAS oncogene family | RAB1A | Hs.310645 | 213440_at |
| RAB22A, member RAS oncogene family | RAB22A | Hs.529044 | 237864_at |
| RAB27A, member RAS oncogene family | RAB27A | Hs.298651 | 209514_s_at |
| RAB3 GTPase-activating protein | RAB3GAP | Hs.306327 | 213530_at |
| RAB3 GTPase-activating protein | RAB3GAP | Hs.306327 | 213531_s_at |
| Rab3 GTPase-activating protein, non-catalytic | RAB3- | Hs.549128 | 202372_at |
| subunit (150 kD) | GAP150 | | |
| RAB31, member RAS oncogene family | RAB31 | Hs.99528 | 217762_s_at |
| RAB3A interacting protein (rabin3) | RAB3IP | Hs.258209 | 223471_at |
| RAB40C, member RAS oncogene family | RAB40C | Hs.459630 | 1569396_at |
| RAB5A, member RAS oncogene family | RAB5A | Hs.475663 | 209089_at |
| RAB5B, member RAS oncogene family | RAB5B | Hs.77690 | 201276_at |
| RAB6 interacting protein 2 | RAB6IP2 | Hs.400431 | 1552663_a_at |
| RAB6A, member RAS oncogene family | RAB6A | Hs.503222 | 201047_x_at |
| RAB7, member RAS oncogene family | RAB7 | Hs.15738 | 211961_s_at |
| RAB8A, member RAS oncogene family | RAB8A | Hs.5947 | 208819_at |
| RAB8B, member RAS oncogene family | RAB8B | Hs.389733 | 219210_s_at |
| Rac GTPase activating protein 1 | RACGAP1 | Hs.505469 | 222077_s_at |
| RAD17 homolog (S. pombe) | RAD17 | Hs.16184 | 207405_s_at |
| RAD17 homolog (S. pombe) | RAD17 | Hs.16184 | 211228_s_at |
| RAD21 homolog (S. pombe) | RAD21 | Hs.81848 | 200607_s_at |
| RAD23 homolog B (S. cerevisiae) | RAD23B | Hs.521640 | 201222_s_at |
| RAD51-like 3 (S. cerevisiae) | RAD51L3 | Hs.125244 | 209965_s_at |
| RAD51-like 3 (S. cerevisiae) | RAD51L3 | Hs.125244 | 37793_r_at |
| RAD9 homolog A (S. pombe) | RAD9A | Hs.240457 | 204828_at |
| radical S-adenosyl methionine and flavodoxin | RSAFD1 | Hs.520917 | 218347_at |
| domains 1 | | | |
| Radical S-adenosyl methionine and flavodoxin | RSAFD1 | Hs.520917 | 240800_x_at |
| domains 1 | | | |
| radical S-adenosyl methionine domain containing 2 | RSAD2 | Hs.17518 | 242625_at |
| RAE1 RNA export 1 homolog (S. pombe) | RAE1 | Hs.371698 | 201558_at |
| Ral GEF with PH domain and SH3 binding motif 2 | RALGPS2 | Hs.549173 | 220338_at |
| ralA binding protein 1 | RALBP1 | Hs.528993 | 202845_s_at |
| RAN binding protein 2 | RANBP2 | Hs.199561 | 201711_x_at |
| RAN binding protein 2 | RANBP2 | Hs.199561 | 201713_s_at |
| RAN binding protein 2-like 1 | RANBP2L1 | Hs.469630 | 210676_x_at |
| RAN binding protein 2-like 1 | RANBP2L1 | Hs.469630 | 211324_s_at |
| RAN binding protein 2-like 1 | RANBP2L1 | Hs.469630 | 232511_at |
| RAN binding protein 2-like 1 /// similar to Ran- | RANBP2L1 | Hs.469630 | 212842_x_at |
| binding protein 2 /// similar to RAN-binding protein | /// | | |
| 2-like 1 | DKFZp686P0288 | | |
| | /// | | |
| | LOC375258 | | |
| RAN binding protein 5 | RANBP5 | Hs.513057 | 211953_s_at |
| RAN binding protein 5 | RANBP5 | Hs.513057 | 211955_at |
| RAN binding protein 9 | RANBP9 | Hs.306242 | 202582_s_at |
| RAN binding protein 9 | RANBP9 | Hs.306242 | 202583_s_at |
| RAN binding protein 9 | RANBP9 | Hs.306242 | 242143_at |
| RAN binding protein 9 | RANBP9 | Hs.306242 | 244474_at |
| Ran GTPase activating protein 1 | RANGAP1 | Hs.183800 | 212125_at |
| Rap guanine nucleotide exchange factor (GEF) 2 | RAPGEF2 | Hs.113912 | 203096_s_at |
| Rap guanine nucleotide exchange factor (GEF) 6 | RAPGEF6 | — | 239438_at |
| RAP1 interacting factor homolog (yeast) | RIF1 | Hs.536537 | 241820_at |
| RAP1, GTP-GDP dissociation stimulator 1 | RAP1GDS1 | Hs.132858 | 209444_at |
| RAP1B, member of RAS oncogene family | RAP1B | Hs.369920 | 200833_s_at |
| rap2 interacting protein x | RIPX | Hs.7972 | 210251_s_at |
| RAP2A, member of RAS oncogene family | RAP2A | Hs.508480 | 225585_at |
| RAP2B, member of RAS oncogene family | RAP2B | Hs.98643 | 213923_at |
| RA-regulated nuclear matrix-associated protein | RAMP | Hs.126774 | 218585_s_at |
| RA-regulated nuclear matrix-associated protein | RAMP | Hs.126774 | 222680_s_at |
| RAR-related orphan receptor A | RORA | Hs.511626 | 210426_x_at |
| RAR-related orphan receptor A | RORA | Hs.511626 | 210479_s_at |
| RAS and EF hand domain containing | RASEF | Hs.129136 | 1553185_at |
| RAS and EF hand domain containing | RASEF | Hs.129136 | 1553186_x_at |
| Ras association (RalGDS/AF-6) and pleckstrin | RAPH1 | Hs.471162 | 243012_at |
| homology domains 1 | | | |
| Ras homolog enriched in brain | RHEB | Hs.283521 | 201453_x_at |
| Ras homolog enriched in brain | RHEB | Hs.283521 | 213404_s_at |
| Ras homolog enriched in brain | RHEB | Hs.283521 | 213409_s_at |
| ras homolog gene family, member A /// ras | RHOA | Hs.247077 | 200059_s_at |
| homolog gene family, member A | | | |
| ras homolog gene family, member F (in filopodia) | RHOF | Hs.223617 | 1554538_at |
| ras homolog gene family, member G (rho G) | RHOG | Hs.501728 | 203175_at |
| ras homolog gene family, member Q | RHOQ | Hs.549125 | 212117_at |
| ras homolog gene family, member Q | RHOQ | Hs.549125 | 212119_at |
| Ras homolog gene family, member Q | RHOQ | Hs.549125 | 212120_at |
| ras homolog gene family, member T1 | RHOT1 | Hs.462742 | 218323_at |
| ras responsive element binding protein 1 | RREB1 | Hs.298248 | 215032_at |
| ras responsive element binding protein 1 | RREB1 | Hs.298248 | 242297_at |
| Ras-associated protein Rap1 | RBJ | Hs.434993 | 223504_at |
| Ras-GTPase activating protein SH3 domain- | G3BP2 | Hs.303676 | 206383_s_at |
| binding protein 2 | | | |
| Ras-GTPase activating protein SH3 domain- | G3BP2 | Hs.303676 | 208840_s_at |
| binding protein 2 | | | |
| Ras-GTPase activating protein SH3 domain- | G3BP2 | Hs.303676 | 208841_s_at |
| binding protein 2 | | | |
| Ras-GTPase-activating protein SH3-domain- | G3BP | Hs.3353 | 225007_at |
| binding protein | | | |
| RAS-like, family 11, member A | RASL11A | Hs.192131 | 238353_at |
| Ras-related GTP binding C | RRAGC | Hs.532461 | 218088_s_at |
| RB1-inducible coiled-coil 1 | RB1CC1 | Hs.196102 | 202033_s_at |
| RB1-inducible coiled-coil 1 | RB1CC1 | Hs.196102 | 237626_at |
| RCD1 required for cell differentiation1 homolog (S. pombe) | RQCD1 | Hs.148767 | 213179_at |
| rearranged L-myc fusion sequence | RLF | Hs.205627 | 204243_at |
| receptor associated protein 80 | RAP80 | Hs.232721 | 220746_s_at |
| recombining binding protein suppressor of hairless | RBPSUH | Hs.479396 | 211974_x_at |
| (Drosophila) | | | |
| RecQ protein-like (DNA helicase Q1-like) | RECQL | Hs.235069 | 212917_x_at |
| reelin | RELN | Hs.132439 | 205923_at |
| regenerating islet-derived 3 alpha | REG3A | Hs.423 | 205815_at |
| regucalcin gene promotor region related protein | RGPR | Hs.451846 | 1555289_at |
| regulator of G-protein signalling 22 | RGS22 | Hs.120021 | 223691_at |
| regulatory factor X, 1 (influences HLA class II | RFX1 | Hs.73677 | 226786_at |
| expression) | | | |
| regulatory factor X, 3 (influences HLA class II | RFX3 | Hs.136829 | 207234_at |
| expression) | | | |
| Regulatory factor X, 3 (influences HLA class II | RFX3 | Hs.136829 | 217671_at |
| expression) | | | |
| Regulatory factor X, 3 (influences HLA class II | RFX3 | Hs.136829 | 240867_at |
| expression) | | | |
| regulatory solute carrier protein, family 1, member 1 | RSC1A1 | Hs.239459 | 214583_at |
| related RAS viral (r-ras) oncogene homolog 2 | RRAS2 | Hs.502004 | 212590_at |
| replication protein A1, 70 kDa | RPA1 | Hs.461925 | 201529_s_at |
| replication protein A2, 32 kDa | RPA2 | Hs.79411 | 201756_at |
| Replication protein A3, 14 kDa | RPA3 | Hs.487540 | 240601_at |
| RER1 retention in endoplasmic reticulum 1 | RER1 | Hs.525527 | 202296_s_at |
| homolog (S. cerevisiae) | | | |
| REST corepressor 1 | RCOR1 | Hs.510521 | 212612_at |
| REST corepressor 3 | RCOR3 | Hs.356399 | 218344_s_at |
| restin (Reed-Steinberg cell-expressed intermediate | RSN | Hs.524809 | 201975_at |
| filament-associated protein) | | | |
| Restin (Reed-Steinberg cell-expressed | RSN | Hs.524809 | 242287_at |
| intermediate filament-associated protein) | | | |
| restin-like 2 | RSNL2 | Hs.122927 | 219944_at |
| Restin-like 2 | RSNL2 | Hs.122927 | 242710_at |
| ret finger protein 2 | RFP2 | Hs.436922 | 203659_s_at |
| ret finger protein-like 3 | RFPL3 | Hs.167751 | 207936_x_at |
| ret finger protein-like 3 antisense | RFPL3S | Hs.274285 | 214409_at |
| ret finger protein-like 3 antisense /// ret finger | RFPL3S | Hs.274285 | 214408_s_at |
| protein-like 1 antisense | /// | | |
| | RFPL1S | | |
| reticulocalbin 1, EF-hand calcium binding domain | RCN1 | Hs.97887 | 201063_at |
| reticulocalbin 2, EF-hand calcium binding domain | RCN2 | Hs.79088 | 201485_s_at |
| reticulon 3 | RTN3 | Hs.473761 | 224564_s_at |
| reticulon 4 | RTN4 | Hs.429581 | 210968_s_at |
| reticulon 4 | RTN4 | Hs.429581 | 211509_s_at |
| reticulon 4 | RTN4 | Hs.429581 | 214629_x_at |
| Retinitis pigmentosa 9 (autosomal dominant) | RP9 | Hs.326805 | 227849_at |
| retinitis pigmentosa GTPase regulator | RPGR | Hs.61438 | 207624_s_at |
| retinitis pigmentosa GTPase regulator interacting | RPGRIP1 | Hs.126035 | 206608_s_at |
| protein 1 | | | |
| retinoblastoma binding protein 4 | RBBP4 | Hs.16003 | 210371_s_at |
| retinoblastoma binding protein 6 | RBBP6 | Hs.188553 | 1552329_at |
| retinoblastoma binding protein 6 | RBBP6 | Hs.188553 | 212781_at |
| retinoblastoma binding protein 6 | RBBP6 | Hs.188553 | 223802_s_at |
| retinoblastoma binding protein 7 | RBBP7 | Hs.495755 | 201092_at |
| retinoblastoma binding protein 8 | RBBP8 | Hs.546282 | 203344_s_at |
| retinoblastoma-associated factor 600 | RBAF600 | Hs.148078 | 211950_at |
| Retinoblastoma-associated factor 600 | RBAF600 | Hs.148078 | 240108_at |
| retinoblastoma-associated protein 140 | RAP140 | Hs.549121 | 209284_s_at |
| retinoblastoma-like 2 (p130) | RBL2 | Hs.513609 | 212331_at |
| retinoic acid induced 1 | RAI1 | Hs.552583 | 226143_at |
| Retinoic acid receptor responder (tazarotene | RARRES1 | Hs.131269 | 240936_at |
| induced) 1 | | | |
| retinoic acid receptor responder (tazarotene | RARRES3 | Hs.17466 | 204070_at |
| induced) 3 | | | |
| Retinoic acid receptor, alpha | RARA | Hs.535499 | 228037_at |
| retinoid X receptor, beta | RXRB | Hs.388034 | 209148_at |
| retinol dehydrogenase 10 (all-trans) | RDH10 | Hs.244940 | 227467_at |
| retinol dehydrogenase 14 (all-trans and 9-cis) | RDH14 | Hs.288880 | 222203_s_at |
| REV3-like, catalytic subunit of DNA polymerase | REV3L | Hs.232021 | 238736_at |
| zeta (yeast) | | | |
| Rho family GTPase 3 | RND3 | Hs.6838 | 212724_at |
| Rho GTPase activating protein 10 | ARHGAP10 | Hs.368631 | 239567_at |
| Rho GTPase activating protein 22 | ARHGAP22 | Hs.435063 | 1556460_a_at |
| Rho GTPase activating protein 23 | ARHGAP23 | Hs.374446 | 230196_x_at |
| Rho GTPase activating protein 26 | ARHGAP26 | Hs.293593 | 1557688_at |
| Rho GTPase activating protein 28 | ARHGAP28 | Hs.183114 | 227911_at |
| Rho GTPase activating protein 5 | ARHGAP5 | Hs.525287 | 242110_at |
| Rho guanine nucleotide exchange factor (GEF) 15 | ARHGEF15 | Hs.443109 | 217348_x_at |
| Rho guanine nucleotide exchange factor (GEF) 7 | ARHGEF7 | Hs.508738 | 1562271_x_at |
| Rho guanine nucleotide exchange factor (GEF) 7 | ARHGEF7 | Hs.508738 | 202547_s_at |
| Rho guanine nucleotide exchange factor (GEF) 7 | ARHGEF7 | Hs.508738 | 202548_s_at |
| rhomboid, veinlet-like 7 (Drosophila) | RHBDL7 | Hs.488827 | 222995_s_at |
| Rho-related BTB domain containing 3 | RHOBTB3 | Hs.445030 | 225202_at |
| ribonuclease H1 | RNASEH1 | Hs.502765 | 218497_s_at |
| ribonuclease, RNase A family, 11 (non-active) | RNASE11 | Hs.112761 | 231603_at |
| ribonuclease/angiogenin inhibitor | RNH | Hs.530687 | 1555529_at |
| ribonucleotide reductase M1 polypeptide | RRM1 | Hs.383396 | 201476_s_at |
| ribonucleotide reductase M1 polypeptide | RRM1 | Hs.383396 | 201477_s_at |
| ribonucleotide reductase M2 B (TP53 inducible) | RRM2B | Hs.512592 | 223342_at |
| ribonucleotide reductase M2 polypeptide | RRM2 | Hs.226390 | 209773_s_at |
| ribophorin II | RPN2 | Hs.370895 | 208689_s_at |
| ribosomal protein L10a | RPL10A | Hs.148340 | 229563_s_at |
| Ribosomal protein L10a | RPL10A | Hs.148340 | 242188_at |
| ribosomal protein L12 /// ribosomal protein L12 | RPL12 | Hs.408054 | 200088_x_at |
| ribosomal protein L13 | RPL13 | Hs.410817 | 212933_x_at |
| ribosomal protein L14 | RPL14 | Hs.446522 | 213588_x_at |
| ribosomal protein L15 | RPL15 | Hs.381219 | 221476_s_at |
| ribosomal protein L15 /// similar to ribosomal | RPL15 | Hs.381219 | 217266_at |
| protein L15 | /// | | |
| | LOC136321 | | |
| ribosomal protein L17 | RPL17 | Hs.374588 | 212270_x_at |
| ribosomal protein L21 /// ribosomal protein L21 | RPL21 | Hs.381123 | 200012_x_at |
| ribosomal protein L22 | RPL22 | Hs.515329 | 208768_x_at |
| ribosomal protein L22 | RPL22 | Hs.515329 | 214042_s_at |
| ribosomal protein L22 | RPL22 | Hs.515329 | 220960_x_at |
| ribosomal protein L22 | RPL22 | Hs.515329 | 221775_x_at |
| ribosomal protein L23 | RPL23 | Hs.406300 | 200888_s_at |
| ribosomal protein L23a | RPL23A | Hs.419463 | 203012_x_at |
| ribosomal protein L23a | RPL23A | Hs.419463 | 208825_x_at |
| ribosomal protein L23a | RPL23A | Hs.419463 | 208834_x_at |
| ribosomal protein L23a | RPL23A | Hs.419463 | 213084_x_at |
| ribosomal protein L26-like 1 | RPL26L1 | Hs.546390 | 218830_at |
| ribosomal protein L27 /// ribosomal protein L27 | RPL27 | Hs.514196 | 200025_s_at |
| ribosomal protein L30 /// ribosomal protein L30 | RPL30 | Hs.400295 | 200062_s_at |
| ribosomal protein L31 | RPL31 | Hs.469473 | 200963_x_at |
| ribosomal protein L34 /// ribosomal protein L34 /// | RPL34 | Hs.512199 | 200026_at |
| similar to ribosomal protein L34; 60S ribosomal | /// | | |
| protein L34 /// similar to ribosomal protein L34; 60S | LOC342994 | | |
| ribosomal protein L34 | | | |
| ribosomal protein L35 /// ribosomal protein L35 | RPL35 | Hs.182825 | 200002_at |
| ribosomal protein L35a | RPL35A | Hs.529631 | 213687_s_at |
| Ribosomal protein L35a | RPL35A | Hs.529631 | 215208_x_at |
| ribosomal protein L35a | RPL35A | Hs.529631 | 225190_x_at |
| ribosomal protein L37 /// ribosomal protein L37 | RPL37 | Hs.80545 | 200092_s_at |
| ribosomal protein L37a | RPL37A | Hs.433701 | 201429_s_at |
| ribosomal protein L38 | RPL38 | Hs.380953 | 202029_x_at |
| ribosomal protein L39 | RPL39 | Hs.300141 | 208695_s_at |
| Ribosomal protein L4 | RPL4 | Hs.432898 | 226190_at |
| Ribosomal protein L4 | RPL4 | Hs.432898 | 226556_at |
| ribosomal protein L4 /// ribosomal protein L4 | RPL4 | Hs.186350 | 200089_s_at |
| ribosomal protein L41 | RPL41 | Hs.381172 | 201492_s_at |
| ribosomal protein L5 | RPL5 | Hs.532359 | 200937_s_at |
| ribosomal protein L5 | RPL5 | Hs.532359 | 213080_x_at |
| ribosomal protein L6 /// ribosomal protein L6 | RPL6 | Hs.528668 | 200034_s_at |
| ribosomal protein L7 | RPL7 | Hs.421257 | 200717_x_at |
| ribosomal protein L7 | RPL7 | Hs.421257 | 212042_x_at |
| ribosomal protein L7a | RPL7A | Hs.499839 | 224930_x_at |
| ribosomal protein L7-like 1 /// similar to RPL7L1 | RPL7L1 | Hs.550564 | 233013_x_at |
| protein | /// | | |
| | LOC401197 | | |
| ribosomal protein L9 /// ribosomal protein L9 | RPL9 | Hs.412370 | 200032_s_at |
| ribosomal protein S10 | RPS10 | Hs.406620 | 200817_x_at |
| ribosomal protein S10 | RPS10 | Hs.406620 | 211542_x_at |
| Ribosomal protein S10 | RPS10 | Hs.406620 | 237703_at |
| ribosomal protein S10 /// ribosomal protein S10 | RPS10 | Hs.406620 | 200095_x_at |
| ribosomal protein S13 /// ribosomal protein S13 | RPS13 | Hs.446588 | 200018_at |
| ribosomal protein S14 | RPS14 | Hs.381126 | 208645_s_at |
| ribosomal protein S15a /// ADP-ribosylation factor- | RPS15A | Hs.370504 | 200781_s_at |
| like 6 interacting protein | /// | | |
| | ARL6IP | | |
| ribosomal protein S16 | RPS16 | Hs.397609 | 226131_s_at |
| ribosomal protein S20 | RPS20 | Hs.8102 | 200949_x_at |
| ribosomal protein S20 | RPS20 | Hs.8102 | 214003_x_at |
| ribosomal protein S21 | RPS21 | Hs.190968 | 200834_s_at |
| ribosomal protein S23 | RPS23 | Hs.386384 | 200926_at |
| ribosomal protein S25 /// ribosomal protein S25 | RPS25 | Hs.512676 | 200091_s_at |
| ribosomal protein S26 | RPS26 | Hs.355957 | 217753_s_at |
| ribosomal protein S27 (metallopanstimulin 1) | RPS27 | Hs.546291 | 200741_s_at |
| ribosomal protein S27-like | RPS27L | Hs.108957 | 218007_s_at |
| ribosomal protein S27-like | RPS27L | Hs.108957 | 222487_s_at |
| ribosomal protein S28 | RPS28 | Hs.153177 | 208904_s_at |
| ribosomal protein S29 | RPS29 | Hs.156367 | 201094_at |
| ribosomal protein S3 | RPS3 | Hs.546286 | 208692_at |
| ribosomal protein S3A | RPS3A | Hs.356572 | 201257_x_at |
| ribosomal protein S3A | RPS3A | Hs.356572 | 212391_x_at |
| ribosomal protein S3A /// ribosomal protein S3A | RPS3A | Hs.356572 | 200099_s_at |
| ribosomal protein S4, X-linked | RPS4X | Hs.446628 | 200933_x_at |
| ribosomal protein S4, X-linked | RPS4X | Hs.446628 | 213347_x_at |
| ribosomal protein S6 | RPS6 | Hs.408073 | 201254_x_at |
| ribosomal protein S6 | RPS6 | Hs.408073 | 209134_s_at |
| Ribosomal protein S6 | RPS6 | Hs.408073 | 238156_at |
| ribosomal protein S6 /// ribosomal protein S6 | RPS6 | Hs.408073 | 200081_s_at |
| ribosomal protein S6 kinase, 52 kDa, polypeptide 1 | RPS6KC1 | Hs.30352 | 218909_at |
| ribosomal protein S6 kinase, 90 kDa, polypeptide 2 | RPS6KA2 | Hs.147119 | 204906_at |
| ribosomal protein S6 kinase, 90 kDa, polypeptide 5 | RPS6KA5 | Hs.510225 | 204635_at |
| ribosomal protein S7 | RPS7 | Hs.534346 | 213941_x_at |
| ribosomal protein S8 | RPS8 | Hs.512675 | 200858_s_at |
| ribosomal protein, large, P0 | RPLP0 | Hs.448226 | 201033_x_at |
| ribosomal protein, large, P1 | RPLP1 | Hs.356502 | 200763_s_at |
| ribosome binding protein 1 homolog 180 kDa (dog) | RRBP1 | Hs.472213 | 201205_at |
| rififylin | RFFL | Hs.13680 | 1552649_a_at |
| rififylin | RFFL | Hs.13680 | 228980_at |
| ring finger and CHY zinc finger domain containing 1 | RCHY1 | Hs.48297 | 214281_s_at |
| ring finger and KH domain containing 1 | RKHD1 | Hs.436495 | 91816_f_at |
| ring finger and KH domain containing 2 | RKHD2 | Hs.465144 | 1556874_a_at |
| ring finger and KH domain containing 2 | RKHD2 | Hs.465144 | 222567_s_at |
| ring finger and KH domain containing 3 | RKHD3 | Hs.104744 | 223627_at |
| ring finger protein 11 | RNF11 | Hs.309641 | 208924_at |
| Ring finger protein 12 | RNF12 | Hs.122121 | 242121_at |
| ring finger protein 13 | RNF13 | Hs.12333 | 201779_s_at |
| Ring finger protein 13 | RNF13 | Hs.12333 | 237264_at |
| Ring finger protein 130 | RNF130 | Hs.484363 | 239886_at |
| ring finger protein 133 | RNF133 | Hs.549267 | 231373_at |
| Ring finger protein 138 pseudogene 1 | RNF138P1 | Hs.551596 | 243442_x_at |
| ring finger protein 139 | RNF139 | Hs.492751 | 209510_at |
| ring finger protein 14 | RNF14 | Hs.508993 | 201823_s_at |
| ring finger protein 141 | RNF141 | Hs.44685 | 219104_at |
| ring finger protein 146 | RNF146 | Hs.267120 | 223886_s_at |
| ring finger protein 148 | RNF148 | Hs.529656 | 242925_at |
| ring finger protein 168 | RNF168 | Hs.518396 | 1553127_a_at |
| ring finger protein 170 | RNF170 | Hs.491626 | 1554312_at |
| ring finger protein 19 | RNF19 | Hs.292882 | 223085_at |
| Ring finger protein 19 | RNF19 | Hs.292882 | 230599_at |
| ring finger protein 20 | RNF20 | Hs.168095 | 222683_at |
| ring finger protein 32 /// ring finger protein 32 | RNF32 | Hs.490715 | 220991_s_at |
| ring finger protein 34 | RNF34 | Hs.292804 | 219035_s_at |
| ring finger protein 38 | RNF38 | Hs.333503 | 218528_s_at |
| ring finger protein 38 | RNF38 | Hs.333503 | 222660_s_at |
| ring finger protein 7 | RNF7 | Hs.134623 | 218286_s_at |
| ring finger protein 7 /// ring finger protein 7 | RNF7 | Hs.134623 | 224394_at |
| ring finger protein 7 /// ring finger protein 7 | RNF7 | Hs.134623 | 224395_s_at |
| ring finger protein 7 /// ring finger protein 7 | RNF7 | Hs.134623 | 224439_x_at |
| RING1 and YY1 binding protein | RYBP | Hs.7910 | 201844_s_at |
| RING1 and YY1 binding protein | RYBP | Hs.7910 | 201846_s_at |
| RING1 and YY1 binding protein | RYBP | Hs.7910 | 237456_at |
| ring-box 1 | RBX1 | Hs.474949 | 218117_at |
| RIO kinase 3 (yeast) /// RIO kinase 3 (yeast) | RIOK3 | Hs.445511 | 202129_s_at |
| RIO kinase 3 (yeast) /// RIO kinase 3 (yeast) | RIOK3 | Hs.445511 | 202130_at |
| RNA binding motif protein 12 | RBM12 | Hs.246413 | 212168_at |
| RNA binding motif protein 15 | RBM15 | Hs.435947 | 1555762_s_at |
| RNA binding motif protein 15 | RBM15 | Hs.435947 | 219286_s_at |
| RNA binding motif protein 15B | RBM15B | Hs.476291 | 202689_at |
| RNA binding motif protein 15B | RBM15B | Hs.476291 | 226987_at |
| RNA binding motif protein 16 | RBM16 | Hs.227602 | 203250_at |
| RNA binding motif protein 17 | RBM17 | Hs.498548 | 224780_at |
| RNA binding motif protein 25 | RBM25 | Hs.531106 | 1557081_at |
| RNA binding motif protein 25 | RBM25 | Hs.531106 | 212027_at |
| RNA binding motif protein 27 | RBM27 | Hs.61441 | 225326_at |
| RNA binding motif protein 4 | RBM4 | Hs.533712 | 200997_at |
| RNA binding motif protein 5 | RBM5 | Hs.439480 | 201395_at |
| RNA binding motif protein 6 | RBM6 | Hs.188879 | 1556672_a_at |
| RNA binding motif protein 6 | RBM6 | Hs.188879 | 228030_at |
| RNA binding motif protein 8A | RBM8A | Hs.552581 | 214113_s_at |
| RNA binding motif protein 8A | RBM8A | Hs.552581 | 222443_s_at |
| RNA binding motif protein, X-linked | RBMX | Hs.380118 | 225310_at |
| RNA binding motif, single stranded interacting | RBMS3 | Hs.221436 | 206767_at |
| protein | | | |
| RNA binding motif, single stranded interacting | RBMS1 | Hs.470412 | 215127_s_at |
| protein 1 | | | |
| RNA binding motif, single stranded interacting | RBMS1 | Hs.470412 | 225265_at |
| protein 1 | | | |
| RNA binding protein S1, serine-rich domain /// | RNPS1 | Hs.355643 | 200060_s_at |
| RNA binding protein S1, serine-rich domain | | | |
| RNA binding protein with multiple splicing | RBPMS | Hs.334587 | 1557223_at |
| RNA binding protein with multiple splicing | RBPMS | Hs.334587 | 209487_at |
| RNA guanylyltransferase and 5′-phosphatase | RNGTT | Hs.127219 | 204207_s_at |
| RNA guanylyltransferase and 5′-phosphatase | RNGTT | Hs.127219 | 211387_x_at |
| RNA methyltransferase like 1 | RNMTL1 | Hs.182729 | 218993_at |
| RNA processing factor 1 | RPF1 | Hs.481202 | 218462_at |
| RNA, U17D small nucleolar | RNU17D | — | 215011_at |
| RNA-binding region (RNP1, RRM) containing 2 | RNPC2 | Hs.282901 | 208720_s_at |
| ROD1 regulator of differentiation 1 (S. pombe) | ROD1 | Hs.269988 | 207223_s_at |
| ropporin, rhophilin associated protein 1 | ROPN1 | Hs.458304 | 224191_x_at |
| ropporin, rhophilin associated protein 1 | ROPN1 | Hs.458304 | 231535_x_at |
| ropporin, rhophilin associated protein 1 | ROPN1 | Hs.458304 | 233203_at |
| ropporin, rhophilin associated protein 1B | ROPN1B | Hs.528203 | 220425_x_at |
| rotatin | RTTN | Hs.31931 | 1557388_at |
| rotavirus X protein associated with NSP3 | RoXaN | Hs.474970 | 206169_x_at |
| round spermatid basic protein 1 | RSBN1 | Hs.486285 | 222789_at |
| RP42 homolog | RP42 | Hs.104613 | 218583_s_at |
| RP42 homolog | RP42 | Hs.104613 | 222679_s_at |
| RP42 homolog | RP42 | Hs.104613 | 240150_at |
| RP42 homolog | RP42 | Hs.104613 | 242428_at |
| RPA interacting protein | RIP | Hs.550538 | 1556088_at |
| RPA interacting protein | RIP | Hs.550538 | 228183_s_at |
| RPE-spondin | RPESP | Hs.439040 | 235209_at |
| RPE-spondin | RPESP | Hs.439040 | 235210_s_at |
| RRN3 RNA polymerase I transcription factor | RRN3 | Hs.460078 | 216902_s_at |
| homolog (yeast) | | | |
| RUN and FYVE domain containing 2 | RUFY2 | Hs.549177 | 233191_at |
| RUN and TBC1 domain containing 1 | RUTBC1 | Hs.513861 | 217538_at |
| RUN and TBC1 domain containing 3 | RUTBC3 | Hs.474914 | 214779_s_at |
| runt-related transcription factor 3 | RUNX3 | Hs.170019 | 204197_s_at |
| RWD domain containing 1 | RWDD1 | Hs.532164 | 219598_s_at |
| ryanodine receptor 3 | RYR3 | Hs.549061 | 206306_at |
| RYK receptor-like tyrosine kinase | RYK | Hs.245869 | 214172_x_at |
| S100 calcium binding protein A10 (annexin II | S100A10 | Hs.143873 | 200872_at |
| ligand, calpactin I, light polypeptide (p11)) | | | |
| S100 calcium binding protein A10 (annexin II | S100A10 | Hs.143873 | 238909_at |
| ligand, calpactin I, light polypeptide (p11)) | | | |
| S100 calcium binding protein A8 (calgranulin A) | S100A8 | Hs.416073 | 202917_s_at |
| S-adenosylhomocysteine hydrolase | AHCY | Hs.388004 | 200903_s_at |
| S-adenosylhomocysteine hydrolase-like 1 | AHCYL1 | Hs.485365 | 200850_s_at |
| SAM and SH3 domain containing 1 | SASH1 | Hs.193133 | 240113_at |
| SAR1a gene homolog 1 (S. cerevisiae) | SARA1 | Hs.499960 | 201542_at |
| sarcoglycan, beta (43 kDa dystrophin-associated | SGCB | Hs.438953 | 205120_s_at |
| glycoprotein) | | | |
| sarcolipin | SLN | Hs.334629 | 205374_at |
| Sarcoma antigen NY-SAR-27 | FLJ10330 | Hs.342307 | 230270_at |
| sarcoma antigen NY-SAR-91 | FLJ11730 | Hs.17118 | 218165_at |
| scavenger receptor class B, member 2 | SCARB2 | Hs.349656 | 201647_s_at |
| scavenger receptor class B, member 2 | SCARB2 | Hs.349656 | 215754_at |
| scavenger receptor class B, member 2 | SCARB2 | Hs.349656 | 224983_at |
| Scm-like with four mbt domains 1 | SFMBT1 | Hs.343679 | 213370_s_at |
| scotin | SCOTIN | Hs.414579 | 222986_s_at |
| scrapie responsive protein 1 | SCRG1 | Hs.7122 | 205475_at |
| SCRL protein | SCRL | Hs.414175 | 240559_at |
| SCY1-like 2 (S. cerevisiae) | SCYL2 | Hs.506481 | 224960_at |
| SCY1-like 3 (S. cerevisiae) | PACE-1 | Hs.435560 | 231111_at |
| SDA1 domain containing 1 | SDAD1 | Hs.270107 | 218607_s_at |
| SDA1 domain containing 1 | SDAD1 | Hs.270107 | 228408_s_at |
| Sec1 family domain containing 1 | SCFD1 | Hs.369168 | 241775_at |
| sec1 family domain containing 2 | SCFD2 | Hs.302287 | 226923_at |
| SEC10-like 1 (S. cerevisiae) | SEC10L1 | Hs.365863 | 225084_at |
| SEC11-like 1 (S. cerevisiae) | SEC11L1 | Hs.9534 | 201290_at |
| SEC14 and spectrin domains 1 | SESTD1 | Hs.370668 | 226763_at |
| SEC14-like 1 (S. cerevisiae) | SEC14L1 | Hs.464184 | 202082_s_at |
| SEC14-like 1 (S. cerevisiae) | SEC14L1 | Hs.464184 | 202083_s_at |
| SEC15-like 2 (S. cerevisiae) | SEC15L2 | Hs.303454 | 1569200_at |
| SEC15-like 2 (S. cerevisiae) | SEC15L2 | Hs.303454 | 1569202_x_at |
| SEC22 vesicle trafficking protein-like 1 (S. cerevisiae) | SEC22L1 | Hs.534212 | 214257_s_at |
| SEC22 vesicle trafficking protein-like 2 (S. cerevisiae) | SEC22L2 | Hs.477361 | 218703_at |
| SEC22 vesicle trafficking protein-like 3 (S. cerevisiae) | SEC22L3 | Hs.445892 | 225460_at |
| Sec23 homolog B (S. cerevisiae) | SEC23B | Hs.369373 | 210293_s_at |
| SEC23 interacting protein | SEC23IP | Hs.435004 | 209175_at |
| SEC24 related gene family, member B (S. cerevisiae) | SEC24B | Hs.292472 | 1569713_at |
| SEC24 related gene family, member C (S. cerevisiae) | SEC24C | Hs.81964 | 202361_at |
| SEC31-like 1 (S. cerevisiae) | SEC31L1 | Hs.370024 | 210616_s_at |
| SEC31-like 1 (S. cerevisiae) | SEC31L1 | Hs.370024 | 215009_s_at |
| SEC3-like 1 (S. cerevisiae) | SEC3L1 | Hs.269665 | 222127_s_at |
| Sec61 alpha 2 subunit (S. cerevisiae) | SEC61A2 | Hs.112955 | 228747_at |
| Sec61 alpha 2 subunit (S. cerevisiae) | SEC61A2 | Hs.112955 | 230215_at |
| Sec61 beta subunit | SEC61B | Hs.191887 | 203133_at |
| SEC63-like (S. cerevisiae) | SEC63 | Hs.529957 | 201916_s_at |
| SEC8-like 1 (S. cerevisiae) | SEC8L1 | Hs.321273 | 224926_at |
| SEC8-like 1 (S. cerevisiae) | SEC8L1 | Hs.321273 | 239264_at |
| SECIS binding protein 2 | SECISBP2 | Hs.59804 | 218265_at |
| SECIS binding protein 2 | SECISBP2 | Hs.59804 | 224250_s_at |
| secretory carrier membrane protein 1 | SCAMP1 | Hs.482587 | 1552978_a_at |
| secretory carrier membrane protein 1 | SCAMP1 | Hs.482587 | 212416_at |
| selenocysteine lyase | SCLY | Hs.512606 | 221575_at |
| selenocysteine lyase | SCLY | Hs.512606 | 59705_at |
| selenophosphate synthetase 1 | SEPHS1 | Hs.124027 | 208939_at |
| selenophosphate synthetase 2 | SEPHS2 | Hs.118725 | 200961_at |
| selenoprotein K | SELK | Hs.58471 | 223070_at |
| selenoprotein O | SELO | Hs.365405 | 233168_s_at |
| selenoprotein W, 1 | SEPW1 | Hs.529132 | 1555851_s_at |
| selenoprotein W, 1 | SEPW1 | Hs.529132 | 201194_at |
| sema domain, immunoglobulin domain (Ig), short | SEMA3C | Hs.269109 | 203788_s_at |
| basic domain, secreted, (semaphorin) 3C | | | |
| Sema domain, transmembrane domain (TM), and | SEMA6A | Hs.156967 | 215479_at |
| cytoplasmic domain, (semaphorin) 6A | | | |
| septin 10 | 10-Sep | Hs.469615 | 214720_x_at |
| septin 2 | 2-Sep | Hs.335057 | 200778_s_at |
| septin 2 /// septin 2 | 2-Sep | Hs.335057 | 200015_s_at |
| septin 7 | 7-Sep | Hs.191346 | 213151_s_at |
| serine (or cysteine) proteinase inhibitor, clade B | SERPINB11 | Hs.350958 | 1552463_at |
| (ovalbumin), member 11 | | | |
| serine (or cysteine) proteinase inhibitor, clade B | SERPINB8 | Hs.368077 | 1554616_at |
| (ovalbumin), member 8 | | | |
| serine (or cysteine) proteinase inhibitor, clade E | SERPINE2 | Hs.38449 | 212190_at |
| (nexin, plasminogen activator inhibitor type 1), | | | |
| member 2 | | | |
| Serine (or cysteine) proteinase inhibitor, clade E | SERPINE2 | Hs.38449 | 236599_at |
| (nexin, plasminogen activator inhibitor type 1), | | | |
| member 2 | | | |
| Serine hydroxymethyltransferase 1 (soluble) | SHMT1 | Hs.513987 | 227305_s_at |
| serine palmitoyltransferase, long chain base | SPTLC1 | Hs.90458 | 1554053_at |
| subunit 1 | | | |
| serine PI Kazal type 5-like 3 | SPINK5L3 | Hs.483771 | 233340_at |
| serine protease inhibitor-like, with Kunitz and WAP | SPINLW1 | Hs.121084 | 206318_at |
| domains 1 (eppin) | | | |
| serine racemase | SRR | Hs.461954 | 219205_at |
| serine threonine kinase 39 (STE20/SPS1 homolog, | STK39 | Hs.276271 | 202786_at |
| yeast) | | | |
| serine/arginine repetitive matrix 1 | SRRM1 | Hs.18192 | 201224_s_at |
| serine/arginine repetitive matrix 1 | SRRM1 | Hs.18192 | 201225_s_at |
| serine/arginine repetitive matrix 2 | SRRM2 | Hs.433343 | 207435_s_at |
| serine/arginine repetitive matrix 2 | SRRM2 | Hs.433343 | 208610_s_at |
| serine/threonine kinase 17a (apoptosis-inducing) | STK17A | Hs.268887 | 202693_s_at |
| serine/threonine kinase 19 | STK19 | Hs.485102 | 36019_at |
| serine/threonine kinase 24 (STE20 homolog, | STK24 | Hs.508514 | 208855_s_at |
| yeast) | | | |
| Serine/threonine kinase 25 (STE20 homolog, | STK25 | Hs.516807 | 242932_at |
| yeast) | | | |
| Serine/threonine kinase 3 (STE20 homolog, yeast) | STK3 | Hs.492333 | 233779_x_at |
| serine/threonine kinase 33 | STK33 | Hs.501833 | 228086_at |
| serine/threonine kinase 35 | STK35 | Hs.100057 | 225648_at |
| serine/threonine kinase 35 | STK35 | Hs.100057 | 225649_s_at |
| serine/threonine kinase 38 like | STK38L | Hs.184523 | 212572_at |
| serine/threonine kinase 4 | STK4 | Hs.472838 | 1569791_at |
| serine/threonine kinase 4 | STK4 | Hs.472838 | 223746_at |
| serine/threonine kinase 4 | STK4 | Hs.472838 | 236259_at |
| serine/threonine kinase 4 | STK4 | Hs.472838 | 243981_at |
| serine/threonine kinase receptor associated | STRAP | Hs.504895 | 200870_at |
| protein | | | |
| serologically defined breast cancer antigen 84 | SDBCAG84 | Hs.472558 | 216032_s_at |
| serologically defined colon cancer antigen 1 | SDCCAG1 | Hs.388584 | 1569594_a_at |
| serologically defined colon cancer antigen 10 | SDCCAG10 | Hs.371372 | 1555495_a_at |
| serologically defined colon cancer antigen 8 | SDCCAG8 | Hs.549104 | 243772_at |
| SERTA domain containing 1 | SERTAD1 | Hs.269898 | 223394_at |
| SERTA domain containing 2 | SERTAD2 | Hs.77293 | 202656_s_at |
| SERTA domain containing 3 | SERTAD3 | Hs.515412 | 219382_at |
| serum amyloid A4, constitutive | SAA4 | Hs.512677 | 207096_at |
| Sestrin 1 | SESN1 | Hs.59554 | 1556287_a_at |
| sestrin 3 | SESN3 | Hs.120633 | 235683_at |
| SET binding factor 2 | SBF2 | Hs.438794 | 233914_s_at |
| SET domain, bifurcated 2 | SETDB2 | Hs.446217 | 235338_s_at |
| SET translocation (myeloid leukemia-associated) | SET | Hs.436687 | 200630_x_at |
| SET translocation (myeloid leukemia-associated) | SET | Hs.436687 | 210231_x_at |
| seven in absentia homolog 1 (Drosophila) | SIAH1 | Hs.295923 | 202981_x_at |
| sex comb on midleg-like 1 (Drosophila) | SCML1 | Hs.109655 | 222747_s_at |
| SFRS protein kinase 1 | SRPK1 | Hs.443861 | 202199_s_at |
| SFRS protein kinase 1 | SRPK1 | Hs.443861 | 202200_s_at |
| SFRS protein kinase 2 | SRPK2 | Hs.285197 | 1558254_s_at |
| SFRS protein kinase 2 | SRPK2 | Hs.285197 | 203181_x_at |
| SFRS protein kinase 2 | SRPK2 | Hs.285197 | 203182_s_at |
| SFRS protein kinase 2 | SRPK2 | Hs.285197 | 214931_s_at |
| SFRS protein kinase 2 | SRPK2 | Hs.285197 | 230091_at |
| SFRS protein kinase 2 | SRPK2 | Hs.285197 | 239811_at |
| SGT1, suppressor of G2 allele of SKP1 (S. cerevisiae) | SUGT1 | Hs.281902 | 223329_x_at |
| SGT1, suppressor of G2 allele of SKP1 (S. cerevisiae) | SUGT1 | Hs.281902 | 224309_s_at |
| SH2 domain containing 4B | SH2D4B | Hs.147643 | 1563849_at |
| SH3 and multiple ankyrin repeat domains 2 | SHANK2 | Hs.268726 | 213307_at |
| SH3 multiple domains 1 | SH3MD1 | Hs.159368 | 224817_at |
| SH3 multiple domains 2 | SH3MD2 | Hs.301804 | 225589_at |
| SH3-domain GRB2-like 3 | SH3GL3 | Hs.270055 | 205636_at |
| SH3-domain GRB2-like 3 | SH3GL3 | Hs.270055 | 205637_s_at |
| SH3-domain GRB2-like endophilin B1 | SH3GLB1 | Hs.136309 | 209090_s_at |
| SH3-domain kinase binding protein 1 | SH3KBP1 | Hs.444770 | 1553588_at |
| SH3-domain kinase binding protein 1 | SH3KBP1 | Hs.444770 | 223082_at |
| shugoshin-like 1 (S. pombe) | SGOL1 | Hs.105153 | 1553690_at |
| shugoshin-like 2 (S. pombe) | SGOL2 | Hs.44269 | 235425_at |
| sialic acid binding Ig-like lectin 6 | SIGLEC6 | Hs.397255 | 210796_x_at |
| sialoadhesin | SN | Hs.31869 | 44673_at |
| sialophorin (gpL115, leukosialin, CD43) | SPN | Hs.461934 | 1568964_x_at |
| SID1 transmembrane family, member 1 | SIDT1 | Hs.272416 | 219734_at |
| SID1 transmembrane family, member 2 | SIDT2 | Hs.410977 | 56256_at |
| sidekick homolog 1 (chicken) | SDK1 | Hs.155959 | 229912_at |
| sideroflexin 4 | SFXN4 | Hs.397062 | 225143_at |
| signal peptidase complex subunit 1 homolog (S. cerevisiae) | SPCS1 | Hs.11125 | 217927_at |
| signal peptidase complex subunit 2 homolog (S. cerevisiae) | SPCS2 | Hs.282700 | 201240_s_at |
| signal peptidase complex subunit 3 homolog (S. cerevisiae) | SPCS3 | Hs.42194 | 218817_at |
| signal peptide peptidase-like 2A | SPPL2A | Hs.401537 | 226353_at |
| signal recognition particle 14 kDa (homologous Alu | SRP14 | Hs.533732 | 200007_at |
| RNA binding protein) /// signal recognition particle | | | |
| 14 kDa (homologous Alu RNA binding protein) | | | |
| signal recognition particle 19 kDa | SRP19 | Hs.483090 | 205335_s_at |
| signal recognition particle 54 kDa | SRP54 | Hs.167535 | 203605_at |
| signal recognition particle 68 kDa | SRP68 | Hs.514495 | 224607_s_at |
| signal recognition particle 72 kDa | SRP72 | Hs.237825 | 208801_at |
| Signal recognition particle 72 kDa | SRP72 | Hs.237825 | 235833_at |
| signal recognition particle 9 kDa | SRP9 | Hs.511425 | 201273_s_at |
| signal recognition particle receptor, B subunit | SRPRB | Hs.12152 | 218140_x_at |
| Signal recognition particle receptor, B subunit | SRPRB | Hs.12152 | 227737_at |
| signal sequence receptor, alpha (translocon- | SSR1 | Hs.114033 | 200891_s_at |
| associated protein alpha) | | | |
| Signal sequence receptor, alpha (translocon- | SSR1 | Hs.114033 | 225435_at |
| associated protein alpha) | | | |
| signal sequence receptor, gamma (translocon- | SSR3 | Hs.518346 | 222411_s_at |
| associated protein gamma) | | | |
| signal transducer and activator of transcription 2, | STAT2 | Hs.530595 | 205170_at |
| 113 kDa | | | |
| signal transducer and activator of transcription 3 | STAT3 | Hs.463059 | 208991_at |
| (acute-phase response factor) | | | |
| signal transducer and activator of transcription 3 | STAT3 | Hs.463059 | 243213_at |
| (acute-phase response factor) | | | |
| signal transducer and activator of transcription 4 | STAT4 | Hs.80642 | 206118_at |
| signal-induced proliferation-associated 1 like 2 | SIPA1L2 | Hs.268774 | 233587_s_at |
| signal-transducing adaptor protein-2 | STAP2 | Hs.194385 | 221610_s_at |
| Similar to 40S ribosomal protein S3 | — | Hs.461453 | 1556595_at |
| Similar to 60S ribosomal protein L23a | — | Hs.440962 | 229420_at |
| similar to 60S ribosomal protein L7a (Surfeit locus | LOC441422 | Hs.99081 | 236453_at |
| protein 3) (PLA-X polypeptide) | | | |
| similar to Ab2-183 | LOC158830 | Hs.443936 | 231377_at |
| Similar to amyloid beta (A4) precursor protein- | — | Hs.178186 | 243971_x_at |
| binding, family B, member 1 interacting protein; | | | |
| proline-rich protein 73; Rap1-interacting adaptor | | | |
| molecule | | | |
| Similar to ankyrin repeat domain 20A | — | Hs.552629 | 242766_at |
| Similar to ankyrin-repeat protein Nrarp | MGC61598 | Hs.535075 | 226499_at |
| similar to aquaporin 11 /// aquaporin 12 | LOC285192 | Hs.534650 | 1554344_s_at |
| | /// | | |
| | AQP12 | | |
| similar to aspartate beta hydroxylase (ASPH) | LOC57168 | Hs.550525 | 227014_at |
| similar to aspartate beta hydroxylase (ASPH) | LOC57168 | Hs.550525 | 227015_at |
| similar to CDC10 (cell division cycle 10, | LOC441601 | Hs.144683 | 214381_at |
| S. cerevisiae, homolog) | | | |
| similar to cell division cycle 10 | LOC285961 | Hs.406568 | 1556222_at |
| similar to cell division cycle 10 homolog | LOC442537 | — | 228147_at |
| similar to cell division cycle 10 homolog | LOC441220 | Hs.520804 | 230355_at |
| similar to centaurin, gamma-like family, member 1; | LOC439983 | Hs.364233 | 221971_x_at |
| ARF GTPase-activating protein; Em: AC012044.1 | | | |
| similar to CG12314 gene product | LOC201164 | Hs.31652 | 227037_at |
| similar to CG32662-PA | MGC88374 | Hs.412138 | 237205_at |
| similar to CG5435-PA | LOC127003 | Hs.446962 | 231044_at |
| similar to CG6405 gene product | LOC137392 | Hs.125038 | 235391_at |
| Similar to Cohesin subunit SA-3 (Stromal antigen | — | Hs.488746 | 243960_x_at |
| 3) (Stromalin 3) (SCC3 homolog 3) | | | |
| similar to dJ753D5.2 (novel protein similar to | LOC442216 | — | 216348_at |
| RPS17 (40S ribosomal protein S17)) | | | |
| similar to DNA segment, Chr 11, Brigham & | MGC71993 | Hs.206824 | 224573_at |
| Womens Genetics 0434 expressed | | | |
| similar to eukaryotic translation elongation factor 1 | LOC389223 | Hs.531473 | 213583_x_at |
| alpha 1-like 14 /// similar to eukaryotic translation | /// | | |
| elongation factor 1 alpha 1; eukaryotic translation | LOC440595 | | |
| elongation factor 1 alpha 1-like 14; CTCL tumor | | | |
| antigen; translation elongation factor 1 alpha 1-like | | | |
| 14; prostate tumor-inducing protein 1; EF1a-like | | | |
| protein; gl . . . | | | |
| similar to F-box only protein 25 /// similar to F-box | LOC159176 | Hs.471887 | 232953_at |
| only protein 25 /// similar to bA476I15.3 (novel | /// | | |
| protein similar to septin) /// similar to bA476I15.3 | LOC388578 | | |
| (novel protein similar to septin) /// similar to | /// | | |
| bA476I15.3 (novel protein similar to septin) /// | LOC400725 | | |
| similar to cytoskeletal beta actin /// similar to | /// | | |
| bA476I15.3 (novel protein similar to septin) /// | LOC440546 | | |
| similar to bA476I15.3 (novel protein similar to | /// | | |
| septin) | LOC440550 | | |
| | /// | | |
| | LOC440720 | | |
| | /// | | |
| | LOC440739 | | |
| | /// | | |
| | LOC440773 | | |
| Similar to F-box only protein 25 isoform 3; F-box | — | Hs.446531 | 1562546_at |
| protein Fbx25; F-box only protein 25 | | | |
| Similar to fer-1 like protein 3 | LOC90342 | Hs.534025 | 1561871_at |
| similar to FKSG60 | LOC441067 | — | 224288_x_at |
| similar to FLJ16518 protein | LOC440288 | Hs.458905 | 239792_at |
| similar to FLJ32921 protein | LOC153441 | Hs.127963 | 231434_at |
| similar to FLJ43276 protein | LOC440302 | Hs.546645 | 244498_x_at |
| Similar to FRG1 protein (FSHD region gene 1 | MGC72104 | Hs.529357 | 234949_at |
| protein) | | | |
| Similar to GA binding protein transcription factor, | — | Hs.520882 | 1561916_at |
| alpha subunit (60 kD); GA-binding protein | | | |
| transcription factor, alpha subunit (60 kD); human | | | |
| nuclear respiratory factor-2 subunit alpha | | | |
| similar to Golgi autoantigen, golgin subfamily A | LOC388137 | Hs.458932 | 232813_s_at |
| member 6 (Golgin linked to PML) (Golgin-like | | | |
| protein) | | | |
| Similar to hypothetical protein | — | Hs.496753 | 231043_at |
| Similar to hypothetical protein 628 | — | Hs.112420 | 230314_at |
| similar to hypothetical protein 9630041N07 | DKFZp686E2433 | Hs.445740 | 230421_at |
| Similar to hypothetical protein BC009489 | — | Hs.512214 | 239359_at |
| similar to hypothetical protein FLJ10883 | LOC115294 | Hs.308480 | 232382_s_at |
| Similar to hypothetical protein FLJ10883 | LOC115294 | Hs.308480 | 238902_at |
| similar to hypothetical protein FLJ36144 /// similar | LOC440243 | Hs.534889 | 1561171_a_at |
| to hypothetical protein FLJ36144 | /// | | |
| | LOC440247 | | |
| Similar to hypothetical protein FLJ38281 | — | Hs.296731 | 233775_x_at |
| Similar to hypothetical protein FLJ38374 | — | Hs.516952 | 215487_x_at |
| Similar to hypothetical protein FLJ38374 | — | Hs.516952 | 222205_x_at |
| Similar to Hypothetical protein KIAA0563 | — | Hs.462543 | 1562408_at |
| Similar to hypothetical protein MGC27019 | LOC285458 | Hs.487562 | 227330_x_at |
| similar to hypothetical protein MGC39725 | LOC389124 | Hs.517992 | 231525_at |
| similar to hypothetical protein MGC50809 | LOC440956 | Hs.444316 | 236661_at |
| Similar to hypothetical protein, MGC: 7199 | — | Hs.380803 | 229640_x_at |
| similar to hypothetical testis protein from macaque | LOC352909 | Hs.351582 | 230033_at |
| similar to implantation-associated protein | LOC388620 | Hs.527515 | 234763_at |
| Similar to KIAA0454 protein | — | Hs.429365 | 227384_s_at |
| similar to KIAA0752 protein | LOC389347 | Hs.368516 | 215133_s_at |
| similar to KIF27C | LOC389765 | Hs.160561 | 230815_at |
| Similar to large low complexity protein with | — | Hs.549386 | 1553538_s_at |
| proline/alanine-rich repeat | | | |
| similar to MGC52679 protein | LOC440836 | Hs.531314 | 238327_at |
| Similar to Microneme antigen | — | Hs.550578 | 222380_s_at |
| Similar to microtubule-associated proteins 1A/1B | — | Hs.213541 | 212114_at |
| light chain 3 | | | |
| similar to mouse 1700027M21Rik gene | LOC493861 | — | 231292_at |
| similar to mouse 2310016A09Rik gene | LOC134147 | Hs.192586 | 234981_x_at |
| similar to OK/SW-CL.16 | LOC440552 | Hs.549387 | 238199_x_at |
| Similar to phosphoglucomutase 5 | — | Hs.178400 | 231024_at |
| similar to protein of fungal metazoan origin like | LOC440574 | Hs.466662 | 224867_at |
| (11.1 kD)(2C514) | | | |
| similar to RIKEN cDNA 1700016G05 | LOC136242 | Hs.98947 | 231572_at |
| Similar to RIKEN cDNA 2700049P18 gene | MGC57827 | Hs.339665 | 225834_at |
| similar to RIKEN cDNA 4632412N22 gene | LOC165186 | Hs.47647 | 236717_at |
| similar to RIKEN cDNA 4921524J17 | LOC388272 | Hs.406551 | 226608_at |
| similar to RIKEN cDNA 4930549C01 | LOC127309 | Hs.236896 | 231551_at |
| similar to RIKEN cDNA A630077B13 gene; RIKEN | LOC441168 | Hs.381220 | 229390_at |
| cDNA 2810048G17 | | | |
| Similar to RIKEN cDNA E030024N20 gene | — | Hs.142074 | 235536_at |
| similar to Rpl7a protein | LOC442159 | Hs.535174 | 234512_x_at |
| Similar to RUN and FYVE domain-containing 2; | — | Hs.518801 | 227802_at |
| Run- and FYVE-domain containing protein | | | |
| similar to splicing factor, arginine/serine-rich 4 | FLJ11021 | Hs.432996 | 202301_s_at |
| similar to splicing factor, arginine/serine-rich 4 | FLJ11021 | Hs.432996 | 202302_s_at |
| Similar to Ten-m2 | — | Hs.484029 | 1560525_at |
| similar to transcription factor BTF3 | MGC23908 | Hs.429839 | 225976_at |
| similar to ubiquitin binding protein | UBPH | — | 205687_at |
| similar to ubiquitin binding protein | UBPH | — | 224878_at |
| similar to Zinc finger protein Rlf (Rearranged L-myc | LOC440971 | Hs.167614 | 1556743_at |
| fusion gene protein) (Zn-15 related protein) | | | |
| similar to Zinc finger protein Rlf (Rearranged L-myc | LOC440971 | Hs.167614 | 1556744_a_at |
| fusion gene protein) (Zn-15 related protein) | | | |
| single stranded DNA binding protein 3 | SSBP3 | Hs.175551 | 217991_x_at |
| single stranded DNA binding protein 3 | SSBP3 | Hs.175551 | 223635_s_at |
| Single stranded DNA binding protein 3 | SSBP3 | Hs.175551 | 232469_x_at |
| Single-stranded DNA binding protein 2 | SSBP2 | Hs.102735 | 1557813_at |
| sirtuin (silent mating type information regulation 2 | SIRT1 | Hs.369779 | 218878_s_at |
| homolog) 1 (S. cerevisiae) | | | |
| Sirtuin (silent mating type information regulation 2 | SIRT2 | Hs.466693 | 1558331_at |
| homolog) 2 (S. cerevisiae) | | | |
| sirtuin (silent mating type information regulation 2 | SIRT5 | Hs.282331 | 219185_at |
| homolog) 5 (S. cerevisiae) | | | |
| Sjogren syndrome antigen A2 (60 kDa, | SSA2 | Hs.288178 | 210438_x_at |
| ribonucleoprotein autoantigen SS-A/Ro) | | | |
| Sjogren syndrome antigen A2 (60 kDa, | SSA2 | Hs.288178 | 212852_s_at |
| ribonucleoprotein autoantigen SS-A/Ro) | | | |
| SKB1 homolog (S. pombe) | SKB1 | Hs.367854 | 1556899_at |
| SKI interacting protein | SKIIP | Hs.445498 | 201575_at |
| SLAC2-B | SLAC2-B | Hs.269591 | 214734_at |
| SLAM family member 6 | SLAMF6 | Hs.492348 | 1552497_a_at |
| SLIT-ROBO Rho GTPase activating protein 2 | SRGAP2 | Hs.497575 | 227649_s_at |
| SMA4 | SMA4 | Hs.482411 | 214850_at |
| SMA4 | SMA4 | Hs.482411 | 238446_at |
| SMAD specific E3 ubiquitin protein ligase 2 | SMURF2 | Hs.515011 | 205596_s_at |
| SMAD, mothers against DPP homolog 1 | SMAD1 | Hs.549050 | 210993_s_at |
| (Drosophila) | | | |
| SMAD, mothers against DPP homolog 1 | SMAD1 | Hs.549050 | 227798_at |
| (Drosophila) | | | |
| SMAD, mothers against DPP homolog 2 | SMAD2 | Hs.12253 | 203075_at |
| (Drosophila) | | | |
| SMAD, mothers against DPP homolog 2 | SMAD2 | Hs.12253 | 203076_s_at |
| (Drosophila) | | | |
| SMAD, mothers against DPP homolog 4 | SMAD4 | Hs.75862 | 235725_at |
| (Drosophila) | | | |
| small acidic protein /// small acidic protein | SMAP | Hs.546339 | 200084_at |
| small EDRK-rich factor 2 /// Huntingtin interacting | SERF2 | Hs.424126 | 233746_x_at |
| protein K | /// HYPK | | |
| small fragment nuclease | DKFZP566E144 | Hs.7527 | 218194_at |
| small membrane protein 1 | SMP1 | Hs.523054 | 222401_s_at |
| small nuclear ribonucleoprotein D3 polypeptide | SNRPD3 | Hs.356549 | 202567_at |
| 18 kDa | | | |
| small nuclear ribonucleoprotein polypeptide A′ | SNRPA1 | Hs.528763 | 206055_s_at |
| Small nuclear ribonucleoprotein polypeptide A′ | SNRPA1 | Hs.528763 | 242146_at |
| small nuclear ribonucleoprotein polypeptide E | SNRPE | Hs.334612 | 203316_s_at |
| small nuclear ribonucleoprotein polypeptide F | SNRPF | Hs.105465 | 203832_at |
| small nuclear ribonucleoprotein polypeptide G | SNRPG | Hs.516076 | 205644_s_at |
| small nuclear RNA activating complex, polypeptide | SNAPC3 | Hs.546299 | 210465_s_at |
| 3, 50 kDa | | | |
| small nuclear RNA activating complex, polypeptide | SNAPC5 | Hs.30174 | 1554093_a_at |
| 5, 19 kDa | | | |
| Small nuclear RNA activating complex, polypeptide | SNAPC5 | Hs.30174 | 213203_at |
| 5, 19 kDa | | | |
| small proline-rich protein 1B (cornifin) | SPRR1B | Hs.1076 | 205064_at |
| small proline-rich protein 2C | SPRR2C | Hs.2421 | 220664_at |
| SMC1 structural maintenance of chromosomes 1- | SMC1L1 | Hs.211602 | 201589_at |
| like 1 (yeast) | | | |
| SMC2 structural maintenance of chromosomes 2- | SMC2L1 | Hs.119023 | 204240_s_at |
| like 1 (yeast) | | | |
| SMC6 structural maintenance of chromosomes 6- | SMC6L1 | Hs.526728 | 218781_at |
| like 1 (yeast) | | | |
| SMT3 suppressor of mif two 3 homolog 1 (yeast) /// | SUMO1 | Hs.81424 | 211069_s_at |
| SMT3 suppressor of mif two 3 homolog 1 (yeast) | | | |
| SMT3 suppressor of mif two 3 homolog 2 (yeast) | SUMO2 | Hs.380973 | 208739_x_at |
| SMT3 suppressor of mif two 3 homolog 2 (yeast) | SUMO2 | Hs.546298 | 213879_at |
| SMT3 suppressor of mif two 3 homolog 2 (yeast) | SUMO2 | Hs.380973 | 213881_x_at |
| SMT3 suppressor of mif two 3 homolog 2 (yeast) /// | SUMO2 | Hs.380973 | 208738_x_at |
| similar to SMT3 suppressor of mif two 3 homolog 2 | /// | | |
| | LOC441512 | | |
| SMT3 suppressor of mif two 3 homolog 4 (yeast) | SUMO4 | — | 215452_x_at |
| SNF related kinase | SNRK | Hs.476052 | 209481_at |
| SNF1-like kinase 2 | SNF1LK2 | Hs.552588 | 1557459_at |
| SNF1-like kinase 2 | SNF1LK2 | Hs.552588 | 223430_at |
| SNF2 histone linker PHD RING helicase | SHPRH | Hs.124537 | 243964_at |
| SNRPN upstream reading frame | SNRPN | Hs.525700 | 228370_at |
| soc-2 suppressor of clear homolog (C. elegans) | SHOC2 | Hs.104315 | 202777_at |
| sodium channel, voltage-gated, type XI, alpha | SCN11A | Hs.186877 | 220791_x_at |
| soluble liver antigen/liver pancreas antigen | SLA/LP | Hs.253305 | 1553167_a_at |
| solute carrier family 1 (glial high affinity glutamate | SLC1A2 | Hs.502338 | 208389_s_at |
| transporter), member 2 | | | |
| solute carrier family 1 (glutamate/neutral amino | SLC1A4 | Hs.323878 | 212811_x_at |
| acid transporter), member 4 | | | |
| Solute carrier family 1 (high affinity | SLC1A6 | Hs.515217 | 1554593_s_at |
| aspartate/glutamate transporter), member 6 | | | |
| solute carrier family 12 (potassium/chloride | SLC12A6 | Hs.4876 | 220740_s_at |
| transporters), member 6 | | | |
| solute carrier family 12 (potassium/chloride | SLC12A6 | Hs.4876 | 223596_at |
| transporters), member 6 | | | |
| solute carrier family 12 (potassium/chloride | SLC12A7 | Hs.172613 | 218066_at |
| transporters), member 7 | | | |
| solute carrier family 12 (sodium/potassium/chloride | SLC12A2 | Hs.162585 | 204404_at |
| transporters), member 2 | | | |
| solute carrier family 15, member 4 | SLC15A4 | Hs.507260 | 225043_at |
| solute carrier family 16 (monocarboxylic acid | SLC16A1 | Hs.75231 | 202236_s_at |
| transporters), member 1 | | | |
| solute carrier family 16 (monocarboxylic acid | SLC16A7 | Hs.439643 | 207057_at |
| transporters), member 7 | | | |
| solute carrier family 16 (monocarboxylic acid | SLC16A7 | Hs.439643 | 210807_s_at |
| transporters), member 7 | | | |
| solute carrier family 17 (anion/sugar transporter), | SLC17A5 | Hs.485760 | 223441_at |
| member 5 | | | |
| Solute carrier family 2 (facilitated glucose | SLC2A3 | Hs.419240 | 236571_at |
| transporter), member 3 | | | |
| Solute carrier family 22 (extraneuronal monoamine | SLC22A3 | Hs.242721 | 242578_x_at |
| transporter), member 3 | | | |
| solute carrier family 22 (organic cation transporter), | SLC22A14 | Hs.165559 | 207408_at |
| member 14 | | | |
| solute carrier family 22 (organic cation transporter), | SLC22A16 | Hs.520319 | 232233_at |
| member 16 | | | |
| solute carrier family 22 (organic cation transporter), | SLC22A18 | Hs.50868 | 204981_at |
| member 18 | | | |
| solute carrier family 25 (carnitine/acylcarnitine | SLC25A20 | Hs.13845 | 203658_at |
| translocase), member 20 | | | |
| solute carrier family 25 (mitochondrial carrier; | SLC25A31 | Hs.149030 | 221037_s_at |
| adenine nucleotide translocator), member 31 /// | | | |
| solute carrier family 25 (mitochondrial carrier; | | | |
| adenine nucleotide translocator), member 31 | | | |
| solute carrier family 25 (mitochondrial carrier; | SLC25A5 | Hs.522767 | 200657_at |
| adenine nucleotide translocator), member 5 | | | |
| solute carrier family 25 (mitochondrial carrier; | SLC25A16 | Hs.180408 | 210686_x_at |
| Graves disease autoantigen), member 16 | | | |
| solute carrier family 25 (mitochondrial carrier; | SLC25A2 | Hs.97647 | 224166_at |
| ornithine transporter) member 2 | | | |
| Solute carrier family 25 (mitochondrial carrier; | SLC25A24 | Hs.132553 | 244481_at |
| phosphate carrier), member 24 | | | |
| solute carrier family 25 (mitochondrial carrier; | SLC25A3 | Hs.290404 | 200030_s_at |
| phosphate carrier), member 3 /// solute carrier | | | |
| family 25 (mitochondrial carrier; phosphate carrier), | | | |
| member 3 | | | |
| solute carrier family 3 (cystine, dibasic and neutral | SLC3A1 | Hs.112916 | 205799_s_at |
| amino acid transporters, activator of cystine, | | | |
| dibasic and neutral amino acid transport), member 1 | | | |
| Solute carrier family 30 (zinc transporter), member 7 | SLC30A7 | Hs.533903 | 243524_at |
| solute carrier family 30 (zinc transporter), member 9 | SLC30A9 | Hs.479634 | 202614_at |
| solute carrier family 33 (acetyl-CoA transporter), | SLC33A1 | Hs.478031 | 1554148_a_at |
| member 1 | | | |
| solute carrier family 33 (acetyl-CoA transporter), | SLC33A1 | Hs.478031 | 203164_at |
| member 1 | | | |
| solute carrier family 35 (UDP-galactose | SLC35A2 | Hs.21899 | 209326_at |
| transporter), member A2 | | | |
| Solute carrier family 35, member D2 | SLC35D2 | Hs.494556 | 231437_at |
| solute carrier family 35, member E1 | SLC35E1 | Hs.134074 | 220796_x_at |
| Solute carrier family 35, member E4 | SLC35E4 | Hs.526928 | 241078_at |
| Solute carrier family 35, member F3 | SLC35F3 | Hs.158748 | 1568655_a_at |
| solute carrier family 38, member 1 | SLC38A1 | Hs.533770 | 218237_s_at |
| solute carrier family 39 (zinc transporter), member | SLC39A13 | Hs.523664 | 1552295_a_at |
| 13 | | | |
| solute carrier family 39 (zinc transporter), member 6 | SLC39A6 | Hs.79136 | 202088_at |
| solute carrier family 39 (zinc transporter), member 6 | SLC39A6 | Hs.79136 | 202089_s_at |
| Solute carrier family 4 (anion exchanger), member | SLC4A1AP | Hs.306000 | 242030_at |
| 1, adaptor protein | | | |
| solute carrier family 4, sodium bicarbonate | SLC4A7 | Hs.250072 | 207604_s_at |
| cotransporter, member 7 | | | |
| solute carrier family 43, member 1 | SLC43A1 | Hs.444159 | 204394_at |
| solute carrier family 5 (inositol transporters), | SLC5A3 | Hs.534348 | 213164_at |
| member 3 | | | |
| solute carrier family 6 (amino acid transporter), | SLC6A14 | Hs.522109 | 219795_at |
| member 14 | | | |
| solute carrier family 6 (neurotransmitter | SLC6A8 | Hs.540696 | 210854_x_at |
| transporter, creatine), member 8 | | | |
| solute carrier family 6 (neurotransmitter | SLC6A8 | Hs.540696 | 213843_x_at |
| transporter, creatine), member 8 | | | |
| Solute carrier family 6 (neurotransmitter | SLC6A6 | Hs.529488 | 239474_at |
| transporter, taurine), member 6 | | | |
| Solute carrier family 6, member 16 | SLC6A16 | Hs.130949 | 1569940_at |
| solute carrier family 7 (cationic amino acid | SLC7A1 | Hs.14846 | 212295_s_at |
| transporter, y+ system), member 1 | | | |
| solute carrier family 7 (cationic amino acid | SLC7A8 | Hs.22891 | 216092_s_at |
| transporter, y+ system), member 8 | | | |
| solute carrier organic anion transporter family, | SLCO3A1 | Hs.311187 | 219229_at |
| member 3A1 | | | |
| Solute carrier organic anion transporter family, | SLCO3A1 | Hs.311187 | 227367_at |
| member 3A1 | | | |
| solute carrier organic anion transporter family, | SLCO6A1 | Hs.388874 | 1552745_at |
| member 6A1 | | | |
| SON DNA binding protein | SON | Hs.517262 | 214988_s_at |
| SON DNA binding protein | SON | Hs.517262 | 226465_s_at |
| Son of sevenless homolog 1 (Drosophila) | SOS1 | Hs.278733 | 227426_at |
| Son of sevenless homolog 2 (Drosophila) | SOS2 | Hs.291533 | 233369_at |
| sorcin | SRI | Hs.489040 | 208921_s_at |
| sortilin 1 | SORT1 | Hs.485195 | 212807_s_at |
| sorting nexin 13 | SNX13 | Hs.487648 | 213292_s_at |
| sorting nexin 13 | SNX13 | Hs.487648 | 215820_x_at |
| sorting nexin 16 | SNX16 | Hs.492121 | 219793_at |
| sorting nexin 3 | SNX3 | Hs.12102 | 208781_x_at |
| sorting nexin 3 | SNX3 | Hs.12102 | 210648_x_at |
| sorting nexin 3 | SNX3 | Hs.12102 | 213545_x_at |
| sorting nexin 3/// sorting nexin 3 | SNX3 | Hs.12102 | 200067_x_at |
| sorting nexin 4 | SNX4 | Hs.507243 | 205329_s_at |
| sorting nexin 5 | SNX5 | Hs.316890 | 229980_s_at |
| Sorting nexin associated golgi protein 1 | SNAG1 | Hs.432755 | 226683_at |
| sorting nexin family member 27 | SNX27 | Hs.192326 | 221498_at |
| sorting nexing 24 | SNX24 | Hs.483200 | 239739_at |
| Source of immunodominant MHC-associated | SIMP | Hs.475812 | 231285_at |
| peptides | | | |
| Source of immunodominant MHC-associated | SIMP | Hs.475812 | 238303_at |
| peptides | | | |
| Sp2 transcription factor | SP2 | Hs.514276 | 204367_at |
| Sp3 transcription factor | SP3 | Hs.531587 | 232529_at |
| SPANX family, member C | SPANXC | Hs.343879 | 220217_x_at |
| spastic ataxia of Charlevoix-Saguenay (sacsin) | SACS | Hs.159492 | 213262_at |
| spastic paraplegia 21 (autosomal recessive, Mast | SPG21 | Hs.242458 | 215383_x_at |
| syndrome) | | | |
| spastic paraplegia 3A (autosomal dominant) | SPG3A | Hs.241503 | 223340_at |
| special AT-rich sequence binding protein 1 (binds | SATB1 | Hs.517717 | 203408_s_at |
| to nuclear matrix/scaffold-associating DNA's) | | | |
| speckle-type POZ protein | SPOP | Hs.463382 | 204640_s_at |
| spectrin repeat containing, nuclear envelope 2 | SYNE2 | Hs.525392 | 202761_s_at |
| spectrin, beta, non-erythrocytic 1 | SPTBN1 | Hs.503178 | 200672_x_at |
| spectrin, beta, non-erythrocytic 1 | SPTBN1 | Hs.503178 | 212071_s_at |
| Spectrin, beta, non-erythrocytic 5 | SPTBN5 | Hs.198161 | 1556839_s_at |
| spen homolog, transcriptional regulator | SPEN | Hs.270499 | 201997_s_at |
| (Drosophila) | | | |
| sperm acrosome associated 3 | SPACA3 | Hs.434112 | 243621_at |
| sperm acrosome associated 4 | SPACA4 | Hs.122599 | 231054_at |
| sperm adhesion molecule 1 (PH-20 hyaluronidase, | SPAM1 | Hs.121494 | 210536_s_at |
| zona pellucida binding) | | | |
| sperm adhesion molecule 1 (PH-20 hyaluronidase, | SPAM1 | Hs.121494 | 216989_at |
| zona pellucida binding) | | | |
| Sperm antigen HCMOGT-1 | HCMOGT-1 | Hs.431045 | 240765_at |
| sperm associated antigen 11 | SPAG11 | Hs.2717 | 1553582_a_at |
| sperm associated antigen 4 | SPAG4 | Hs.123159 | 219888_at |
| sperm associated antigen 5 | SPAG5 | Hs.514033 | 203145_at |
| sperm associated antigen 7 /// sperm associated | SPAG7 | Hs.90436 | 200053_at |
| antigen 7 | | | |
| sperm associated antigen 9 | SPAG9 | Hs.463439 | 1554543_at |
| sperm associated antigen 9 | SPAG9 | Hs.463439 | 212468_at |
| sperm associated antigen 9 | SPAG9 | Hs.463439 | 212470_at |
| sperm autoantigenic protein 17 | SPA17 | Hs.286233 | 205406_s_at |
| sperm equatorial segment protein 1 | SPESP1 | — | 229352_at |
| sperm protein associated with the nucleus, X- | SPANXA1 | Hs.334464 | 224032_x_at |
| linked, family member A1 /// SPANX family, | /// | | |
| member A2 | SPANXA2 | | |
| sperm protein associated with the nucleus, X- | SPANXA1 | Hs.343879 | 220922_s_at |
| linked, family member A1 /// SPANX family, | /// | | |
| member B1 /// SPANX family, member A2 /// | SPANXB1 | | |
| SPANX family, member C /// SPANX family, | /// | | |
| member B2 | SPANXA2 | | |
| | /// | | |
| | SPANXC | | |
| | /// | | |
| | SPANXB2 | | |
| sperm protein SSP411 | SSP411 | Hs.103147 | 218164_at |
| Spermatid perinuclear RNA binding protein | STRBP | Hs.287659 | 229513_at |
| spermatid perinuclear RNA binding protein | STRBP | Hs.287659 | 233252_s_at |
| spermatogenesis associated 11 | SPATA11 | Hs.111099 | 227878_s_at |
| spermatogenesis associated 2 | SPATA2 | Hs.48513 | 204434_at |
| spermatogenesis associated 3 | SPATA3 | Hs.121021 | 240340_at |
| spermatogenesis associated 4 | SPATA4 | Hs.481235 | 231178_at |
| spermatogenesis associated 5-like 1 | SPATA5L1 | Hs.369657 | 222163_s_at |
| spermatogenesis associated 6 | SPATA6 | Hs.408467 | 220298_s_at |
| spermatogenesis associated 6 | SPATA6 | Hs.408467 | 220299_at |
| spermatogenesis associated 8 | SPATA8 | Hs.326528 | 231006_at |
| spermatogenesis associated 9 | SPATA9 | Hs.50499 | 223840_s_at |
| spermatogenesis associated 9 | SPATA9 | Hs.50499 | 223841_s_at |
| Spermatogenesis-related protein 7 | — | Hs.372658 | 231586_at |
| spermidine/spermine N1-acetyltransferase | SAT | Hs.28491 | 203455_s_at |
| Spermidine/spermine N1-acetyltransferase | SAT | Hs.28491 | 230333_at |
| spermine synthase | SMS | Hs.529887 | 202043_s_at |
| SPFH domain family, member 2 | SPFH2 | Hs.125849 | 221543_s_at |
| S-phase kinase-associated protein 1A (p19A) | SKP1A | Hs.171626 | 200711_s_at |
| S-phase kinase-associated protein 1A (p19A) | SKP1A | Hs.171626 | 200718_s_at |
| S-phase kinase-associated protein 1A (p19A) | SKP1A | Hs.171626 | 207974_s_at |
| S-phase response (cyclin-related) | SPHAR | Hs.511754 | 206272_at |
| sphingomyelin phosphodiesterase 1, acid | SMPD1 | Hs.498173 | 209420_s_at |
| lysosomal (acid sphingomyelinase) | | | |
| spindle assembly abnormal protein 6 | SAS-6 | Hs.145497 | 231895_at |
| spindlin | SPIN | Hs.146804 | 217813_s_at |
| spire homolog 1 (Drosophila) | SPIRE1 | Hs.515283 | 1559517_a_at |
| spire homolog 1 (Drosophila) | SPIRE1 | Hs.515283 | 224995_at |
| spire homolog 1 (Drosophila) | SPIRE1 | Hs.515283 | 225018_at |
| Spleen tyrosine kinase | SYK | Hs.371720 | 226068_at |
| Spleen tyrosine kinase | SYK | Hs.371720 | 244023_at |
| splicing factor 1 | SF1 | Hs.502829 | 208313_s_at |
| splicing factor 1 | SF1 | Hs.502829 | 210172_at |
| splicing factor 3a, subunit 1, 120 kDa | SF3A1 | Hs.406277 | 201356_at |
| splicing factor 3a, subunit 1, 120 kDa | SF3A1 | Hs.406277 | 216457_s_at |
| Splicing factor proline/glutamine rich | SFPQ | Hs.355934 | 214016_s_at |
| (polypyrimidine tract binding protein associated) | | | |
| splicing factor YT521-B | YT521 | Hs.175955 | 212455_at |
| splicing factor YT521-B | YT521 | Hs.175955 | 228556_at |
| splicing factor, arginine/serine-rich 1 (splicing | SFRS1 | Hs.68714 | 201742_x_at |
| factor 2, alternate splicing factor) | | | |
| splicing factor, arginine/serine-rich 1 (splicing | SFRS1 | Hs.68714 | 208863_s_at |
| factor 2, alternate splicing factor) | | | |
| Splicing factor, arginine/serine-rich 1 (splicing | SFRS1 | Hs.68714 | 226419_s_at |
| factor 2, alternate splicing factor) | | | |
| Splicing factor, arginine/serine-rich 1 (splicing | SFRS1 | Hs.68714 | 227164_at |
| factor 2, alternate splicing factor) | | | |
| splicing factor, arginine/serine-rich 1 (splicing | SFRS1 | Hs.68714 | 211784_s_at |
| factor 2, alternate splicing factor) /// splicing factor, | | | |
| arginine/serine-rich 1 (splicing factor 2, alternate | | | |
| splicing factor) | | | |
| splicing factor, arginine/serine-rich 10 (transformer | SFRS10 | Hs.533122 | 200892_s_at |
| 2 homolog, Drosophila) | | | |
| splicing factor, arginine/serine-rich 10 (transformer | SFRS10 | Hs.533122 | 200893_at |
| 2 homolog, Drosophila) | | | |
| splicing factor, arginine/serine-rich 10 (transformer | SFRS11 | Hs.533122 | 210180_s_at |
| 2 homolog, Drosophila) | | | |
| splicing factor, arginine/serine-rich 11 | SFRS11 | Hs.479693 | 200686_s_at |
| splicing factor, arginine/serine-rich 11 | SFRS11 | Hs.479693 | 213742_at |
| splicing factor, arginine/serine-rich 14 | SFRS14 | Hs.515271 | 214092_x_at |
| splicing factor, arginine/serine-rich 15 | SFRS15 | Hs.17255 | 222310_at |
| splicing factor, arginine/serine-rich 2 | SFRS2 | Hs.73965 | 200753_x_at |
| splicing factor, arginine/serine-rich 2 | SFRS2 | Hs.73965 | 200754_x_at |
| splicing factor, arginine/serine-rich 2 | SFRS2 | Hs.73965 | 214882_s_at |
| splicing factor, arginine/serine-rich 2, interacting | SFRS2IP | Hs.210367 | 213850_s_at |
| protein | | | |
| Splicing factor, arginine/serine-rich 2, interacting | SFRS2IP | Hs.210367 | 232597_x_at |
| protein | | | |
| splicing factor, arginine/serine-rich 3 | SFRS3 | Hs.405144 | 202899_s_at |
| splicing factor, arginine/serine-rich 3 | SFRS3 | Hs.405144 | 208673_s_at |
| Splicing factor, arginine/serine-rich 3 | SFRS3 | Hs.405144 | 237485_at |
| splicing factor, arginine/serine-rich 5 | SFRS5 | Hs.166975 | 203380_x_at |
| splicing factor, arginine/serine-rich 5 | SFRS5 | Hs.166975 | 212266_s_at |
| splicing factor, arginine/serine-rich 7, 35 kDa | SFRS7 | Hs.309090 | 201129_at |
| splicing factor, arginine/serine-rich 7, 35 kDa | SFRS7 | Hs.309090 | 214141_x_at |
| Splicing factor, arginine/serine-rich, 46 kD | SRP46 | Hs.476680 | 214789_x_at |
| Splicing factor, arginine/serine-rich, 46 kD | SRP46 | Hs.476680 | 228760_at |
| split hand/foot malformation (ectrodactyly) type 1 | SHFM1 | Hs.489201 | 202276_at |
| Spondin 1, extracellular matrix protein | SPON1 | Hs.445818 | 217703_x_at |
| squalene epoxidase | SQLE | Hs.71465 | 209218_at |
| SRB7 suppressor of RNA polymerase B homolog | SURB7 | Hs.286145 | 209362_at |
| (yeast) | | | |
| SRB7 suppressor of RNA polymerase B homolog | SURB7 | Hs.286145 | 209363_s_at |
| (yeast) | | | |
| src family associated phosphoprotein 2 | SCAP2 | Hs.200770 | 204361_s_at |
| src family associated phosphoprotein 2 | SCAP2 | Hs.200770 | 216899_s_at |
| Src family associated phosphoprotein 2 | SCAP2 | Hs.200770 | 241331_at |
| Src-like-adaptor | SLA | Hs.75367 | 233111_at |
| SRY (sex determining region Y)-box 30 | SOX30 | Hs.529462 | 1559235_a_at |
| SRY (sex determining region Y)-box 4 | SOX4 | Hs.357901 | 201416_at |
| SRY (sex determining region Y)-box 7 | SOX7 | Hs.213194 | 224013_s_at |
| SRY (sex determining region Y)-box 9 | SOX9 | Hs.2316 | 202935_s_at |
| (campomelic dysplasia, autosomal sex-reversal) | | | |
| SRY (sex determining region Y)-box 9 | SOX9 | Hs.2316 | 202936_s_at |
| (campomelic dysplasia, autosomal sex-reversal) | | | |
| ST3 beta-galactoside alpha-2,3-sialyltransferase 3 | SIAT6 | Hs.132001 | 242511_at |
| ST6 (alpha-N-acetyl-neuraminyl-2,3-beta- | ST6GALNAC2 | Hs.281434 | 204542_at |
| galactosyl-1,3)-N-acetylgalactosaminide alpha-2,6- | | | |
| sialyltransferase 2 | | | |
| ST7 overlapping transcript 2 (antisense non-coding | ST7OT2 | Hs.131297 | 237808_at |
| RNA) | | | |
| STAM binding protein | STAMBP | Hs.183070 | 202811_at |
| Staphylococcal nuclease domain containing 1 | SND1 | Hs.122523 | 228271_at |
| Staphylococcal nuclease domain containing 1 | SND1 | Hs.122523 | 242506_at |
| START domain containing 9 | STARD9 | Hs.122061 | 227108_at |
| stathmin 1/oncoprotein 18 | STMN1 | Hs.209983 | 200783_s_at |
| staufen, RNA binding protein (Drosophila) | STAU | Hs.370187 | 207320_x_at |
| staufen, RNA binding protein (Drosophila) | STAU | Hs.370187 | 213037_x_at |
| Stereocilin | STRC | Hs.550553 | 235452_at |
| Sterile alpha motif and leucine zipper containing | ZAK | Hs.444451 | 1563468_at |
| kinase AZK | | | |
| sterile alpha motif and leucine zipper containing | ZAK | Hs.444451 | 222757_s_at |
| kinase AZK | | | |
| sterile alpha motif domain containing 4 | SAMD4 | Hs.98259 | 215495_s_at |
| Sterile alpha motif domain containing 4 | SAMD4 | Hs.98259 | 235756_at |
| sterile alpha motif domain containing 9 | SAMD9 | Hs.65641 | 228531_at |
| steroid sensitive gene 1 | URB | Hs.477128 | 225242_s_at |
| steroid-5-alpha-reductase, alpha polypeptide 1 (3- | SRD5A1 | Hs.552 | 204675_at |
| oxo-5 alpha-steroid delta 4-dehydrogenase alpha | | | |
| 1) | | | |
| sterol carrier protein 2 | SCP2 | Hs.476365 | 201339_s_at |
| Sterol regulatory element binding transcription | SREBF2 | Hs.443258 | 242748_at |
| factor 2 | | | |
| stomatin (EPB72)-like 2 | STOML2 | Hs.3439 | 215416_s_at |
| stratifin | SFN | Hs.523718 | 33322_i_at |
| stratifin | SFN | Hs.523718 | 33323_r_at |
| stress-associated endoplasmic reticulum protein 1 | SERP1 | Hs.518326 | 200971_s_at |
| Striatin, calmodulin binding protein | STRN | Hs.43590 | 230734_x_at |
| striatin, calmodulin binding protein 4 | STRN4 | Hs.190904 | 217903_at |
| Stromal cell derived factor receptor 1 | SDFR1 | Hs.187866 | 228723_at |
| stromal interaction molecule 2 | STIM2 | Hs.135763 | 225250_at |
| stromal membrane-associated protein 1 | SMAP1 | Hs.485717 | 218137_s_at |
| structural maintenance of chromosomes flexible | SMCHD1 | Hs.8118 | 1558747_at |
| hinge domain containing 1 | | | |
| structural maintenance of chromosomes flexible | SMCHD1 | Hs.8118 | 212579_at |
| hinge domain containing 1 | | | |
| structure specific recognition protein 1 | SSRP1 | Hs.523680 | 200957_s_at |
| succinate dehydrogenase complex, subunit C, | SDHC | Hs.444472 | 215088_s_at |
| integral membrane protein, 15 kDa | | | |
| sucrase-isomaltase (alpha-glucosidase) | SI | Hs.429596 | 206664_at |
| sulfatase 1 | SULF1 | Hs.409602 | 212344_at |
| sulfotransferase family, cytosolic, 1A, phenol- | SULT1A1 | Hs.142 | 203615_x_at |
| preferring, member 1 | | | |
| sulfotransferase family, cytosolic, 1A, phenol- | SULT1A1 | Hs.142 | 215299_x_at |
| preferring, member 1 | | | |
| sulfotransferase family, cytosolic, 1A, phenol- | SULT1A3 | Hs.460587 | 209607_x_at |
| preferring, member 3 | | | |
| sulfotransferase family, cytosolic, 1A, phenol- | SULT1A3 | Hs.460587 | 210580_x_at |
| preferring, member 3 | | | |
| SUMO/sentrin specific protease family member 8 | SENP8 | Hs.513002 | 228398_at |
| SUMO-1 activating enzyme subunit 1 | SAE1 | Hs.515500 | 217946_s_at |
| SUMO1/sentrin specific protease 5 | SENP5 | Hs.533124 | 222110_at |
| superkiller viralicidic activity 2-like 2 (S. cerevisiae) | SKIV2L2 | Hs.274531 | 212896_at |
| superoxide dismutase 1, soluble (amyotrophic | SOD1 | Hs.443914 | 200642_at |
| lateral sclerosis 1 (adult)) | | | |
| superoxide dismutase 2, mitochondrial | SOD2 | Hs.487046 | 215223_s_at |
| superoxide dismutase 2, mitochondrial | SOD2 | Hs.487046 | 216841_s_at |
| Supervillin | SVIL | Hs.499209 | 1569512_at |
| suppression of tumorigenicity 13 (colon carcinoma) | ST13 | Hs.546303 | 207040_s_at |
| (Hsp70 interacting protein) | | | |
| suppression of tumorigenicity 13 (colon carcinoma) | ST13 | Hs.546303 | 208667_s_at |
| (Hsp70 interacting protein) | | | |
| suppression of tumorigenicity 7 | ST7 | Hs.368131 | 207524_at |
| suppression of tumorigenicity 7 like | ST7L | Hs.201921 | 1552738_a_at |
| suppression of tumorigenicity 7 like | ST7L | Hs.201921 | 1552739_s_at |
| suppressor of cytokine signaling 1 | SOCS1 | Hs.50640 | 210001_s_at |
| suppressor of cytokine signaling 5 | SOCS5 | Hs.468426 | 208127_s_at |
| suppressor of cytokine signaling 5 | SOCS5 | Hs.468426 | 209647_s_at |
| suppressor of cytokine signaling 7 | SOCS7 | Hs.514132 | 214015_at |
| suppressor of hairy wing homolog 2 (Drosophila) | SUHW2 | Hs.43834 | 215048_at |
| suppressor of hairy wing homolog 2 (Drosophila) | SUHW2 | Hs.43834 | 229360_at |
| suppressor of hairy wing homolog 2 (Drosophila) | SUHW2 | Hs.43834 | 230789_at |
| suppressor of potassium transport defect 3 | SKD3 | Hs.523877 | 221845_s_at |
| suppressor of Ty 16 homolog (S. cerevisiae) | SUPT16H | Hs.213724 | 217815_at |
| suppressor of Ty 5 homolog (S. cerevisiae) | SUPT5H | Hs.437056 | 201480_s_at |
| suppressor of variegation 4-20 homolog 1 | SUV420H1 | Hs.503001 | 218242_s_at |
| (Drosophila) | | | |
| suppressor of variegation 4-20 homolog 1 | SUV420H1 | Hs.503001 | 222759_at |
| (Drosophila) | | | |
| suppressor of variegation 4-20 homolog 2 | SUV420H2 | Hs.391051 | 224431_s_at |
| (Drosophila) /// suppressor of variegation 4-20 | | | |
| homolog 2 (Drosophila) | | | |
| suppressor of zeste 12 homolog (Drosophila) | SUZ12 | Hs.462732 | 212287_at |
| surfactant, pulmonary-associated protein B | SFTPB | Hs.512690 | 213936_x_at |
| surfactant, pulmonary-associated protein B | SFTPB | Hs.512690 | 214354_x_at |
| survival of motor neuron protein interacting protein 1 | SIP1 | Hs.533862 | 205063_at |
| survival of motor neuron protein interacting protein 1 | SIP1 | Hs.533862 | 211114_x_at |
| survival of motor neuron protein interacting protein 1 | SIP1 | Hs.533862 | 211115_x_at |
| SVAP1 protein | IMAGE3451454 | Hs.325081 | 225244_at |
| SWI/SNF related, matrix associated, actin | SMARCA2 | Hs.298990 | 206542_s_at |
| dependent regulator of chromatin, subfamily a, | | | |
| member 2 | | | |
| SWI/SNF related, matrix associated, actin | SMARCA2 | Hs.298990 | 217707_x_at |
| dependent regulator of chromatin, subfamily a, | | | |
| member 2 | | | |
| SWI/SNF related, matrix associated, actin | SMARCC1 | Hs.476179 | 201073_s_at |
| dependent regulator of chromatin, subfamily c, | | | |
| member 1 | | | |
| SWI/SNF related, matrix associated, actin | SMARCC1 | Hs.476179 | 239238_at |
| dependent regulator of chromatin, subfamily c, | | | |
| member 1 | | | |
| SWI/SNF related, matrix associated, actin | SMARCC2 | Hs.236030 | 201321_s_at |
| dependent regulator of chromatin, subfamily c, | | | |
| member 2 | | | |
| SWI/SNF related, matrix associated, actin | SMARCE1 | Hs.463010 | 211988_at |
| dependent regulator of chromatin, subfamily e, | | | |
| member 1 | | | |
| symplekin | SYMPK | Hs.515475 | 1554595_at |
| synapsin II | SYN2 | Hs.445503 | 210247_at |
| synaptobrevin-like 1 | SYBL1 | Hs.24167 | 202829_s_at |
| synaptojanin 1 | SYNJ1 | Hs.473632 | 207594_s_at |
| synaptojanin 2 | SYNJ2 | Hs.434494 | 240257_at |
| synaptonemal complex protein 1 | SYCP1 | Hs.112743 | 206740_x_at |
| synaptophysin-like protein | SYPL | Hs.80919 | 201259_s_at |
| Synaptopodin 2 | SYNPO2 | Hs.480615 | 215439_x_at |
| synaptosomal-associated protein, 23 kDa | SNAP23 | Hs.511149 | 209130_at |
| synaptotagmin binding, cytoplasmic RNA | SYNCRIP | Hs.472056 | 1555427_s_at |
| interacting protein | | | |
| synaptotagmin binding, cytoplasmic RNA | SYNCRIP | Hs.472056 | 209024_s_at |
| interacting protein | | | |
| synaptotagmin binding, cytoplasmic RNA | SYNCRIP | Hs.472056 | 209025_s_at |
| interacting protein | | | |
| synaptotagmin binding, cytoplasmic RNA | SYNCRIP | Hs.472056 | 217834_s_at |
| interacting protein | | | |
| synaptotagmin VI | SYT6 | Hs.370963 | 244227_at |
| synaptotagmin-like 2 | SYTL2 | Hs.369520 | 232914_s_at |
| Syncoilin, intermediate filament 1 | SYNCOILIN | Hs.550537 | 237333_at |
| syndecan binding protein (syntenin) | SDCBP | Hs.200804 | 200958_s_at |
| synovial sarcoma translocation gene on | SS18L2 | Hs.534454 | 218283_at |
| chromosome 18-like 2 | | | |
| synovial sarcoma translocation, chromosome 18 | SS18 | Hs.404263 | 202816_s_at |
| synovial sarcoma, X breakpoint 2 interacting | SSX2IP | Hs.22587 | 203015_s_at |
| protein | | | |
| syntaxin 8 | STX8 | Hs.431109 | 204690_at |
| syntaxin binding protein 5 (tomosyn) | STXBP5 | Hs.93534 | 226794_at |
| syntrophin, beta 2 (dystrophin-associated protein | SNTB2 | Hs.461117 | 205315_s_at |
| A1, 59 kDa, basic component 2) | | | |
| Syntrophin, beta 2 (dystrophin-associated protein | SNTB2 | Hs.461117 | 232784_at |
| A1, 59 kDa, basic component 2) | | | |
| T cell receptor beta chain (TCRB) mRNA, TCRB- | — | Hs.511522 | 241133_at |
| BV27 J2-4 BC2 allele | | | |
| T cell receptor gamma constant 2 /// T cell receptor | TRGC2 | Hs.534032 | 216920_s_at |
| gamma variable 9 /// similar to T-cell receptor | /// | | |
| gamma chain C region PT-gamma-1/2 /// TCR | TRGV9 | | |
| gamma alternate reading frame protein | /// | | |
| | LOC442532 | | |
| | /// | | |
| | TARP | | |
| TAF10 RNA polymerase II, TATA box binding | TAF10 | Hs.89657 | 235135_at |
| protein (TBP)-associated factor, 30 kDa | | | |
| TAF10 RNA polymerase II, TATA box binding | TAF10 | Hs.89657 | 200055_at |
| protein (TBP)-associated factor, 30 kDa /// TAF10 | | | |
| RNA polymerase II, TATA box binding protein | | | |
| (TBP)-associated factor, 30 kDa | | | |
| TAF15 RNA polymerase II, TATA box binding | TAF15 | Hs.402752 | 202840_at |
| protein (TBP)-associated factor, 68 kDa | | | |
| TAF15 RNA polymerase II, TATA box binding | TAF15 | Hs.402752 | 227884_at |
| protein (TBP)-associated factor, 68 kDa | | | |
| TAF9 RNA polymerase II, TATA box binding | TAF9 | Hs.248941 | 202168_at |
| protein (TBP)-associated factor, 32 kDa | | | |
| TAK1-binding protein 3 | TAB3 | Hs.188256 | 227357_at |
| TANK-binding kinase 1 | TBK1 | Hs.505874 | 218520_at |
| tankyrase, TRF1-interacting ankyrin-related ADP- | TNKS | Hs.370267 | 202561_at |
| ribose polymerase | | | |
| Tankyrase, TRF1-interacting ankyrin-related ADP- | TNKS | Hs.370267 | 233152_x_at |
| ribose polymerase | | | |
| TAO kinase 3 | TAOK3 | — | 221508_at |
| TAO kinase 3 | RPS2 | Hs.368304 | 236002_at |
| TAR DNA binding protein /// TAR DNA binding | TARDBP | Hs.300624 | 200020_at |
| protein | | | |
| TAR DNA binding protein /// TAR DNA binding | TARDBP | Hs.300624 | 221264_s_at |
| protein | | | |
| Tara-like protein | HRIHFB2122 | Hs.533030 | 202795_x_at |
| target of myb1-like 1 (chicken) | TOM1L1 | Hs.153504 | 204485_s_at |
| target of myb1-like 2 (chicken) | TOM1L2 | Hs.462379 | 226198_at |
| TATA element modulatory factor 1 | TMF1 | Hs.267632 | 235566_at |
| TATA element modulatory factor 1 | TMF1 | Hs.267632 | 236829_at |
| TATA element modulatory factor 1 /// Similar to | TMF1 | Hs.267632 /// | 214948_s_at |
| family with sequence similarity 9, member C | | Hs.530171 | |
| tau tubulin kinase 2 | TTBK2 | Hs.549247 | 213922_at |
| Tax1 (human T-cell leukemia virus type I) binding | TAX1BP1 | Hs.34576 | 200976_s_at |
| protein 1 | | | |
| Tax1 (human T-cell leukemia virus type I) binding | TAX1BP1 | Hs.34576 | 200977_s_at |
| protein 1 | | | |
| TBC1 (tre-2/USP6, BUB2, cdc16) domain family, | TBC1D1 | Hs.176503 | 212350_at |
| member 1 | | | |
| TBC1 domain family, member 10B | TBC1D10B | Hs.513498 | 220947_s_at |
| TBC1 domain family, member 15 | TBC1D15 | Hs.284630 | 218268_at |
| TBC1 domain family, member 20 | TBC1D20 | Hs.203514 | 226664_at |
| TBC1 domain family, member 21 | TBC1D21 | Hs.124512 | 231475_at |
| TBC1 domain family, member 22A | TBC1D22A | Hs.435044 | 240238_at |
| TBC1 domain family, member 22A | TBC1D22A | Hs.435044 | 33778_at |
| TBC1 domain family, member 3 /// similar to TBC1 | TBC1D3 | Hs.105891 | 209403_at |
| domain family, member 3, telomeric | /// | | |
| | MGC44903 | | |
| T-box 3 (ulnar mammary syndrome) | TBX3 | Hs.129895 | 225544_at |
| TBP-1 interacting protein | TBPIP | Hs.279032 | 205956_x_at |
| TBP-interacting protein | TIP120B | Hs.343664 | 213547_at |
| TCDD-inducible poly(ADP-ribose) polymerase | TIPARP | Hs.12813 | 1556829_at |
| T-cell activation kelch repeat protein | TA-KRP | Hs.116665 | 239835_at |
| T-cell activation protein phosphatase 2C | TA- | Hs.13854 | 225204_at |
| | PP2C | | |
| T-cell activation protein phosphatase 2C | TA- | Hs.13854 | 225213_at |
| | PP2C | | |
| T-cell lymphoma invasion and metastasis 1 | TIAM1 | Hs.517228 | 213135_at |
| t-complex 1 | TCP1 | Hs.487054 | 208778_s_at |
| t-complex 10 (mouse)-like | TCP10L | Hs.42034 | 230394_at |
| t-complex 11 (mouse) | TCP11 | Hs.435371 | 220378_at |
| t-complex-associated-testis-expressed 1-like | TCTE1L | Hs.446392 | 203303_at |
| t-complex-associated-testis-expressed 1-like 1 | TCTEL1 | Hs.445999 | 201999_s_at |
| TEA domain family member 1 (SV40 | TEAD1 | Hs.546308 | 224955_at |
| transcriptional enhancer factor) | | | |
| Telomeric repeat binding factor 2 | TERF2 | Hs.63335 | 235872_at |
| Tenascin C (hexabrachion) | TNC | Hs.143250 | 216005_at |
| TERF1 (TRF1)-interacting nuclear factor 2 | TINF2 | Hs.496191 | 223776_x_at |
| testes development-related NYD-SP17 | NYD- | Hs.164799 | 244735_at |
| | SP17 | | |
| testes development-related NYD-SP18 | NYD- | Hs.131098 | 236214_at |
| | SP18 | | |
| Testes development-related NYD-SP20 | NYD- | Hs.351068 | 1558668_s_at |
| | SP20 | | |
| testes development-related NYD-SP20 | NYD- | Hs.351068 | 231114_at |
| | SP20 | | |
| testes development-related NYD-SP21 | NYD- | Hs.28514 | 229510_at |
| | SP21 | | |
| testicular soluble adenylyl cyclase | SAC | Hs.320892 | 217305_s_at |
| testis development protein NYD-SP26 | NYD- | Hs.120316 | 231612_at |
| | SP26 | | |
| testis enhanced gene transcript (BAX inhibitor 1) | TEGT | Hs.35052 | 200803_s_at |
| testis expressed sequence 10 | TEX10 | Hs.494648 | 218104_at |
| testis expressed sequence 11 | TEX11 | Hs.121776 | 233514_x_at |
| testis expressed sequence 11 | TEX11 | Hs.121776 | 234296_s_at |
| testis expressed sequence 15 /// testis expressed | TEX15 | Hs.458316 | 221448_s_at |
| sequence 15 | | | |
| testis expressed sequence 27 | TEX27 | Hs.36959 | 218020_s_at |
| testis expressed sequence 27 | TEX27 | Hs.36959 | 222493_s_at |
| Testis expressed sequence 27 | TEX27 | Hs.36959 | 235847_at |
| Testis expressed sequence 27 | TEX27 | Hs.36959 | 243827_at |
| testis nuclear RNA-binding protein | Tenr | Hs.518957 | 231448_at |
| testis specific, 10 | TSGA10 | Hs.120267 | 220623_s_at |
| testis-specific leucine zipper protein nurit | NURIT | Hs.186363 | 1553847_a_at |
| Testis-specific serine kinase 2 | STK22B | Hs.505323 | 239730_at |
| tetraspanin 12 | TSPAN12 | Hs.16529 | 230626_at |
| tetraspanin 15 | TSPAN15 | Hs.499941 | 218693_at |
| tetraspanin 16 | TSPAN16 | Hs.444272 | 242179_s_at |
| tetraspanin 16 | TSPAN16 | Hs.444272 | 242180_at |
| tetraspanin 18 | TSPAN18 | Hs.385634 | 1561449_at |
| tetraspanin 6 | TSPAN6 | Hs.43233 | 209109_s_at |
| tetratricopeptide repeat domain 10 | TTC10 | Hs.187376 | 204703_at |
| tetratricopeptide repeat domain 14 | TTC14 | Hs.43213 | 225180_at |
| tetratricopeptide repeat domain 17 | TTC17 | Hs.191186 | 218972_at |
| tetratricopeptide repeat domain 18 | TTC18 | Hs.549236 | 229170_s_at |
| Tetratricopeptide repeat domain 18 | TTC18 | Hs.549236 | 229388_at |
| tetratricopeptide repeat domain 19 | TTC19 | Hs.462316 | 217964_at |
| tetratricopeptide repeat domain 3 | TTC3 | Hs.368214 | 208073_x_at |
| tetratricopeptide repeat domain 3 | TTC3 | Hs.368214 | 208661_s_at |
| tetratricopeptide repeat domain 3 | TTC3 | Hs.368214 | 208662_s_at |
| tetratricopeptide repeat domain 7A | TTC7A | Hs.370603 | 224924_at |
| Tetratricopeptide repeat domain 7A | TTC7A | Hs.370603 | 240369_at |
| TGF beta-inducible nuclear protein 1 | TINP1 | Hs.482526 | 201922_at |
| TGFB-induced factor (TALE family homeobox) | TGIF | Hs.373550 | 203313_s_at |
| THAP domain containing 10 | THAP10 | Hs.26815 | 219596_at |
| THAP domain containing 5 | THAP5 | Hs.290259 | 244190_at |
| THAP domain containing 5 /// hypothetical gene | THAP5 | Hs.290259 | 227636_at |
| supported by AL833137 | /// | | |
| | LOC285872 | | |
| THAP domain containing 8 | THAP8 | Hs.350209 | 228500_at |
| THAP domain containing 9 | THAP9 | Hs.409270 | 230185_at |
| thiamin pyrophosphokinase 1 | TPK1 | Hs.490454 | 221218_s_at |
| thioesterase domain containing 1 | THEDC1 | Hs.24309 | 219975_x_at |
| thioredoxin | TXN | Hs.435136 | 208864_s_at |
| thioredoxin domain containing 10 | TXNDC10 | Hs.440534 | 225302_at |
| thioredoxin domain containing 2 (spermatozoa) | TXNDC2 | Hs.98712 | 1552657_a_at |
| thioredoxin domain containing 9 | TXNDC9 | Hs.370017 | 203008_x_at |
| thioredoxin reductase 1 | TXNRD1 | Hs.434367 | 201266_at |
| thioredoxin-like 2 | TXNL2 | Hs.42644 | 209080_x_at |
| thioredoxin-like 4A | TXNL4A | Hs.465498 | 202836_s_at |
| thioredoxin-like 4B | TXNL4B | Hs.134406 | 222748_s_at |
| THO complex 1 | THOC1 | Hs.1540 | 226567_at |
| THO complex 2 | THOC2 | Hs.149991 | 230651_at |
| THO complex 3 | THOC3 | Hs.484227 | 236149_at |
| threonyl-tRNA synthetase | TARS | Hs.481860 | 201263_at |
| Threonyl-tRNA synthetase | TARS | Hs.481860 | 240206_at |
| threonyl-tRNA synthetase-like 2 | TARSL2 | Hs.498500 | 227611_at |
| Thrombospondin 1 | THBS1 | Hs.164226 | 215775_at |
| thrombospondin 3 | THBS3 | Hs.169875 | 209561_at |
| THUMP domain containing 1 | THUMPD1 | Hs.460232 | 206555_s_at |
| THUMP domain containing 3 | THUMPD3 | Hs.443081 | 225730_s_at |
| THUMP domain containing 3 | THUMPD3 | Hs.443081 | 242377_x_at |
| Thymidine kinase 2, mitochondrial | TK2 | Hs.512619 | 208246_x_at |
| Thymidylate synthetase | TYMS | Hs.369762 | 243016_at |
| thymine-DNA glycosylase | TDG | Hs.173824 | 203743_s_at |
| thymopoietin | TMPO | Hs.11355 | 209753_s_at |
| thymosin, beta 4, X-linked /// thymosin-like 3 | TMSB4X | Hs.522584 | 216438_s_at |
| | /// | | |
| | TMSL3 | | |
| thymus expressed gene 3-like | MGC15476 | Hs.515490 | 228228_at |
| thyroid hormone receptor associated protein 1 | THRAP1 | Hs.282678 | 201986_at |
| Thyroid hormone receptor associated protein 1 | THRAP1 | Hs.282678 | 201987_at |
| Thyroid hormone receptor associated protein 1 | THRAP1 | Hs.282678 | 236978_at |
| thyroid hormone receptor associated protein 2 | THRAP2 | Hs.159799 | 212207_at |
| thyroid hormone receptor associated protein 2 | THRAP2 | Hs.159799 | 212208_at |
| Thyroid hormone receptor associated protein 2 | THRAP2 | Hs.159799 | 238883_at |
| thyroid hormone receptor associated protein 6 | THRAP6 | Hs.492612 | 227786_at |
| thyroid hormone receptor interactor 12 | TRIP12 | Hs.368985 | 201546_at |
| Thyroid hormone receptor interactor 12 | TRIP12 | Hs.368985 | 242688_at |
| thyroid hormone receptor interactor 13 | TRIP13 | Hs.436187 | 204033_at |
| tigger transposable element derived 7 | TIGD7 | Hs.161287 | 238793_at |
| tight junction protein 1 (zona occludens 1) | TJP1 | Hs.510833 | 202011_at |
| tight junction protein 1 (zona occludens 1) | TJP1 | Hs.510833 | 214168_s_at |
| Tight junction protein 1 (zona occludens 1) | TJP1 | Hs.510833 | 243184_at |
| timeless homolog (Drosophila) | TIMELESS | Hs.118631 | 203046_s_at |
| tissue inhibitor of metalloproteinase 2 | TIMP2 | Hs.104839 | 231579_s_at |
| Titin | TTN | Hs.134602 | 240793_at |
| TL132 protein | LOC220594 | Hs.234573 | 213510_x_at |
| TNT protein | TNT | Hs.120831 | 231076_at |
| toll-interleukin 1 receptor (TIR) domain containing | TIRAP | Hs.549233 | 1554091_a_at |
| adaptor protein | | | |
| toll-like receptor 3 | TLR3 | Hs.29499 | 206271_at |
| toll-like receptor 5 | TLR5 | Hs.114408 | 210166_at |
| topoisomerase (DNA) I | TOP1 | Hs.472737 | 208901_s_at |
| topoisomerase (DNA) II binding protein 1 | TOPBP1 | Hs.53454 | 202633_at |
| topoisomerase I binding, arginine/serine-rich | TOPORS | Hs.535961 | 204071_s_at |
| Topoisomerase I binding, arginine/serine-rich | TOPORS | Hs.535961 | 78383_at |
| tousled-like kinase 1 | TLK1 | Hs.470586 | 202606_s_at |
| tousled-like kinase 1 | TLK1 | Hs.470586 | 210379_s_at |
| tousled-like kinase 2 | TLK2 | Hs.445078 | 212986_s_at |
| TP53TG3 protein | TP53TG3 | Hs.513537 | 220167_s_at |
| TPA regulated locus | TPARL | Hs.479766 | 1560622_at |
| TPTE and PTEN homologous inositol lipid | TPTEps1 | — | 1556876_s_at |
| phosphatase pseudogene | | | |
| TPX2, microtubule-associated protein homolog | TPX2 | Hs.244580 | 210052_s_at |
| (Xenopus laevis) | | | |
| TRAF family member-associated NFKB activator | TANK | Hs.516490 | 207616_s_at |
| TRAF family member-associated NFKB activator | TANK | Hs.516490 | 209451_at |
| trafficking protein particle complex 2 /// | TRAPPC2 | Hs.201054 | 209751_s_at |
| spondyloepiphyseal dysplasia, late, pseudogene | /// | | |
| | SEDLP | | |
| trafficking protein particle complex 4 | TRAPPC4 | Hs.524078 | 217958_at |
| transaldolase 1 /// similar to RPE-spondin | TALDO1 | Hs.356766 | 224915_x_at |
| | /// | | |
| | LOC441951 | | |
| transaldolase 1 /// similar to RPE-spondin | TALDO1 | Hs.356766 | 226227_x_at |
| | /// | | |
| | LOC441951 | | |
| Transcribed locus | — | Hs.388575 | 225444_at |
| Transcribed locus | — | Hs.388575 | 225445_at |
| Transcribed locus | — | Hs.9012 | 227524_at |
| Transcribed locus | — | Hs.293796 | 228151_at |
| Transcribed locus | — | Hs.445588 | 228479_at |
| Transcribed locus | — | Hs.549682 | 228753_at |
| Transcribed locus | — | Hs.67197 | 229066_at |
| Transcribed locus | — | Hs.365365 | 229696_at |
| Transcribed locus | — | Hs.552082 | 229905_at |
| Transcribed locus | — | Hs.88156 | 230003_at |
| Transcribed locus | — | Hs.86941 | 230208_at |
| Transcribed locus | — | Hs.112050 | 230310_at |
| Transcribed locus | — | Hs.23457 | 230503_at |
| Transcribed locus | — | Hs.536825 | 230653_at |
| Transcribed locus | — | Hs.527014 | 230862_at |
| Transcribed locus | — | Hs.116507 | 231116_at |
| Transcribed locus | — | Hs.445843 | 231312_at |
| Transcribed locus | — | Hs.97990 | 231409_at |
| Transcribed locus | — | Hs.112962 | 231611_at |
| Transcribed locus | — | Hs.473374 | 233976_at |
| Transcribed locus | — | Hs.94499 | 235072_s_at |
| Transcribed locus | — | Hs.546736 | 235419_at |
| Transcribed locus | — | Hs.122020 | 235477_at |
| Transcribed locus | — | Hs.47026 | 235635_at |
| Transcribed locus | — | Hs.551066 | 235693_at |
| Transcribed locus | — | Hs.445878 | 235808_at |
| Transcribed locus | — | Hs.182362 | 236161_at |
| Transcribed locus | — | Hs.422318 | 236428_at |
| Transcribed locus | — | Hs.436029 | 236685_at |
| Transcribed locus | — | Hs.97439 | 236703_at |
| Transcribed locus | — | Hs.444348 | 236720_at |
| Transcribed locus | — | Hs.12316 | 236793_at |
| Transcribed locus | — | Hs.553088 | 236992_at |
| Transcribed locus | — | Hs.9394 | 236996_at |
| Transcribed locus | — | Hs.89113 | 237064_x_at |
| Transcribed locus | — | Hs.29178 | 237383_at |
| Transcribed locus | — | Hs.61481 | 237419_at |
| Transcribed locus | — | Hs.106212 | 237436_at |
| Transcribed locus | — | Hs.174746 | 237553_at |
| Transcribed locus | — | Hs.202054 | 237652_at |
| Transcribed locus | — | Hs.128103 | 237812_at |
| Transcribed locus | — | Hs.123362 | 238098_at |
| Transcribed locus | — | Hs.552919 | 238213_at |
| Transcribed locus | — | Hs.493949 | 239069_s_at |
| Transcribed locus | — | Hs.522192 | 239236_at |
| Transcribed locus | — | Hs.440588 | 239455_at |
| Transcribed locus | — | Hs.161318 | 239677_at |
| Transcribed locus | — | Hs.18200 | 239798_at |
| Transcribed locus | — | Hs.117688 | 239842_x_at |
| Transcribed locus | — | Hs.129636 | 240095_at |
| Transcribed locus | — | Hs.129907 | 240153_at |
| Transcribed locus | — | Hs.264236 | 240487_at |
| Transcribed locus | — | Hs.145189 | 240525_at |
| Transcribed locus | — | Hs.129196 | 240620_at |
| Transcribed locus | — | Hs.97753 | 240696_at |
| Transcribed locus | — | Hs.98466 | 240894_at |
| Transcribed locus | — | Hs.143911 | 241095_at |
| Transcribed locus | — | Hs.150792 | 241100_at |
| Transcribed locus | — | Hs.126622 | 241130_at |
| Transcribed locus | — | Hs.116243 | 241204_at |
| Transcribed locus | — | Hs.157003 | 241251_at |
| Transcribed locus | — | Hs.134198 | 241417_at |
| Transcribed locus | — | Hs.432345 | 241421_at |
| Transcribed locus | — | Hs.542861 | 241686_x_at |
| Transcribed locus | — | Hs.164964 | 241841_at |
| Transcribed locus | — | Hs.437802 | 241852_at |
| Transcribed locus | — | Hs.8469 | 241893_at |
| Transcribed locus | — | Hs.117688 | 241936_x_at |
| Transcribed locus | — | Hs.269783 | 242021_at |
| Transcribed locus | — | Hs.137182 | 242032_at |
| Transcribed locus | — | Hs.127380 | 242147_at |
| Transcribed locus | — | Hs.436060 | 242150_at |
| Transcribed locus | — | Hs.498048 | 242316_at |
| Transcribed locus | — | Hs.192124 | 242317_at |
| Transcribed locus | — | Hs.435100 | 242365_at |
| Transcribed locus | — | Hs.257891 | 242421_at |
| Transcribed locus | — | Hs.28170 | 242471_at |
| Transcribed locus | — | Hs.122390 | 242557_at |
| Transcribed locus | — | Hs.271736 | 242607_at |
| Transcribed locus | — | Hs.48100 | 242978_x_at |
| Transcribed locus | — | Hs.178202 | 243095_at |
| Transcribed locus | — | Hs.161434 | 243218_at |
| Transcribed locus | — | Hs.6656 | 243396_at |
| Transcribed locus | — | Hs.531274 | 243571_at |
| Transcribed locus | — | Hs.131244 | 243604_at |
| Transcribed locus | — | Hs.445342 | 243675_at |
| Transcribed locus | — | Hs.119922 | 243794_at |
| Transcribed locus | — | Hs.411959 | 243939_at |
| Transcribed locus | — | Hs.151334 | 243996_at |
| Transcribed locus | — | Hs.188594 | 244007_at |
| Transcribed locus | — | Hs.553278 | 244036_at |
| Transcribed locus | — | Hs.97763 | 244129_at |
| Transcribed locus | — | Hs.170973 | 244397_at |
| Transcribed locus | — | Hs.443381 | 244465_at |
| Transcribed locus | — | Hs.71657 | 244544_at |
| Transcribed locus | — | Hs.209222 | 244636_at |
| Transcribed locus, moderately similar to | — | Hs.498322 | 217643_x_at |
| NP_001888.2 chondroitin sulfate proteoglycan 4 | | | |
| (melanoma-associated) [Homo sapiens ] | | | |
| Transcribed locus, moderately similar to | — | Hs.535736 | 221844_x_at |
| NP_055301.1 neuronal thread protein AD7c-NTP | | | |
| [Homo sapiens ] | | | |
| Transcribed locus, moderately similar to | — | Hs.121749 | 231456_at |
| NP_066288.2 deformed epidermal autoregulatory | | | |
| factor 1 (Drosophila) [Homo sapiens ] | | | |
| Transcribed locus, moderately similar to | — | Hs.191956 | 240970_x_at |
| NP_699194.1 hypothetical protein MGC42415 | | | |
| [Homo sapiens ] | | | |
| Transcribed locus, moderately similar to | — | Hs.135098 | 237732_at |
| NP_780633.1 RIKEN cDNA A030004J04 gene | | | |
| [Mus musculus] | | | |
| Transcribed locus, moderately similar to | — | Hs.535773 | 230957_at |
| XP_226005.2 similar to protocadherin beta 3 | | | |
| [Rattus norvegicus] | | | |
| Transcribed locus, moderately similar to | — | Hs.112899 | 230305_at |
| XP_414333.1 similar to transketolase [Gallus | | | |
| gallus] | | | |
| Transcribed locus, moderately similar to | — | Hs.130561 | 244674_at |
| XP_496299.1 hypothetical protein LOC148206 | | | |
| [Homo sapiens ] | | | |
| Transcribed locus, moderately similar to | — | Hs.549555 | 244775_at |
| XP_496299.1 hypothetical protein LOC148206 | | | |
| [Homo sapiens ] | | | |
| Transcribed locus, moderately similar to | — | Hs.507924 | 237771_s_at |
| XP_497042.1 hypothetical gene supported by | | | |
| AK091508 [Homo sapiens ] | | | |
| Transcribed locus, moderately similar to | — | Hs.263209 | 244296_at |
| XP_509882.1 LOC452831 [Pan troglodytes] | | | |
| Transcribed locus, moderately similar to | — | Hs.514833 | 213637_at |
| XP_517655.1 similar to KIAA0825 protein [Pan | | | |
| troglodytes] | | | |
| Transcribed locus, moderately similar to | — | Hs.522536 | 217674_at |
| XP_517655.1 similar to KIAA0825 protein [Pan | | | |
| troglodytes] | | | |
| Transcribed locus, moderately similar to | — | Hs.445034 | 235423_at |
| XP_517655.1 similar to KIAA0825 protein [Pan | | | |
| troglodytes] | | | |
| Transcribed locus, moderately similar to | — | Hs.121070 | 235513_at |
| XP_517655.1 similar to KIAA0825 protein [Pan | | | |
| troglodytes] | | | |
| Transcribed locus, moderately similar to | — | Hs.324323 | 238431_at |
| XP_517655.1 similar to KIAA0825 protein [Pan | | | |
| troglodytes] | | | |
| Transcribed locus, moderately similar to | — | Hs.547688 | 242491_at |
| XP_519595.1 similar to FLJ46489 protein [Pan | | | |
| troglodytes] | | | |
| Transcribed locus, strongly similar to NP_002137.3 | — | Hs.171806 | 228904_at |
| homeo box B3 [Homo sapiens ] | | | |
| Transcribed locus, strongly similar to XP_509281.1 | — | Hs.125549 | 240919_at |
| similar to SNRPF protein [Pan troglodytes] | | | |
| Transcribed locus, strongly similar to XP_515644.1 | — | Hs.546967 | 242053_at |
| similar to TSGA10 isoform [Pan troglodytes] | | | |
| Transcribed locus, strongly similar to XP_519903.1 | — | Hs.259168 | 237743_at |
| similar to oxidation resistance 1 [Pan troglodytes] | | | |
| Transcribed locus, strongly similar to XP_520590.1 | — | Hs.130729 | 242476_at |
| similar to hypothetical protein FLJ30435 [Pan | | | |
| troglodytes] | | | |
| Transcribed locus, strongly similar to XP_529427.1 | — | Hs.16586 | 237792_at |
| LOC451392 [Pan troglodytes] | | | |
| Transcribed locus, weakly similar to NP_080901.1 | — | Hs.116822 | 237779_at |
| RIKEN cDNA 1700067P10 gene [Mus musculus] | | | |
| Transcribed locus, weakly similar to NP_659411.1 | — | Hs.150065 | 236494_x_at |
| hypothetical protein FLJ31846 [Homo sapiens ] | | | |
| Transcribed locus, weakly similar to NP_703324.1 | — | Hs.445247 | 235556_at |
| Plasmodium falciparum 3D7 MAL1P3.06 gene | | | |
| Transcribed locus, weakly similar to XP_209041.2 | — | Hs.271498 | 240165_at |
| KIAA1503 protein [Homo sapiens ] | | | |
| Transcribed locus, weakly similar to XP_209041.2 | — | Hs.135232 | 242333_at |
| KIAA1503 protein [Homo sapiens ] | | | |
| Transcribed locus, weakly similar to XP_331555.1 | — | Hs.405444 | 228879_at |
| Neurospora crassa NCU09163.1 gene | | | |
| Transcribed locus, weakly similar to XP_331555.1 | — | Hs.131365 | 235474_at |
| Neurospora crassa NCU09163.1 gene | | | |
| Transcribed locus, weakly similar to XP_371841.1 | — | Hs.432337 | 236963_at |
| similar to hypothetical protein (L1H 3 region) — | | | |
| human [Homo sapiens ] | | | |
| Transcribed locus, weakly similar to XP_375935.1 | — | Hs.134812 | 241877_at |
| hypothetical protein LOC284825 [Homo sapiens ] | | | |
| Transcribed locus, weakly similar to XP_376619.2 | — | Hs.552124 | 235884_at |
| hypothetical LOC401357 [Homo sapiens ] | | | |
| Transcribed locus, weakly similar to XP_376619.2 | — | Hs.552124 | 238921_at |
| hypothetical LOC401357 [Homo sapiens ] | | | |
| Transcribed locus, weakly similar to XP_496299.1 | — | Hs.469970 | 242837_at |
| hypothetical protein LOC148206 [Homo sapiens ] | | | |
| Transcribed locus, weakly similar to XP_499236.1 | — | Hs.438801 | 229029_at |
| LOC442377 [Homo sapiens ] | | | |
| Transcribed locus, weakly similar to XP_509882.1 | — | Hs.126660 | 242159_at |
| LOC452831 [Pan troglodytes] | | | |
| Transcribed locus, weakly similar to XP_510104.1 | — | Hs.544085 | 1570566_at |
| similar to hypothetical protein FLJ25224 [Pan | | | |
| troglodytes] | | | |
| Transcribed locus, weakly similar to XP_510104.1 | — | Hs.523129 | 217554_at |
| similar to hypothetical protein FLJ25224 [Pan | | | |
| troglodytes] | | | |
| Transcribed locus, weakly similar to XP_512615.1 | — | Hs.297285 | 242820_at |
| similar to hypothetical protein FLJ32191 [Pan | | | |
| troglodytes] | | | |
| Transcribed locus, weakly similar to XP_515012.1 | — | Hs.505398 | 242412_at |
| similar to ASH2-like protein [Pan troglodytes] | | | |
| Transcribed locus, weakly similar to XP_517454.1 | — | Hs.442978 | 239734_at |
| similar to hypothetical protein MGC45438 [Pan | | | |
| troglodytes] | | | |
| Transcribed locus, weakly similar to XP_522312.1 | — | Hs.148211 | 231147_at |
| similar to voltage-gated calcium channel | | | |
| alpha(2)delta-4 subunit [Pan troglodytes] | | | |
| Transcribed locus, weakly similar to XP_527683.1 | — | Hs.529854 | 240445_at |
| similar to ribosomal protein L13a; 60S ribosomal | | | |
| protein L13a; 23 kD highly basic protein [Pan | | | |
| troglodytes] | | | |
| Transcribed locus, weakly similar to XP_535457.1 | — | Hs.121876 | 231604_at |
| similar to H63 breast cancer expressed gene | | | |
| isoform a [Canis familiaris] | | | |
| transcription elongation factor B (SIII), polypeptide | TCEB1 | Hs.546305 | 202824_s_at |
| 1 (15 kDa, elongin C) | | | |
| transcription elongation factor B polypeptide 3B | TCEB3B | Hs.375035 | 220844_at |
| (elongin A2) | | | |
| transcription elongation regulator 1 | TCERG1 | Hs.443465 | 202396_at |
| transcription factor 12 (HTF4, helix-loop-helix | TCF12 | Hs.511504 | 208986_at |
| transcription factors 4) | | | |
| transcription factor 20 (AR1) | TCF20 | Hs.475018 | 215511_at |
| transcription factor 3 (E2A immunoglobulin | TCF3 | Hs.371282 | 213811_x_at |
| enhancer binding factors E12/E47) | | | |
| transcription factor 4 | TCF4 | Hs.200285 | 203753_at |
| Transcription factor 4 | TCF4 | Hs.200285 | 212386_at |
| Transcription factor 4 | TCF4 | Hs.200285 | 213891_s_at |
| transcription factor 7 (T-cell specific, HMG-box) | TCF7 | Hs.519580 | 205255_x_at |
| transcription factor 7-like 2 (T-cell specific, HMG- | TCF7L2 | Hs.501080 | 212762_s_at |
| box) | | | |
| transcription factor A, mitochondrial | TFAM | Hs.75133 | 203176_s_at |
| transcription factor A, mitochondrial | TFAM | Hs.75133 | 208541_x_at |
| transcription factor AP-2 alpha (activating | TFAP2A | Hs.519880 | 204653_at |
| enhancer binding protein 2 alpha) | | | |
| transcription factor AP-2 epsilon (activating | TFAP2E | Hs.434756 | 1555348_at |
| enhancer binding protein 2 epsilon) | | | |
| transcription factor B1, mitochondrial | TFB1M | Hs.279908 | 228075_x_at |
| transcription factor B2, mitochondrial | TFB2M | Hs.7395 | 218605_at |
| transcription factor CP2-like 2 | TFCP2L2 | Hs.418493 | 222830_at |
| Transcription factor CP2-like 3 | TFCP2L3 | Hs.161160 | 235247_at |
| transcription factor Dp-1 | TFDP1 | Hs.79353 | 212330_at |
| transcription factor MLR1 | MLR1 | Hs.446201 | 235970_at |
| transcription factor MLR1 | MLR1 | Hs.446201 | 240592_at |
| transcription termination factor, RNA polymerase I | TTF1 | Hs.54780 | 204771_s_at |
| transcription termination factor-like protein | LOC80298 | Hs.5009 | 225341_at |
| transcription termination factor-like protein | LOC80298 | Hs.5009 | 225346_at |
| transcriptional activator of the c-fos promoter | CROC4 | Hs.380027 | 205103_at |
| transcriptional intermediary factor 1 | TIF1 | Hs.490287 | 204391_x_at |
| transducer of ERBB2, 1 | TOB1 | Hs.531550 | 202704_at |
| transducer of ERBB2, 1 | TOB1 | Hs.531550 | 228834_at |
| transducer of ERBB2, 2 | TOB2 | Hs.474978 | 222243_s_at |
| transducin (beta)-like 1X-linked receptor 1 | TBL1XR1 | Hs.438970 | 222633_at |
| transducin (beta)-like 1X-linked receptor 1 | TBL1XR1 | Hs.438970 | 223013_at |
| transducin (beta)-like 2 | TBL2 | Hs.52515 | 212685_s_at |
| transducin-like enhancer of split 1 (E(sp1) | TLE1 | Hs.197320 | 203221_at |
| homolog, Drosophila) | | | |
| transducin-like enhancer of split 3 (E(sp1) | TLE3 | Hs.287362 | 212769_at |
| homolog, Drosophila) | | | |
| transducin-like enhancer of split 4 (E(sp1) | TLE4 | Hs.444213 | 214688_at |
| homolog, Drosophila) | | | |
| transducin-like enhancer of split 4 (E(sp1) | TLE4 | Hs.444213 | 235765_at |
| homolog, Drosophila) | | | |
| transferrin receptor 2 | TFR2 | Hs.544932 | 210215_at |
| transformer-2 alpha | TRA2A | Hs.445652 | 204658_at |
| Transformer-2 alpha | TRA2A | Hs.445652 | 213593_s_at |
| Transformer-2 alpha | TRA2A | Hs.445652 | 229574_at |
| transforming growth factor, alpha | TGFA | Hs.170009 | 205016_at |
| transforming growth factor, beta 2 | TGFB2 | Hs.133379 | 209909_s_at |
| Transforming growth factor, beta receptor | TGFBRAP1 | Hs.446350 | 221235_s_at |
| associated protein 1 /// Transforming growth factor, | | | |
| beta receptor associated protein 1 | | | |
| transforming growth factor, beta receptor III | TGFBR3 | Hs.482390 | 204731_at |
| (betaglycan, 300 kDa) | | | |
| Transforming growth factor, beta receptor III | TGFBR3 | Hs.482390 | 226625_at |
| (betaglycan, 300 kDa) | | | |
| trans-golgi network protein 2 | TGOLN2 | Hs.14894 | 203833_s_at |
| Transient receptor potential cation channel, | TRPM1 | Hs.155942 | 240386_at |
| subfamily M, member 1 | | | |
| transition protein 1 (during histone to protamine | TNP1 | Hs.3017 | 206568_at |
| replacement) | | | |
| translation factor sui1 homolog | GC20 | Hs.315230 | 201738_at |
| translocase of inner mitochondrial membrane 10 | TIMM10 | Hs.235750 | 1555764_s_at |
| homolog (yeast) | | | |
| translocase of inner mitochondrial membrane 13 | TIMM13 | Hs.75056 | 218188_s_at |
| homolog (yeast) | | | |
| translocase of inner mitochondrial membrane 23 | TIMM23 | Hs.524308 | 218119_at |
| homolog (yeast) | | | |
| translocase of inner mitochondrial membrane 8 | TIMM8B | Hs.279915 | 218357_s_at |
| homolog B (yeast) | | | |
| translocase of outer mitochondrial membrane 20 | TOMM20 | Hs.533192 | 200662_s_at |
| homolog (yeast) | | | |
| translocase of outer mitochondrial membrane 34 | TOMM34 | Hs.517066 | 201870_at |
| translocase of outer mitochondrial membrane 7 | TOMM7 | Hs.380920 | 201812_s_at |
| homolog (yeast) /// hypothetical protein | /// | | |
| LOC201725 | LOC201725 | | |
| translocated promoter region (to activated MET | TPR | Hs.279640 | 201730_s_at |
| oncogene) | | | |
| translocation associated membrane protein 1 | TRAM1 | Hs.491988 | 201398_s_at |
| translocation associated membrane protein 1 | TRAM1 | Hs.491988 | 201399_s_at |
| translocation protein 1 | TLOC1 | Hs.529591 | 208942_s_at |
| translokin | PIG8 | Hs.101014 | 203491_s_at |
| translokin | PIG8 | Hs.101014 | 209862_s_at |
| Transmembrane 4 superfamily member 9 | TM4SF9 | Hs.118118 | 237194_at |
| Transmembrane 4 superfamily member 9 | TM4SF9 | Hs.118118 | 241060_x_at |
| transmembrane 7 superfamily member 3 | TM7SF3 | Hs.438641 | 222477_s_at |
| transmembrane 9 superfamily member 1 | TM9SF1 | Hs.91586 | 209149_s_at |
| transmembrane 9 superfamily member 1 | TM9SF1 | Hs.91586 | 209150_s_at |
| transmembrane 9 superfamily member 2 | TM9SF2 | Hs.130413 | 201078_at |
| transmembrane 9 superfamily protein member 4 | TM9SF4 | Hs.529360 | 212194_s_at |
| transmembrane anchor protein 1 | TMAP1 | Hs.250723 | 212509_s_at |
| transmembrane and coiled-coil domains 1 | TMCC1 | Hs.477547 | 213351_s_at |
| transmembrane emp24 domain containing 3 | TMED3 | Hs.513058 | 208837_at |
| transmembrane emp24 domain containing 8 | TMED8 | Hs.200413 | 225343_at |
| transmembrane emp24 protein transport domain | TMED4 | Hs.510745 | 1558487_a_at |
| containing 4 | | | |
| transmembrane emp24 protein transport domain | TMED5 | Hs.482873 | 202195_s_at |
| containing 5 | | | |
| transmembrane phosphatase with tensin homology | TPTE | Hs.122986 | 220205_at |
| transmembrane protein 1 | TMEM1 | Hs.126221 | 1555446_s_at |
| transmembrane protein 1 | TMEM1 | Hs.126221 | 215269_at |
| transmembrane protein 14B /// transmembrane | TMEM14B | Hs.273077 | 221452_s_at |
| protein 14B | | | |
| transmembrane protein 14C /// transmembrane | TMEM14C | Hs.519557 | 223105_s_at |
| protein 14B | /// | | |
| | TMEM14B | | |
| Transmembrane protein 23 | TMEM23 | Hs.386215 | 1552829_at |
| transmembrane protein 29 | TMEM29 | Hs.6451 | 223203_at |
| transmembrane protein 31 | TMEM31 | Hs.98843 | 231569_at |
| transmembrane protein 38B | TMEM38B | Hs.411925 | 222736_s_at |
| transmembrane protein 39A | TMEM39A | Hs.434927 | 222690_s_at |
| transmembrane protein 4 | TMEM4 | Hs.8752 | 209797_at |
| Transmembrane protein 41B | TMEM41B | Hs.501865 | 212622_at |
| transmembrane protein 42 | TMEM42 | Hs.432780 | 226361_at |
| transmembrane protein 45A | TMEM45A | Hs.126598 | 219410_at |
| transmembrane protein 47 | TMEM47 | Hs.8769 | 209656_s_at |
| transmembrane protein induced by tumor necrosis | TMPIT | Hs.488835 | 223482_at |
| factor alpha | | | |
| transmembrane protein with EGF-like and two | TMEFF2 | Hs.144513 | 224321_at |
| follistatin-like domains 2 /// transmembrane protein | | | |
| with EGF-like and two follistatin-like domains 2 | | | |
| transportin 1 | TNPO1 | Hs.482497 | 207657_x_at |
| transportin 1 | TNPO1 | Hs.482497 | 209225_x_at |
| transportin 1 | TNPO1 | Hs.482497 | 221829_s_at |
| Transportin 1 | TNPO1 | Hs.482497 | 225765_at |
| transportin 3 | TNPO3 | Hs.193613 | 212318_at |
| trichorhinophalangeal syndrome I | TRPS1 | Hs.253594 | 222651_s_at |
| trinucleotide repeat containing 15 | TNRC15 | Hs.435841 | 212261_at |
| trinucleotide repeat containing 5 | TNRC5 | Hs.414099 | 228369_at |
| trinucleotide repeat containing 6A | TNRC6A | Hs.407740 | 224704_at |
| trinucleotide repeat containing 6A | TNRC6A | Hs.407740 | 224705_s_at |
| triosephosphate isomerase 1 | TPI1 | Hs.524219 | 200822_x_at |
| triosephosphate isomerase 1 | TPI1 | Hs.524219 | 213011_s_at |
| tripartite motif-containing 11 | TRIM11 | Hs.13543 | 226566_at |
| tripartite motif-containing 14 | TRIM14 | Hs.549094 | 203147_s_at |
| tripartite motif-containing 22 | TRIM22 | Hs.501778 | 213293_s_at |
| tripartite motif-containing 33 | TRIM33 | Hs.26837 | 210266_s_at |
| tripartite motif-containing 36 | TRIM36 | Hs.519514 | 231123_at |
| tripartite motif-containing 37 | TRIM37 | Hs.412767 | 213009_s_at |
| tripartite motif-containing 4 | TRIM4 | Hs.50749 | 224159_x_at |
| tripartite motif-containing 59 | TRIM59 | Hs.212957 | 235476_at |
| tripeptidyl peptidase I | TPP1 | Hs.523454 | 200742_s_at |
| tripeptidyl peptidase I | TPP1 | Hs.523454 | 200743_s_at |
| tRNA splicing endonuclease 34 homolog (SEN34, | TSEN34 | Hs.15580 | 218132_s_at |
| S. cerevisiae) | | | |
| trophoblast glycoprotein | TPBG | Hs.82128 | 203476_at |
| trophoblast-derived noncoding RNA | TncRNA | Hs.523789 | 224565_at |
| trophoblast-derived noncoding RNA | TncRNA | Hs.523789 | 224566_at |
| trophoblast-derived noncoding RNA | TncRNA | Hs.523789 | 234989_at |
| tropomodulin 3 (ubiquitous) | TMOD3 | Hs.4998 | 223077_at |
| tropomyosin 1 (alpha) | TPM1 | Hs.133892 | 210987_x_at |
| tropomyosin 3 | TPM3 | Hs.146070 | 222976_s_at |
| tropomyosin 4 | TPM4 | Hs.466088 | 212481_s_at |
| trypsin X3 | TRY1 | Hs.98609 | 237513_at |
| tryptophan rich basic protein | WRB | Hs.198308 | 202749_at |
| TSC22 domain family 1 | TSC22D1 | Hs.450972 | 215111_s_at |
| TSC22 domain family 2 | TSC22D2 | Hs.52526 | 204094_s_at |
| TSPY-like 4 | TSPYL4 | Hs.284141 | 212928_at |
| tubby like protein 2 | TULP2 | Hs.104636 | 206733_at |
| tubby like protein 3 | TULP3 | Hs.524187 | 205854_at |
| Tubby like protein 4 | TULP4 | Hs.486993 | 239742_at |
| tuberoinfundibular 39 residue protein precursor | TIP39 | Hs.339845 | 1555238_at |
| tuberous sclerosis 1 | TSC1 | Hs.370854 | 209390_at |
| tubulin, alpha 1 (testis specific) | TUBA1 | Hs.75318 | 212242_at |
| tubulin, alpha 3 | TUBA3 | Hs.524395 | 209118_s_at |
| tubulin, alpha, ubiquitous | K- | Hs.524390 | 212639_x_at |
| | ALPHA-1 | | |
| tubulin, alpha, ubiquitous /// tubulin, alpha, | K- | Hs.524390 | 211058_x_at |
| ubiquitous | ALPHA-1 | | |
| tubulin, beta 2 | TUBB2 | Hs.512712 | 204141_at |
| tubulin, beta 6 | TUBB6 | Hs.193491 | 209191_at |
| tubulin, beta polypeptide paralog | RP11- | Hs.300701 | 214023_x_at |
| | 506K6.1 | | |
| Tubulin, gamma complex associated protein 2 | TUBGCP2 | Hs.523370 | 244842_x_at |
| tubulin-specific chaperone a | TBCA | Hs.291212 | 203667_at |
| tudor domain containing 6 | TDRD6 | Hs.427227 | 232692_at |
| tudor domain containing 7 | TDRD7 | Hs.193842 | 213361_at |
| tudor domain containing 9 | TDRD9 | Hs.21454 | 228285_at |
| tuftelin 1 | TUFT1 | Hs.489922 | 205807_s_at |
| Tularik gene 1 | T1 | Hs.26814 | 221836_s_at |
| tumor differentially expressed 1 | TDE1 | Hs.272168 | 221473_x_at |
| tumor differentially expressed 1 /// tumor | TDE1 | Hs.272168 | 211769_x_at |
| differentially expressed 1 | | | |
| tumor necrosis factor (ligand) superfamily, member | TNFSF10 | Hs.478275 | 202687_s_at |
| 10 /// tumor necrosis factor (ligand) superfamily, | | | |
| member 10 | | | |
| tumor necrosis factor receptor superfamily, | TNFRSF10A | Hs.401745 | 231775_at |
| member 10a | | | |
| tumor necrosis factor receptor superfamily, | TNFRSF21 | Hs.443577 | 214581_x_at |
| member 21 | | | |
| tumor necrosis factor superfamily, member 5- | TNFSF5IP1 | Hs.464652 | 218467_at |
| induced protein 1 | | | |
| tumor necrosis factor, alpha-induced protein 2 | TNFAIP2 | Hs.525607 | 202510_s_at |
| tumor necrosis factor, alpha-induced protein 3 | TNFAIP3 | Hs.211600 | 202644_s_at |
| tumor necrosis factor, alpha-induced protein 6 | TNFAIP6 | Hs.437322 | 206026_s_at |
| tumor protein D52 | TPD52 | Hs.368433 | 201690_s_at |
| tumor protein p53 inducible nuclear protein 1 | TP53INP1 | Hs.492261 | 225912_at |
| tumor protein p53 inducible nuclear protein 2 | TP53INP2 | Hs.516994 | 224836_at |
| tumor protein, translationally-controlled 1 | TPT1 | Hs.374596 | 211943_x_at |
| tumor protein, translationally-controlled 1 | TPT1 | Hs.374596 | 212284_x_at |
| tumor protein, translationally-controlled 1 | TPT1 | Hs.374596 | 212869_x_at |
| tumor protein, translationally-controlled 1 | TPT1 | Hs.374596 | 214327_x_at |
| tumor rejection antigen (gp96) 1 | TRA1 | Hs.192374 | 200598_s_at |
| tumor suppressor candidate 1 | TUSC1 | Hs.26268 | 227388_at |
| tumor suppressor candidate 2 | TUSC2 | Hs.517981 | 203272_s_at |
| twisted gastrulation homolog 1 (Drosophila) | TWSG1 | Hs.514685 | 225406_at |
| Two pore segment channel 1 | TPCN1 | Hs.524763 | 242108_at |
| type 1 tumor necrosis factor receptor shedding | ARTS-1 | Hs.436186 | 210385_s_at |
| aminopeptidase regulator | | | |
| tyrosine 3-monooxygenase/tryptophan 5- | YWHAB | Hs.279920 | 217717_s_at |
| monooxygenase activation protein, beta | | | |
| polypeptide | | | |
| tyrosine 3-monooxygenase/tryptophan 5- | YWHAB | Hs.279920 | 217718_s_at |
| monooxygenase activation protein, beta | | | |
| polypeptide | | | |
| tyrosine 3-monooxygenase/tryptophan 5- | YWHAE | Hs.513851 | 210317_s_at |
| monooxygenase activation protein, epsilon | | | |
| polypeptide | | | |
| Tyrosine 3-monooxygenase/tryptophan 5- | YWHAE | Hs.513851 | 213655_at |
| monooxygenase activation protein, epsilon | | | |
| polypeptide | | | |
| tyrosine 3-monooxygenase/tryptophan 5- | YWHAH | Hs.226755 | 201020_at |
| monooxygenase activation protein, eta polypeptide | | | |
| tyrosine 3-monooxygenase/tryptophan 5- | YWHAG | Hs.520974 | 222985_at |
| monooxygenase activation protein, gamma | | | |
| polypeptide | | | |
| tyrosine 3-monooxygenase/tryptophan 5- | YWHAQ | Hs.74405 | 212426_s_at |
| monooxygenase activation protein, theta | | | |
| polypeptide | | | |
| tyrosine 3-monooxygenase/tryptophan 5- | YWHAQ | Hs.74405 | 200693_at |
| monooxygenase activation protein, theta | /// MIB1 | | |
| polypeptide /// mindbomb homolog 1 (Drosophila) | | | |
| tyrosine 3-monooxygenase/tryptophan 5- | YWHAQ | Hs.74405 | 213699_s_at |
| monooxygenase activation protein, theta | /// MIB1 | | |
| polypeptide /// mindbomb homolog 1 (Drosophila) | | | |
| tyrosine 3-monooxygenase/tryptophan 5- | YWHAZ | Hs.492407 | 200638_s_at |
| monooxygenase activation protein, zeta | | | |
| polypeptide | | | |
| tyrosine 3-monooxygenase/tryptophan 5- | YWHAZ | Hs.492407 | 200639_s_at |
| monooxygenase activation protein, zeta | | | |
| polypeptide | | | |
| tyrosine 3-monooxygenase/tryptophan 5- | YWHAZ | Hs.492407 | 200640_at |
| monooxygenase activation protein, zeta | | | |
| polypeptide | | | |
| tyrosine 3-monooxygenase/tryptophan 5- | YWHAZ | Hs.492407 | 200641_s_at |
| monooxygenase activation protein, zeta | | | |
| polypeptide | | | |
| U2 (RNU2) small nuclear RNA auxiliary factor 2 | U2AF2 | Hs.528007 | 218381_s_at |
| U2AF homology motif (UHM) kinase 1 | UHMK1 | Hs.127310 | 224691_at |
| ubinuclein 1 | UBN1 | Hs.440219 | 209088_s_at |
| ubiquilin 2 | UBQLN2 | Hs.522668 | 215884_s_at |
| ubiquilin 3 | UBQLN3 | Hs.189184 | 220422_at |
| ubiquinol-cytochrome c reductase binding protein | UQCRB | Hs.131255 | 205849_s_at |
| ubiquinol-cytochrome c reductase binding protein | UQCRB | Hs.131255 | 209066_x_at |
| ubiquinol-cytochrome c reductase complex (7.2 kD) | UCRC | Hs.284292 | 218190_s_at |
| ubiquinol-cytochrome c reductase core protein II | UQCRC2 | Hs.528803 | 212600_s_at |
| ubiquinol-cytochrome c reductase hinge protein | UQCRH | Hs.481571 | 202233_s_at |
| ubiquinol-cytochrome c reductase, Rieske iron- | UQCRFS1 | Hs.170107 | 208909_at |
| sulfur polypeptide 1 | | | |
| ubiquitin A-52 residue ribosomal protein fusion | UBA52 | Hs.5308 | 221700_s_at |
| product 1 /// ubiquitin A-52 residue ribosomal | | | |
| protein fusion product 1 | | | |
| ubiquitin associated protein 2 | UBAP2 | Hs.493739 | 219192_at |
| ubiquitin associated protein 2 | UBAP2 | Hs.493739 | 221839_s_at |
| ubiquitin associated protein 2-like | UBAP2L | Hs.490551 | 214695_at |
| ubiquitin B /// ubiquitin B | UBB | Hs.356190 | 200633_at |
| ubiquitin C | UBC | Hs.520348 | 208980_s_at |
| ubiquitin C | UBC | Hs.520348 | 211296_x_at |
| ubiquitin protein ligase E3 component n-recognin 2 | UBR2 | Hs.529925 | 212756_s_at |
| ubiquitin protein ligase E3A (human papilloma | UBE3A | Hs.22543 | 211285_s_at |
| virus E6-associated protein, Angelman syndrome) | | | |
| ubiquitin protein ligase E3A (human papilloma | UBE3A | Hs.22543 | 211575_s_at |
| virus E6-associated protein, Angelman syndrome) | | | |
| ubiquitin protein ligase E3A (human papilloma | UBE3A | Hs.22543 | 213128_s_at |
| virus E6-associated protein, Angelman syndrome) | | | |
| ubiquitin protein ligase E3B | UBE3B | Hs.374067 | 212403_at |
| ubiquitin protein ligase E3C | UBE3C | Hs.118351 | 1554794_a_at |
| ubiquitin specific protease 1 | USP1 | Hs.35086 | 202412_s_at |
| ubiquitin specific protease 1 | USP1 | Hs.35086 | 202413_s_at |
| ubiquitin specific protease 10 | USP10 | Hs.136778 | 209137_s_at |
| ubiquitin specific protease 13 (isopeptidase T-3) | USP13 | Hs.175322 | 205356_at |
| ubiquitin specific protease 14 (tRNA-guanine | USP14 | Hs.464416 | 201672_s_at |
| transglycosylase) | | | |
| ubiquitin specific protease 15 | USP15 | Hs.434951 | 210681_s_at |
| Ubiquitin specific protease 15 | USP15 | Hs.434951 | 233473_x_at |
| ubiquitin specific protease 16 | USP16 | Hs.99819 | 218386_x_at |
| ubiquitin specific protease 16 | USP16 | Hs.99819 | 228822_s_at |
| ubiquitin specific protease 19 | USP19 | Hs.255596 | 226357_at |
| ubiquitin specific protease 2 | USP2 | Hs.524085 | 229337_at |
| ubiquitin specific protease 22 /// ubiquitin specific | USP22 | Hs.462492 | 200083_at |
| protease 22 | | | |
| ubiquitin specific protease 24 | USP24 | Hs.477009 | 212381_at |
| ubiquitin specific protease 25 | USP25 | Hs.473370 | 1563497_at |
| ubiquitin specific protease 25 | USP25 | Hs.473370 | 220419_s_at |
| ubiquitin specific protease 3 | USP3 | Hs.458499 | 221654_s_at |
| ubiquitin specific protease 31 | USP31 | Hs.183817 | 226035_at |
| Ubiquitin specific protease 31 | USP31 | Hs.183817 | 239348_at |
| ubiquitin specific protease 32 /// ubiquitin specific | USP32 | Hs.132868 | 211702_s_at |
| protease 32 | | | |
| ubiquitin specific protease 34 | USP34 | Hs.468663 | 207365_x_at |
| ubiquitin specific protease 34 | USP34 | Hs.468663 | 212066_s_at |
| Ubiquitin specific protease 34 | USP34 | Hs.468663 | 233595_at |
| Ubiquitin specific protease 36 | USP36 | Hs.464243 | 227093_at |
| ubiquitin specific protease 37 | USP37 | Hs.166068 | 226730_s_at |
| ubiquitin specific protease 37 | USP37 | Hs.166068 | 232033_at |
| ubiquitin specific protease 38 | USP38 | Hs.480848 | 223289_s_at |
| ubiquitin specific protease 4 (proto-oncogene) | USP4 | Hs.77500 | 202682_s_at |
| ubiquitin specific protease 4 (proto-oncogene) | USP4 | Hs.77500 | 211800_s_at |
| ubiquitin specific protease 40 | USP40 | Hs.96513 | 225089_at |
| Ubiquitin specific protease 42 | USP42 | Hs.31856 | 226172_at |
| ubiquitin specific protease 42 | USP42 | Hs.31856 | 226176_s_at |
| Ubiquitin specific protease 42 | USP42 | Hs.31856 | 228982_s_at |
| ubiquitin specific protease 43 | USP43 | Hs.23935 | 237439_at |
| ubiquitin specific protease 44 | USP44 | Hs.506394 | 224048_at |
| ubiquitin specific protease 46 | USP46 | Hs.331478 | 203870_at |
| ubiquitin specific protease 47 | USP47 | Hs.501924 | 221518_s_at |
| ubiquitin specific protease 6 (Tre-2 oncogene) | USP6 | Hs.448851 | 1555063_at |
| ubiquitin specific protease 6 (Tre-2 oncogene) | USP6 | Hs.448851 | 1555065_x_at |
| Ubiquitin specific protease 7 (herpes virus- | USP7 | Hs.386939 | 201498_at |
| associated) | | | |
| ubiquitin specific protease 7 (herpes virus- | USP7 | Hs.386939 | 201499_s_at |
| associated) | | | |
| Ubiquitin specific protease 7 (herpes virus- | USP7 | Hs.386939 | 230761_at |
| associated) | | | |
| ubiquitin-activating enzyme E1-domain containing 1 | UBE1DC1 | Hs.170737 | 222578_s_at |
| ubiquitin-conjugating enzyme E2 variant 1 /// | UBE2V1 | Hs.420529 | 201001_s_at |
| ubiquitin-conjugating enzyme E2 variant 1 | /// Kua- | | |
| | UEV | | |
| ubiquitin-conjugating enzyme E2 variant 1 /// | UBE2V1 | Hs.420529 | 201002_s_at |
| ubiquitin-conjugating enzyme E2 variant 1 | /// Kua- | | |
| | UEV | | |
| ubiquitin-conjugating enzyme E2, J1 (UBC6 | UBE2J1 | Hs.163776 | 222435_s_at |
| homolog, yeast) | | | |
| ubiquitin-conjugating enzyme E2A (RAD6 | UBE2A | Hs.379466 | 201899_s_at |
| homolog) | | | |
| ubiquitin-conjugating enzyme E2B (RAD6 | UBE2B | Hs.385986 | 202333_s_at |
| homolog) | | | |
| ubiquitin-conjugating enzyme E2B (RAD6 | UBE2B | Hs.385986 | 228588_s_at |
| homolog) | | | |
| ubiquitin-conjugating enzyme E2D 2 (UBC4/5 | UBE2D2 | Hs.108332 | 201343_at |
| homolog, yeast) | | | |
| ubiquitin-conjugating enzyme E2D 2 (UBC4/5 | UBE2D2 | Hs.108332 | 201344_at |
| homolog, yeast) | | | |
| ubiquitin-conjugating enzyme E2D 2 (UBC4/5 | UBE2D2 | Hs.108332 | 201345_s_at |
| homolog, yeast) | | | |
| Ubiquitin-conjugating enzyme E2D 2 (UBC4/5 | UBE2D2 | Hs.108332 | 215604_x_at |
| homolog, yeast) | | | |
| Ubiquitin-conjugating enzyme E2D 2 (UBC4/5 | UBE2D2 | Hs.108332 | 240139_at |
| homolog, yeast) | | | |
| Ubiquitin-conjugating enzyme E2D 3 (UBC4/5 | UBE2D3 | Hs.518773 | 1562878_at |
| homolog, yeast) | | | |
| ubiquitin-conjugating enzyme E2D 3 (UBC4/5 | UBE2D3 | Hs.472031 | 200667_at |
| homolog, yeast) | | | |
| ubiquitin-conjugating enzyme E2D 3 (UBC4/5 | UBE2D3 | Hs.472031 | 200668_s_at |
| homolog, yeast) | | | |
| ubiquitin-conjugating enzyme E2D 3 (UBC4/5 | UBE2D3 | Hs.472031 | 200669_s_at |
| homolog, yeast) | | | |
| Ubiquitin-conjugating enzyme E2D 3 (UBC4/5 | UBE2D3 | Hs.518773 | 230171_at |
| homolog, yeast) | | | |
| Ubiquitin-conjugating enzyme E2D 3 (UBC4/5 | UBE2D3 | Hs.518773 | 233303_at |
| homolog, yeast) | | | |
| Ubiquitin-conjugating enzyme E2D 3 (UBC4/5 | UBE2D3 | Hs.518773 | 243046_at |
| homolog, yeast) | | | |
| ubiquitin-conjugating enzyme E2E 1 (UBC4/5 | UBE2E1 | Hs.164853 | 212519_at |
| homolog, yeast) | | | |
| Ubiquitin-conjugating enzyme E2E 2 (UBC4/5 | UBE2E2 | Hs.475688 | 244610_x_at |
| homolog, yeast) | | | |
| ubiquitin-conjugating enzyme E2G 1 (UBC7 | UBE2G1 | Hs.462035 | 209141_at |
| homolog, C. elegans) | | | |
| ubiquitin-conjugating enzyme E2G 1 (UBC7 | UBE2G1 | Hs.462035 | 209142_s_at |
| homolog, C. elegans) | | | |
| Ubiquitin-conjugating enzyme E2H (UBC8 | UBE2H | Hs.344165 | 229483_at |
| homolog, yeast) | | | |
| Ubiquitin-conjugating enzyme E2H (UBC8 | UBE2H | Hs.344165 | 239721_at |
| homolog, yeast) | | | |
| ubiquitin-conjugating enzyme E2L 6 | UBE2L6 | Hs.425777 | 201649_at |
| ubiquitin-conjugating enzyme E2N (UBC13 | UBE2N | Hs.524630 | 201523_x_at |
| homolog, yeast) | | | |
| ubiquitin-conjugating enzyme E2N (UBC13 | UBE2N | Hs.524630 | 201524_x_at |
| homolog, yeast) | | | |
| ubiquitin-conjugating enzyme E2N (UBC13 | UBE2N | Hs.524630 | 212751_at |
| homolog, yeast) | | | |
| ubiquitin-conjugating enzyme E2R 2 | UBE2R2 | Hs.11184 | 223014_at |
| ubiquitin-conjugating enzyme E2U (putative) | UBE2U | Hs.388746 | 1553776_at |
| ubiquitin-fold modifier 1 | Ufm1 | Hs.524969 | 218050_at |
| Ubiquitin-fold modifier 1 | Ufm1 | Hs.524969 | 242669_at |
| ubiquitin-like 3 | UBL3 | Hs.145575 | 201534_s_at |
| ubiquitin-like, containing PHD and RING finger | UHRF2 | Hs.493401 | 225610_at |
| domains, 2 | | | |
| UBX domain containing 1 | UBXD1 | Hs.435255 | 220757_s_at |
| UBX domain containing 2 | UBXD2 | Hs.350806 | 1562741_at |
| UBX domain containing 2 | UBXD2 | Hs.350806 | 212007_at |
| UBX domain containing 4 | UBXD4 | Hs.516018 | 235327_x_at |
| UDP glycosyltransferase 1 family, polypeptide A10 | UGT1A10 | Hs.124112 | 204532_x_at |
| /// UDP glycosyltransferase 1 family, polypeptide | /// | | |
| A8 /// UDP glycosyltransferase 1 family, | UGT1A8 | | |
| polypeptide A7 /// UDP glycosyltransferase 1 | /// | | |
| family, polypeptide A6 /// UDP glycosyltransferase | UGT1A7 | | |
| 1 family, polypeptide A5 /// UDP | /// | | |
| glycosyltransferase 1 family, polypeptide A9 /// | UGT1A6 | | |
| UDP glycosyltransferase 1 family, polypeptide A4 | /// | | |
| /// UDP glycosyltransferase 1 family, polypeptide | UGT1A5 | | |
| A1 /// UDP glycosyltransferase 1 family, | /// | | |
| polypeptide A3 | UGT1A9 | | |
| | /// | | |
| | UGT1A4 | | |
| | /// | | |
| | UGT1A1 | | |
| | /// | | |
| | UGT1A3 | | |
| UDP glycosyltransferase 1 family, polypeptide A6 | UGT1A6 | Hs.124112 | 206094_x_at |
| UDP glycosyltransferase 1 family, polypeptide A9 | UGT1A6 | Hs.124112 | 230953_at |
| UDP-Gal:betaGlcNAc beta 1,4- | B4GALT5 | Hs.370487 | 1559391_s_at |
| galactosyltransferase, polypeptide 5 | | | |
| UDP-Gal: betaGlcNAc beta 1,4- | B4GALT5 | Hs.370487 | 221484_at |
| galactosyltransferase, polypeptide 5 | | | |
| UDP-Gal: betaGlcNAc beta 1,4- | B4GALT5 | Hs.370487 | 221485_at |
| galactosyltransferase, polypeptide 5 | | | |
| UDP-glucose ceramide glucosyltransferase | UGCG | Hs.304249 | 204881_s_at |
| UDP-glucose ceramide glucosyltransferase | UGCG | Hs.304249 | 224967_at |
| UDP-glucose ceramide glucosyltransferase-like 1 | UGCGL1 | Hs.34180 | 218257_s_at |
| UDP-glucose ceramide glucosyltransferase-like 2 | UGCGL2 | Hs.193226 | 1558467_a_at |
| UDP-glucose pyrophosphorylase 2 | UGP2 | Hs.516217 | 205480_s_at |
| UDP-N-acetyl-alpha-D-galactosamine: polypeptide | GALNT1 | Hs.514806 | 201722_s_at |
| N-acetylgalactosaminyltransferase 1 (GalNAc-T1) | | | |
| UDP-N-acetyl-alpha-D-galactosamine: polypeptide | GALNT1 | Hs.514806 | 201723_s_at |
| N-acetylgalactosaminyltransferase 1 (GalNAc-T1) | | | |
| UDP-N-acetyl-alpha-D-galactosamine: polypeptide | GALNT11 | Hs.329978 | 219013_at |
| N-acetylgalactosaminyltransferase 11 (GalNAc- | | | |
| T11) | | | |
| UDP-N-acetyl-alpha-D-galactosamine: polypeptide | GALNT2 | Hs.300834 | 223991_s_at |
| N-acetylgalactosaminyltransferase 2 (GalNAc-T2) | | | |
| UDP-N-acetyl-alpha-D-galactosamine: polypeptide | GALNT8 | Hs.511985 | 220929_at |
| N-acetylgalactosaminyltransferase 8 (GalNAc-T8) | | | |
| UDP-N-acetyl-alpha-D-galactosamine: polypeptide | GALNTL5 | Hs.131311 | 233150_at |
| N-acetylgalactosaminyltransferase-like 5 | | | |
| Unactive progesterone receptor, 23 kD | TEBP | Hs.50425 | 238895_at |
| Unc-5 homolog C (C. elegans)-like | UNC5CL | Hs.158357 | 231008_at |
| unc-51-like kinase 4 (C. elegans) | ULK4 | Hs.326883 | 231149_s_at |
| unc-51-like kinase 4 (C. elegans) | ULK4 | Hs.326883 | 244168_s_at |
| uncharacterized hypothalamus protein HT008 | HT008 | Hs.175414 | 218099_at |
| Unknown mRNA | — | Hs.159188 | 235360_at |
| Unknown mRNA sequence | — | Hs.112482 | 228655_at |
| UPF3 regulator of nonsense transcripts homolog A | UPF3A | Hs.514737 | 206958_s_at |
| (yeast) | | | |
| UPF3 regulator of nonsense transcripts homolog A | UPF3A | Hs.514737 | 214323_s_at |
| (yeast) | | | |
| upregulated during skeletal muscle growth 5 | USMG5 | Hs.496344 | 225413_at |
| upstream binding transcription factor, RNA | UBTF | Hs.89781 | 225982_at |
| polymerase I | | | |
| upstream of NRAS | UNR | Hs.69855 | 202646_s_at |
| upstream of NRAS | UNR | Hs.69855 | 219939_s_at |
| upstream of NRAS | UNR | Hs.69855 | 222975_s_at |
| Upstream of NRAS | UNR | Hs.69855 | 224985_at |
| uridine monophosphate synthetase (orotate | UMPS | Hs.2057 | 215165_x_at |
| phosphoribosyl transferase and orotidine-5′- | | | |
| decarboxylase) | | | |
| uridine phosphorylase 2 | UPP2 | Hs.128427 | 233155_at |
| uridine-cytidine kinase 1 | UCK1 | Hs.9597 | 223141_at |
| uroplakin 1A | UPK1A | Hs.159309 | 214624_at |
| uroplakin 1B | UPK1B | Hs.271580 | 210064_s_at |
| USP6 N-terminal like | USP6NL | Hs.498661 | 204761_at |
| utrophin (homologous to dystrophin) | UTRN | Hs.133135 | 225093_at |
| uveal autoantigen with coiled-coil domains and | UACA | Hs.108049 | 236715_x_at |
| ankyrin repeats | | | |
| Vaccinia related kinase 1 | VRK1 | Hs.422662 | 241737_x_at |
| vaccinia related kinase 3 | VRK3 | Hs.443330 | 218022_at |
| vacuolar protein sorting 13A (yeast) | VPS13A | Hs.459790 | 214785_at |
| vacuolar protein sorting 13B (yeast) | VPS13B | Hs.191540 | 236254_at |
| Vacuolar protein sorting 13C (yeast) | VPS13C | HS.511668 | 235023_at |
| vacuolar protein sorting 13D (yeast) | VPS13D | Hs.439381 | 220221_at |
| vacuolar protein sorting 24 (yeast) | VPS24 | Hs.255015 | 217837_s_at |
| vacuolar protein sorting 35 (yeast) | VPS35 | Hs.447547 | 217727_x_at |
| vacuolar protein sorting 35 (yeast) | VPS35 | Hs.447547 | 222387_s_at |
| vacuolar protein sorting 35 (yeast) | VPS35 | Hs.447547 | 233864_s_at |
| vacuolar protein sorting 37C (yeast) | VPS37C | Hs.523715 | 1560060_s_at |
| vacuolar protein sorting 37C (yeast) | VPS37C | Hs.523715 | 219053_s_at |
| Vacuolar protein sorting 37C (yeast) | VPS37C | Hs.523715 | 233696_at |
| vacuolar protein sorting 4B (yeast) | VPS4B | Hs.126550 | 218171_at |
| vacuolar protein sorting 54 (yeast) | VPS54 | Hs.48499 | 218423_x_at |
| vacuolar protein sorting 54 (yeast) | VPS54 | Hs.48499 | 233656_s_at |
| v-akt murine thymoma viral oncogene homolog 3 | AKT3 | Hs.498292 | 212607_at |
| (protein kinase B, gamma) | | | |
| v-akt murine thymoma viral oncogene homolog 3 | AKT3 | Hs.498292 | 219393_s_at |
| (protein kinase B, gamma) | | | |
| Valosin containing protein (p97)/p47 complex | VCIP135 | Hs.205550 | 1556228_a_at |
| interacting protein 1 | | | |
| valosin-containing protein | VCP | Hs.529782 | 208648_at |
| VAMP (vesicle-associated membrane protein)- | VAPA | Hs.165195 | 208780_x_at |
| associated protein A, 33 kDa | | | |
| variable charge, X-linked /// variable charge, X- | VCX /// | Hs.278906 | 207281_x_at |
| linked 2 /// variable charge, X-linked 3A /// variably | VCX2 /// | | |
| charged X—C | VCX3A | | |
| | /// VCX-C | | |
| variable charge, X-linked 2 | VCX2 | Hs.279737 | 211403_x_at |
| vascular cell adhesion molecule 1 | VCAM1 | Hs.109225 | 203868_s_at |
| vascular endothelial growth factor | VEGF | Hs.73793 | 210512_s_at |
| vasculin | DKFZp761C169 | Hs.444279 | 223705_s_at |
| vasculin | DKFZp761C169 | Hs.444279 | 224648_at |
| v-crk sarcoma virus CT10 oncogene homolog | CRKL | Hs.5613 | 212180_at |
| (avian)-like | | | |
| v-erb-b2 erythroblastic leukemia viral oncogene | ERBB3 | Hs.118681 | 202454_s_at |
| homolog 3 (avian) | | | |
| very low density lipoprotein receptor | VLDLR | Hs.370422 | 209822_s_at |
| Vesicle docking protein p115 | VDP | Hs.292689 | 222316_at |
| vesicle transport through interaction with t- | VTI1B | Hs.144868 | 209452_s_at |
| SNAREs homolog 1B (yeast) | | | |
| vesicle-associated membrane protein 4 | VAMP4 | Hs.6651 | 213480_at |
| V-ets erythroblastosis virus E26 oncogene | ETS1 | Hs.369438 | 241435_at |
| homolog 1 (avian) | | | |
| V-ets erythroblastosis virus E26 oncogene | ETS2 | Hs.517296 | 241193_at |
| homolog 2 (avian) | | | |
| V-ets erythroblastosis virus E26 oncogene | ETS2 | Hs.517296 | 242784_at |
| homolog 2 (avian) | | | |
| vinculin | VCL | Hs.500101 | 200931_s_at |
| vitelliform macular dystrophy 2-like 3 | VMD2L3 | Hs.280782 | 237003_at |
| v-Ki-ras2 Kirsten rat sarcoma viral oncogene | KRAS | Hs.505033 | 204009_s_at |
| homolog | | | |
| v-Ki-ras2 Kirsten rat sarcoma viral oncogene | KRAS | Hs.505033 | 214352_s_at |
| homolog | | | |
| v-maf musculoaponeurotic fibrosarcoma oncogene | MAFF | Hs.517617 | 36711_at |
| homolog F (avian) | | | |
| v-maf musculoaponeurotic fibrosarcoma oncogene | MAFK | Hs.520612 | 226206_at |
| homolog K (avian) | | | |
| voltage-dependent anion channel 1 | VDAC1 | Hs.202085 | 212038_s_at |
| voltage-dependent anion channel 2 /// voltage- | VDAC2 | Hs.355927 | 211662_s_at |
| dependent anion channel 2 | | | |
| voltage-dependent anion channel 3 | VDAC3 | Hs.491597 | 208845_at |
| Vpr-binding protein | VprBP | Hs.118738 | 204377_s_at |
| v-raf murine sarcoma viral oncogene homolog B1 | BRAF | Hs.490366 | 206044_s_at |
| v-raf murine sarcoma viral oncogene homolog B1 | BRAF | Hs.490366 | 243829_at |
| v-ral simian leukemia viral oncogene homolog A | RALA | Hs.6906 | 214435_x_at |
| (ras related) | | | |
| v-ral simian leukemia viral oncogene homolog A | RALA | Hs.6906 | 224880_at |
| (ras related) | | | |
| v-yes-1 Yamaguchi sarcoma viral related | LYN | Hs.491767 | 202626_s_at |
| oncogene homolog /// v-yes-1 Yamaguchi sarcoma | | | |
| viral related oncogene homolog | | | |
| WAP four-disulfide core domain 2 | WFDC2 | Hs.2719 | 203892_at |
| WAP four-disulfide core domain 3 | WFDC3 | Hs.419126 | 232602_at |
| WAP four-disulfide core domain 5 | WFDC5 | Hs.375031 | 242204_at |
| WAS protein family, member 3 | WASF3 | Hs.82318 | 204042_at |
| WD repeat and FYVE domain containing 3 | WDFY3 | Hs.480116 | 238660_at |
| WD repeat and SOCS box-containing 1 | WSB1 | Hs.446017 | 201294_s_at |
| WD repeat and SOCS box-containing 1 | WSB1 | Hs.446017 | 201296_s_at |
| WD repeat and SOCS box-containing 1 | WSB1 | Hs.446017 | 210561_s_at |
| WD repeat and SOCS box-containing 1 | WSB1 | Hs.446017 | 227501_at |
| WD repeat domain 10 | WDR10 | Hs.477537 | 216678_at |
| WD repeat domain 11 | WDR11 | Hs.144447 | 229694_at |
| WD repeat domain 20 | WDR20 | Hs.36859 | 227541_at |
| WD repeat domain 22 | WDR22 | Hs.509780 | 224696_s_at |
| WD repeat domain 23 | WDR23 | Hs.525251 | 201886_at |
| WD repeat domain 26 | WDR26 | Hs.497873 | 218107_at |
| WD repeat domain 32 | WDR32 | Hs.118394 | 222804_x_at |
| WD repeat domain 37 | WDR37 | Hs.188495 | 211383_s_at |
| WD repeat domain 37 | WDR37 | Hs.188495 | 240785_at |
| WD repeat domain 4 | WDR4 | Hs.248815 | 221632_s_at |
| WD repeat domain 40A | WDR40A | Hs.493750 | 224789_at |
| WD repeat domain 41 | WDR41 | Hs.482573 | 222503_s_at |
| WD repeat domain 42A | WDR42A | Hs.492236 | 243318_at |
| WD repeat domain 45 | WDR45 | Hs.156701 | 215553_x_at |
| WD repeat domain 47 | WDR47 | Hs.546347 | 203855_at |
| WD repeat domain 50 | WDR50 | Hs.463465 | 203721_s_at |
| WD repeat domain 52 | WDR52 | Hs.551546 | 221103_s_at |
| WD repeat domain 53 | WDR53 | Hs.385865 | 227814_at |
| WD repeat domain 54 | WDR54 | Hs.323537 | 225898_at |
| WD repeat domain 55 | WDR55 | Hs.286261 | 219809_at |
| WD repeat domain 55 | WDR55 | Hs.286261 | 223833_at |
| WD repeat domain 8 | WDR8 | Hs.31714 | 236382_at |
| WD40 repeat protein Interacting with | WIPI49 | Hs.463964 | 213836_s_at |
| phospholnositides of 49 kDa | | | |
| WDR45-like | WDR45L | Hs.201390 | 209076_s_at |
| Werner syndrome | WRN | Hs.512715 | 205667_at |
| Williams Beuren syndrome chromosome region 19 | WBSCR19 | Hs.488157 | 232964_at |
| Williams Beuren syndrome chromosome region 22 | WBSCR22 | Hs.413036 | 207628_s_at |
| Wilms tumor 1 associated protein | WTAP | Hs.446091 | 227621_at |
| Wilms tumor 1 associated protein | WTAP | Hs.446091 | 229630_s_at |
| wingless-type MMTV integration site family, | WNT3 | Hs.445884 | 231743_at |
| member 3 | | | |
| wingless-type MMTV integration site family, | WNT5A | Hs.152213 | 205990_s_at |
| member 5A | | | |
| WIPI49-like protein 2 | WIPI-2 | Hs.122363 | 202031_s_at |
| WIPI49-like protein 2 | WIPI-2 | Hs.122363 | 204710_s_at |
| WIPI49-like protein 2 | WIPI-2 | Hs.122363 | 214699_x_at |
| WIRE protein | WIRE | Hs.421622 | 212050_at |
| Wiskott-Aldrich syndrome protein interacting | WASPIP | Hs.128067 | 202665_s_at |
| protein | | | |
| Wiskott-Aldrich syndrome-like /// Ribosomal protein | WASL /// | Hs.143728 /// | 224738_x_at |
| L7-like 1 /// Similar to RIKEN cDNA 2310039H08 | RPL7L1 | Hs.520133 /// | |
| | /// | Hs.550564 | |
| | LOC441150 | | |
| WNK lysine deficient protein kinase 3 | WNK3 | Hs.92423 | 1555248_a_at |
| WNT1 inducible signaling pathway protein 3 | WISP3 | Hs.549081 | 210861_s_at |
| Wolf-Hirschhorn syndrome candidate 1-like 1 | WHSC1L1 | Hs.32099 | 218173_s_at |
| Wolf-Hirschhorn syndrome candidate 1-like 1 | WHSC1L1 | Hs.32099 | 224076_s_at |
| Wolf-Hirschhorn syndrome candidate 2 | WHSC2 | Hs.21771 | 34225_at |
| Wolfram syndrome 1 (wolframin) | WFS1 | Hs.518602 | 202908_at |
| WW domain containing adaptor with coiled-coil | WAC | Hs.435610 | 217742_s_at |
| WW domain containing adaptor with coiled-coil | WAC | Hs.435610 | 222389_s_at |
| WW domain containing E3 ubiquitin protein ligase 1 | WWP1 | Hs.533440 | 212638_s_at |
| WW domain containing E3 ubiquitin protein ligase 2 | WWP2 | Hs.408458 | 204022_at |
| WW domain containing oxidoreductase | WWOX | Hs.549163 | 210695_s_at |
| x 006 protein | MDS006 | Hs.47668 | 223401_at |
| X-box binding protein 1 | XBP1 | Hs.437638 | 200670_at |
| Xeroderma pigmentosum, complementation group A | XPA | Hs.288867 | 1559515_at |
| XIAP associated factor-1 | HSXIAPAF1 | Hs.441975 | 228617_at |
| X-ray repair complementing defective repair in | XRCC2 | Hs.129727 | 207598_x_at |
| Chinese hamster cells 2 | | | |
| xylosyltransferase II | XYLT2 | Hs.463416 | 219401_at |
| xylulokinase homolog (H. influenzae) | XYLB | Hs.356820 | 1569683_at |
| yippee-like 5 (Drosophila) | YPEL5 | Hs.515890 | 217783_s_at |
| yippee-like 5 (Drosophila) | YPEL5 | Hs.515890 | 222408_s_at |
| YLP motif containing 1 | YLPM1 | Hs.531111 | 212787_at |
| YLP motif containing 1 | YLPM1 | Hs.531111 | 214659_x_at |
| YME1-like 1 (S. cerevisiae) | YME1L1 | Hs.499145 | 201351_s_at |
| YME1-like 1 (S. cerevisiae) | YME1L1 | Hs.499145 | 216304_x_at |
| YTH domain containing 2 | YTHDC2 | Hs.231942 | 213077_at |
| YTH domain family, member 1 | YTHDF1 | Hs.11747 | 221741_s_at |
| YTH domain family, member 2 | YTHDF2 | Hs.532286 | 217812_at |
| YTH domain family, member 2 | YTHDF2 | Hs.532286 | 222430_s_at |
| YY1 associated protein 1 | YAP | Hs.516746 | 232516_x_at |
| YY1 associated protein 1 | YAP | Hs.516746 | 244803_at |
| YY1 transcription factor | YY1 | Hs.388927 | 201901_s_at |
| YY1 transcription factor /// YY1 transcription factor | YY1 | Hs.388927 | 200047_s_at |
| zinc finger and BTB domain containing 1 | ZBTB1 | Hs.400802 | 213376_at |
| Zinc finger and BTB domain containing 10 | ZBTB10 | Hs.205742 | 228562_at |
| Zinc finger and BTB domain containing 10 | ZBTB10 | Hs.205742 | 233899_x_at |
| zinc finger and BTB domain containing 2 | ZBTB2 | Hs.520073 | 226284_at |
| zinc finger and BTB domain containing 26 | ZBTB26 | Hs.5638 | 1554973_a_at |
| zinc finger and BTB domain containing 26 | ZBTB26 | Hs.5638 | 227162_at |
| zinc finger and BTB domain containing 33 | ZBTB33 | Hs.143604 | 226255_at |
| zinc finger and BTB domain containing 37 | ZBTB37 | Hs.535229 | 231547_at |
| zinc finger and BTB domain containing 38 | ZBTB38 | Hs.518301 | 219221_at |
| zinc finger and BTB domain containing 38 | ZBTB38 | Hs.518301 | 236557_at |
| zinc finger and BTB domain containing 4 | ZBTB4 | Hs.35096 | 225629_s_at |
| zinc finger and BTB domain containing 5 | ZBTB5 | Hs.161276 | 203026_at |
| zinc finger and BTB domain containing 8 | ZBTB8 | Hs.546479 | 235142_at |
| zinc finger CCCH type domain containing 1 | ZC3HDC1 | Hs.12646 | 218543_s_at |
| zinc finger CCCH type domain containing 5 | ZC3HDC5 | Hs.201859 | 1562434_at |
| zinc finger CCCH type domain containing 7 | ZC3HDC7 | Hs.371856 | 226897_s_at |
| zinc finger CCCH type, antiviral 1 | ZC3HAV1 | Hs.133512 | 213051_at |
| zinc finger DAZ interacting protein 3 | DZIP3 | Hs.409210 | 207231_at |
| zinc finger DAZ interacting protein 3 | DZIP3 | Hs.409210 | 213186_at |
| zinc finger homeobox 1b | ZFHX1B | Hs.34871 | 203603_s_at |
| zinc finger homeodomain 4 | ZFHX4 | Hs.458973 | 219779_at |
| zinc finger protein 10 (KOX 1) | ZNF10 | Hs.507355 | 235366_at |
| zinc finger protein 131 (clone pHZ-10) | ZNF131 | Hs.97845 | 221842_s_at |
| zinc finger protein 136 (clone pHZ-20) | ZNF136 | Hs.479874 | 206240_s_at |
| zinc finger protein 148 (pHZ-52) | ZNF148 | Hs.380334 | 203318_s_at |
| Zinc finger protein 148 (pHZ-52) | ZNF148 | Hs.380334 | 228545_at |
| zinc finger protein 160 | ZNF160 | Hs.467236 | 214715_x_at |
| zinc finger protein 161 | ZNF161 | Hs.463569 | 202171_at |
| zinc finger protein 161 | ZNF161 | Hs.463569 | 202173_s_at |
| zinc finger protein 165 | ZNF165 | Hs.55481 | 206683_at |
| zinc finger protein 185 (LIM domain) | ZNF185 | Hs.16622 | 203585_at |
| zinc finger protein 19 (KOX 12) | ZNF19 | Hs.513705 | 228958_at |
| Zinc finger protein 198 | ZNF198 | Hs.507433 | 1569091_at |
| zinc finger protein 198 | ZNF198 | Hs.507433 | 202778_s_at |
| Zinc finger protein 198 | ZNF198 | Hs.507433 | 243992_at |
| zinc finger protein 20 (KOX 13) | ZNF20 | Hs.263600 | 213916_at |
| zinc finger protein 200 | ZNF200 | Hs.513288 | 214706_at |
| zinc finger protein 204 | ZNF204 | Hs.8198 | 214823_at |
| zinc finger protein 207 | ZNF207 | Hs.500775 | 200829_x_at |
| zinc finger protein 207 | ZNF207 | Hs.500775 | 231848_x_at |
| Zinc finger protein 207 | ZNF207 | Hs.500775 | 239937_at |
| zinc finger protein 213 | ZNF213 | Hs.115284 | 227207_x_at |
| zinc finger protein 232 | ZNF232 | Hs.279914 | 219123_at |
| zinc finger protein 236 | ZNF236 | Hs.189826 | 47571_at |
| zinc finger protein 238 | ZNF238 | Hs.69997 | 212774_at |
| zinc finger protein 239 | ZNF239 | Hs.25040 | 206261_at |
| zinc finger protein 24 (KOX 17) | ZNF24 | Hs.514802 | 1554045_at |
| zinc finger protein 24 (KOX 17) | ZNF24 | Hs.514802 | 203247_s_at |
| Zinc finger protein 24 (KOX 17) | ZNF24 | Hs.514802 | 212534_at |
| zinc finger protein 259 /// zinc finger protein 259 | ZNF259 | Hs.7165 | 200054_at |
| zinc finger protein 265 | ZNF265 | Hs.194718 | 223016_x_at |
| zinc finger protein 265 | ZNF265 | Hs.194718 | 223716_s_at |
| zinc finger protein 273 | ZNF273 | Hs.386264 | 215239_x_at |
| zinc finger protein 274 | ZNF274 | Hs.83761 | 232436_at |
| zinc finger protein 275 | ZNF275 | Hs.348963 | 225383_at |
| zinc finger protein 277 | ZNF277 | Hs.489722 | 215887_at |
| Zinc finger protein 281 | ZNF281 | Hs.59757 | 222619_at |
| zinc finger protein 285 | ZNF285 | Hs.515454 | 236328_at |
| zinc finger protein 291 | ZNF291 | Hs.458986 | 215848_at |
| zinc finger protein 294 | ZNF294 | Hs.288773 | 233819_s_at |
| zinc finger protein 297B | ZNF297B | Hs.355581 | 204180_s_at |
| zinc finger protein 30 (KOX 28) | ZNF30 | Hs.135052 | 232014_at |
| Zinc finger protein 302 | ZNF302 | Hs.436350 | 228392_at |
| Zinc finger protein 302 | ZNF302 | Hs.436350 | 228393_s_at |
| zinc finger protein 31 (KOX 29) | ZNF31 | Hs.442705 | 1552499_a_at |
| zinc finger protein 330 | ZNF330 | Hs.120766 | 209814_at |
| zinc finger protein 330 | ZNF330 | Hs.120766 | 213760_s_at |
| zinc finger protein 331 | ZNF331 | Hs.185674 | 219228_at |
| zinc finger protein 336 | ZNF336 | Hs.28921 | 225884_s_at |
| zinc finger protein 339 /// zinc finger protein 339 | ZNF339 | Hs.546418 | 211778_s_at |
| zinc finger protein 343 | ZNF343 | Hs.516846 | 227986_at |
| zinc finger protein 346 | ZNF346 | Hs.484259 | 236267_at |
| zinc finger protein 35 (clone HF.10) | ZNF35 | Hs.288658 | 206096_at |
| Zinc finger protein 354A | ZNF354A | Hs.484324 | 217715_x_at |
| zinc finger protein 36, C3H type-like 1 | ZFP36L1 | Hs.85155 | 211962_s_at |
| zinc finger protein 364 | ZNF364 | Hs.523550 | 212742_at |
| zinc finger protein 397 | ZNF397 | Hs.464896 | 228927_at |
| zinc finger protein 403 | ZNF403 | Hs.514116 | 218079_s_at |
| zinc finger protein 403 | ZNF403 | Hs.514116 | 233936_s_at |
| zinc finger protein 403 | ZNF403 | Hs.514116 | 233937_at |
| zinc finger protein 410 | ZNF410 | Hs.270869 | 202010_s_at |
| zinc finger protein 410 | ZNF410 | Hs.270869 | 209944_at |
| zinc finger protein 415 | ZNF415 | Hs.147765 | 205514_at |
| Zinc finger protein 426 | ZNF426 | Hs.324978 | 1556777_a_at |
| zinc finger protein 43 (HTF6) | ZNF43 | Hs.534365 | 206695_x_at |
| zinc finger protein 44 (KOX 7) | ZNF44 | Hs.24120 | 215359_x_at |
| zinc finger protein 444 | ZNF444 | Hs.24545 | 50376_at |
| zinc finger protein 451 | ZNF451 | Hs.485628 | 215012_at |
| zinc finger protein 462 | ZNF462 | Hs.370379 | 226575_at |
| zinc finger protein 486 | ZNF486 | Hs.515691 | 1553901_x_at |
| zinc finger protein 505 | ZNF505 | Hs.301059 | 215758_x_at |
| zinc finger protein 514 | ZNF514 | Hs.274414 | 235729_at |
| Zinc finger protein 518 | ZNF518 | Hs.147895 | 244132_x_at |
| zinc finger protein 532 | ZNF532 | Hs.529023 | 225021_at |
| zinc finger protein 541 | ZNF541 | Hs.14161 | 232604_at |
| zinc finger protein 542 | ZNF542 | Hs.467326 | 235121_at |
| Zinc finger protein 543 | ZNF543 | Hs.202544 | 240191_at |
| zinc finger protein 544 | ZNF544 | Hs.438994 | 218735_s_at |
| zinc finger protein 548 | ZNF548 | Hs.301940 | 1553718_at |
| zinc finger protein 548 | ZNF548 | Hs.301940 | 1558622_a_at |
| zinc finger protein 550 | ZNF550 | Hs.180257 | 228099_at |
| zinc finger protein 553 | ZNF553 | Hs.513501 | 227470_at |
| Zinc finger protein 555 | ZNF555 | Hs.47712 | 1556188_a_at |
| zinc finger protein 562 | ZNF562 | Hs.531179 | 219163_at |
| zinc finger protein 564 | ZNF564 | Hs.534574 | 1570364_at |
| zinc finger protein 565 | ZNF565 | Hs.20369 | 228305_at |
| zinc finger protein 570 | ZNF570 | Hs.350875 | 1552562_at |
| zinc finger protein 573 | ZNF573 | Hs.290703 | 217627_at |
| zinc finger protein 585A | ZNF585A | Hs.390568 | 243790_at |
| zinc finger protein 587 | ZNF587 | Hs.288995 | 219981_x_at |
| Zinc finger protein 599 | ZNF599 | Hs.368375 | 232679_at |
| Zinc finger protein 599 | ZNF599 | Hs.368375 | 243747_at |
| zinc finger protein 606 | ZNF606 | Hs.287629 | 219635_at |
| zinc finger protein 62 homolog (mouse) | ZFP62 | Hs.509227 | 227796_at |
| Zinc finger protein 638 | ZNF638 | Hs.434401 | 235493_at |
| Zinc finger protein 64 homolog (mouse) | ZFP64 | Hs.473082 | 242759_at |
| zinc finger protein 644 | ZNF644 | Hs.173001 | 222580_at |
| Zinc finger protein 650 | ZNF650 | Hs.379548 | 1566165_at |
| Zinc finger protein 652 | ZNF652 | Hs.463375 | 225266_at |
| Zinc finger protein 652 | ZNF652 | Hs.463375 | 235577_at |
| zinc finger protein 664 | ZNF664 | Hs.524828 | 224593_at |
| zinc finger protein 672 | ZNF672 | Hs.521151 | 218068_s_at |
| zinc finger protein 677 | ZNF677 | Hs.20506 | 1569039_s_at |
| zinc finger protein 682 | ZNF682 | Hs.306298 | 242915_at |
| zinc finger protein 683 | ZNF683 | Hs.353208 | 230756_at |
| Zinc finger protein 83 (HPF1) | ZNF83 | Hs.467210 | 236429_at |
| Zinc finger protein 84 (HPF2) | ZNF84 | Hs.445019 | 228630_at |
| Zinc finger protein 90 homolog (mouse) | ZFP90 | Hs.461074 | 241818_at |
| zinc finger protein 91 homolog (mouse) | ZFP91 | Hs.524920 | 224631_at |
| zinc finger protein 95 homolog (mouse) | ZFP95 | Hs.110839 | 203730_s_at |
| zinc finger protein like | ZFPL | Hs.502344 | 241704_x_at |
| Zinc finger protein Zec | ZEC | Hs.525209 | 1563344_at |
| zinc finger protein, Y-linked | ZFY | Hs.522845 | 207246_at |
| zinc finger RNA binding protein | ZFR | Hs.435231 | 201856_s_at |
| zinc finger RNA binding protein | ZFR | Hs.435231 | 201857_at |
| zinc finger RNA binding protein | ZFR | Hs.435231 | 33148_at |
| zinc finger, A20 domain containing 2 | ZA20D2 | Hs.406096 | 217741_s_at |
| zinc finger, A20 domain containing 3 | ZA20D3 | Hs.306329 | 221613_s_at |
| Zinc finger, A20 domain containing 3 | ZA20D3 | Hs.306329 | 222186_at |
| zinc finger, CCHC domain containing 11 | ZCCHC11 | Hs.476164 | 212704_at |
| Zinc finger, CCHC domain containing 11 | ZCCHC11 | Hs.476164 | 230713_at |
| zinc finger, CCHC domain containing 12 | ZCCHC12 | Hs.21417 | 228715_at |
| zinc finger, CCHC domain containing 2 | ZCCHC2 | Hs.114191 | 219062_s_at |
| zinc finger, CCHC domain containing 3 | ZCCHC3 | Hs.28608 | 225091_at |
| Zinc finger, CCHC domain containing 6 | ZCCHC6 | Hs.136398 | 236155_at |
| Zinc finger, CCHC domain containing 6 | ZCCHC6 | Hs.136398 | 236243_at |
| zinc finger, CCHC domain containing 9 | ZCCHC9 | Hs.15536 | 225538_at |
| zinc finger, CSL domain containing 2 | ZCSL2 | Hs.388087 | 225195_at |
| Zinc finger, CSL domain containing 3 | ZCSL3 | Hs.187269 | 242562_at |
| zinc finger, CW type with coiled-coil domain 1 | ZCWCC1 | Hs.552585 | 203956_at |
| zinc finger, DHHC domain containing 11 | ZDHHC11 | Hs.368851 | 221646_s_at |
| zinc finger, DHHC domain containing 13 | ZDHHC13 | Hs.188569 | 219296_at |
| Zinc finger, DHHC domain containing 13 | ZDHHC13 | Hs.188569 | 232440_at |
| zinc finger, DHHC domain containing 2 | ZDHHC2 | Hs.443852 | 222730_s_at |
| zinc finger, DHHC domain containing 20 | ZDHHC20 | Hs.24283 | 243786_at |
| zinc finger, DHHC domain containing 23 | ZDHHC23 | Hs.21902 | 226912_at |
| zinc finger, DHHC domain containing 3 | ZDHHC3 | Hs.61430 | 218077_s_at |
| zinc finger, DHHC domain containing 6 | ZDHHC6 | Hs.196990 | 218249_at |
| zinc finger, imprinted 2 | ZIM2 | — | 220653_at |
| zinc finger, matrin type 2 | ZMAT2 | Hs.350194 | 224782_at |
| zinc finger, MYND domain containing 11 | ZMYND11 | Hs.292265 | 202136_at |
| zinc finger, RAN-binding domain containing 1 | ZRANB1 | Hs.550520 | 225131_at |
| zinc finger, RAN-binding domain containing 1 | ZRANB1 | Hs.550520 | 225138_at |
| Zinc finger, ZZ type with ankyrin repeat domain 1 | ZZANK1 | Hs.135805 | 241541_at |
| zinc finger, ZZ type with EF hand domain 1 | ZZEF1 | Hs.277624 | 207190_at |
| zinc fingers and homeoboxes 1 | ZHX1 | Hs.521264 | 223213_s_at |
| zinc ribbon domain containing, 1 | ZNRD1 | Hs.57813 | 228009_x_at |
| zona pellucida binding protein 2 | ZPBP2 | Hs.367245 | 231442_at |
| ZW10 interactor | ZWINT | Hs.42650 | 204026_s_at |
| zygote arrest 1 | ZAR1 | Hs.276457 | 1555775_a_at |

Affymetrix Data Processing

Affymetrix based analyses of spermatozoan RNA from normal individuals and affected individuals were imaged using the Affymetrix gene-ship software. Images were further processed at Wayne State University using Dchip (Li et al., DNA-Chip analyzer (dChip) in The Analysis of Gene Expression Data: Methods and Software, Ed. By Parmigiani et al., 2002. A range of other approaches were explored, including GCOS, RMA and GCRMA, but they were found to be not substanti ally better than Dchip as assessed by dataset concordance when compared to previous data in U.S. Ser. No. 10/264,231 and Ostrmeier et al., ibid. Dchip was used to assess stability of the detected transcripts, while median signal values were used to summarize the transcript profiles in the following manner:

A. Rejection of potentially impacted arrays was carried out in a conservative manner on the basis of any array having >1.75% probeset outliers relative to the other arrays after MBEI modeling. No arrays were rejected on the basis of a high number of single outliers.

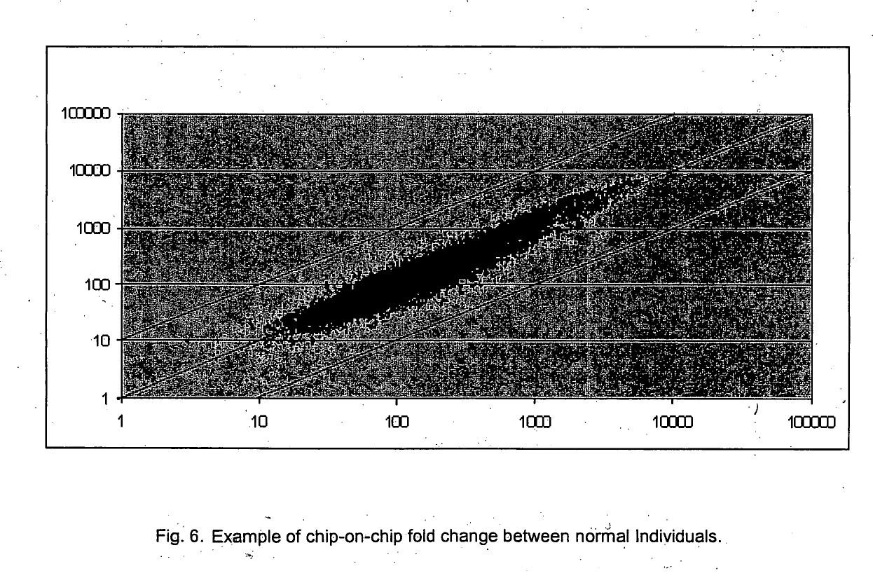

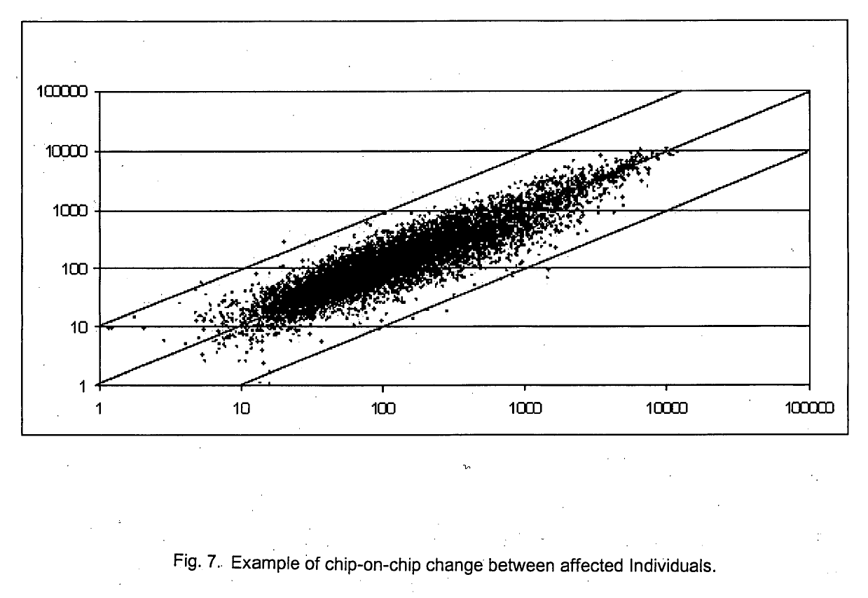

B. Variance between chips was used to assess a lower bound signal cutoff. As illustrated in FIGS. 6 and 7, variance between ships was relatively low. Variance was higher between affected individuals as compared to that observed between the normal individuals. Based on the observed array versus array variance, a lower threshold of consistent signal was set to a value of 20. This approximates the median signal from those probesets below the present criterion cutoff. This criterion groups the vast majority of transcripts among normal individuals as consistently stable between individuals. Similarly, the vast majority of transcripts among affected individuals are grouped as consistently stable.

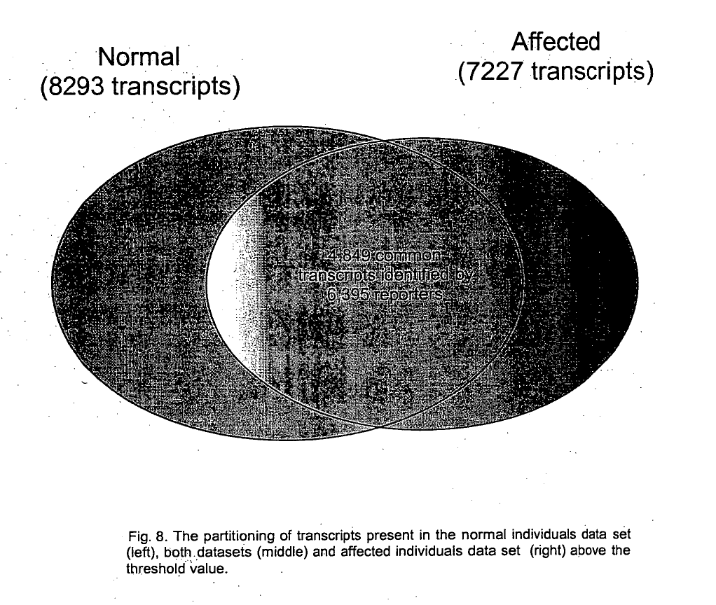

The transcripts identified as present in 13 normal samples (12/13 or 13/13 present) and in the eight affected samples (7/8 or 8/8 present) are illustrated in FIG. 8. A total of 12,221 probesets were identified as present in all chips from normal individuals. The median signal among this set was 108. After threshold filtering for low abundance transcripts, 8,293 transcripts were identified as consistently present. In comparison, a total of 10,626 probesets were identified as present in all chips from the affected individuals. The median signal amongst these transcripts was 93. After threshold filtering for low abundance transcripts, 7,227 transcripts were consistently identified among affected individuals.

Analysis of Transcripts Detected in Normal and Affected Individuals

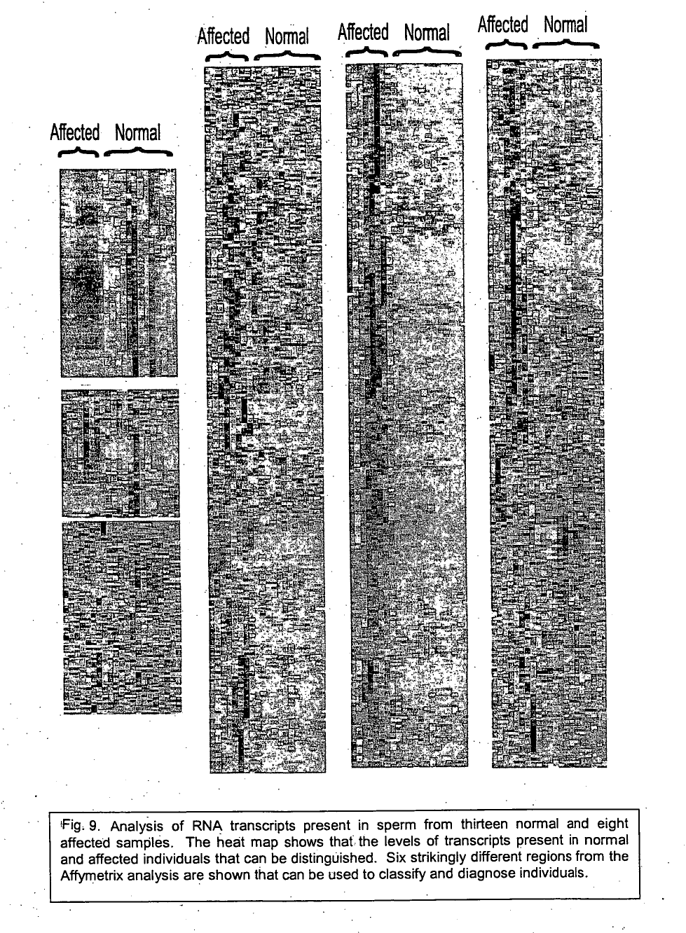

As shown in FIG. 9, the sperm transcripts identified as present in both affected and normal individuals, when compared using the Cluster 3.0 analysis software (de Hoon et al., Bioinformatics 20(9): 1453-1454, 2004) cluster into two distinct groups by their relative levels. The segregation of affection from normal individuals is clearly evidenced by the differential levels of the transcripts shown in the six heat map panels that were extracted from the entire array. A complete listing of the transcripts and probesets consistently present in both the normal and affected individuals is included in Table 1. Marked differences in the levels of these transcripts are clearly evident. This permits-the classification of any individual as either normal or affected.

A dataset of transcripts consistently present in normal yet absent in affected individuals has been derived that can unambiguously distinguish between normal and affected individuals. This list is shown in Table 2. The following transcripts absent in affected individuals yet present in normal individuals include but are not limited to the following: zona pellucida binding protein; zona pellucida glycoprotein 3 (sperm receptor);POM (POM121 homolog, rat) and ZP3 fus-ion; transition protein 2 (during histone to protamine replacement); sperm specific antigen 2; spermatogenesis and centriole associated 1; spermatogenesis associated 8; spermatogenesis associated 7;spermatogenesis associated 13; spermatogenesis associated 12; spermatogenesis associated 1; sperm associated antigen 6; sperm associated antigen 4; Round spermatid basic protein 1-like;motile sperm domain containing 3; sperm-specific protein Izumo; glyceraldehyde-3-phosphate dehydrogenase, spermatogenic; degenerative spermatocyte homolog 1, lipid desaturase (Drosophila);deleted in azoospermia-like; deleted in azoospermia 1; deleted in azoospermia 3; deleted in azoospermia 2; deleted in azoospermia 4; cation channel, sperm associated 3.

Cross Platform Validation

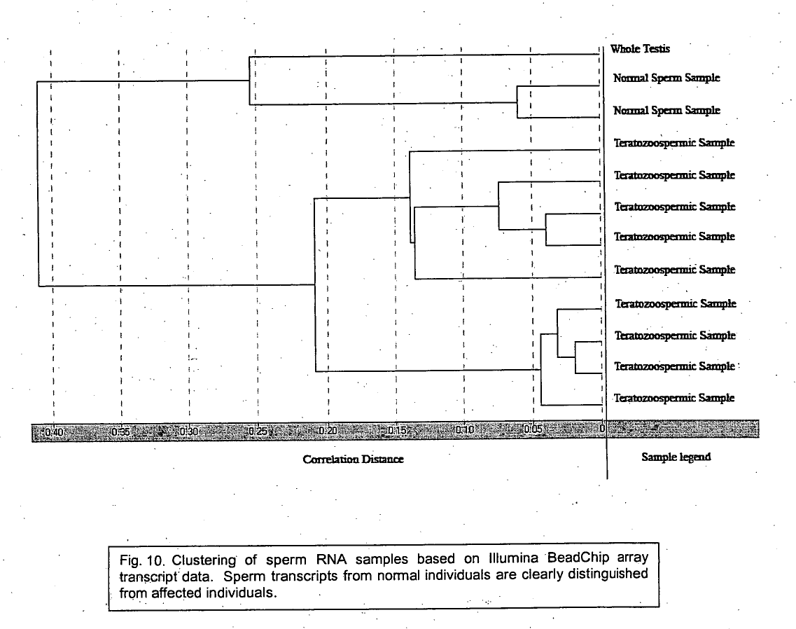

Additional platforms were also evaluated, including the Illumina Centrix Human-6 Expression BeadChip, as alternative cost effective technologies. As shown in FIG. 10, the Illumina Centrix Human-6 Expression BeadChip was also able to distinguish the transcript profiles of normal individuals among teratozoospermic affected individuals. Interestingly, unlike the Affymetrix assay, the BeadStudio analysis partitioned the affected individuals into two groups, consistent with the view that the Illumina Centrix Human-6 Expression BeadChip assay may provide a higher level of biological resolution.

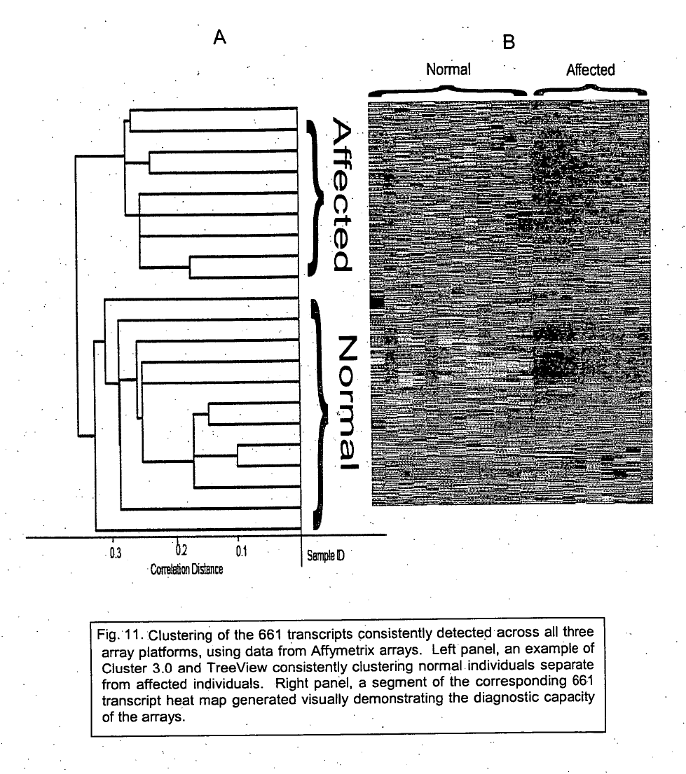

The transcripts from both the Affymetrix-based analysis and the Illumina Centrix Human-6 Expression BeadChip analysis were ranked to reflect their stability of detection using the various transcripts previously validated by RT-PCR (Ostermeier, G. C:,-Dix, D. J., Miller, D., Khatri, P. and Krawetz, S.A. (2002) Spermatozoal RNA profiles of normal fertile men. The Lancet. 360:773-777; Ostermeier, G. C., Miller, D., Huntriss, J. D., Diamond, M. P. and Krawetz, S. A. (2004) Delivering spermatozoan RNA to the oocyte. Nature 429:154) and an additional RT-PCR validated set of transcripts shown in CD9; DATFl; Delc2; EIF4E2; FLJ23878; GANAB; GNA11; GPX4; HNRPU; HSPC128; KIAA0251; KPNA6; LTBP2; MAPREL; MRPS18B; NFATC3; NFKB1; ODF2; PHF7; PIP5K2A; PPM1G; PRPSAP1; PSMF1; RANGAPL; RFX3; RND2; RPL3S; SAMD4; SDHC; SEC61B; SESN3; SPOP; SRPRB; SUV42OH1; TMPIT; TOB1; and VPS37C. The heat map of showing the relative abundance of these 661 transcripts as detected on the Affymetrix platform is shown in FIG. 11. Based on this stable set of transcripts one can again determine which individuals are normal and affected using an array-based system.

In addition, we have compared our previous spotted microarray data (Ostermeier GC, Dix DJ, Miller D, Khatri P, Krawetz SA. Spermatozoal Lancet. 2002 Sep 7;360(9335):772-7.RNA profiles of normal fertile men) with that obtained from both the Illumina and Affymetrix platforms. A total of 661 transcripts are stably detected across all three platforms. The list of stable transcripts. is listed in Table 4 including APBB3.

In one embodiment of the present invention, a set of 27,016 different expressed sequence tag probes (ESTs) was interrogated using cDNAs from testes and both pooled and single ejaculate spermatozoal mRNAs. The testes cDNAs hybridized to 7157 unique ESTs. This population contained all of the 3281 ESTs identified by the cDNAs of the pooled-ejaculate probe, which in turn contained 2780 ESTs identified by the cDNAs of the individual ejaculate probe. The data from testes and spermatozoa are coincident and define a spermatozoal mRNA fingerprint representative of a normal fertile male. As a result, the ejaculate spermatozoa can be used as a proxy for testes infertility investigations.

The biological complexity of the spermatozoal mRNAs was determined. Interestingly, a subset of these mRNAs was found to be associated with embryo development. This sub-population complemented that of the oocyte and was found to be unique to spermatozoa. The data suggest that, in addition to delivering the paternal genome, spermatozoa provide a greater role than had been believed in the orchestration of normal embryo development.

Spermatozoal mRNAs encapsulate the gene expression of spermatogenesis. The mRNAs observed in spermatozoa coincide with those found. in the testes. Comparison of the human spermatozoal and tested mRNA fingerprints by microarray analysis was selected as the primary means to address this issue. Messenger RNAs were isolated from testes and ejaculate spermatozoa, and the corresponding cDNAs were hybridized to a series of microarrays containing 30,892 Expressed Sequence Tag probes (ESTs), of which-27,016 are unique. To define the fingerprints, a gene product is considered present if its hybridization signal is at least four-fold above background intensity.

Materials and Methods

Human ejaculates were obtained from ten healthy volunteers of proven fertility and of normal semen quality as assessed by World Health Organization criteria. Nine of the samples were obtained from the Assisted Conception Unit at Leeds General Infirmary, Leeds, England. One sample was obtained from the normal fertile donor program at the Hutzel Hospital in Detroit, Mich. All samples were obtained following full ethical approval and consent from each of the subjects.

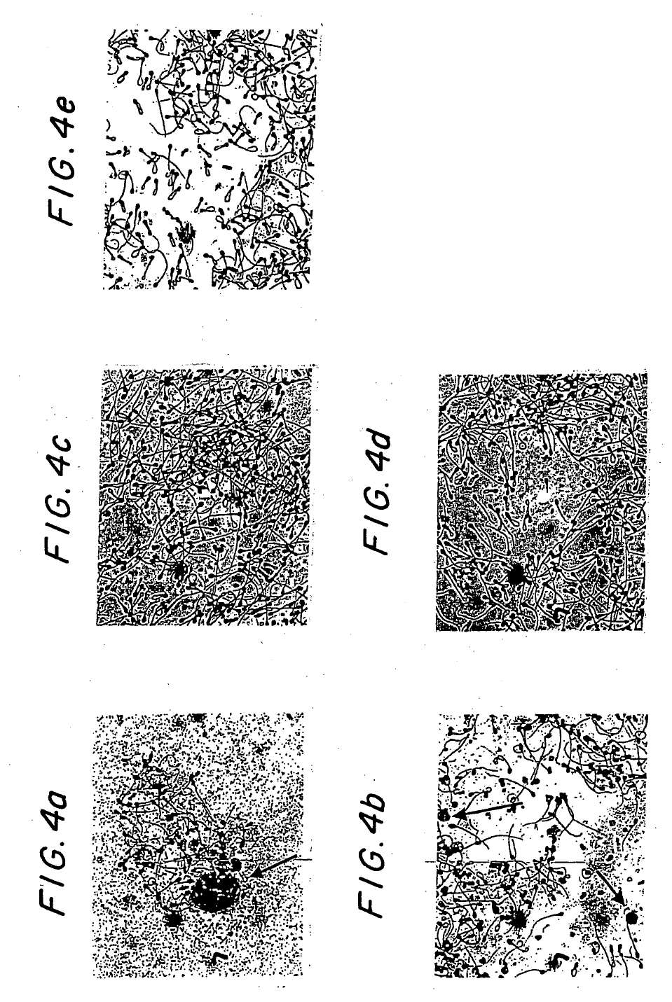

To select fertile spermatozoa and remove somatic contaminants, the nine samples from Leeds were individually purified by two sequential centrifugations through 40:80-discontinuous Percoll gradients in the following manner. Subsequent to the first centrifugation through the Percoll gradient, the pellet was resuspended, then centrifuged through a second 40:80 discontinuous gradient of Percoll. The nine spermatozoal-enriched pellets were then pooled. In the unlikely event that any residual somatic contaminants were carried though, both the pooled and individual ejaculate spermatozoa were washed in a solution of 0.5% Triton X-100. The efficacy of this regimen was histochemically verified, as shown in FIG. 4a-e. The virtual absence of ribosomal RNAs shown in FIG. 4f confirmed the lack of somatic contaminants.

In FIG. 4, a representative field of crude semen and the 40:80% Percoll interface are respectively shown in (A) and (B). Somatic cell contaminants are often and clearly observed in the crude semen (arrows). These are essentially excluded from the pellets in the first round of centrifugation (C) and are not observed in the pellets after the second round of centrifugation either before (D) or after hypotonic treatment (E).

FIG. 4F illustrates the fidelity of spermatozoal RNA preparations. Ribonucleic acid was isolated from both spermatozoa and a somatic tissue (kidney). A 5 microgram aliquot of total RNA from each preparation was loaded into separate wells of a 1.8% agarose gel. Following electrophoresis, the gel was stained with ethidium bromide. The virtual absence of 28 s and 182 rRNAs in the spermatozoal preparation confirms the lack of somatic contamination.

Poly (A+) RNA was exclusively isolated from the pooled spermatozoal RNA using oligo (dT)-coated magnetic beads, as described by the manufacturer (Dynal Corp., UK). Any residual DNA was removed by treating the isolated total RNA with RNase-free DNase 1. The purity and integrity of both preparations of spermatozoal RNAs was verified by RT-PCR using the intron spanning protamine 2 (PRM-2) primer pair. As previously shown, the sole existence of the intronless PRM-2 amplicon verified the integrity of both the poly (A+) enriched and total RNA preparations and demonstrated that they were essentially free of DNA (Miller et al., 1999).

Complementary DNA from pooled histologically normal human testes RNAs, from 19 trauma victims ranging in age-from 19 to 61 years, was purchased from Clontech Laboratories, Palo Alto, Calif. These preparations essentially contained >70% spermatogenic cells (Kramer et al., 2000). Complementary DNAs for microarray analysis were prepared from the testes and spermatozoal RNAs by reverse-transcription of 2 microgram total or poly (A+) RNA using an oligo deoxythymidine (dT) primer in the presence of 20 microliters [α-³³p]P-dCTP (3000 Ci/mmol, ICN Pharmaceuticals Inc., Costa Mesa, Calif.), according to the array manufacturer's protocol (Research Genetics, Inc., Huntsville, Ala.). Labeled cDNA from 2 micrograms of total or poly (A+) RNA was evenly distributed between six arrays for hybridization.

Human Genefilter® microarrays 200, 201, 202, 203, 204 and 211 were purchased from Research Genetics, since they provided a sufficient coverage depth of the human genome and are subject to stringent quality control. This filter set contained over 30,000 sequence verified human cDNAs, each representing at least a 1 kb region of the 3′ UTR (Taylor et al., 2001; Wang et al., 2000). Probes were hybridized to the filters as described by the manufacturer (http://www.resgen.com/products/GF200-protocol/php3). The filters were washed and exposed to Kodak phosphorimaging screens for three to seven days. Images were captured using a Molecular Imager FX (Bio-Rad Laboratories, Hercules, Calif.). After control point insertion (Reid et al, 2000), the images were analyzed using Pathways software version 3.0 for Windows or UNIX (Research Genetics; Huntsville, Ala.). Hybridization of each of the three RNA samples was carried out on each of six arrays, i.e., three samples, six arrays, 18 hybridizations.

An EST was designated as present if it was at least four fold above background. This provided an efficient means to discern abundant mRNAs. The resulting binomial distribution (Conover, 1980) was then used to calculate confidence intervals and to determine the measurement error for the number of ESTs identified.

The hybridization error rate was estimated by obtaining a summation of positive hybridization signals within each of 2994 sets of ESTs that were spotted at least two times across the entire set of filters. The percent of positive hybridization signals for each set was calculated by dividing the sum of positive signals by the total number of times that the specific EST was spotted. The error rate for each set of duplicate ESTs was determined by subtracting the percent positive from 1. If an EST was spotted multiple times and all hybridization signals were negative the percent positive was set to 100, leading to an error rate of 0%.

To identify the number of unique ESTs and overlapping gene clusters in the testes and spermatozoal samples, the accession codes, gene cluster Ids and gene manes for the positive ESTs were analyzed using the Statistical Analysis Software package (SAS various 7-1; SAS Institute, Cary, N.C.). Using the sort command within SAS, duplicate accession codes within and across filters were deleted for each sample. The unique accession codes within each sample were then compared among all samples using a Boolean search strategy, and the number of shared observations was determined (Ostermeier et al., 2002, in press).

Onto-Express, a JAVA based program developed for this study (Khatri et al., 2002), was used to mine the current databases to classify the biological expression profiles of each EST. In brief, locus link was queried (ftp://ncbi.nim.nih.gov/refseg/LocusLink/LLtmpl) and the biochemical function, cellular component, and biological process of the corresponding protein was obtained.

Results and Discussion

Throughout the multifaceted developmental program of spermatogenesis, many testis-specific mRNAs are synthesized and placed under stringent translational control to ensure appropriate temporal and spatial expression (Hecht, 1998). It has been suggested that the mRNAs observed in mature spermatozoa are remnants of untranslated sorts, and that these provide a historic record or fingerprint of spermatogenesis (Miller et al., 1994). If correct, the mRNAs observed in spermatozoa would coincide with those found in testes. Comparison of the human spermatozoal and testes mRNA fingerprints by microarray analysis was selected as the primary means to test this tenet and validate the dataset.

A wide range of mRNAs, shown in FIG. 4f, were isolated from pure preparations of spermatozoa, FIG. 4d-e (Miller et al., 1999). The virtual absence of rRNAs (Bettach et al., 1976) in the spermatozoal preparation in comparison with the kidney control, indicates its quality. In previous studies using both differential display and gene-specific RT-PCR (Kramer et al., 1997), the presence of spermatozoal-specific RNAs in the fertile ejaculate was demonstrated by their absence in the ejaculates of vasectomized men. This and more recent data suggest that any RNAs, arise from pure populations of spermatozoa when they are processed in sufficient numbers, as was the case in the studies reported herein.

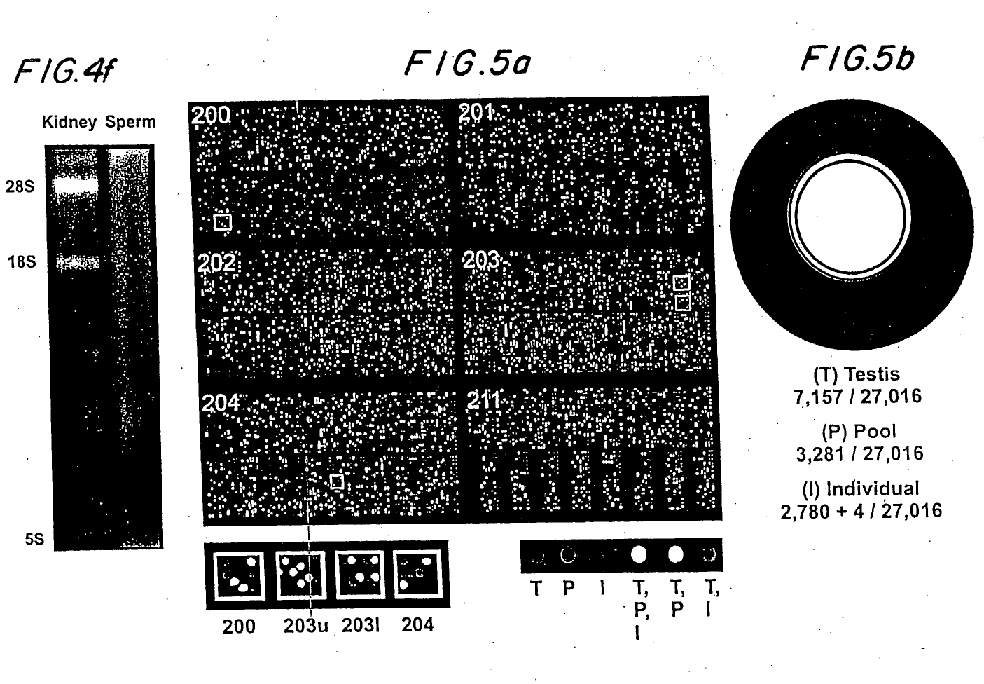

The corresponding cDNAs prepared from testes and ejaculate spermatozoa mRNAs were hybridized to a series of microarrays containing 30,892 Expressed Sequence Tag (EST) probes, of which 27,016 were unique. To define the fingerprints, an mRNA was considered present if its hybridization signal was at least four-fold above the background intensity. A summary is presented in FIG. 5a.

The hybridization error was estimated asia function of the sum of the hybridization signal present or completely absent from each of the 2994 sets of ESTs that were spotted at least twice across the entire set of filters. This analysis supported the view that the likelihood of incorrectly identifying a positive signal was 8%. To verify hybridization specificity independently, all testis associated cluster identification numbers obtained from the UniGene database (http://www.ncbi.nim.nih.gov/UniGene/) were compared to those identified on the microarray with the testes probes. Of the 9052 testis-expressed UniGene cluster identification numbers represented on the filter, a total of 3205, or 35.4%, were identified by the testis cDNA, with a hybridization signal threshold of at least four fold above background. This directly reflects the distribution of abundant mRNAs that were identified using a strict cutoff of at least four fold above background. The data are consistent with the view that the estimation of the number of transcripts constituting the testis transcription was both conservative and reliable.

To determine the extent of similarity between the testis and spermatozoal mRNAs, the unique ESTs identified using the pooled-ejaculate spermatozoa cDNA probe from nine individuals was compared to the ESTs identified using the 10 individual pooled-testes cDNA probe from a single individual. Any EST considered positive in the pooled-ejaculate and not in the testes, or identified in the individual ejaculate but not in the pooled-ejaculate and testes, were noted. The testes probe identified 7157 unique ESTs. This population fully described those identified in spermatozoa when either the pool of poly(A+) enriched RNAs or total RNA from an individual ejaculate was used as the probe. All but four of the ESTs from the 2784 identified in the individual ejaculate were contained within the 3281 ESTs-identified by the pooled-ejaculate, as shown in FIG. 5B.

To ensure that this observation truly reflected spermatozoal RNAs, the purity of the spermatozoa RNA was independently assessed by comparing the spermatozoa RNAs to RNAs of lymphocyte origin (http://www.ncbi.nim.nih.gov/UniGene/;). of the 865 lymphocyte-expressed UniGene cluster identification numbers represented by the ESTs on the microarray filters, only 5% were shared with the 2906 UniGene cluster identification numbers identified with the pooled spermatozoa probe. As expected, the gene products represented by these shared cluster identification numbers corresponded to products of ubiquitously expressed “house-keeping” genes. This indicates an essentially pure population of spermatozoal RNAs free of contamination from lymphocytes or other somatic cells. The concordance displayed between the testes and spermatozoal RNAs supports the view the spermatozoal RNAs can be used to monitor past events of gene expression during spermatogenesis.

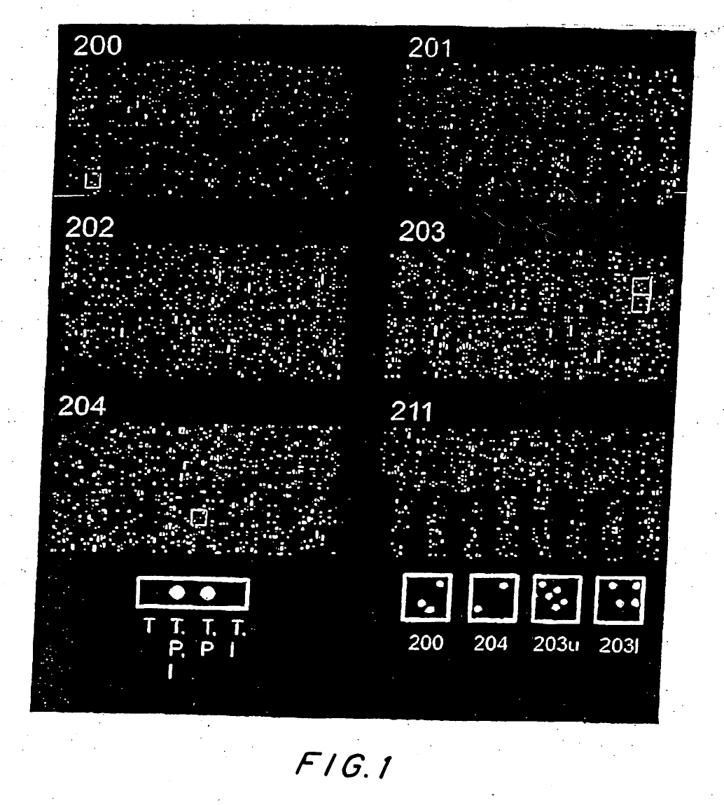

To obtain the data shown in FIG. 1, complementary DNAs, representing the mRNAs isolated from human testes and both pooled and individual ejaculate spermatozoal samples were hybridized to a series of microarrays. Each numbered panel identified the specific Gene Filter® (Research Genetics). Those expressed sequence tags (ESTs) hybridized by tested cDNAs (T) are shown in red, those hybridized by the pooled-ejaculate cDNAs (P) are shown in green, while those hybridized by the individual-ejaculate cDNAs (I) are shown in blue. When the T, P, or I filter overlap, specific colors are generated, as shown by the color-keys at the bottom of each image.

The white boxes shown in FIG. 1 show the four ESTs that hybridized to the individual but not to the pooled ejaculate cDNAs. These regions are enlarged and labeled by their corresponding Gene Filter® in the bottom right corner of FIG. 1. The upper (u) and lower (1) boxes on Gene Filter® 203 are indicated therein.

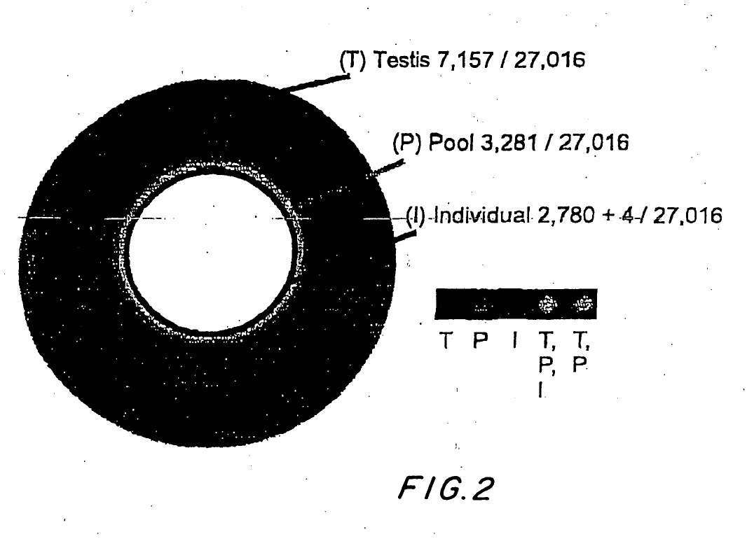

FIG. 2 shows the distribution of testes and spermatozoal RNAs. Of the 27,016 unique ESTs scanned, 7157 were identified as testes (T) cDNAs (red). The testes population contained all 3281 ESTs hybridized by the pooled ejaculate (P) cDNAs (green), which in turn contained 2780 ESTs hybridized by the individual ejaculate (I) cDNA (blue). The four ESTs identified by the individual ejaculate cDNAs but not pooled are contained within the testes population.

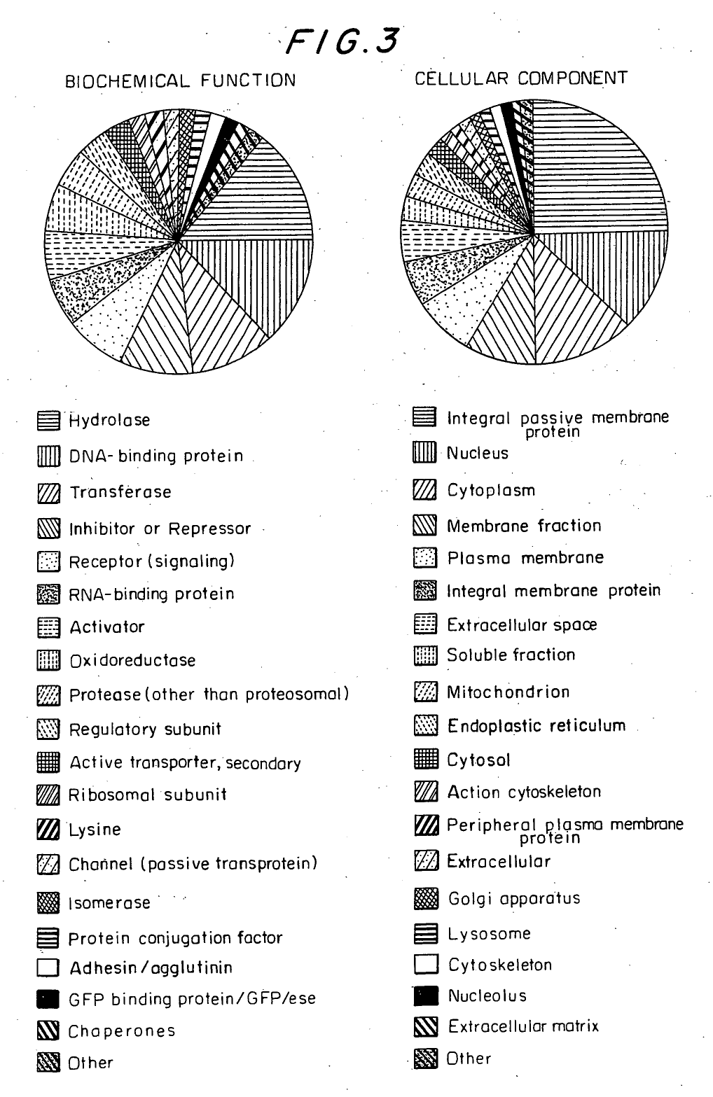

FIG. 3 illustrates spermatozoal RNA ontogeny. The biological activity of the proteins that represent each expressed sequence tag identified by the pooled-7ejaculate spermatozoal cDNA was data mined using Onto-Express. The biochemical function delineates the principal structure, regulatory, or enzymatic function of the protein. The cellular component describes the location in the cell in which the protein is active. The term “other” indicates protein groups with fewer than 14 observations within each group.

To determine the extent of similarity between the testes and spermatozoal mRNAs, the unique ESTs from a pooled-ejaculate spermatozoal cDNA probe from nine individuals, were compared to ESTs identified by a pooled-testes cDNA probe from 19 individuals, and to the ESTs identified using a spermatozoa cDNA probe from a single individual. Using the sort command within the Statistical Analysis Software (SAS version 7-1; SAS Institute, Cary, N.C.), duplicate accession codes within and across filters were deleted for each sample. The unique accession codes within a sample were compared among the samples using a Boolean search strategy, and the number of observations shared was determined. Any EST considered positive in the pooled-ejaculate and not in the testes cDNAs or identified in the individual-ejaculate but not in the pooled-ejaculate and testes cDNAs was noted. The testes cDNAs identified 7157 unique ESTs. This population fully described those identified in spermatozoa when either the pool of poly(A+) enriched RNAs or total RNA from an individual ejaculate was used as the probe. All but four of the ESTs from the 2784 identified in the individual ejaculate cDNAs were contained within the 3281 ESTs identified by the pooled-ejaculate cDNAs, as shown in FIG. 2. These data support the view that spermatozoal RNAs can be used to monitor past events, such as gene expression or spermatogenesis.

The measurement error of the spermatozoal mRNA fingerprint, at the 99% confidence level, was calculated to be within 0.80% of the ESTs identified by the pooled-ejaculate cDNA. When the population of the ESTs identified by pooled-ejaculate cDNAs was compared to those of the single ejaculate, the observed error was only four ESTs. This value is six-fold less than the calculated measurement error, indicating that a maximum number of ESTs were identified by the pooled-ejaculate cDNAs. Of the possible 27,016 unique ESTs, the individual ejaculate cDNA identified 2784 shared ESTs. Thus, it is predicted with 99% confidence that cDNAs derived from a normal fertile man's ejaculate spermatozoa hybridize to at least 2686, but to no more than 2882, of the possible 27,016 ESTs. Accordingly, a specific population and range of ESTs have been defined for this set of Gene Filter® arrays. These transcripts represent the spermatozoal fingerprint for the normal fertile male. Furthermore, these fingerprints have rapidly defined those transcripts present in spermatozoa, without constructing or sequencing the corresponding cDNA library. Thus, the present invention can be used to describe the distribution of transcripts in never before described cell populations.

Characterization of the fingerprint of the normal fertile male using OntoExpress (Khatri et al., 2002) was undertaken to address why mature spermatozoa, that are transcriptionally dormant, contain this complement of mRNAs. The biological function, cellular component, and biological process of the translated proteins corresponding to the spermatozoal mRNAs were defined for each of the hybridizing ESTs (http://compbio.med.wayne.edu/microarray). The majority of spermatozoal mRNAs participate in signal transduction, oncogenesis and cell proliferation corresponding to nuclear and plasma membrane proteins (Balhorn et al., 1999). Genes expressed early in spermatogenesis were also identified in mature spermatozoa.

In one embodiment of the present invention, characterization of the fingerprint of the normal fertile male using Onto-Express can be undertaken to shed light on the basis behind mature spermatozoa, which are transcriptionally dormant and have no rRNAs, yet contain mRNAs. Onto-Express, a JAVA based program developed for the present study, was used to mine the current databases for ontogeny and the biological expression profiles of each EST.

In brief, the locus link is queried and the biochemical function, cellular component, and biological process of the corresponding protein are obtained. The term “UNKNOWN” indicates that the biochemical function, cellular component, or biological process had not been determined. If either the cluster identification or locus link could not be obtained, the data are returned as “UNAVAILABLE.”

The biological function; cellular component, and biological process of the translated proteins corresponding to the spermatozoal mRNAs are defined for each of the hybridizing ESTs. As shown in FIG. 3, hydrolyases and DNA-binding proteins are the functional biological groups having the largest number of identified members. This is consistent with spermatozoal mRNAs encapsulating spermatogenic gene expression, as hydrolytic enzymes found in the acrosomes are translated late in spermatogenesis, and spermatid chromatin undergoes significant restructuring.

The cellular compartments represented by the largest number of identified proteins are the plasma membrane, nucleus, and cytoplasm. The concentration of cytoplasmic protein encoding mRNAs was unexpected, considering that mature spermatozoa have little cytoplasm. This can be reconciled in the following manner. First, proteins localizing to the cytoplasm may function in the developing germ cell wall before the cytoplasmic reduction at spermiation. Several genes expressed early in spermatogenesis have been identified in mature spermatozoa. Testis specific protein Y-linked, an early expressed gene, and testis IN, a gene expressed prior to meiosis, are identified in the testes and both the pooled-ejaculate and single-ejaculate probes.

Examples of additional mRNAs expressed relatively early in spermatogenesis and identified both in the testes and spermatozoal cDNA probes include: tubulin, development. These proteins include a group associated with fertilization; several heat shock response products, which.are important for embryo development; a series that function in embryogenesis and morphogenesis as well as implantation. This was found to be rather intriguing, considering that spermatozoa were believed to contribute little more than the paternal genome, a calcium bob for activating oocytes, and centrioles.

TABLE 2
Spermatozoal mRNA and their function in early development
Biological Process

| | | Embryogenesis | |
|---|---|---|---|
| | Heat Shock | and | |
| Fertilization | Response | Morphogenesis | Implantation |
| CLU^{a} | HSF2^{e} | MID1^{l} | RPL2^{r} |
| CLGN^{b} | HSPA1B^{f} | NLVCF^{k} | |
| AKAP4^{c} | DNAJB1^{g} | CYR61^{l} | |
| GNP1^{d} | HSBP1^{h} | EYA3^{m} | |
| | DUSP5ⁱ | FOXG1Bⁿ | |
| | | WNT5A^{o} | |
| | | WHSC1^{p} | |
| | | SOX13^{q} | |

¹The biological “objective” to which the protein contributes;
^{a}Clusterin (component lysis inhibitor, SP-40, 40 sulfated glycoprotein 2, testosterone-repressed prostate message 2, apolipoprotein j);
^{b}Calmegin;
^{c}A kinase (PRKA) anchor protein 4;
^{d}Glucosamine-6-phosphate isomerase;
^{e}Heat shock transcription factor 2;
^{f}Heat shock 70 kD protein 1B;
^{g}DnaJ (Hsp40) homolog, subfamily B, member 1;
^{h}Heat shock factor binding protein 1;
ⁱDual specificity phosphate 5;
^{j}Midline 1;
^{k}Nuclear localization signal deleted in velocardiofacial syndrome;
^{l}Cysteine-rich, angiogenic inducer, 61;
^{m}Eyes abesent (Drosophila) homolog;
ⁿForkhead box G1B;
^{o}Wingless-type MMTV integration site family, member 5a;
^{p}Wolf-Hirschhorn syndrome candiate 1;
^{q}SRY (sex determining region Y)box 13;
^{r}Ribosomal protein L29

Even though mammalian male and female pronuclei can be considered genetically equivalent, studies indicate that they are indeed functionally different. Human females can present with a hydatidiform mole, in which the fetus is absent and the placental tissue is abnormally enlarged. A majority of these moles arise when a haploid spermatozoa fertilizes and oocyte lacking the maternal pronucleus. Furthermore, when mouse androgenones and gynegenones are produced, the embryos do not develop far beyond the blastocyst stage and fail from their respective deficiencies in chorion and embryo proper. This supports the view.that the spermatozoa and oocyte contribute distinct functionalities to the developing embryo that go beyond imprinting.

To test whether or nor spermatozoal mRNAs are required for zygotic and/or embryonic development, the spermatozoal mRNAs were compared to the population of mRNAs previously identified in oocytes. It is reasoned that if spermatozoa mRNAs are queried, they would be absent in oocytes. When the Unigene cluster identification numbers (representing spermatozoal mRNAs) were compared to cluster identification numbers from oocyte mRNAs, no duplicate values were identified. This indicates that spermatozoa provide novel transcripts distinct from those of the oocyte consistent with the view that they are essential for zygotic and/or embryonic development.

Furthermore, when the UniGene database was searched for mouse homologues corresponding to these human transcripts, no evidence was found to indicate that these populate the female gamete. This suggests that spermatozoa provide novel transcripts distinct from those of the oocyte. Indeed, when polymerase chain reactions were carried out using cDNA pools obtained from zygotes that failed in vitro fertilization, all of the in silicon identified transcripts but A kinase (PRKA) anchor protein 4 were present. Thus, in addition to encapsulating spermatogenic gene expression, spermatozoa mRNAs may provide a function similar to that established for the population of stored oocyte mRNAs (Latham, 1999). They may be necessary for sustaining zygotic and/or embryonic viability prior to or subsequent to the activation of the embryonic genome. This function is consistent with the major biological processes identified for the spermatozoal RNAs. Accordingly, this store of mRNAs may enable men to play a greater role in human development than has previously been considered.

Now that the spermatozoal RNA fingerprint of the normal human fertile male has been identified, it is now possible to identify and diagnose idiopathic infertilities using spermatozoal mRNA fingerprints. The normal fertile male spermatozoal fingerprint can serve as a standard to inform on the underlying causes of male factor infertility.

According to the present invention, sperm from normal individuals can be distinguished from sperm from affected individuals by providing a list of transcripts found in-normal fertile sperm that are consistently detected-across array platforms. Then, a list of transcripts from a subject is obtained, from which is compiled a list of high confidence transcripts. The list of high confidence transcripts from the subject is then compared with the list of transcripts found in normal sperm. If the list of high confidence transcripts from the sperm of the subject is similar to that of the list of transcripts found in normal sperm, the subject is normal. If not, the subject is affected, e.g., teratozoospermia.

In one embodiment, the lists of transcripts are compared using a chip, although any conventionally acceptable method and apparatus for comparing the transcripts can be used. One skilled in the art can readily determine what apparatus is best for comparing transcripts.

The present invention also provides a method for testing for male factor infertility. In this method, a microarray of DNA probes is contacted with a sample of spermatozoa to determine the mRNA fingerprints of the sample. The mRNA fingerprints, generally in the form of expressed sequence tags, of the sample are compared with the mRNA fingerprints of normal fertile male spermatozoa, generally in the form of expressed sequence tags. Any anomalies between the mRNA fingerprints indicate male factor infertility.

Any conventional method can be used for identifying the expressed sequence tags or other forms of the mRNA fingerprints. Among these are fluorescent labeling, radiolabeling, illumination labeling.

A DNA probe is considered to be addressed if its hybridization signal is at least about four-fold above background intensity when using radiolabelled targets. When the targets are fluorometrically labeled, the DNA probe is considered to be addressed if-its hybridization signal has a probability of being called present of greater than or equal to approximately 0.99. The DNA probe is considered to be addressed if its hybridization signal is above about 110 signal units when using an Affymetrix-based platform (i.e., the lower boundary of reliable validated present signal is between 50 and 150 signal units). Using an Illumina-based platform, the DNA probe is considered to be addressed if its hybridization signal is above about 50 signal units.

The foregoing description of the specific embodiments will so fully reveal the general nature of the invention that others can, by applying current knowledge, readily modify and/or adapt for various applications such specific embodiments without departing from the generic concept, and, therefore, such adaptions and modifications should and are intended to be comprehended within the meaning and range of equivalents of the disclosed embodiments. It is to be understood that the phraseology or terminology employed herein is for the purpose of description and not of limitation.

### REFERENCES

Balhorn, R. et al. Protamine mediated condensation of DNA in mammalian sperm. “In: The Male Gamete: From Basic Science to Clinical Applications”, (Edt. By Gagnon C.) cache River Press, pp. 55-70 (1999)
Beitlach C J, “Erickson R P 28 S and 18 S Ribonucleic Acid from Mammalian spermatozoa”, J Exp Zool, 198:49-55 (1976)
Bhasin S; DeKrester D M, Baker HWG, “Clinical Review 64: Pathophysiology and Natural History of Male Fertility, J. Clin, Endocrinol Metab. 1994 79:1525-1529
Bittner M, Meltzer P., Chen Y, Jiang Y, Seftor E, Hendrix M, Radmacher M, Simon R, Yakhini Z, Ben-Dor A, Sampas N, Dougherty E Wang E, Marincola F, Gooden C, Lueders J, Glargelter A, Pollock P. Carpten J, Gilanders E, Leja D, Dietrich K, Beaudry C, Berens M, Alberts D, Sondak V, “Molecular Classification of Cutaneous Malignant Melanoma by Gene Expression Profiling”, Nature 406: 536-540 (2000)
Conover W J “Confidence Interval for a Probability in: Practical Nonparametric Statistics, 2nd Edition” John Wiley& Sons, pp. 99-105 (1980)
Dix, D. J., et al., “Inhibition of hsp70-1 and hsp70-3 expression disrupts preimplantation embryogenesis and heightens embryo sensitivity to arsenic”, Mol Reprod Dev., 51:373-80 (1998)
Hecht N., “Molecular Mechanisms of Male Germ Cell Differentiation”, Bioassays, 20: 555-561 (1998)
Iver V R, Eisen M B, Ross D T, Schuler G, Moore T, Lee J C, Trent J M, Staudt L M, Hudson H Jr, Boguski M S, Lashari D, Shalon D, Botstein D, Brown P O, “The Transcriptional Program in the Response of human Fibroblasts to Serum”, Science 283:83-87 (1999)
Jacobs, P. A., et al., “Mechanism of origin of complete hydatidifom mole” Nature, 286:714-716 (1980)
Khatri P, Draghici S, Ostermeier G C,. Krawetz S A, “Profiling Gene Expression Utilizing Onto-Express”, Genomics, 79:166-270 (2002)
Kramer J., Krawetz S A, “RNA in Sperm: Implications within the Sperm Genome”, Miol. Hum. Reprod., 3:473-478 (1997)
Kramer J A, McCarrey J R, Djakiew D, Krawetz S A, “Human Spermatogenesis as a Model to Examine Gene Potentiation”, Molecular Reproduction and Development, 56: 254-258 (2000)
Kuroda-Kawaguchi T, Skaletsky H, Brown L G, Minx P J, Cordumn H S, Waterston R H, Wilson R K, Silber S, Oates, Rozen S, Page D C, “The AZFcregion of the Y Chromosome Features Massive Palindromes and Uniform Recurrent Deletions in Infertile Men”, Nat. Genet 29: 279-286 (2001)
Latham, K.E., “Mechanisms and control of embryonic genome activation in mammalian embryos”, Int Rev Cytol., 193:71 (1999)
Lissens, W., “Genetics of Male Reproductive Dysfunction in Molecular biology in Reproductive Medicine”, Ed. Fauser, BCJM, McGrath, J., et al. Completion of mouse embryogenesis requires both maternal and paternal genomes, Cell, 37:179-183 (1984)
Miller D, Briigs D, Snowden H, Hamlington J, Follison S, Lilford R, Krawetz S A., “A Complex Population of RNAs in Human Ejaculate Spermatozoa: In placations for Understanding Molecular Aspects of Spermiogenesis”, Gene 237:385-392 (1999)
Miller D, Tang P Z, Skinner C, Lilford R J, “Differential RNA Fingerprinting as a Tool in the Analysis of Spermatozoal Gene Expression”, Hum Reprod, 9:864-869 (1995)
Neilson, L., et al., “Molecular phenotype of the human oocyte by PCR-SAGE”, Genomics, 63:13-24 (2000)
Ostermeier G C, Dix D J, Krawetz S A., “A Bioinformatic Strategy to Rapidly Characterize cDNA Libraries”, Bioinformatics 2002 (in press)
Ostermeier, G C., “Sperm Nuclear Morphology and its Relationship with bull Fertility”, Madison, Wiss.: University of Wisconsin; Ph.D. Dissertation (2000)
Reid R, Dix D J, Miller D, Krawetz S A, “Recovering Filter-based Microarray Data for Pathways Analysis Using a Multipoint Alignment Strategy”, Biotechniques, 30:2-6 (2001)
Richter W, Dettmer D, Glander H J, “Detection of mRNA Transcripts of Cyclic Nucleotide Phosphodiesterase Subtypes in Ejaculated Spermatozoa”, Mol Hum. Reprod., 5: 732-736 (1999)
Sathananthan, A. H., et al., “The sperm centriole: its inheritance, replication and perpetuation in early human embryos”, Hum Reprod., 11:345-56, (1996)
Simerly, C., et al., “The paternal inheritance of the centrosome, the cell's microtubule-organizing center, in humans, and the implications for infertility”, Nat Med., 1(1):47-52 (1995)
Stanton, J. L., et al., “A set of 840 mouse oocyte gene with well-matched human homologues”, Mol Human Repro., 7:521-543 (2001)
Swann, K., et al., “Mechanism of Ca2+ release at fertilization in mammals”, J Exp Zool., 285:267-75 (1999)
Taylor E, Cogdell D, Coombes K, Hu L, Ramdas L, Tabor A, Hamilton S, Zhang W, “Sequence Verification as Quality-control step for Production of cDNA Microarrays”, Biotechniques, 31:62--65 (2001)
Van Assche E, Bonduelle M, Tournaye H, Joris H, Verheyen G, Devroey P, Van Stierteghem A, “Liebaers I Cytogenitics of infertile Men”, Hum. Reprod.; Suppl 4:1-26 (1996)
Wang E, Miller L D, Ohnmacht G A, Liu E T, Marincola F M, “High-Fidelity mRNA Amplification for Gene Profiling”, Nature Biotech., 18:457-459 (2000)

What is claimed is:

1. A method for testing for distinguishing between sperm from normal and affected individuals comprising:
(a) providing a list of transcripts found in normal fertile sperm that are consistently detected across array platforms;
(b) obtaining a list of transcripts from sperm of a subject and compiling a list of high confidence transcripts;
(c) comparing the list of high confidence transcripts from sperm of a subject with the list of transcripts found in normal sperm; and
(d) if the list of high confidence transcripts from the sperm of-the subject is similar to that of the list of transcripts found in normal sperm, the subject is normal; if not, the subject is affected.

2. The method according to claim 1 wherein the lists of transcripts are compared using a chip.

3. A method for testing for male factor infertility comprising:
(a) contacting a microarray of DNA probes with a sample of spermatozoa to determine-the mRNA fingerprints as expressed sequence tags of the sample; and
(b) comparing the mRNA fingerprints of the sample with the mRNA fingerprints as expressed sequence tags of normal fertile male spermatozoa.

4. The method according to claim 3 wherein the male is a human male.

5. The method according to claim 3 wherein the expressed sequence tags are identified fluorometrically.

6. The method according to claim 3 wherein radiolabeled targets are used, and wherein a DNA probe is considered to be addressed if its hybridization signal is at least four-fold above background intensity.

7. A method for testing for exposure to toxins that interfere with male reproduction comprising:
(a) obtaining a sample of spermatozoa;
(b) contacting a microarray of expressed sequence tag probes with the sample to determine the mRNA fingerprints of the sample; and
(c) comparing the mRNA fingerprints of the sample with mRNA fingerprints of normal fertile male spermatozoa to determine which, if any, mRNA has been damaged by exposure to toxins.

8. A method for identifying mRNAs that are paternally derived comprising applying a sample of ejaculate spermatozoa to a microarray of mRNAs that are paternally derived and detecting which mRNAs are addressed.

9. The method according to claim 8 wherein the mRNAS are selected from the group consisting of Hs.27695;
Hs.19500; Hs.8867; Hs.46925; Hs.2714; Hs.152213; Hs.18195;
Hs.274402. Hs.250899; Hs.2128; Hs.75106; Hs.86368; Hs.97633;
and combinations thereof.

10. A kit for assay of spermatozoa comprising a DNA microarray comprising the identified mRNAs of normal fertile sperm.
